(19) United States
(12) Patent Application Publication
(10) Pub. No.: US 2024/0431134 A1
(43) Pub. Date: Dec. 26, 2024

(54) LIGHT EMITTING ELEMENT, COMPOUND INCLUDING NITROGEN FOR THE SAME, AND DISPLAY DEVICE INCLUDING THE LIGHT EMITTING ELEMENT

(71) Applicant: Samsung Display Co., Ltd., Yongin-si (KR)

(72) Inventors: DONGJUN KIM, Yongin-si (KR); BEOMJOON KIM, Yongin-si (KR); HANKYU PAK, Yongin-si (KR); HYUNBIN PARK, Yongin-si (KR); JINYOUNG YUN, Yongin-si (KR); HAN NA CHA, Yongin-si (KR); SANGHYUN HAN, Yongin-si (KR)

(73) Assignee: Samsung Display Co., Ltd., Yongin-si (KR)

(21) Appl. No.: 18/632,518

(22) Filed: Apr. 11, 2024

(30) Foreign Application Priority Data
Jun. 13, 2023 (KR) 10-2023-0075798

(51) Int. Cl. H10K 50/16; H10K 85/60

(52) U.S. Cl. CPC H10K 50/16; H10K 85/615; H10K 85/654

(57) ABSTRACT

Embodiments provide a nitrogen-containing compound and a light emitting element. The light emitting element includes a first electrode, a second electrode facing the first electrode, and at least one functional layer disposed between the first electrode and the second electrode, wherein the functional layer includes the nitrogen-containing compound. The nitrogen-containing compound is represented by Formula 1, which is explained in the specification:

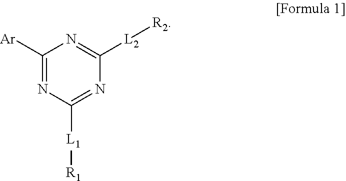

### CROSS-REFERENCE TO RELATED APPLICATION(S)

This application claims priority to and benefits of Korean Patent Application No. 10-2023-0075798 under 35 U.S.C. § 119, filed on Jun. 13, 2023, in the Korean Intellectual Property Office, the entire contents of which are incorporated herein by reference.

### BACKGROUND

### 1. Technical Field

The disclosure relates to a light emitting element, a nitrogen-containing compound used therein, and a display device including the same.

### 2. Description of the Related Art

Active development continues for an organic electroluminescence display device as an image display device. In contrast to liquid crystal display devices and the like, an organic electroluminescence display device is a so-called self-luminescent display device in which holes and electrons respectively injected from a first electrode and a second electrode recombine in an emission layer, so that a luminescent material including an organic compound in the emission layer emits light to achieve display.

In the application of an organic electroluminescence element to a display device, there is a demand for an organic electroluminescence element having high luminous efficiency and a long service life, and continuous development is required on materials for a light emitting element that are capable of stably achieving such characteristics.

In order to implement a light emitting element having high efficiency and a long service life, development on materials for an electron transport region having excellent electron transport properties and stability is presently being conducted.

It is to be understood that this background of the technology section is, in part, intended to provide useful background for understanding the technology. However, this background of the technology section may also include ideas, concepts, or recognitions that were not part of what was known or appreciated by those skilled in the pertinent art prior to a corresponding effective filing date of the subject matter disclosed herein.

### SUMMARY

The disclosure provides a light emitting element in which luminous efficiency and an element service life are improved.

The disclosure also provides a nitrogen-containing compound that is capable of improving luminescence characteristics and an element service life of the light emitting element.

The disclosure also provides a display device including the light emitting element in which luminous efficiency and an element service life are improved.

Embodiments provide a light emitting element which may include a first electrode, a hole transport region disposed on the first electrode, an emission layer disposed on the hole transport region, an electron transport region disposed on the emission layer, and a second electrode disposed on the electron transport region, wherein the electron transport region may include a nitrogen-containing compound represented by Formula 1, and the emission layer may include an organometallic compound represented by Formula D-1:

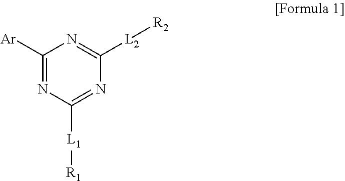

In Formula 1, Ar may be a substituted or unsubstituted aryl group having 6 to 30 ring-forming carbon atoms or a substituted or unsubstituted heteroaryl group having 2 to 30 ring-forming carbon atoms; L₁ and L₂ may each independently be a direct linkage or a substituted or unsubstituted aryl group having 6 to 30 ring-forming carbon atoms; R₁ may be a group represented by Formula 2; and R₂ may be a group represented by Formula 3-1, Formula 3-2, or Formula 3-3:

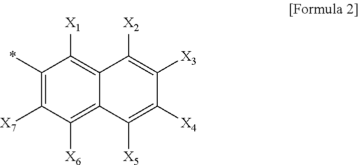

In Formula 2, X₁ to X₇ may each independently be a hydrogen atom, a deuterium atom, a halogen atom, a cyano group, a hydroxy group, a substituted or unsubstituted oxy group, a substituted or unsubstituted thio group, a substituted or unsubstituted boron group, a substituted or unsubstituted silyl group, a substituted or unsubstituted amine group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, except that X₁ or X₇ may be a substituted or unsubstituted phenyl group; and

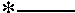 [SMILES: *C]

is a position linked to L₁ in Formula 1:

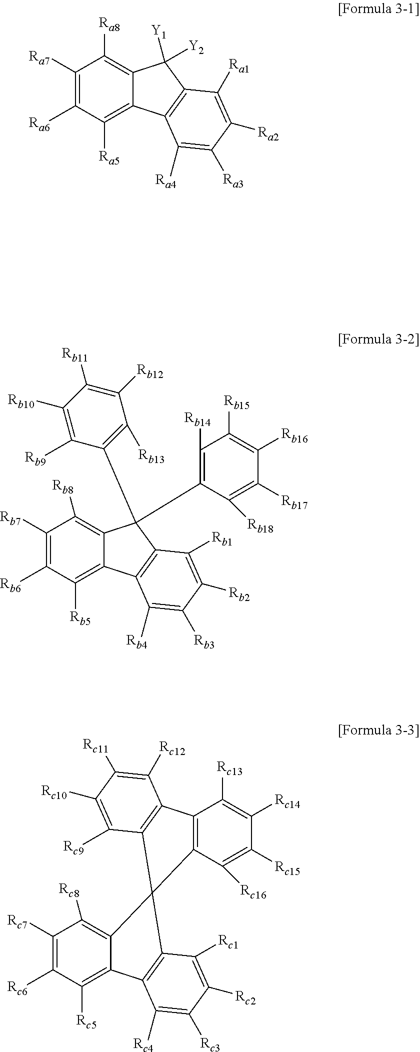

In Formula 3-1, Y₁ and Y₂ may each independently be a hydrogen atom, a deuterium atom, a halogen atom, a cyano group, a hydroxy group, a substituted or unsubstituted alkyl group having 1 to 20 carbon atoms; in Formula 3-1 to Formula 3-3, Rₐ₁ to Rₐ₈, R_{b1} to R_{b18}, and R_{c1} to R_{c16} may each independently be a hydrogen atom, a deuterium atom, a halogen atom, a cyano group, a hydroxy group, a substituted or unsubstituted oxy group, a substituted or unsubstituted thio group, a substituted or unsubstituted boron group, a substituted or unsubstituted silyl group, a substituted or unsubstituted amine group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms; except that when R₂ in Formula 1 is a group represented by Formula 3-1, any one of Rₐ₁ to Rₐ₈ in Formula 3-1 may be a position linked to L₂ in Formula 1, when R₂ in Formula 1 is a group represented by Formula 3-2, any one of R_{b1} to R_{b18} in Formula 3-2 may be a position linked to L₂ in Formula 1, and when R₂ in Formula 1 is a group represented by Formula 3-3, any one of R_{c1} to R_{c16} in Formula 3-3 may be a position linked to L₂ in Formula 1.

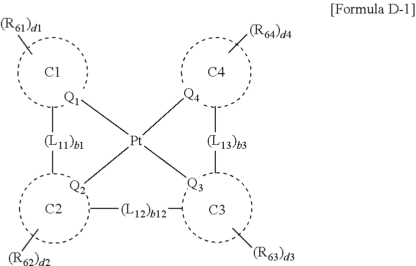

In Formula D-1, Q₁ to Q₄ may each independently be C or N; C1 to C4 may each independently be a substituted or unsubstituted hydrocarbon ring having 5 to 30 ring-forming carbon atoms or a substituted or unsubstituted heterocycle having 2 to 30 ring-forming carbon atoms; L₁₁ to L₁₃ may each independently be a direct linkage,

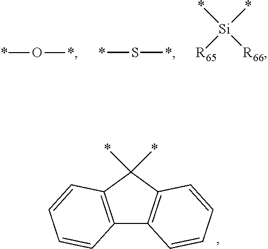

a substituted or unsubstituted alkylene group having 1 to 20 carbon atoms, a substituted or unsubstituted arylene group having 6 to 30 ring-forming carbon atoms, or a substituted or unsubstituted heteroarylene group having 2 to 30 ring-forming carbon atoms; b1 to b3 may each independently be 0 or 1; R₆₁ to R₆₆ may each independently be a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 carbon atoms, a substituted or unsubstituted aryl group having 6 to 60 ring-forming carbon atoms, or a substituted or unsubstituted heteroaryl group having 2 to 60 ring-forming carbon atoms; and d1 to d4 may each independently be an integer from 0 to 4.

In an embodiment, the electron transport region may include an electron transport layer disposed on the emission layer and an electron injection layer disposed on the electron transport layer; and the electron transport layer may include the nitrogen-containing compound.

In an embodiment, L₁ may be a direct linkage or a substituted or unsubstituted phenyl group; and L₂ may be a direct linkage.

In an embodiment, Ar may be a substituted or unsubstituted phenyl group, a substituted or unsubstituted biphenyl group, a substituted or unsubstituted terphenyl group, or a substituted or unsubstituted naphthyl group.

In an embodiment, the nitrogen-containing compound may be represented by any one of Formula 1-1 to Formula 1-4:

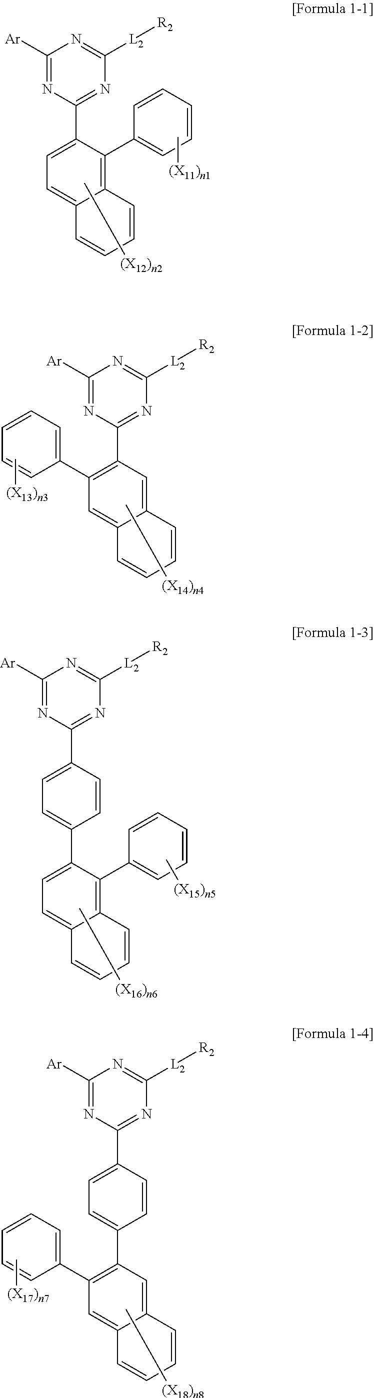

In Formula 1-1 to Formula 1-4, X₁₁ to X₁₈ may each independently be a hydrogen atom or a deuterium atom; n1, n3, n5, and n7 may each independently be an integer from 0 to 5; n2, n4, n6, and n8 may each independently be an integer from 0 to 6; and Ar, L₂ and R₂ are the same as defined in Formula 1.

In an embodiment, X₁₁ to X₁₈ may each be a hydrogen atom.

In an embodiment, the nitrogen-containing compound may be represented by any one of Formula 1-5 to Formula 1-9:

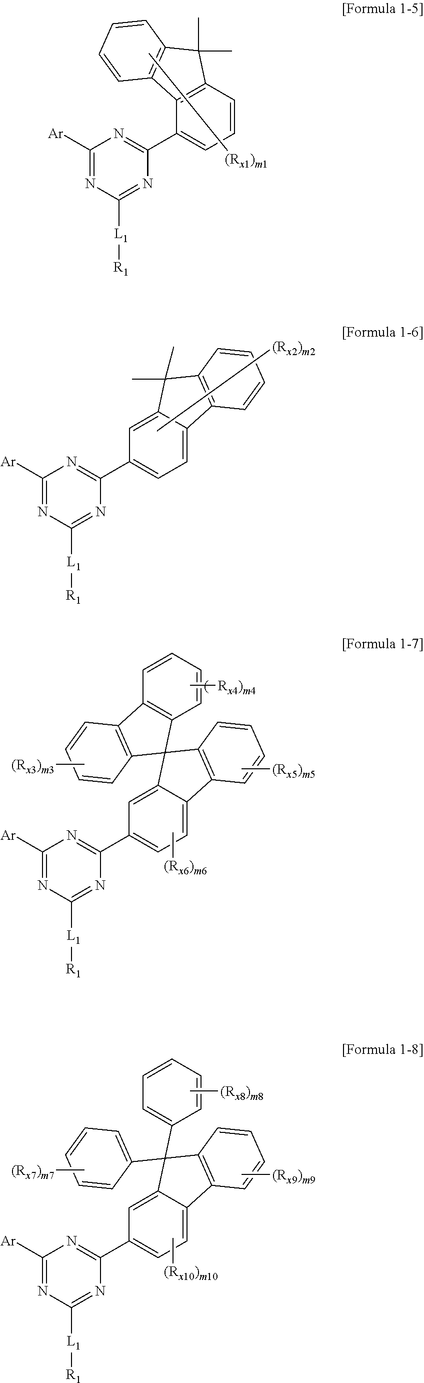

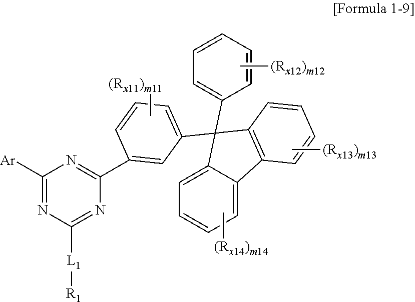

In Formula 1-5 to Formula 1-9, Rₓ₁ to Rₓ₁₄ may each independently be a hydrogen atom, a deuterium atom, a halogen atom, a cyano group, or a substituted or unsubstituted aryl group having 6 to 12 ring-forming carbon atoms; m1 and m2 may each independently be an integer from 0 to 7; m3 to m5, m9, m11, m13, and m14 may each independently be an integer from 0 to 4; m6 and m10 may each independently be an integer from 0 to 3; m7, m8, and m12 may each independently be an integer from 0 to 5; and Ar, L₁, and R₁ are the same as defined in Formula 1.

In an embodiment, Rₓ₁ to Rₓ₃ and Rₓ₅ to Rₓ₁₄ may each be a hydrogen atom; and Rₓ₄ may be a hydrogen atom or an unsubstituted phenyl group.

In an embodiment, the nitrogen-containing compound may be represented by any one of Formula 1-10 to Formula 1-14:

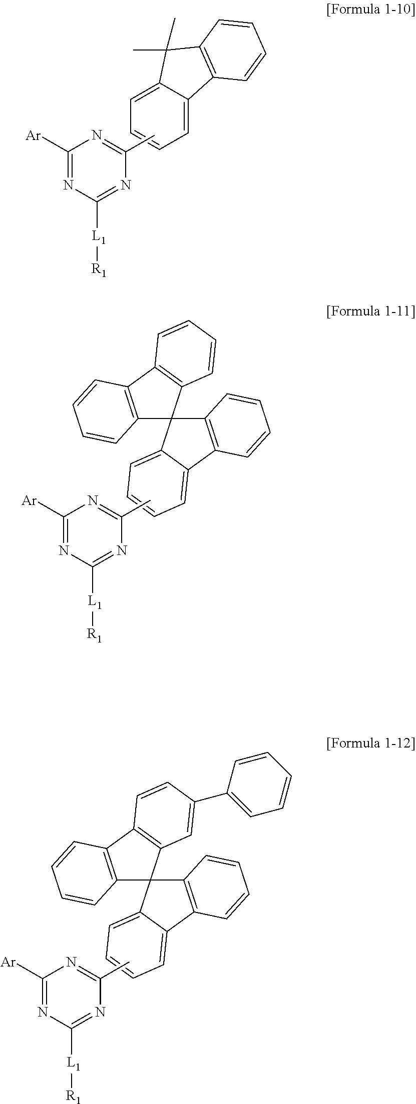

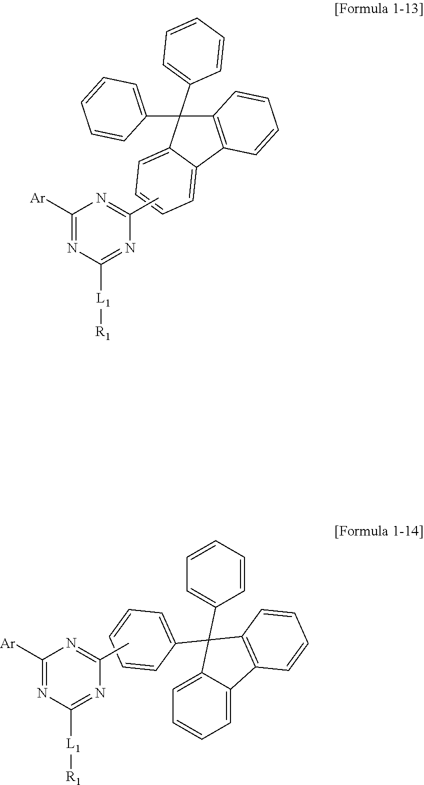

In Formula 1-10 to Formula 1-14, Ar, L₁, and R₁ are the same as defined in Formula 1.

In an embodiment, the nitrogen-containing compound may include at least one compound selected from Compound Group 1, which is explained below.

In an embodiment, the emission layer may further include a compound represented by Formula F-c, which is explained below.

Embodiments provide a nitrogen-containing compound, which may be represented by Formula 1, which is explained herein.

In an embodiment, L₁ may be a direct linkage or a substituted or unsubstituted phenyl group; and L₂ may be a direct linkage.

In an embodiment, Ar may be a substituted or unsubstituted phenyl group, a substituted or unsubstituted biphenyl group, a substituted or unsubstituted terphenyl group, or a substituted or unsubstituted naphthyl group.

In an embodiment, the nitrogen-containing compound represented by Formula 1 may be represented by any one of Formula 1-1 to Formula 1-4, which are explained herein.

In an embodiment, the nitrogen-containing compound represented by Formula 1 may be represented by any one of Formula 1-5 to Formula 1-9, which are explained herein.

In an embodiment, the nitrogen-containing compound represented by Formula 1 may be represented by any one of Formula 1-10 to Formula 1-14, which are explained herein.

In an embodiment, the nitrogen-containing compound represented by Formula 1 may be selected from Compound Group 1, which is explained below.

Embodiments provide a display device which may include a circuit layer disposed on a base layer, and a display element layer disposed on the circuit layer and including a light emitting element. The light emitting element may include a first electrode, a second electrode disposed on the first electrode, and an electron transport region layer disposed between the first electrode and the second electrode; and the electron transport region layer may include the nitrogen-containing compound represented by Formula 1, which is explained herein.

It is to be understood that the embodiments above are described in a generic and explanatory sense only and not for the purposes of limitation, and the disclosure is not limited to the embodiments described above.

### BRIEF DESCRIPTION OF THE DRAWINGS

The accompanying drawings are included to provide a further understanding of the embodiments, and are incorporated in and constitute a part of this specification. The drawings illustrate embodiments of the disclosure and principles thereof. The above and other aspects and features of the disclosure will become more apparent by describing in detail embodiments thereof with reference to the accompanying drawings, in which:

FIG. 1 is a schematic plan view of a display device according to an embodiment;

FIG. 2 is a schematic cross-sectional view of a display device according to an embodiment;

FIG. 3 is a schematic cross-sectional view of a light emitting element according to an embodiment;

FIG. 4 is a schematic cross-sectional view of a light emitting element according to an embodiment;

FIG. 5 is a schematic cross-sectional view of a light emitting element according to an embodiment;

FIG. 6 is a schematic cross-sectional view of a light emitting element according to an embodiment;

FIG. 7 is a schematic cross-sectional view of a display device according to an embodiment;

FIG. 8 is a schematic cross-sectional view of a display device according to an embodiment.

FIG. 9 is a schematic cross-sectional view of a display device according to an embodiment;

FIG. 10 is a schematic cross-sectional view of a display device according to an embodiment; and

FIG. 11 is a schematic perspective view of a vehicle in which display devices are disposed according to an embodiment.

### DETAILED DESCRIPTION OF THE EMBODIMENTS

The disclosure will now be described more fully hereinafter with reference to the accompanying drawings, in which embodiments are shown. This disclosure may, however, be embodied in different forms and should not be construed as limited to the embodiments set forth herein. Rather, these embodiments are provided so that this disclosure will be thorough and complete, and will fully convey the scope of the disclosure to those skilled in the art.

In the drawings, the sizes, thicknesses, ratios, and dimensions of the elements may be exaggerated for ease of description and for clarity. Like reference numbers and reference characters refer to like elements throughout.

In the specification, it will be understood that when an element (or region, layer, part, etc.) is referred to as being “on”, “connected to”, or “coupled to” another element, it can be directly on, connected to, or coupled to the other element, or one or more intervening elements may be present therebetween. In a similar sense, when an element (or region, layer, part, etc.) is described as “covering” another element, it can directly cover the other element, or one or more intervening elements may be present therebetween.

In the specification, when an element is “directly on,” “directly connected to,” or “directly coupled to” another element, there are no intervening elements present. For example, “directly on” may mean that two layers or two elements are disposed without an additional element such as an adhesion element therebetween.

As used herein, the expressions used in the singular such as “a,” “an,” and “the,” are intended to include the plural forms as well, unless the context clearly indicates otherwise.

As used herein, the term “and/or” includes any and all combinations of one or more of the associated listed items. For example, “A and/or B” may be understood to mean “A, B, or A and B.” The terms “and” and “or” may be used in the conjunctive or disjunctive sense and may be understood to be equivalent to “and/or”.

In the specification and the claims, the term “at least one of” is intended to include the meaning of “at least one selected from the group consisting of” for the purpose of its meaning and interpretation. For example, “at least one of A, B, and C” may be understood to mean A only, B only, C only, or any combination of two or more of A, B, and C, such as ABC, ACC, BC, or CC. When preceding a list of elements, the term, “at least one of,” modifies the entire list of elements and does not modify the individual elements of the list.

It will be understood that, although the terms first, second, etc. may be used herein to describe various elements, these elements should not be limited by these terms. These terms are only used to distinguish one element from another element. Thus, a first element could be termed a second element without departing from the teachings of the disclosure. Similarly, a second element could be termed a first element, without departing from the scope of the disclosure.

The spatially relative terms “below”, “beneath”, “lower”, “above”, “upper”, or the like, may be used herein for ease of description to describe the relations between one element or component and another element or component as illustrated in the drawings. It will be understood that the spatially relative terms are intended to encompass different orientations of the device in use or operation, in addition to the orientation depicted in the drawings. For example, in the case where a device illustrated in the drawing is turned over, the device positioned “below” or “beneath” another device may be placed “above” another device. Accordingly, the illustrative term “below” may include both the lower and upper positions. The device may also be oriented in other directions and thus the spatially relative terms may be interpreted differently depending on the orientations.

The terms “about” or “approximately” as used herein is inclusive of the stated value and means within an acceptable range of deviation for the recited value as determined by one of ordinary skill in the art, considering the measurement in question and the error associated with measurement of the recited quantity (i.e., the limitations of the measurement system). For example, “about” may mean within one or more standard deviations, or within ±20%, ±10%, or ±5% of the stated value.

It should be understood that the terms “comprises,” “comprising,” “includes,” “including,” “have,” “having,” “contains,” “containing,” and the like are intended to specify the presence of stated features, integers, steps, operations, elements, components, or combinations thereof in the disclosure, but do not preclude the presence or addition of one or more other features, integers, steps, operations, elements, components, or combinations thereof.

Unless otherwise defined or implied herein, all terms (including technical and scientific terms) used have the same meaning as commonly understood by those skilled in the art to which this disclosure pertains. It will be further understood that terms, such as those defined in commonly used dictionaries, should be interpreted as having a meaning that is consistent with their meaning in the context of the relevant art and should not be interpreted in an ideal or excessively formal sense unless clearly defined in the specification.

In the specification, the term “substituted or unsubstituted” may describe a group that is substituted or unsubstituted with at least one substituent selected from the group consisting of a deuterium atom, a halogen atom, a cyano group, a nitro group, an amino group, an amine group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. Each of the substituents listed above may itself be substituted or unsubstituted. For example, a biphenyl group may be interpreted as an aryl group, or it may be interpreted as a phenyl group substituted with a phenyl group.

In the specification, the term “bonded to an adjacent group to form a ring” may be interpreted as a group that is bonded to an adjacent group to form a substituted or unsubstituted hydrocarbon ring or a substituted or unsubstituted heterocycle. The hydrocarbon ring may be aliphatic or aromatic. The heterocycle may be aliphatic or aromatic. The hydrocarbon ring and the heterocycle may each independently be monocyclic or polycyclic. A ring that is formed by adjacent groups being bonded to each other may itself be connected to another ring to form a spiro structure.

In the specification, the term “adjacent group” may be interpreted as a substituent that is substituted for an atom which is directly linked to an atom substituted with a corresponding substituent, as another substituent that is substituted for an atom which is substituted with a corresponding substituent, or as a substituent that is sterically positioned at the nearest position to a corresponding substituent. For example, two methyl groups in 1,2-dimethylbenzene may be interpreted as “adjacent groups” to each other, and two ethyl groups in 1,1-diethylcyclopentane may be interpreted as “adjacent groups” to each other. For example, two methyl groups in 4,5-dimethylphenanthrene may be interpreted as “adjacent groups” to each other.

In the specification, examples of a halogen atom may include a fluorine atom, a chlorine atom, a bromine atom, or an iodine atom.

In the specification, an alkyl group may be linear or branched. The number of carbon atoms in an alkyl group may be 1 to 50, 1 to 30, 1 to 20, 1 to 10, or 1 to 6. Examples of an alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, etc., but embodiments are not limited thereto.

In the specification, a cycloalkyl group may be a cyclic alkyl group. The number of carbon atoms in a cycloalkyl group may be 3 to 50, 3 to 30, 3 to 20, or 3 to 10. Examples of a cycloalkyl group may include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, etc., but embodiments are not limited thereto.

In the specification, an alkenyl group may be a hydrocarbon group that includes at least one carbon-carbon double bond in the middle or at a terminus of an alkyl group having 2 or more carbon atoms. An alkenyl group may be linear or branched. The number of carbon atoms in an alkenyl group is not particularly limited, and may be 2 to 30, 2 to 20, or 2 to 10. Examples of an alkenyl group may include a vinyl group, a 1-butenyl group, a 1-pentenyl group, a 1,3-butadienyl aryl group, a styrenyl group, a styryl vinyl group, etc., but embodiments are not limited thereto.

In the specification, an alkynyl group may be a hydrocarbon group that includes at least one carbon-carbon triple bond in the middle or at a terminus of an alkyl group having 2 or more carbon atoms. An alkynyl group may be linear or branched. The number of carbon atoms in an alkynyl group is not particularly limited, and may be 2 to 30, 2 to 20, or 2 to 10. Examples of an alkynyl group may include an ethynyl group, a propynyl group, etc., but embodiments are not limited thereto.

In the specification, a hydrocarbon ring group may be any functional group or substituent derived from an aliphatic hydrocarbon ring. For example, a hydrocarbon ring group may be a saturated hydrocarbon ring group having 5 to 20 ring-forming carbon atoms.

In the specification, an aryl group may be any functional group or substituent derived from an aromatic hydrocarbon ring. An aryl group may be monocyclic or polycyclic. The number of ring-forming carbon atoms in an aryl group may be 6 to 30, 6 to 20, or 6 to 15. Examples of an aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, etc., but embodiments are not limited thereto.

In the specification, a fluorenyl group may be substituted, and two substituents may be bonded to each other to form a spiro structure. Examples of a substituted fluorenyl group may include the groups shown below. However, embodiments are not limited thereto.

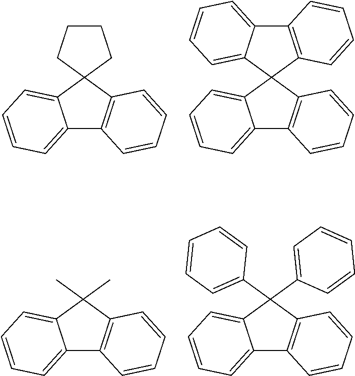

In the specification, a heterocyclic group may be any functional group or substituent derived from a ring that includes at least one of B, O, N, P, Si, S, and Se as a heteroatom. A heterocyclic group may be aliphatic or aromatic. An aromatic heterocyclic group may be a heteroaryl group. An aliphatic heterocycle and an aromatic heterocycle may each independently be monocyclic or polycyclic.

In the specification, a heterocyclic group may include at least one of B, O, N, P, Si, S, and Se as a heteroatom. If a heterocyclic group includes two or more heteroatoms, the two or more heteroatoms may be the same as or different from each other. A heterocyclic group may be monocyclic or polycyclic, and a heterocyclic group may be a heteroaryl group. The number of ring-forming carbon atoms in a heterocyclic group may be 2 to 30, 2 to 20, or 2 to 10.

In the specification, an aliphatic heterocyclic group may include at least one of B, O, N, P, Si, S, and Se as a heteroatom. The number of ring-forming carbon atoms in an aliphatic heterocyclic group may be 2 to 30, 2 to 20, or 2 to 10. Examples of an aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., but embodiments are not limited thereto.

In the specification, a heteroaryl group may include at least one of B, O, N, P, Si, S, and Se as a heteroatom. If a heteroaryl group includes two or more heteroatoms, the two or more heteroatoms may be the same as or different from each other. A heteroaryl group may be monocyclic or polycyclic. The number of ring-forming carbon atoms in a heteroaryl group may be 2 to 30, 2 to 20, or 2 to 10. Examples of a heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzoimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, etc., but embodiments are not limited thereto.

In the specification, the above description of an aryl group may be applied to an arylene group, except that an arylene group is a divalent group. In the specification, the above description of a heteroaryl group may be applied to a heteroarylene group, except that a heteroarylene group is a divalent group.

In the specification, a silyl group may be an alkylsilyl group or an arylsilyl group. Examples of a silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., but embodiments are not limited thereto.

In the specification, the number of carbon atoms in a carbonyl group is not particularly limited, and may be 1 to 40, 1 to 30, or 1 to 20. For example, a carbonyl group may have one of the following structures, but embodiments are not limited thereto.

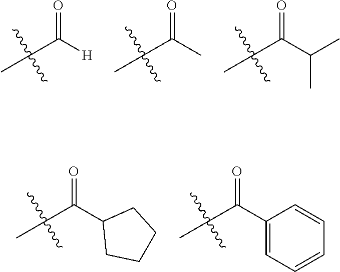

In the specification, the number of carbon atoms in a sulfinyl group or in a sulfonyl group is not particularly limited, and may be 1 to 30. A sulfinyl group may be an alkyl sulfinyl group or an aryl sulfinyl group. A sulfonyl group may be an alkyl sulfonyl group or an aryl sulfonyl group.

In the specification, a thio group may be an alkylthio group or an arylthio group. A thio group may be a sulfur atom that is bonded to an alkyl group or an aryl group as defined above. Examples of a thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, etc., but embodiments are not limited thereto.

In the specification, an oxy group may be an oxygen atom that is bonded to an alkyl group or an aryl group as defined above. An oxy group may be an alkoxy group or an aryl oxy group. An alkoxy group may be linear, branched, or cyclic. The number of carbon atoms in an oxy group is not particularly limited, and may be, for example, 1 to 20 or 1 to 10. Examples of an oxy group may include a methoxy group, an ethoxy group, an n-propoxy group, an isopropoxy group, a butoxy group, a pentyloxy group, a hexyloxy group, an octyloxy group, a nonyloxy group, a decyloxy group, a benzyloxy group, etc., but embodiments are not limited thereto.

In the specification, a boron group may be a boron atom that is bonded to an alkyl group or an aryl group as defined above. A boron group may be an alkyl boron group or an aryl boron group. Examples of a boron group may include a dimethylboron group, a trimethylboron group, a t-butyldimethylboron group, a diphenylboron group, a phenylboron group, etc., but embodiments are not limited thereto.

In the specification, the number of carbon atoms in an amine group is not particularly limited, and may be 1 to 30. An amine group may be an alkyl amine group or an aryl amine group. Examples of an amine group may include a methylamine group, a dimethylamine group, a phenylamine group, a diphenylamine group, a naphthylamine group, a 9-methyl-anthracenylamine group, etc., but embodiments are not limited thereto.

In the specification, an alkyl group within an alkylthio group, an alkylsulfoxy group, an alkylaryl group, an alkylamino group, an alkyl boron group, an alkyl silyl group, or an alkyl amine group may be the same as an example of an alkyl group as described above.

In the specification, an aryl group within an aryloxy group, an arylthio group, an arylsulfoxy group, an arylamino group, an arylboron group, an arylsilyl group, or an arylamine group may be the same as an example of an aryl group as described above.

In the specification, a direct linkage may be a single bond.

In the specification, the symbols

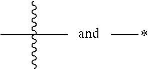

each represent a bond to a neighboring atom in a corresponding formula or moiety.

Hereinafter, embodiments will be described with reference to the accompanying drawings.

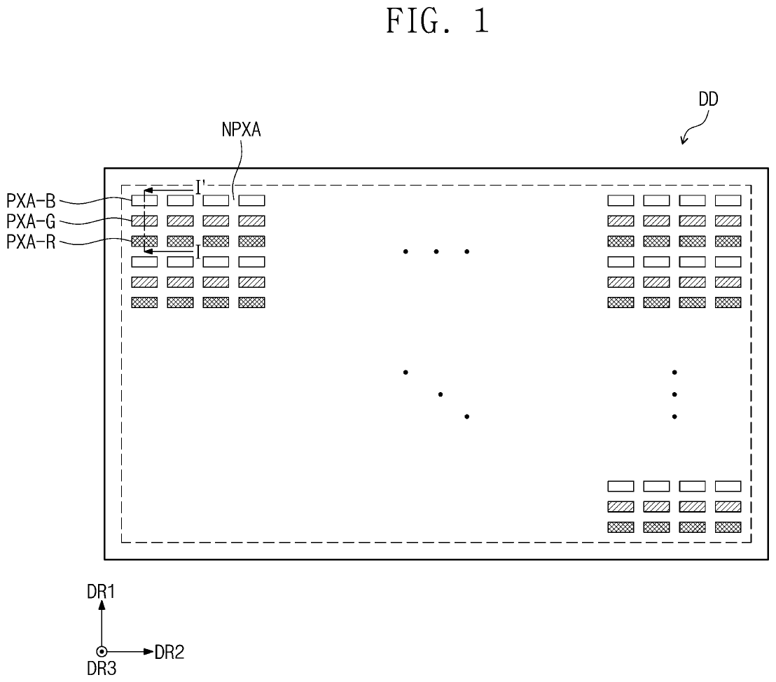

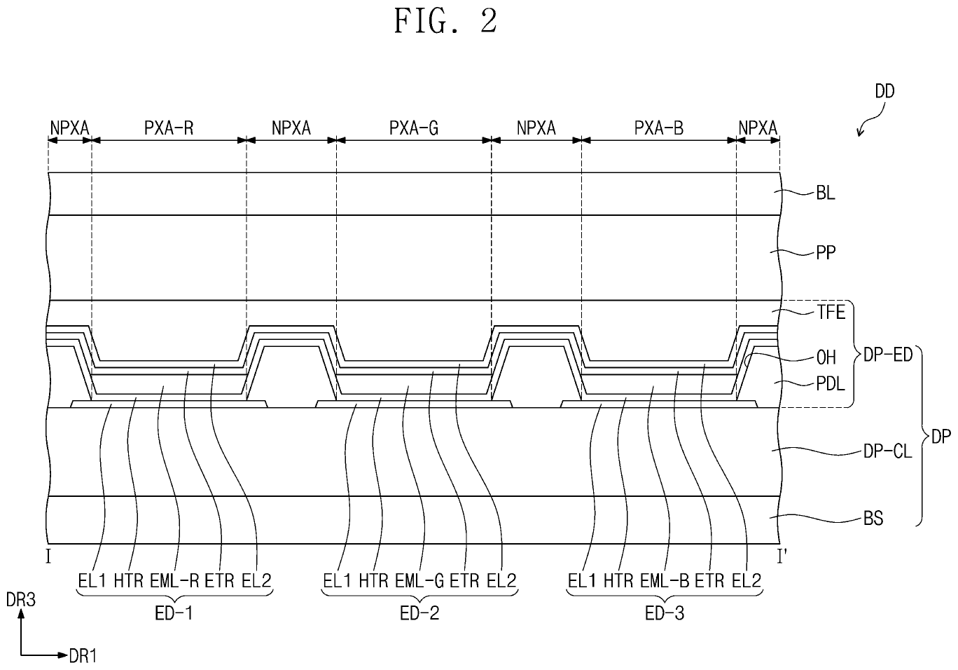

FIG. 1 is a schematic plan view of a display device DD according to an embodiment. FIG. 2 is a schematic cross-sectional view of a display device DD according to an embodiment. FIG. 2 is a schematic cross-sectional view illustrating a part taken along line I-I′ in FIG. 1.

The display device DD may include a display panel DP and an optical layer PP disposed on the display panel DP. The display panel DP includes light emitting elements ED-1, ED-2, and ED-3. The display device DD may include multiples of each of the light emitting elements ED-1, ED-2, and ED-3. The optical layer PP may be disposed on the display panel DP to control light that is reflected at the display panel DP from an external light. The optical layer PP may include, for example, a polarization layer or a color filter layer. Although not shown in the drawings, in an embodiment, the optical layer PP may be omitted from the display device DD.

A base substrate BL may be disposed on the optical layer PP. The base substrate BL may provide a base surface on which the optical layer PP is disposed. The base substrate BL may be a glass substrate, a metal substrate, a plastic substrate, etc. However, embodiments are not limited thereto, and the base substrate BL may include an inorganic layer, an organic layer, or a composite material layer. Although not shown in the drawings, in an embodiment, the base substrate BL may be omitted.

The display device DD according to an embodiment may further include a filling layer (not shown). The filling layer (not shown) may be disposed between a display device layer DP-ED and the base substrate BL. The filling layer (not shown) may be an organic material layer. The filling layer (not shown) may include at least one of an acrylic-based resin, a silicone-based resin, and an epoxy-based resin.

The display panel DP may include a base layer BS, a circuit layer DP-CL provided on the base layer BS, and the display device layer DP-ED. The display device layer DP-ED may include a pixel defining film PDL, light emitting elements ED-1, ED-2, and ED-3 disposed between portions of the pixel defining film PDL, and an encapsulation layer TFE disposed on the light emitting elements ED-1, ED-2, and ED-3.

The base layer BS may provide a base surface on which the display device layer DP-ED is disposed. The base layer BS may be a glass substrate, a metal substrate, a plastic substrate, etc. However, embodiments are not limited thereto, and the base layer BS may include an inorganic layer, an organic layer, or a composite material layer.

In an embodiment, the circuit layer DP-CL is disposed on the base layer BS, and the circuit layer DP-CL may include transistors (not shown). The transistors (not shown) may each include a control electrode, an input electrode, and an output electrode. For example, the circuit layer DP-CL may include a switching transistor and a driving transistor for driving the light emitting elements ED-1, ED-2, and ED-3 of the display device layer DP-ED.

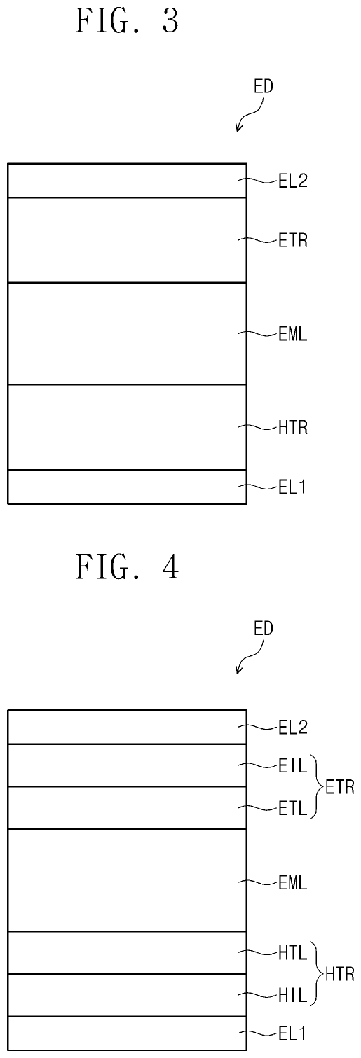

The light emitting elements ED-1, ED-2, and ED-3 may each have a structure of a light emitting element ED of an embodiment according to any of FIGS. 3 to 6, which will be described later. the light emitting elements ED-1, ED-2, and ED-3 may each include a first electrode EL1, a hole transport region HTR, emission layers EML-R, EML-G, and EML-B, an electron transport region ETR, and a second electrode EL2.

FIG. 2 illustrates an embodiment in which the emission layers EML-R, EML-G, and EML-B of the light emitting elements ED-1, ED-2, and ED-3 are disposed in openings OH defined in the pixel defining film PDL, and the hole transport region HTR, the electron transport region ETR, and the second electrode EL2 are each provided as a common layer for the light emitting elements ED-1, ED-2, and ED-3. However, embodiments are not limited thereto. Although not illustrated in FIG. 2, in an embodiment, the hole transport region HTR and the electron transport region ETR may each be provided by being patterned in the openings OH defined in the pixel defining film PDL. For example, in an embodiment, the hole transport region HTR, the emission layers EML-R, EML-G, and EML-B, and the electron transport region ETR of the light emitting elements ED-1, ED-2, and ED-3 may each be provided by being patterned through an inkjet printing method.

The encapsulation layer TFE may cover the light emitting elements ED-1, ED-2, and ED-3. The encapsulation layer TFE may seal the display device layer DP-ED. The encapsulation layer TFE may be a thin film encapsulation layer. The encapsulation layer TFE may be formed of a single layer or of multiple layers. The encapsulation layer TFE may include at least one insulation layer. The encapsulation layer TFE according to an embodiment may include at least one inorganic film (hereinafter, an encapsulation-inorganic film). In an embodiment, the encapsulation layer TFE may include at least one organic film (hereinafter, an encapsulation-organic film) and at least one encapsulation-inorganic film.

The encapsulation-inorganic film protects the display device layer DP-ED from moisture and/or oxygen, and the encapsulation-organic film protects the display device layer DP-ED from foreign substances such as dust particles. The encapsulation-inorganic film may include silicon nitride, silicon oxynitride, silicon oxide, titanium oxide, aluminum oxide, or the like, but embodiments are not limited thereto. The encapsulation-organic film may include an acrylic-based compound, an epoxy-based compound, or the like. The encapsulation-organic film may include a photopolymerizable organic material, but embodiments are not limited thereto.

The encapsulation layer TFE may be disposed on the second electrode EL2 and may be disposed to the openings OH.

Referring to FIGS. 1 and 2, the display device DD may include non-light emitting regions NPXA and light emitting regions PXA-R, PXA-G, and PXA-B. The light emitting regions PXA-R, PXA-G, and PXA-B may each be a region that emits light respectively generated by the light emitting elements ED-1, ED-2, and ED-3. The light emitting regions PXA-R, PXA-G, and PXA-B may be spaced apart from each other in a plan view.

The light emitting regions PXA-R, PXA-G, and PXA-B may be regions that are separated from each other by the pixel defining film PDL. The non-light emitting regions NPXA may be areas between the adjacent light emitting regions PXA-R, PXA-G, and PXA-B, and which may correspond to the pixel defining film PDL. In an embodiment, the light emitting regions PXA-R, PXA-G, and PXA-B may each correspond to a pixel. The pixel defining film PDL may separate the light emitting elements ED-1, ED-2, and ED-3. The emission layers EML-R, EML-G, and EML-B of the light emitting elements ED-1, ED-2, and ED-3 may be disposed in openings OH defined in the pixel defining film PDL and separated from each other.

The light emitting regions PXA-R, PXA-G, and PXA-B may be arranged into groups according to the color of light generated from the light emitting elements ED-1, ED-2, and ED-3. In the display device DD according to an embodiment illustrated in FIGS. 1 and 2, three light emitting regions PXA-R, PXA-G, and PXA-B, which respectively emit red light, green light, and blue light, are illustrated as an example. For example, the display device DD may include a red light emitting region PXA-R, a green light emitting region PXA-G, and a blue light emitting region PXA-B, which are distinct from each other.

In the display device DD according to an embodiment, the light emitting elements ED-1, ED-2 and ED-3 may emit light having wavelengths that are different from each other. For example, in an embodiment, the display device DD may include a first light emitting element ED-1 that emits red light, a second light emitting element ED-2 that emits green light, and a third light emitting element ED-3 that emits blue light. For example, the red light emitting region PXA-R, the green light emitting region PXA-G, and the blue light emitting region PXA-B of the display device DD may respectively correspond to the first light emitting element ED-1, the second light emitting element ED-2, and the third light emitting element ED-3.

However, embodiments are not limited thereto, and the first to third light emitting elements ED-1, ED-2, and ED-3 may emit light in a same wavelength range or at least one light emitting element may emit light in a wavelength range that is different from the remainder. For example, the first to third light emitting elements ED-1, ED-2, and ED-3 may each emit blue light.

The light emitting regions PXA-R, PXA-G, and PXA-B in the display device DD according to an embodiment may be arranged in a stripe configuration. Referring to FIG. 1, the red light emitting regions PXA-R, the green light emitting regions PXA-G, and the blue light emitting regions PXA-B may be respectively arranged along a second directional axis DR2. In another embodiment, the red light emitting region PXA-R, the green light emitting region PXA-G, and the blue light emitting region PXA-B may be arranged in this repeating order along a first directional axis DR1.

FIGS. 1 and 2 illustrate that the light emitting regions PXA-R, PXA-G, and PXA-B all have a similar area, but embodiments are not limited thereto. In an embodiment, the light emitting regions PXA-R, PXA-G, and PXA-B may be different in size or shape from each other, according to a wavelength range of emitted light. The areas of the light emitting regions PXA-R, PXA-G, and PXA-B may be areas in a plan view that are defined by the first directional axis DR1 and the second directional axis DR2.

An arrangement of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in various combinations, according to the display quality characteristics that are required for the display device DD. For example, the light emitting regions PXA-R, PXA-G, and PXA-B may be arranged in a pentile configuration (such as PENTILE®) or in a diamond configuration (such as Diamond Pixel@).

The areas of the light emitting regions PXA-R, PXA-G, and PXA-B may be different in size from each other. For example, in an embodiment, an area of a green light emitting region PXA-G may be smaller than an area of a blue light emitting region PXA-B, but embodiments are not limited thereto.

Hereinafter, FIG. 3 to FIG. 6 are each a schematic cross-sectional view of a light emitting element according to an embodiment. Embodiments provide a light emitting element ED which may include a first electrode EL1, a second electrode EL2 disposed on the first electrode EL1, and at least one functional layer disposed between the first electrode EL1 and the second electrode EL2. The light emitting element ED may include a nitrogen-containing compound according to an embodiment, which will be described below, in the at least one functional layer. In an embodiment, the light emitting element ED may include a first electrode EL1, a hole transport region HTR, an emission layer EML, an electron transport region ETR, and a second electrode EL2.

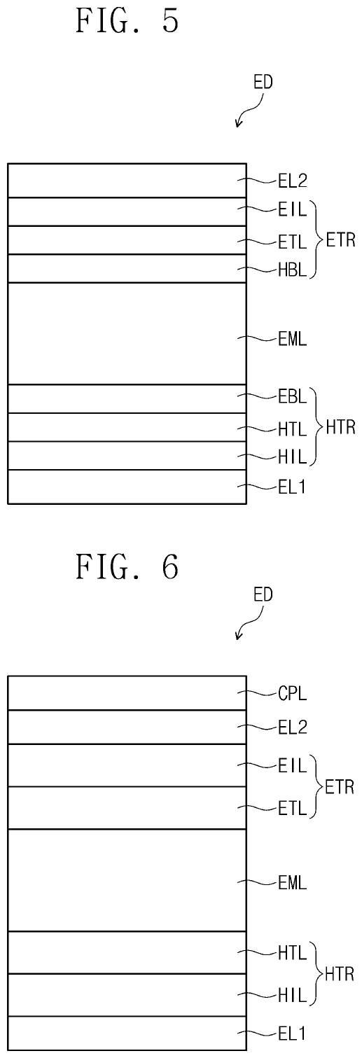

In comparison to FIG. 3, FIG. 4 is a schematic cross-sectional view of a light emitting element ED according to an embodiment, in which a hole transport region HTR includes a hole injection layer HIL and a hole transport layer HTL, and an electron transport region ETR includes an electron injection layer EIL and an electron transport layer ETL. In comparison to FIG. 3, FIG. 5 is a schematic cross-sectional view of a light emitting element ED according to an embodiment, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. In comparison to FIG. 4, FIG. 6 is a schematic cross-sectional view of a light emitting element ED according to an embodiment that includes a capping layer CPL disposed on a second electrode EL2.

The first electrode EL1 has conductivity. The first electrode EL1 may be formed of a metal material, a metal alloy, or a conductive compound. The first electrode EL1 may be an anode or a cathode. However, embodiments are not limited thereto. In an embodiment, the first electrode EL1 may be a pixel electrode. The first electrode EL1 may be a transmissive electrode, a transflective electrode, or a reflective electrode. The first electrode EL1 may include at least one of Ag, Mg, Cu, Al, Pt, Pd, Au, Ni, Nd, Ir, Cr, Li, Ca, LiF, Mo, Ti, W, In, Sn, Zn, an oxide thereof, a compound thereof, and a mixture thereof.

If the first electrode EL1 is a transmissive electrode, the first electrode EL1 may include a transparent metal oxide such as indium tin oxide (ITO), indium zinc oxide (IZO), zinc oxide (ZnO), or indium tin zinc oxide (ITZO). If the first electrode EL1 is a transflective electrode or a reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, a compound thereof, or a mixture thereof (e.g., a mixture of Ag and Mg). In another embodiment, the first electrode EL1 may have a multilayer structure including a reflective film or a transflective film formed of the above-described materials, and a transparent conductive film formed of ITO, IZO, ZnO, ITZO, etc. For example, the first electrode EL1 may have a three-layer structure of ITO/Ag/ITO, but embodiments are not limited thereto. In an embodiment, the first electrode EL1 may include the above-described metal materials, combinations of at least two metal materials of the above-described metal materials, oxides of the above-described metal materials, or the like. A thickness of the first electrode EL1 may be in a range of about 700 Å to about 10,000 Å. For example, the thickness of the first electrode EL1 may be in a range of about 1,000 Å to about 3,000 Å.

The hole transport region HTR may be provided on the first electrode EL1. The hole transport region HTR may include at least one of a hole injection layer HIL, a hole transport layer HTL, a buffer layer (not shown), an emission-auxiliary layer (not shown), and an electron blocking layer EBL. A thickness of the hole transport region HTR may be, for example, in a range of about 50 Å to about 15,000 Å.

The hole transport region HTR may be a layer consisting of a single material, a layer including different materials, or a structure including multiple layers including different materials.

In embodiments, the hole transport region HTR may have a single layer structure of a hole injection layer HIL or a hole transport layer HTL, or may have a single layer structure formed of a hole injection material and a hole transport material. In embodiments, the hole transport region HTR may have a single layer structure formed of different materials, or may have a structure in which a hole injection layer HIL/hole transport layer HTL, a hole injection layer HIL/hole transport layer HTL/buffer layer (not shown), a hole injection layer HIL/buffer layer (not shown), a hole transport layer HTL/buffer layer (not shown), or a hole injection layer HIL/hole transport layer HTL/electron blocking layer EBL are stacked in its respective stated order from the first electrode EL1, but embodiments are not limited thereto.

The hole transport region HTR may be formed using various methods such as a vacuum deposition method, a spin coating method, a cast method, a Langmuir-Blodgett (LB) method, an inkjet printing method, a laser printing method, and a laser induced thermal imaging (LITI) method.

In the light emitting element ED according to an embodiment, the hole transport region HTR may include a compound represented by Formula H-1:

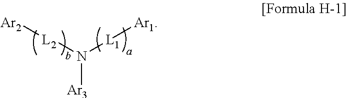

In Formula H-1, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. In Formula H-1, a and b may each independently be an integer from 0 to 10. When a or b is 2 or greater, multiple L₁ groups or multiple L₂ groups may each independently be a substituted or unsubstituted arylene group having 6 to 30 ring-forming carbon atoms or a substituted or unsubstituted heteroarylene group having 2 to 30 ring-forming carbon atoms.

In Formula H-1, Ar₁ and Ar₂ may each independently be a substituted or unsubstituted aryl group having 6 to 30 ring-forming carbon atoms or a substituted or unsubstituted heteroaryl group having 2 to 30 ring-forming carbon atoms. In Formula H-1, Ar₃ may be a substituted or unsubstituted aryl group having 6 to 30 ring-forming carbon atoms or a substituted or unsubstituted heteroaryl group having 2 to 30 ring-forming carbon atoms.

In an embodiment, the compound represented by Formula H-1 may be a monoamine compound. In another embodiment, the compound represented by Formula H-1 may be a diamine compound in which at least one of Ar₁ to Ar₃ includes an amine group as a substituent. In yet another embodiment, the compound represented by Formula H-1 may be a carbazole-based compound in which at least one of Ar₁ and Ar₂ includes a substituted or unsubstituted carbazole group, or may be a fluorene-based compound in which at least one of Ar₁ and Ar₂ includes a substituted or unsubstituted fluorene group.

The compound represented by Formula H-1 may be any compound selected from Compound Group H. However, the compounds listed in Compound Group H are only examples, and a compound represented by Formula H-1 is not limited to Compound Group H:

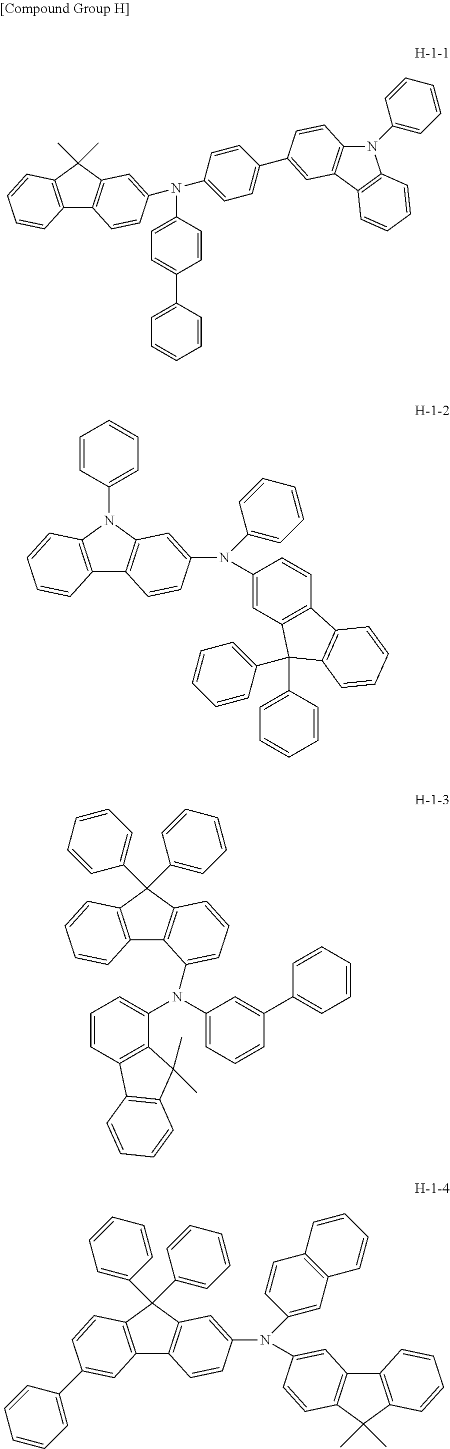 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

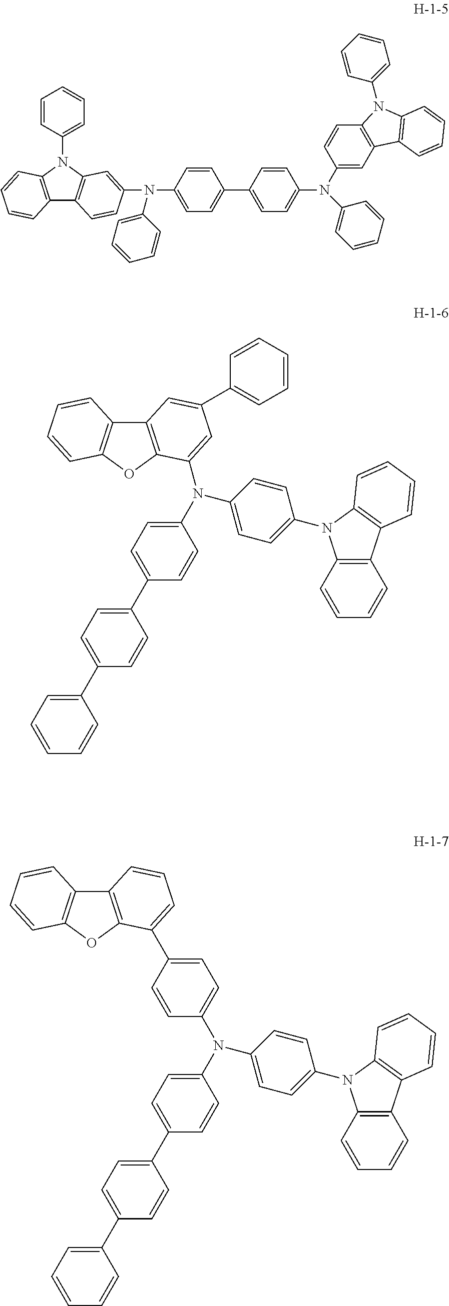 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

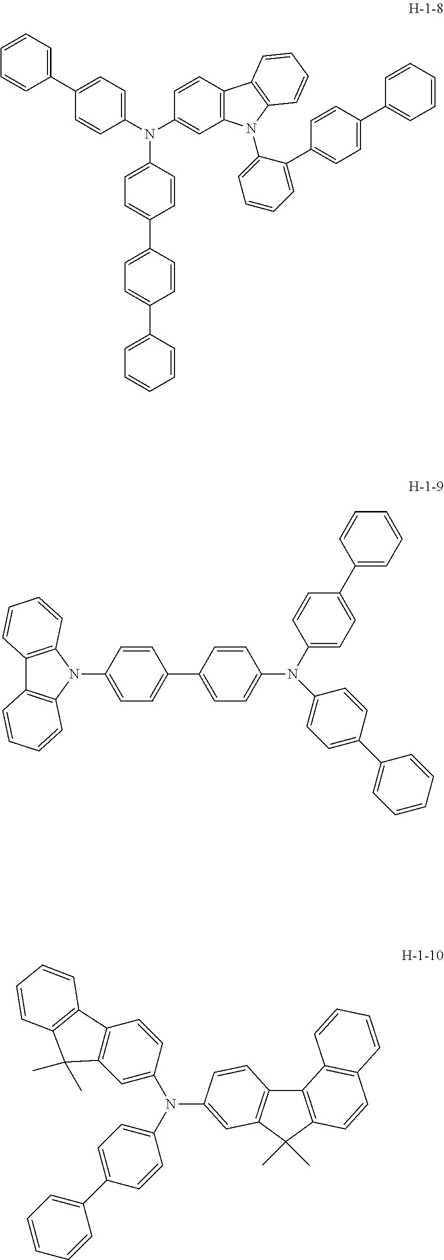 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

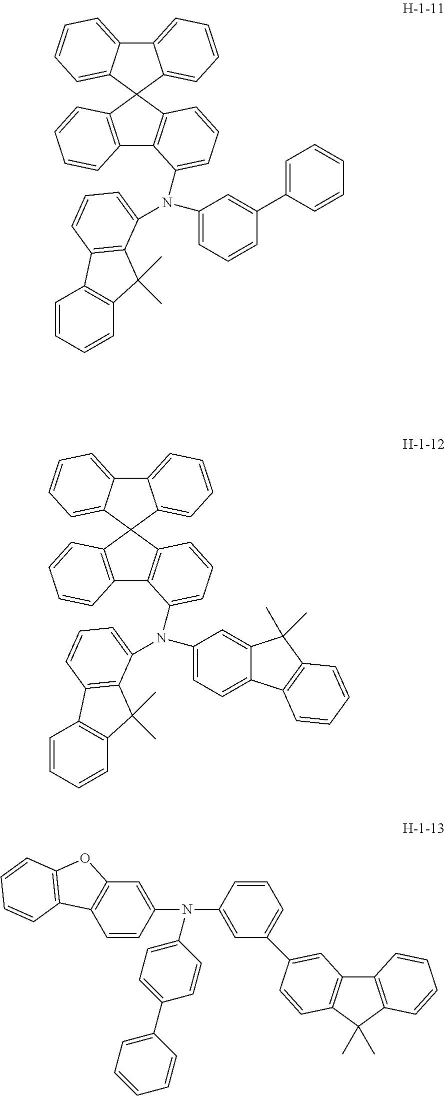 [SMILES: CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

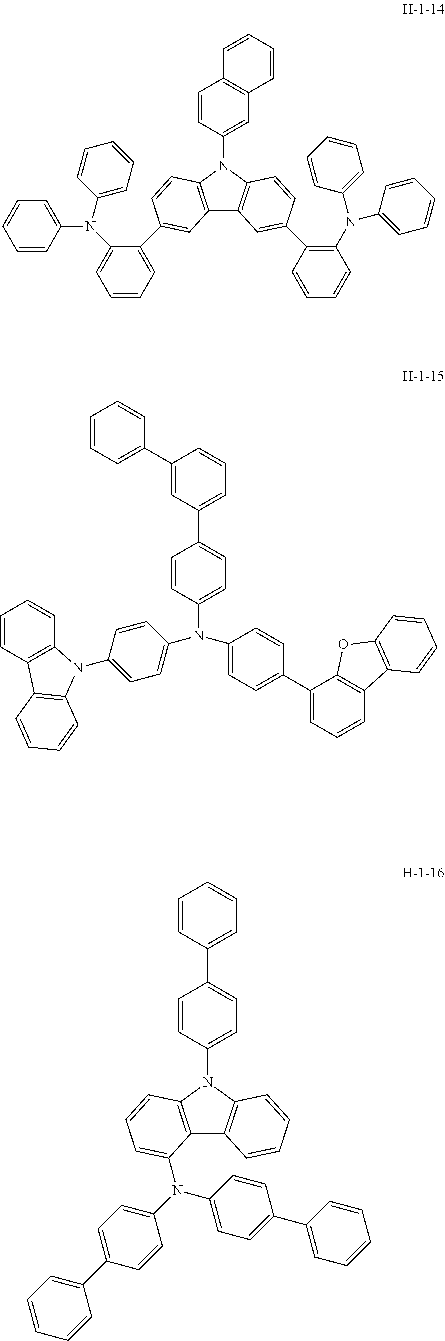 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

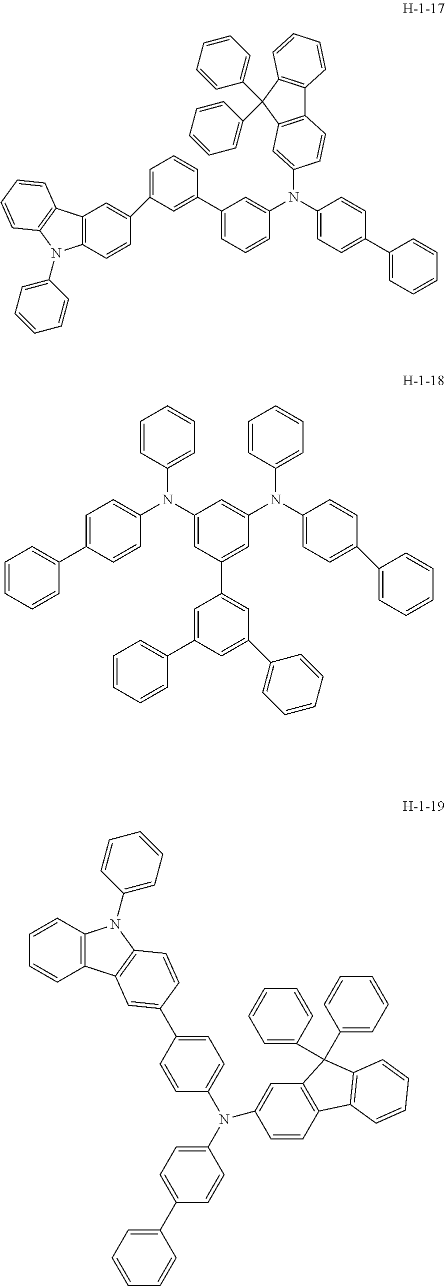 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may include a phthalocyanine compound such as copper phthalocyanine; N¹,N^{1′}-([1,1′-biphenyl]-4,4′-diyl)bis(N-phenyl-N4,N4-di-m-tolylbenzene-1,4-diamine) (DNTPD), 4,4′,4″-[tris(3-methylphenyl)phenylamino]triphenylamine (m-MTDATA), 4,4′,4″-tris(N,N-diphenylamino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalene-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], dipyrazino[2,3-f: 2′,3′-h] quinoxaline-2,3,6,7,10,11-hexacarbonitrile (HA TCN), etc.

The hole transport region HTR may include a carbazole-based derivative such as N-phenyl carbazole or polyvinyl carbazole, a fluorene-based derivative, a triphenylamine-based derivative such as N,N′-bis(3-methylphenyl)-N,N′-diphenyl-[1,1-biphenyl]-4,4′-diamine (TPD) or 4,4′,4″-tris(N-carbazolyl)triphenylamine (TCTA), N,N′-di(naphthalene-1-yl)-N,N′is (TAPC), 4,4′-bis[N,N′-(3-tolyl)amino]-3,3′-dimethylbiphenyl (HMTPD), 1,3-bis(N-carbazolyl)benzene (mCP), etc.

The hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include the above-described compounds of the hole transport region in at least one of a hole injection layer HIL, a hole transport layer HTL, and an electron blocking layer EBL.

A thickness of the hole transport region HTR may be in a range of about 100 Å to about 10,000 Å. For example, the thickness of the hole transport region HTR may be in a range of about 100 Å to about 5,000 Å. When the hole transport region HTR includes a hole injection layer HIL, the hole injection layer HIL may have a thickness in a range of about 30 Å to about 1,000 Å. When the hole transport region HTR includes a hole transport layer HTL, the hole transport layer HTL may have a thickness in a range of about 250 Å to about 1,000 Å. When the hole transport region HTR includes an electron blocking layer EBL, the electron blocking layer EBL may have a thickness in a range of about 10 Å to about 1,000 Å. If the thicknesses of the hole transport region HTR, the hole injection layer HIL, the hole transport layer HTL and the electron blocking layer EBL satisfy the above-described ranges, satisfactory hole transport properties may be achieved without a substantial increase in driving voltage.

The hole transport region HTR may further include a charge generating material to increase conductivity, in addition to the above-described materials. The charge generating material may be dispersed uniformly or non-uniformly in the hole transport region HTR. The charge generating material may be, for example, a p-dopant. The p-dopant may include at least one of a halogenated metal compound, a quinone derivative, a metal oxide, and a cyano group-containing compound, but embodiments are not limited thereto.

For example, the p-dopant may include a metal halide compound such as CuI or RbI; a quinone derivative such as tetracyanoquinodimethane (TCNQ) or 2,3,5,6-tetrafluoro-7,7′8,8-tetracyanoquinodimethane (F4-TCNQ); a metal oxide such as tungsten oxide or molybdenum oxide; a cyano group-containing compound such as dipyrazino[2,3-f: 2′,3′-h] quinoxaline-2,3,6,7,10,11-hexacarbonitrile (HATCN) or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9); etc., but embodiments are not limited thereto.

As described above, the hole transport region HTR may further include at least one of a buffer layer (not shown) and an electron blocking layer EBL, in addition to a hole injection layer HIL and a hole transport layer HTL. The buffer layer (not shown) may compensate for a resonance distance according to a wavelength of light emitted from the emission layer EML and may thus increase light emission efficiency. A material that may be included in the hole transport region HTR may be used as a material in the buffer layer (not shown). The electron blocking layer EBL may prevent injection of electrons from an electron transport region ETR to the hole transport region HTR.

An emission layer EML may be provided on the hole transport region HTR. The emission layer EML may have a thickness in a range of about 100 Å to about 1,000 Å. For example, the emission layer EML may have a thickness in a range of about 100 Å to about 300 Å. The emission layer EML may be a layer consisting of a single material, a layer including different materials, or a structure including multiple layers including different materials.

In the light emitting element ED, the emission layer EML may include an anthracene derivative, a pyrene derivative, a fluoranthene derivative, a chrysene derivative, a dihydrobenzanthracene derivative, or a triphenylene derivative. For example, the emission layer EML may include an anthracene derivative or a pyrene derivative.

In the light emitting element ED according to embodiments as shown in FIG. 3 to FIG. 6, the emission layer EML may include a host and a dopant.

In embodiments, the emission layer EML may include a first compound represented by one of Formula F-a to Formula F-c. The compound represented by one of Formula F-a to Formula F-c may be used as a fluorescent dopant material. For example, the compound represented by Formula F-c may be used as a fluorescence dopant material in the emission layer EML.

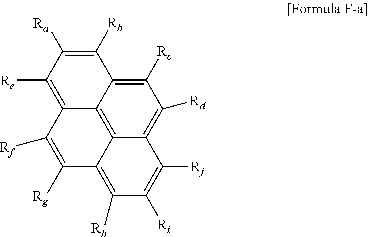

In Formula F-a, two of Rₐ to Rⱼ may each independently be substituted with a group represented by

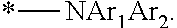

The remainder of Rₐ to Rⱼ which are not substituted with the group represented by

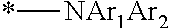

may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In the group represented by

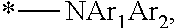

Ar₁ and Ar₂ may each independently be a substituted or unsubstituted aryl group of 6 to 30 ring-forming carbon atoms or a substituted or unsubstituted heteroaryl group of 2 to 30 ring-forming carbon atoms. For example, at least one of Ar₁ and Ar₂ may each independently be a heteroaryl group including O or S as a ring-forming atom.

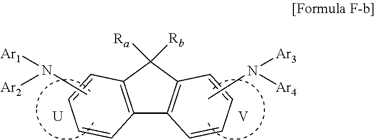

In Formula F-b, Rₐ and Rb may each independently be a hydrogen atom, a deuterium atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, a substituted or unsubstituted heteroaryl group of 2 to 30 ring-forming carbon atoms, or bonded to an adjacent group to form a ring. In Formula F-b, Ar to Ar₄ may each independently be a substituted or unsubstituted aryl group of 6 to 30 ring-forming carbon atoms or a substituted or unsubstituted heteroaryl group of 2 to 30 ring-forming carbon atoms. For example, at least one of Ar₁ to Ar₄ may each independently be a heteroaryl group including O or S as a ring-forming atom.

In Formula F-b, U and V may each independently be a substituted or unsubstituted hydrocarbon ring of 5 to 30 ring-forming carbon atoms or a substituted or unsubstituted heterocycle of 2 to 30 ring-forming carbon atoms.

In Formula F-b, the number of rings represented by U and V may each independently be 0 or 1. If the number of U or V is 1, a fused ring may be present at the portion indicated by U or V, and if the number of U or V is 0, a fused ring may not be present at the portion indicated by U or V. If the number of U is 0 and the number of V is 1, or if the number of U is 1 and the number of V is 0, a fused ring having the fluorene core of Formula F-b may be a cyclic compound with four rings. If the number of U and V is each 0, a fused ring having the fluorene core of Formula F-b may be a cyclic compound with three rings. If the number of U and V is each 1, a fused ring having the fluorene core of Formula F-b may be a cyclic compound with five rings.

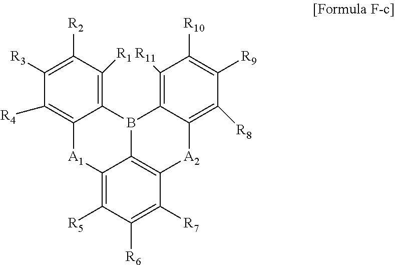

In Formula F-c, A₁ and A₂ may each independently be O, S, Se, or N(Rₘ); and Rₘ may be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. In Formula F-c, R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group of 1 to 20 carbon atoms, a substituted or unsubstituted aryl group of 6 to 30 ring-forming carbon atoms, a substituted or unsubstituted heteroaryl group of 2 to 30 ring-forming carbon atoms, or bonded to an adjacent group to form a ring.

In Formula F-c, A₁ and A₂ may each independently be combined with a substituent of an adjacent ring to form a fused ring. For example, if A₁ and A₂ are each independently N(Rₘ), A₁ may be bonded to R₄ or R₅ to form a ring. For example, A₂ may be bonded to R₇ or R₈ to form a ring.

In an embodiment, the emission layer EML may include a compound represented by Formula E-1. The compound represented by Formula E-1 may be used as a fluorescence host material.

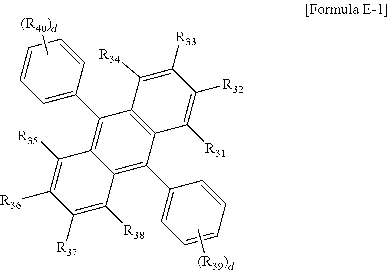

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, a substituted or unsubstituted heteroaryl group of 2 to 30 ring-forming carbon atoms, or bonded to an adjacent group to form a ring. For example, at least one of R₃₁ to R₄₀ may be combined with an adjacent group to form a saturated hydrocarbon ring, an unsaturated hydrocarbon ring, a saturated heterocycle, or an unsaturated heterocycle.

In Formula E-1, c and d may each independently be an integer from 0 to 5.

In an embodiment, the compound represented by Formula E-1 may be any compound selected from Compound E1 to Compound E19:

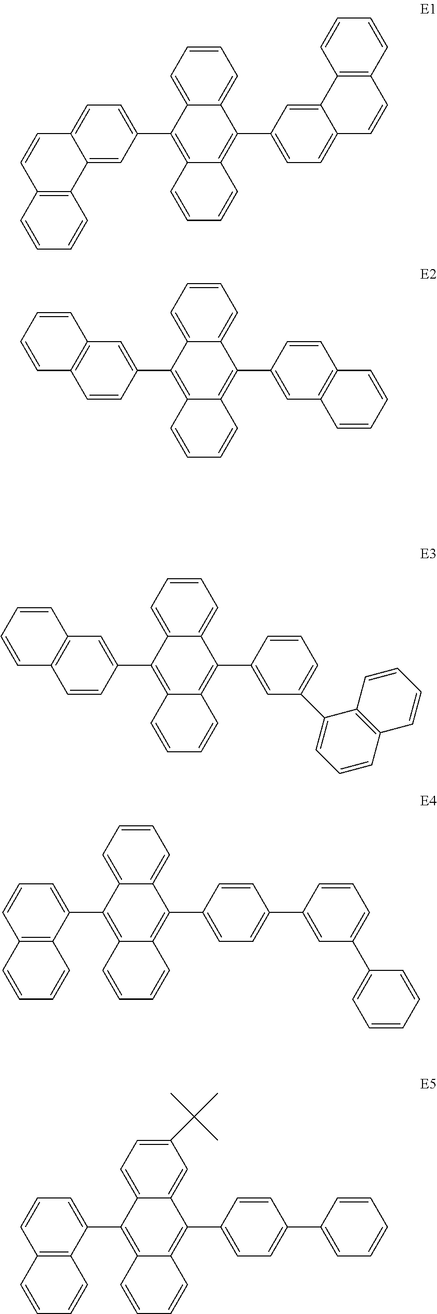

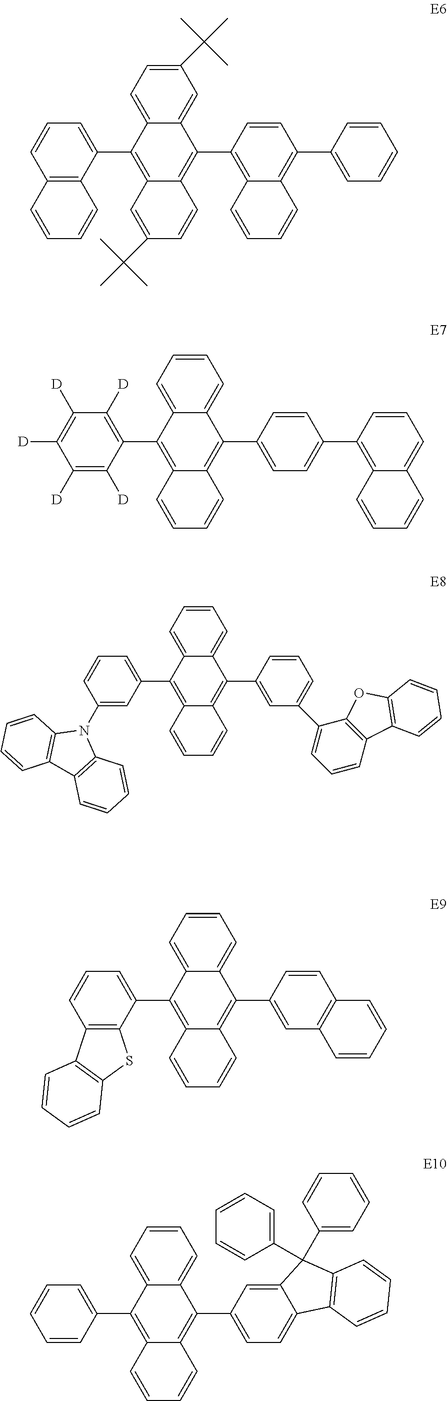

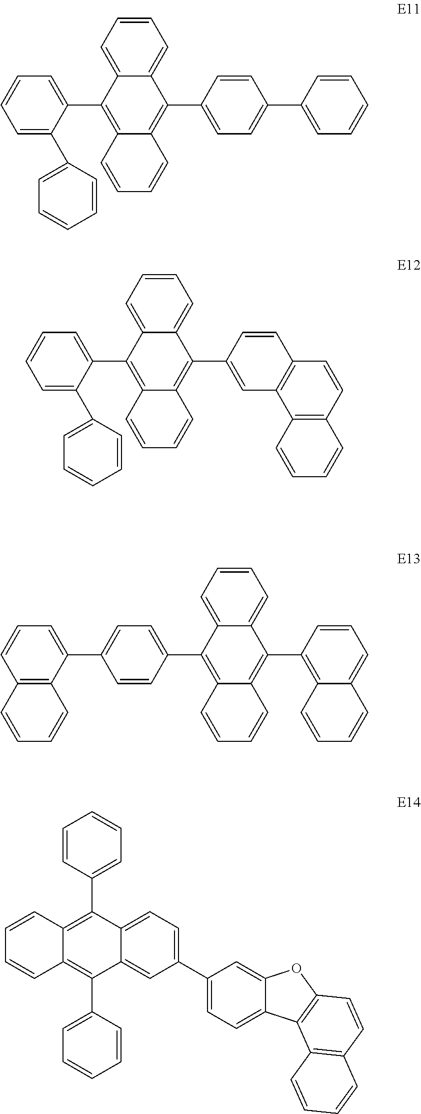

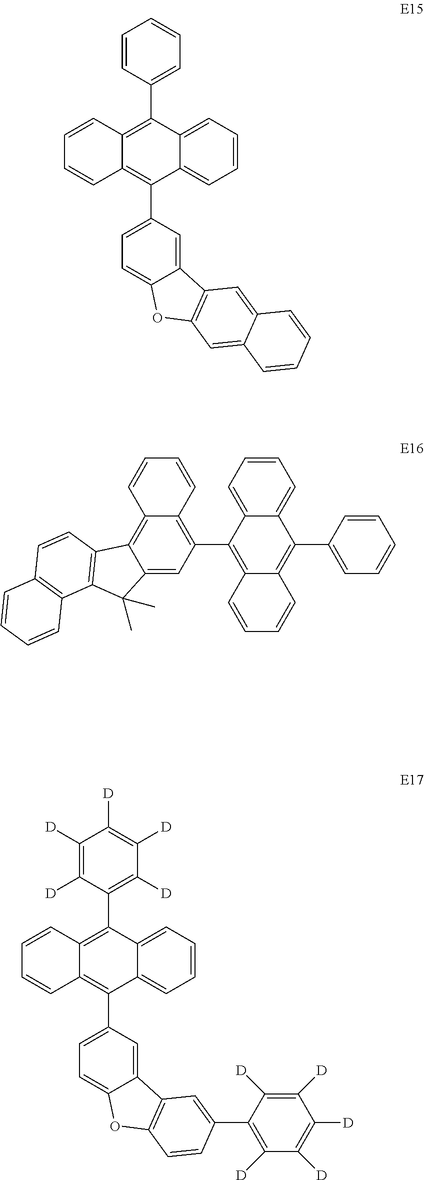

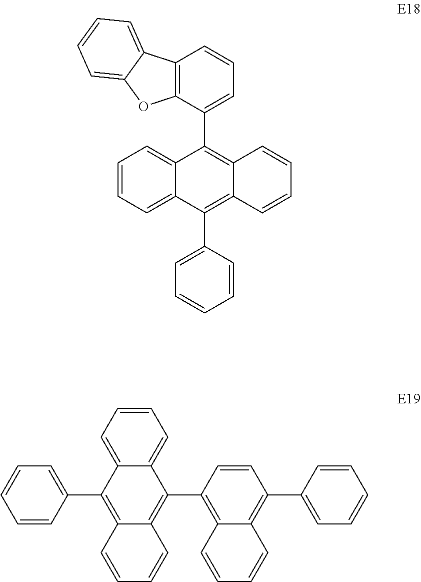

In an embodiment, the emission layer EML may include: a first compound represented by one Formula F-a to Formula F-c; and at least one of a second compound represented by Formula HT-1, a third compound represented by Formula ET-1, and a fourth compound represented by Formula D-1.

In an embodiment, the emission layer EML may further include a second compound represented by Formula HT-1. In an embodiment, the second compound may be used as a hole transport host material in the emission layer EML.

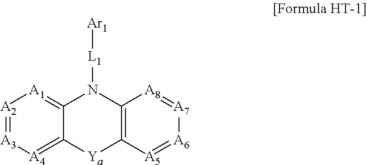

In Formula HT-1, A₁ to A₈ may each independently be N or C(R₅₁). For example, A₁ to A₈ may each independently be C(R₅₁). A₈ another example, one of A₁ to A₈ may be N, and the remainder of A₁ to A₈ may each independently be C(R₅₁).

In Formula HT-1, L₁ may be a direct linkage, a substituted or unsubstituted arylene group of 6 to 30 ring-forming carbon atoms, or a substituted or unsubstituted heteroarylene group of 2 to 30 ring-forming carbon atoms. For example, L₁ may be a direct linkage, a substituted or unsubstituted phenylene group, a substituted or unsubstituted divalent biphenyl group, a substituted or unsubstituted divalent carbazole group, or the like, but embodiments are not limited thereto.

In Formula HT-1, Yₐ may be a direct linkage, C(R₅₂)(R₅₃), or Si(R₅₄)(R₅₅). For example, the two aromatic rings that are bonded to the nitrogen atom of Formula HT-1 may be

directly connected to each other via a direct linkage,

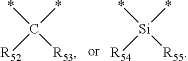

In Formula HT-1, if Yₐ is a direct linkage, the second compound represented by Formula HT-1 may include a carbazole moiety.

In Formula HT-1, Ar may be a substituted or unsubstituted aryl group of 6 to 30 ring-forming carbon atoms or a substituted or unsubstituted heteroaryl group of 2 to 30 ring-forming carbon atoms. For example, Ar may be a substituted or unsubstituted carbazole group, a substituted or unsubstituted dibenzofuran group, a substituted or unsubstituted dibenzothiophene group, or a substituted or unsubstituted biphenyl group, but embodiments are not limited thereto.

In Formula HT-1, R₅₁ to R₅₅ may each independently be a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group of 1 to 20 carbon atoms, a substituted or unsubstituted alkenyl group of 2 to 20 ring-forming carbon atoms, a substituted or unsubstituted aryl group of 6 to 60 ring-forming carbon atoms, a substituted or unsubstituted heteroaryl group of 2 to 60 ring-forming carbon atoms, or bonded to an adjacent group to form a ring. For example, R₅₁ to R₅₅ may each independently be a hydrogen atom or a deuterium atom. For example, R₅₁ to R₅₅ may each independently be an unsubstituted methyl group or an unsubstituted phenyl group.

In an embodiment, the second compound represented by Formula HT-1 may be selected from Compound Group 2. In an embodiment, in the light emitting element ED, the second compound may include at least one compound selected from Compound Group 2:

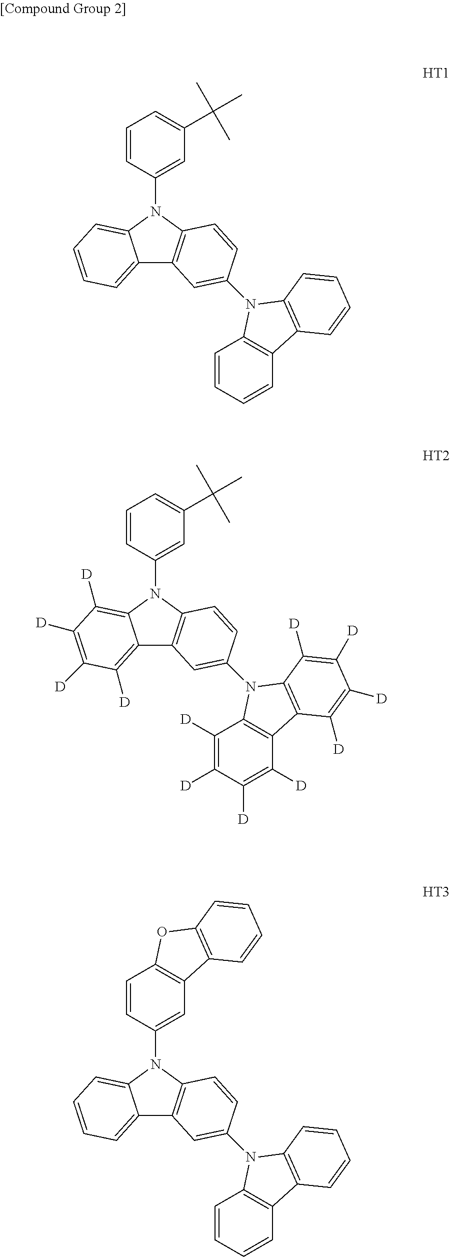 [SMILES: CC(C)(C)c1cccc(-n2c3ccccc3c3cc(-n4c5ccccc5c5ccccc54)ccc32)c1 ; [2H]c1c([2H])c([2H])c2c(c1[2H])c1cc(-n3c4c([2H])c([2H])c([2H])c([2H])c4c4c([2H])c([2H])c([2H])c([2H])c43)ccc1n2-c1cccc(C(C)(C)C)c1 ; c1ccc2c(c1)oc1ccc(-n3c4ccccc4c4cc(-n5c6ccccc6c6ccccc65)ccc43)cc12]

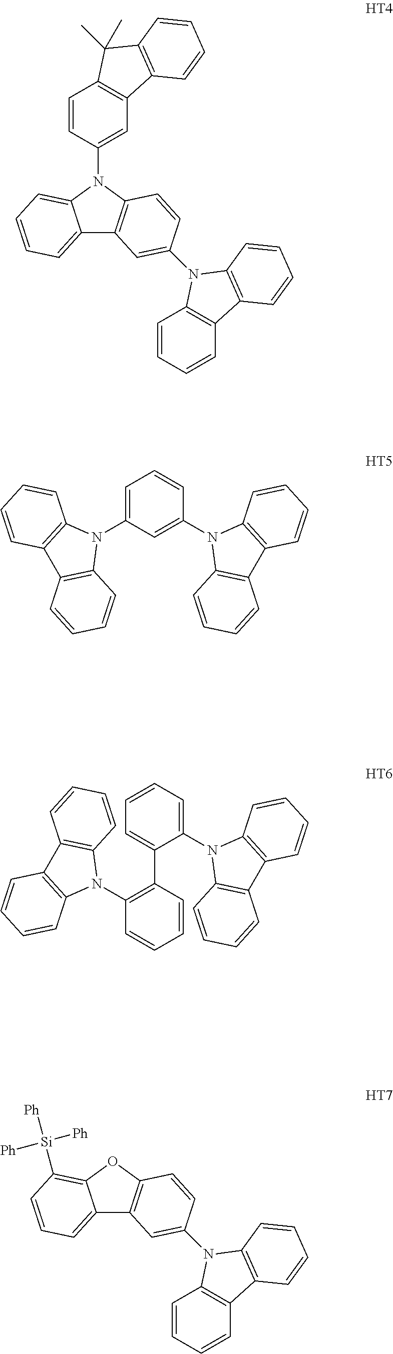 [SMILES: CC1(C)c2ccccc2-c2cc(-n3c4ccccc4c4cc(-n5c6ccccc6c6ccccc65)ccc43)ccc21 ; c1cc(-n2c3ccccc3c3ccccc32)cc(-n2c3ccccc3c3ccccc32)c1 ; c1ccc(-n2c3ccccc3c3ccccc32)c(-c2ccccc2-n2c3ccccc3c3ccccc32)c1 ; c1ccc([Si](c2ccccc2)(c2ccccc2)c2cccc3c2oc2ccc(-n4c5ccccc5c5ccccc54)cc23)cc1]

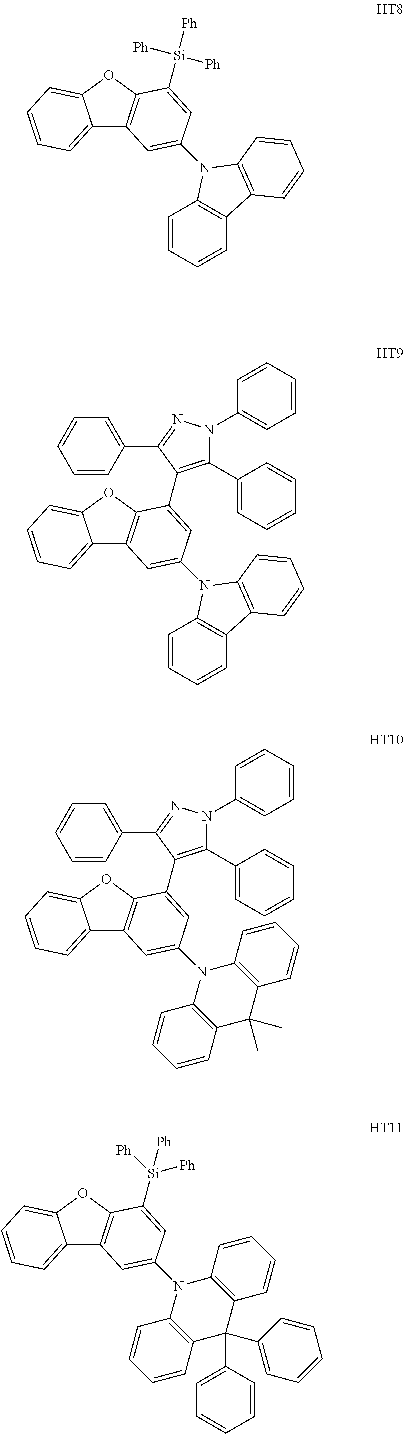 [SMILES: c1ccc([Si](c2ccccc2)(c2ccccc2)c2cc(-n3c4ccccc4c4ccccc43)cc3c2oc2ccccc23)cc1 ; c1ccc(-c2nn(-c3ccccc3)c(-c3ccccc3)c2-c2cc(-n3c4ccccc4c4ccccc43)cc3c2oc2ccccc23)cc1 ; CC1(C)c2ccccc2N(c2cc(-c3c(-c4ccccc4)nn(-c4ccccc4)c3-c3ccccc3)c3oc4ccccc4c3c2)c2ccccc21 ; c1ccc(C2(c3ccccc3)c3ccccc3N(c3cc([Si](c4ccccc4)(c4ccccc4)c4ccccc4)c4oc5ccccc5c4c3)c3ccccc32)cc1]

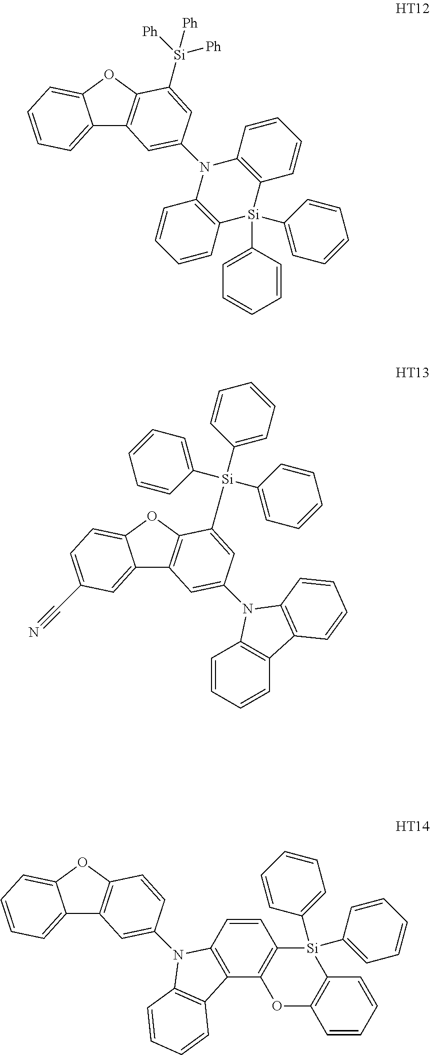 [SMILES: c1ccc([Si]2(c3ccccc3)c3ccccc3N(c3cc([Si](c4ccccc4)(c4ccccc4)c4ccccc4)c4oc5ccccc5c4c3)c3ccccc32)cc1 ; N#Cc1ccc2oc3c([Si](c4ccccc4)(c4ccccc4)c4ccccc4)cc(-n4c5ccccc5c5ccccc54)cc3c2c1 ; c1ccc([Si]2(c3ccccc3)c3ccccc3Oc3c2ccc2c3c3ccccc3n2-c2ccc3oc4ccccc4c3c2)cc1]

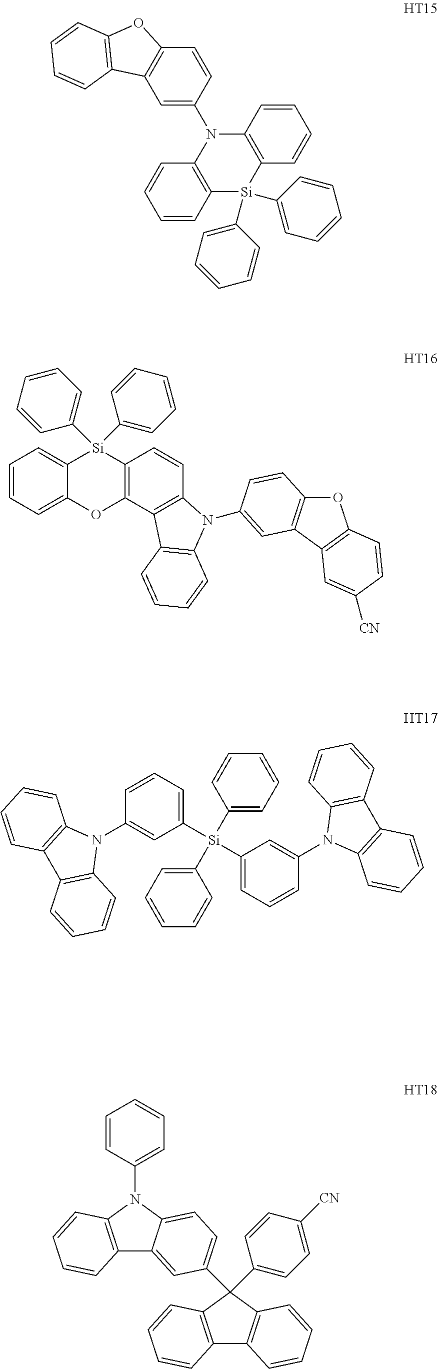 [SMILES: c1ccc([Si]2(c3ccccc3)c3ccccc3N(c3ccc4oc5ccccc5c4c3)c3ccccc32)cc1 ; N#Cc1ccc2oc3ccc(-n4c5ccccc5c5c6c(ccc54)[Si](c4ccccc4)(c4ccccc4)c4ccccc4O6)cc3c2c1 ; c1ccc([Si](c2ccccc2)(c2cccc(-n3c4ccccc4c4ccccc43)c2)c2cccc(-n3c4ccccc4c4ccccc43)c2)cc1 ; N#Cc1ccc(C2(c3ccc4c(c3)c3ccccc3n4-c3ccccc3)c3ccccc3-c3ccccc32)cc1]

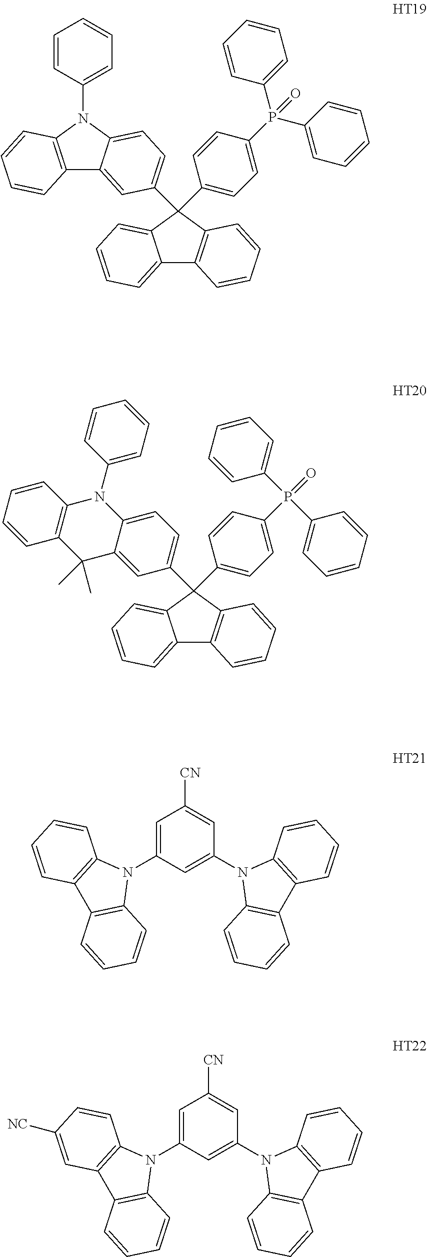 [SMILES: O=P(c1ccccc1)(c1ccccc1)c1ccc(C2(c3ccc4c(c3)c3ccccc3n4-c3ccccc3)c3ccccc3-c3ccccc32)cc1 ; CC1(C)c2ccccc2N(c2ccccc2)c2ccc(C3(c4ccc(P(=O)(c5ccccc5)c5ccccc5)cc4)c4ccccc4-c4ccccc43)cc21 ; N#Cc1cc(-n2c3ccccc3c3ccccc32)cc(-n2c3ccccc3c3ccccc32)c1 ; N#Cc1cc(-n2c3ccccc3c3ccccc32)cc(-n2c3ccccc3c3cc(C#N)ccc32)c1]

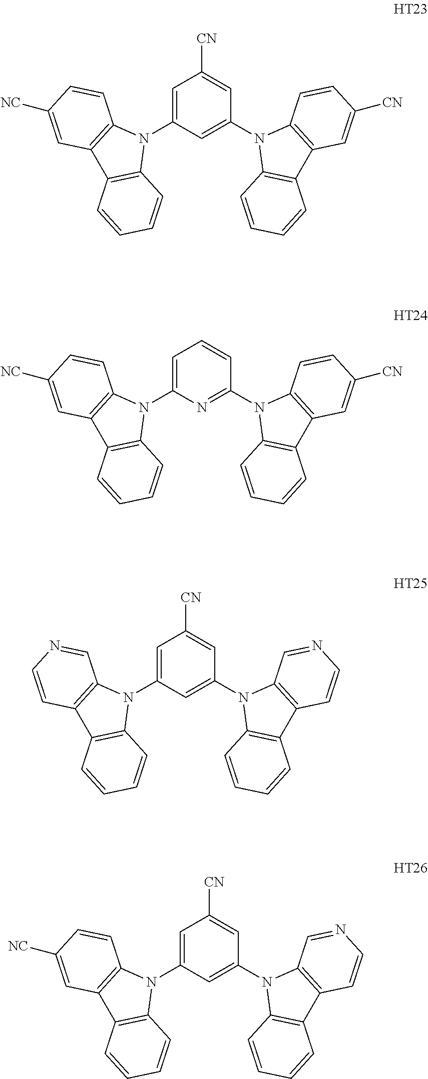 [SMILES: N#Cc1cc(-n2c3ccccc3c3cc(C#N)ccc32)cc(-n2c3ccccc3c3cc(C#N)ccc32)c1 ; N#Cc1ccc2c(c1)c1ccccc1n2-c1cccc(-n2c3ccccc3c3cc(C#N)ccc32)n1 ; N#Cc1cc(-n2c3ccccc3c3ccncc32)cc(-n2c3ccccc3c3ccncc32)c1 ; N#Cc1cc(-n2c3ccccc3c3cc(C#N)ccc32)cc(-n2c3ccccc3c3ccncc32)c1]

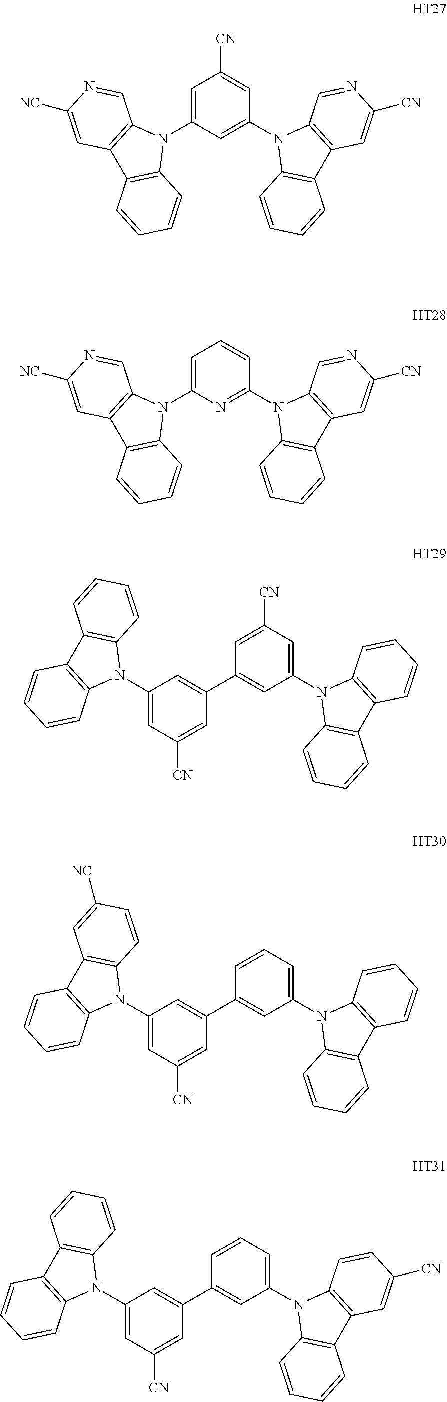 [SMILES: N#Cc1cc(-n2c3ccccc3c3cc(C#N)ncc32)cc(-n2c3ccccc3c3cc(C#N)ncc32)c1 ; N#Cc1cc2c3ccccc3n(-c3cccc(-n4c5ccccc5c5cc(C#N)ncc54)n3)c2cn1 ; N#Cc1cc(-c2cc(C#N)cc(-n3c4ccccc4c4ccccc43)c2)cc(-n2c3ccccc3c3ccccc32)c1 ; N#Cc1cc(-c2cccc(-n3c4ccccc4c4ccccc43)c2)cc(-n2c3ccccc3c3cc(C#N)ccc32)c1 ; N#Cc1cc(-c2cccc(-n3c4ccccc4c4cc(C#N)ccc43)c2)cc(-n2c3ccccc3c3ccccc32)c1]

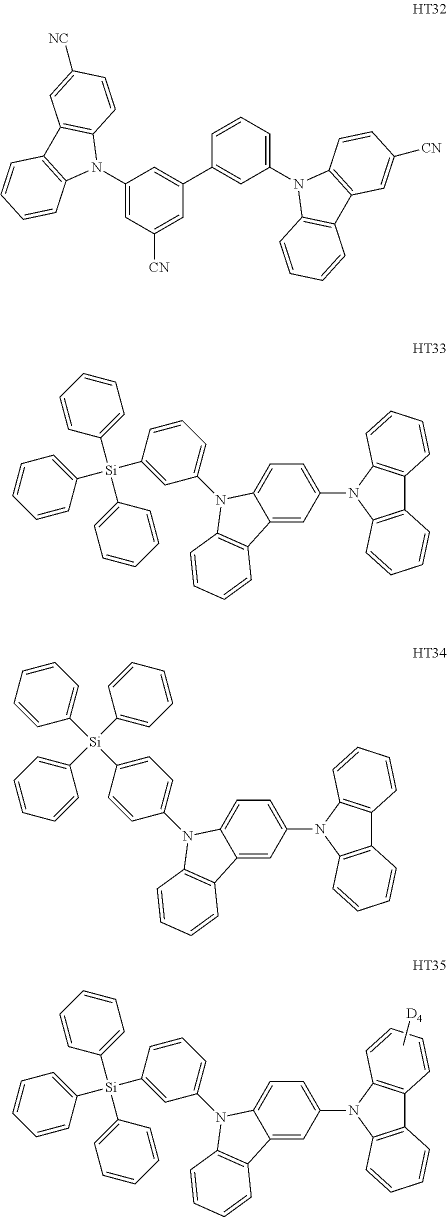

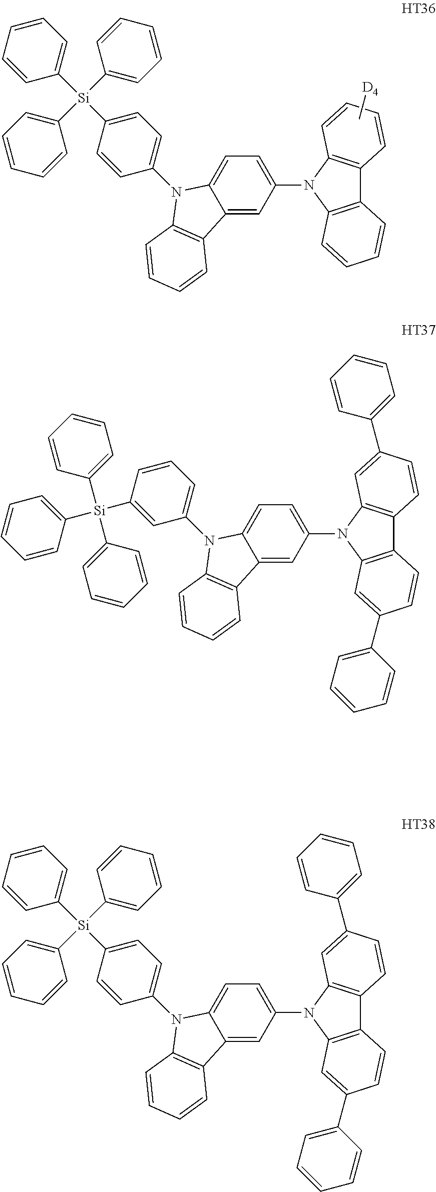

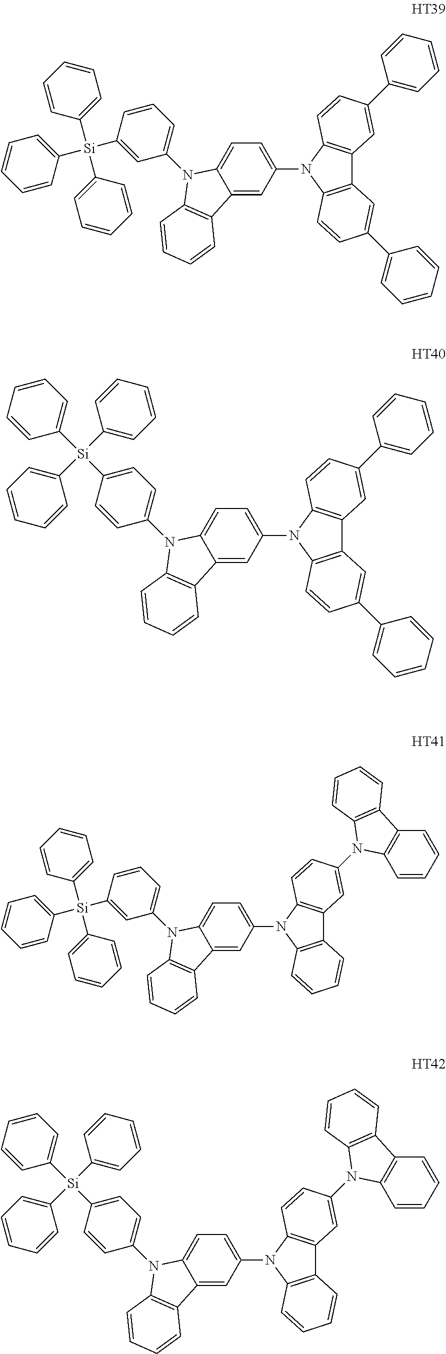 [SMILES: c1ccc(-c2ccc3c(c2)c2cc(-c4ccccc4)ccc2n3-c2ccc3c(c2)c2ccccc2n3-c2cccc([Si](c3ccccc3)(c3ccccc3)c3ccccc3)c2)cc1 ; c1ccc(-c2ccc3c(c2)c2cc(-c4ccccc4)ccc2n3-c2ccc3c(c2)c2ccccc2n3-c2ccc([Si](c3ccccc3)(c3ccccc3)c3ccccc3)cc2)cc1 ; c1ccc([Si](c2ccccc2)(c2ccccc2)c2cccc(-n3c4ccccc4c4cc(-n5c6ccccc6c6cc(-n7c8ccccc8c8ccccc87)ccc65)ccc43)c2)cc1 ; c1ccc([Si](c2ccccc2)(c2ccccc2)c2ccc(-n3c4ccccc4c4cc(-n5c6ccccc6c6cc(-n7c8ccccc8c8ccccc87)ccc65)ccc43)cc2)cc1]

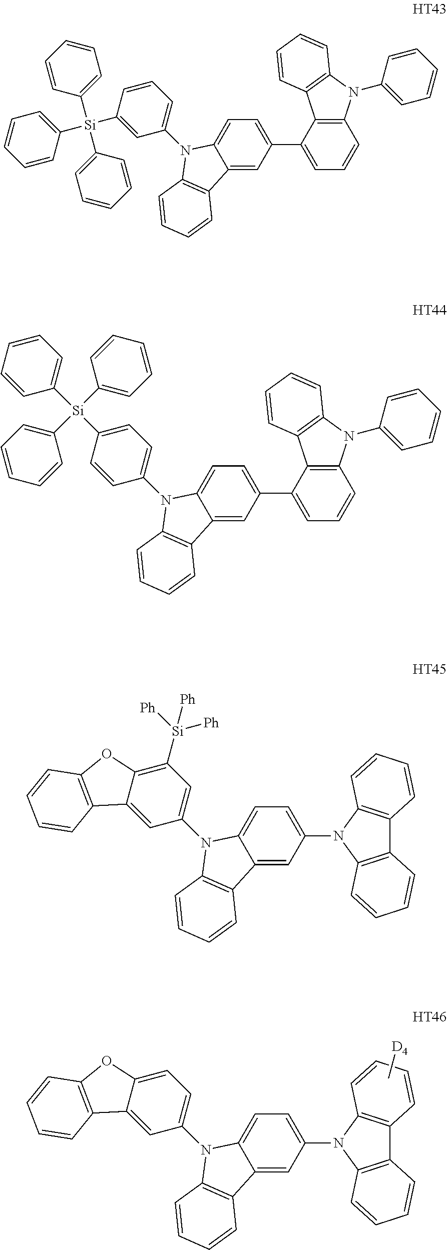

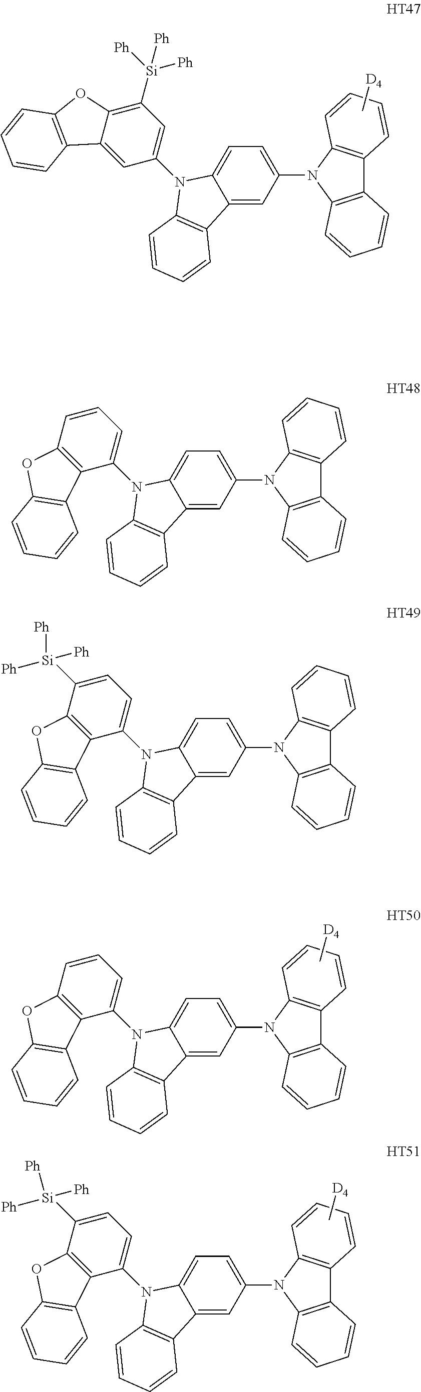

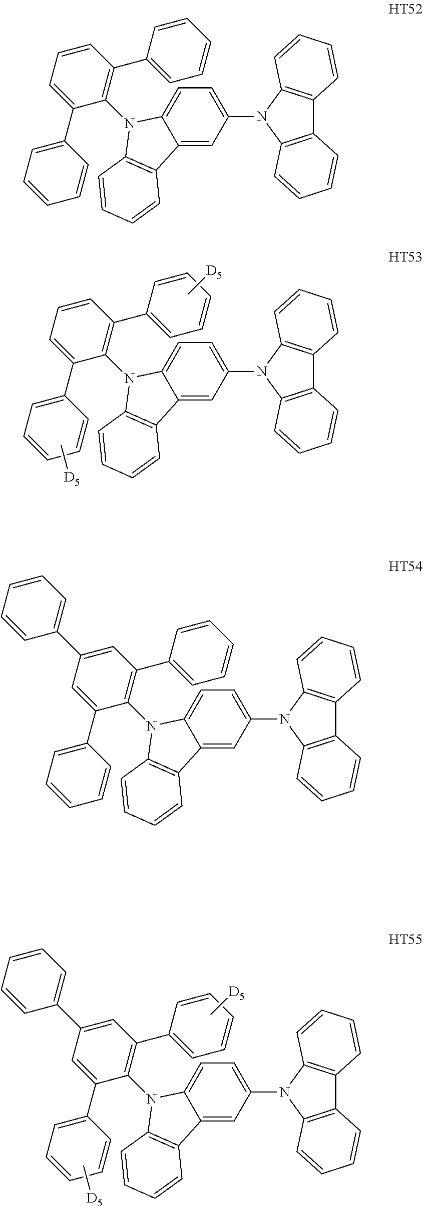

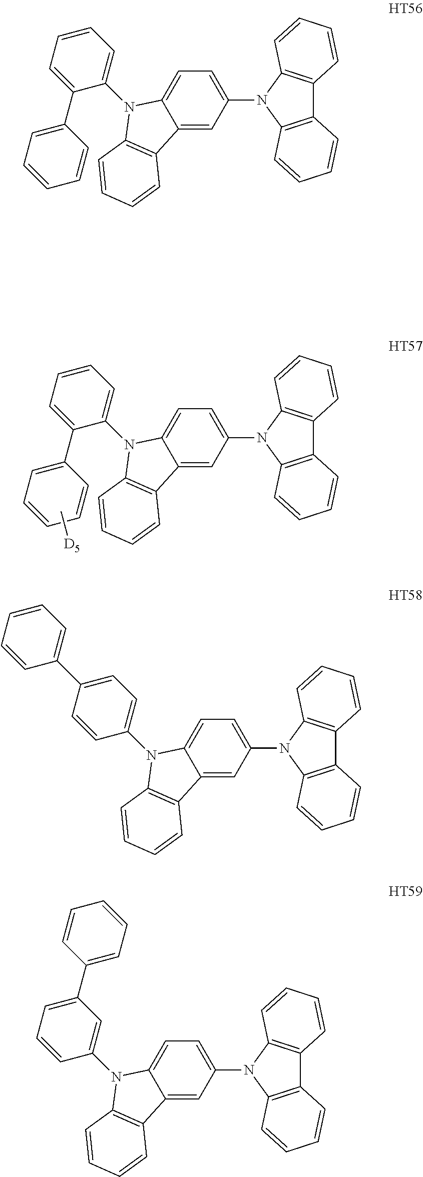

In Compound Group 2, D represents a deuterium atom, and Ph represents a substituted or unsubstituted phenyl group. For example, in Compound Group 2, Ph may represent an unsubstituted phenyl group.

In an embodiment, the emission layer EML may further include a third compound represented by Formula ET-1. In an embodiment, the third compound may be used as an electron transport host material in the emission layer EML.

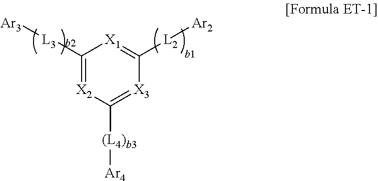

In Formula ET-1, at least one of X₁ to X₃ may each be N, and the remainder of X₁ to X₃ may each independently be C(R₅₆). For example, one of X₁ to X₃ may be N, and the remainder of X₁ to X₃ may each independently be C(R₅₆). Thus, the third compound represented by Formula ET-1 may include a pyridine moiety. A₈ another example, two of X₁ to X₃ may each be N, and the remainder of X₁ to X₃ may be C(R₅₆). Thus, the third compound represented by Formula ET-1 may include a pyrimidine moiety. A₈ yet another example, X₁ to X₃ may each be N. Thus, the third compound represented by Formula ET-1 may include a triazine moiety.

In Formula ET-1, R₅₆ may be a hydrogen atom, a deuterium atom, a substituted or unsubstituted alkyl group of 1 to 20 carbon atoms, a substituted or unsubstituted aryl group of 6 to 60 ring-forming carbon atoms, or a substituted or unsubstituted heteroaryl group of 2 to 60 ring-forming carbon atoms.

In Formula ET-1, b1 to b3 may each independently be an integer from 0 to 10.

In Formula ET-1, Ar₂ to Ar₄ may each independently be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, Ar₂ to Ar₄ may each independently be a substituted or unsubstituted phenyl group or a substituted or unsubstituted carbazole group.

In Formula ET-1, L₂ to L₄ may each independently be a direct linkage, a substituted or unsubstituted arylene group of 6 to 30 ring-forming carbon atoms, or a substituted or unsubstituted heteroarylene group of 2 to 30 ring-forming carbon atoms. If b1 to b3 are each 2 or more, multiple groups of each of L₂ to L₄ may each independently be a substituted or unsubstituted arylene group of 6 to 30 ring-forming carbon atoms or a substituted or unsubstituted heteroarylene group of 2 to 30 ring-forming carbon atoms.

In an embodiment, the third compound represented by Formula ET-1 may be selected from Compound Group 3. In an embodiment, in the light emitting element ED, the third compound may include at least one compound selected from Compound Group 3:

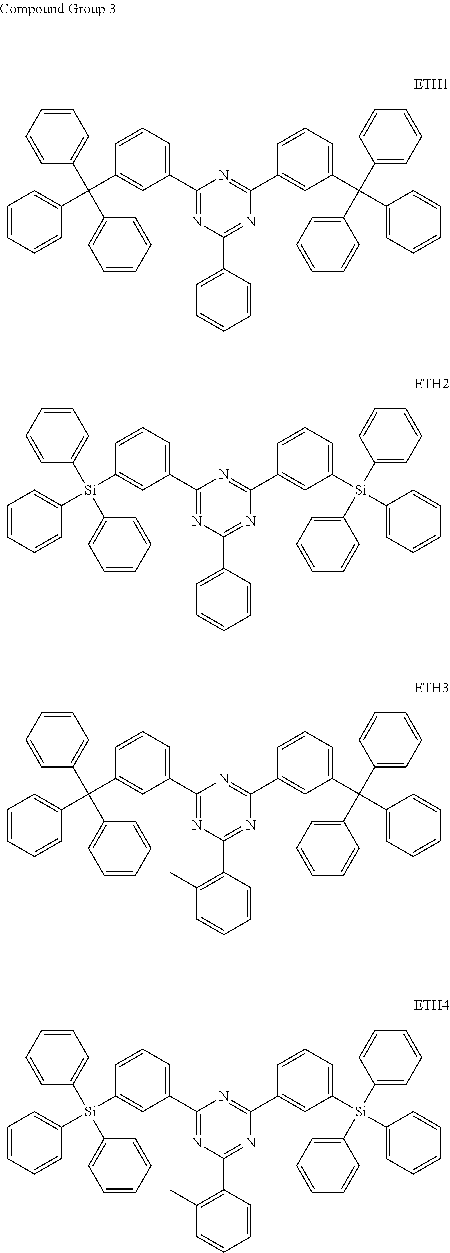 [SMILES: c1ccc(-c2nc(-c3cccc(C(c4ccccc4)(c4ccccc4)c4ccccc4)c3)nc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)nc(-c2cccc([Si](c3ccccc3)(c3ccccc3)c3ccccc3)c2)n1]

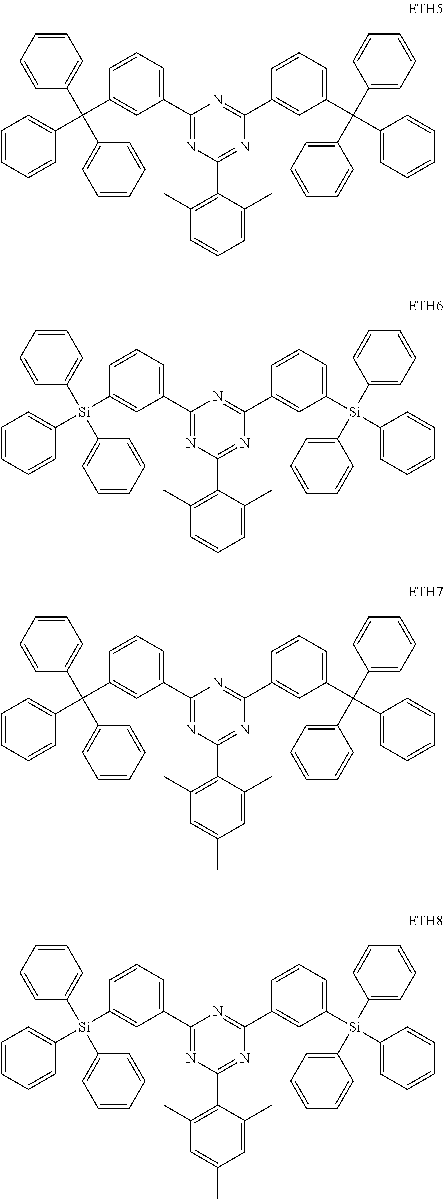 [SMILES: Cc1cccc(C)c1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)nc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)nc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)nc(-c3cccc([Si](c4ccccc4)(c4ccccc4)c4ccccc4)c3)n2)c(C)c1]

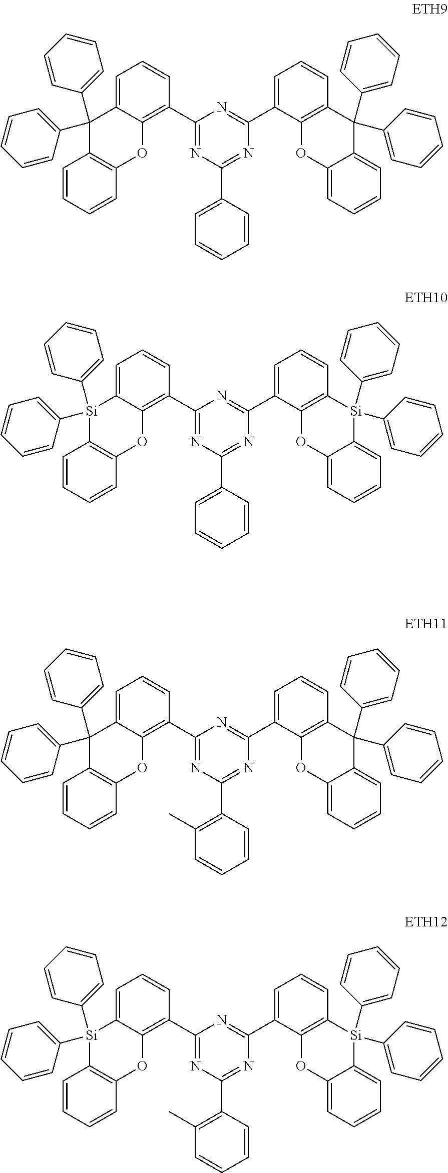 [SMILES: c1ccc(-c2nc(-c3cccc4c3Oc3ccccc3C4(c3ccccc3)c3ccccc3)nc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1]

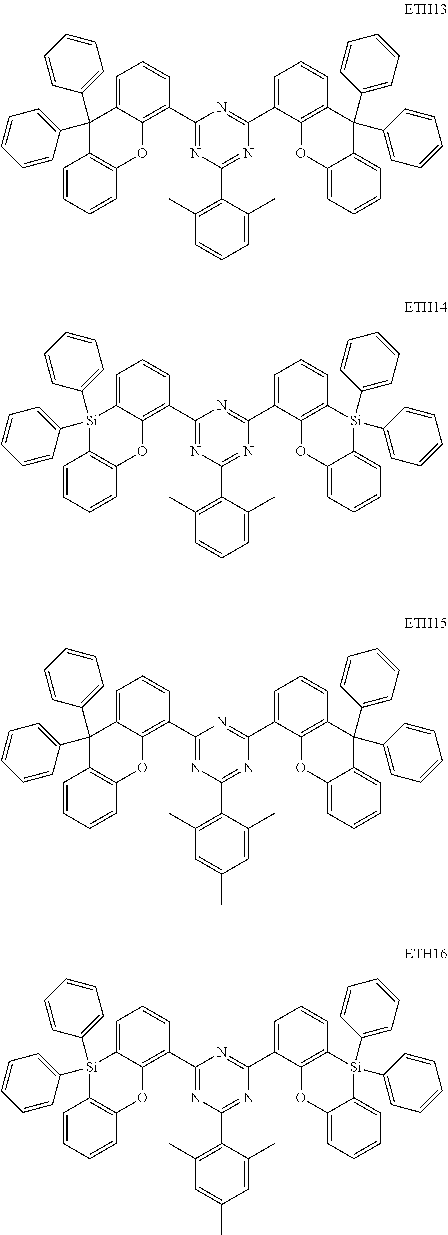 [SMILES: Cc1cccc(C)c1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)nc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)c(C)c1]

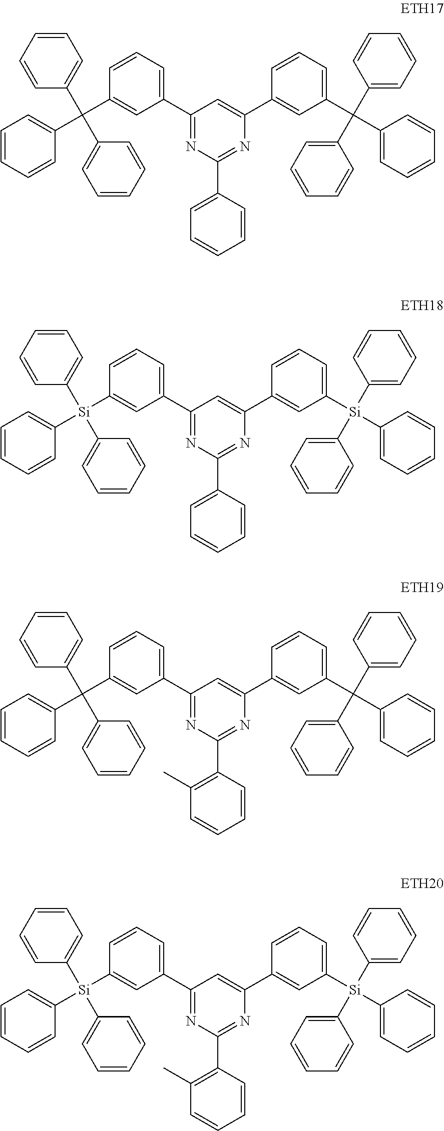 [SMILES: c1ccc(-c2nc(-c3cccc(C(c4ccccc4)(c4ccccc4)c4ccccc4)c3)cc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)cc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)cc(-c2cccc([Si](c3ccccc3)(c3ccccc3)c3ccccc3)c2)n1]

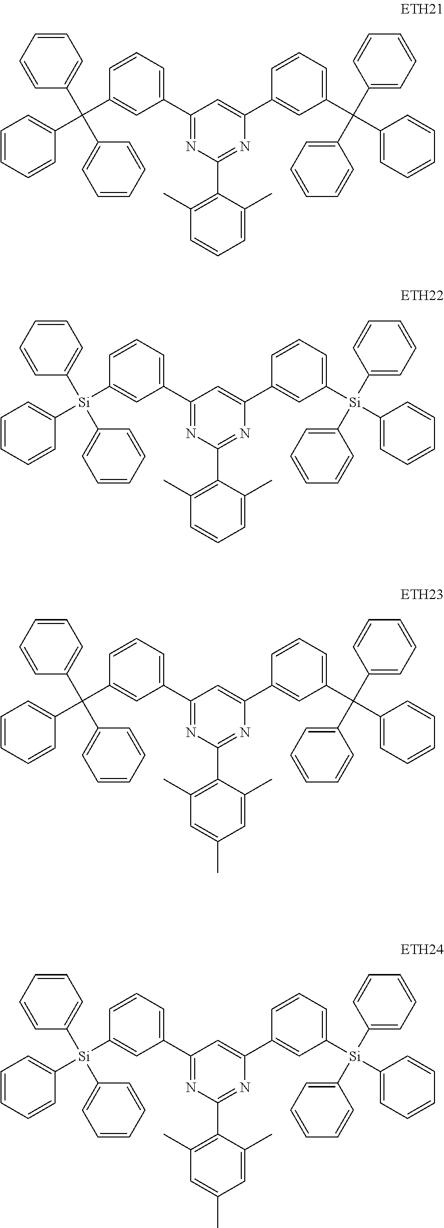 [SMILES: Cc1cccc(C)c1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)cc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)cc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)cc(-c3cccc([Si](c4ccccc4)(c4ccccc4)c4ccccc4)c3)n2)c(C)c1]

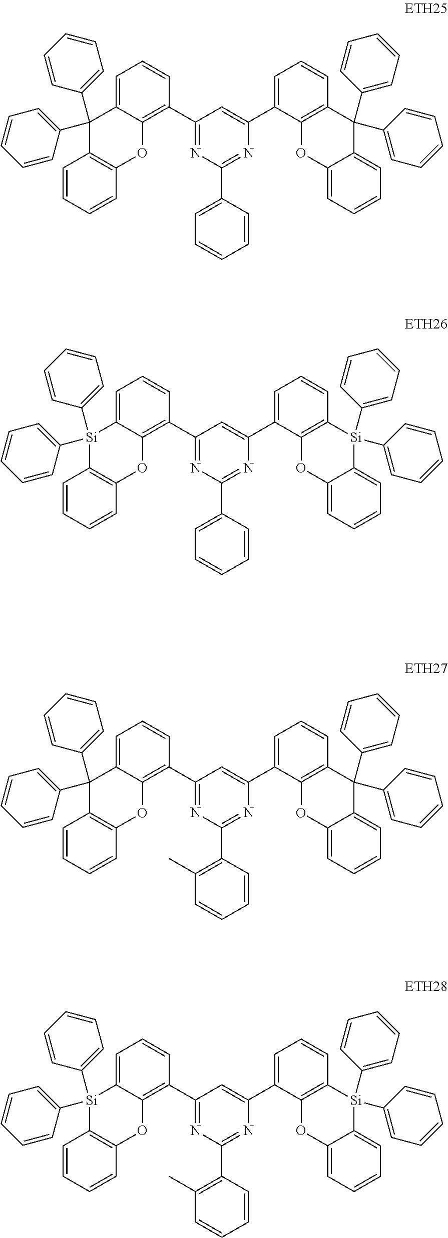 [SMILES: c1ccc(-c2nc(-c3cccc4c3Oc3ccccc3C4(c3ccccc3)c3ccccc3)cc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1]

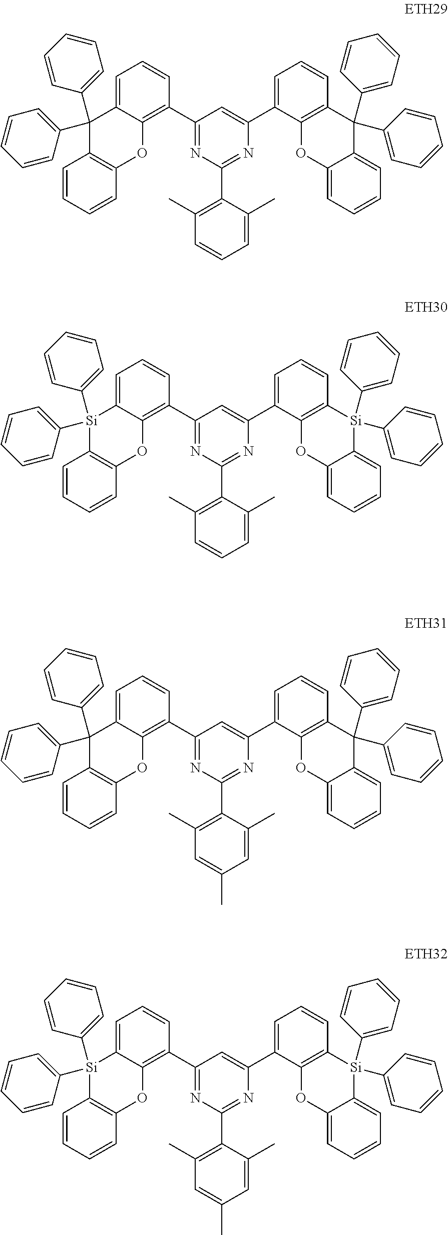 [SMILES: Cc1cccc(C)c1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)cc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)c(C)c1]

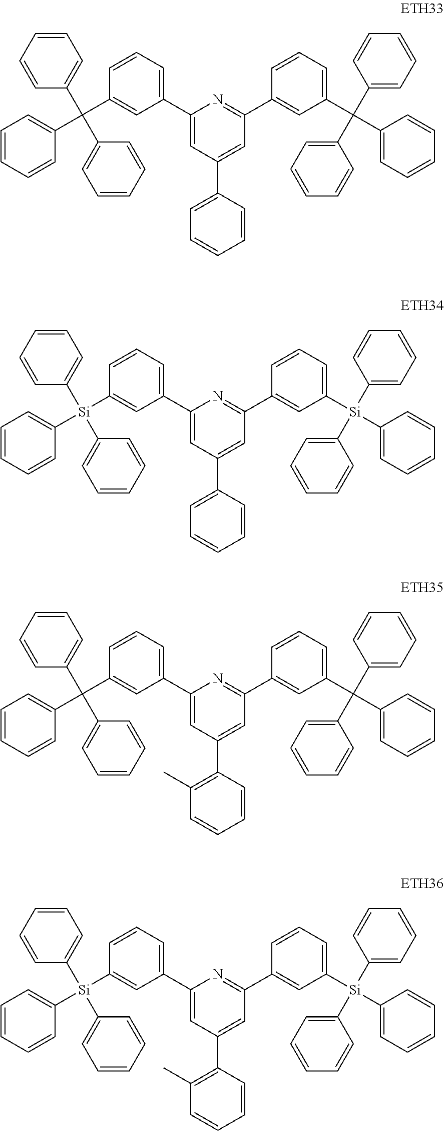 [SMILES: c1ccc(-c2cc(-c3cccc(C(c4ccccc4)(c4ccccc4)c4ccccc4)c3)nc(-c3cccc(C(c4ccccc4)(c4ccccc4)c4ccccc4)c3)c2)cc1 ; c1ccc(-c2cc(-c3cccc([Si](c4ccccc4)(c4ccccc4)c4ccccc4)c3)nc(-c3cccc([Si](c4ccccc4)(c4ccccc4)c4ccccc4)c3)c2)cc1 ; Cc1ccccc1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1ccccc1-c1cc(-c2cccc([Si](c3ccccc3)(c3ccccc3)c3ccccc3)c2)nc(-c2cccc([Si](c3ccccc3)(c3ccccc3)c3ccccc3)c2)c1]

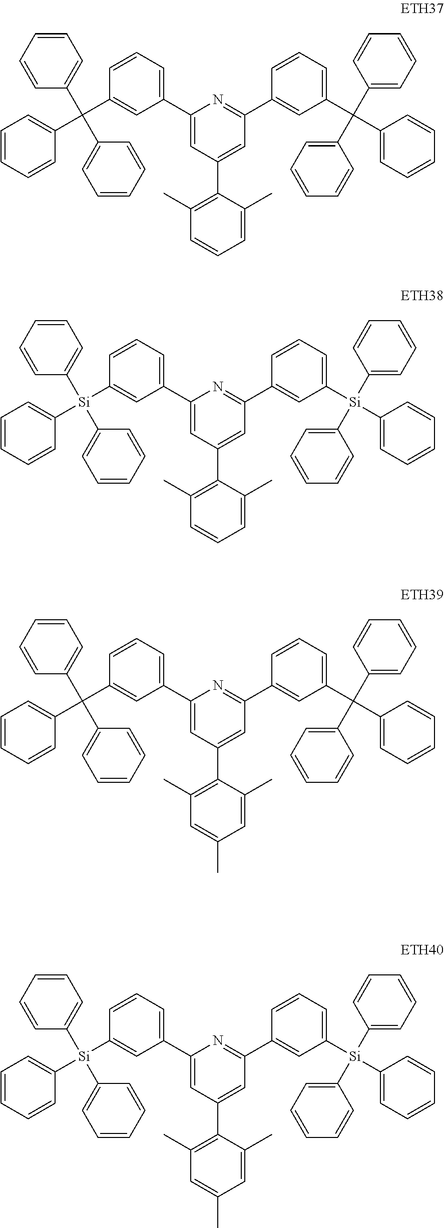 [SMILES: Cc1cccc(C)c1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1cccc(C)c1-c1cc(-c2cccc([Si](c3ccccc3)(c3ccccc3)c3ccccc3)c2)nc(-c2cccc([Si](c3ccccc3)(c3ccccc3)c3ccccc3)c2)c1 ; Cc1cc(C)c(-c2cc(-c3cccc(C(c4ccccc4)(c4ccccc4)c4ccccc4)c3)nc(-c3cccc(C(c4ccccc4)(c4ccccc4)c4ccccc4)c3)c2)c(C)c1 ; Cc1cc(C)c(-c2cc(-c3cccc([Si](c4ccccc4)(c4ccccc4)c4ccccc4)c3)nc(-c3cccc([Si](c4ccccc4)(c4ccccc4)c4ccccc4)c3)c2)c(C)c1]

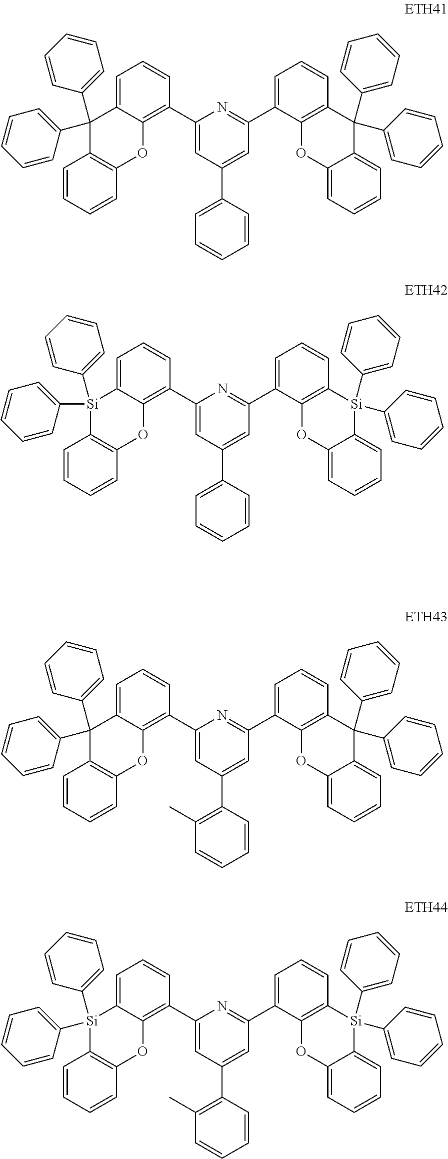 [SMILES: c1ccc(-c2cc(-c3cccc4c3Oc3ccccc3C4(c3ccccc3)c3ccccc3)nc(-c3cccc4c3Oc3ccccc3C4(c3ccccc3)c3ccccc3)c2)cc1 ; c1ccc(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)cc1 ; Cc1ccccc1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1ccccc1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1]

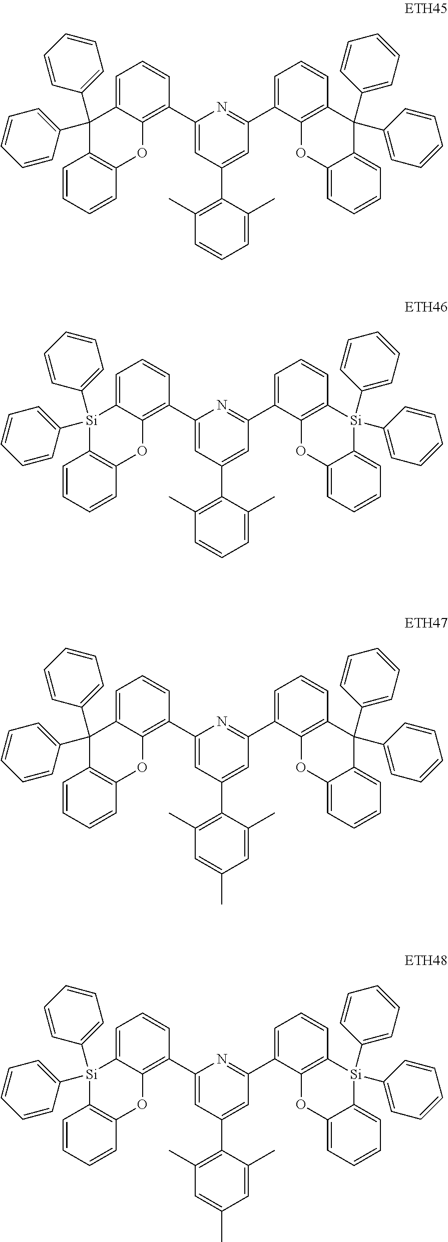 [SMILES: Cc1cccc(C)c1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1cccc(C)c1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1 ; Cc1cc(C)c(-c2cc(-c3cccc4c3Oc3ccccc3C4(c3ccccc3)c3ccccc3)nc(-c3cccc4c3Oc3ccccc3C4(c3ccccc3)c3ccccc3)c2)c(C)c1 ; Cc1cc(C)c(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)c(C)c1]

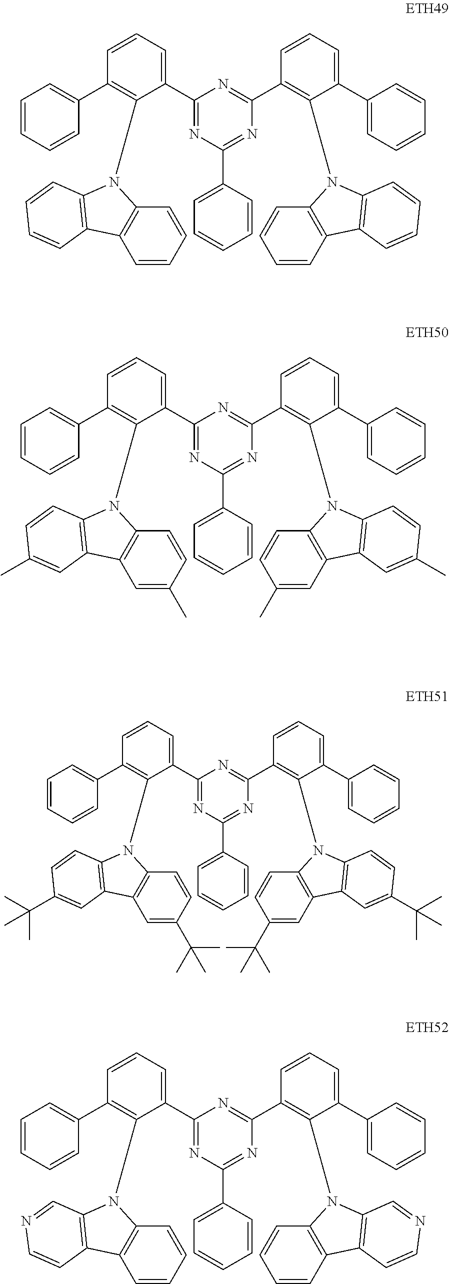

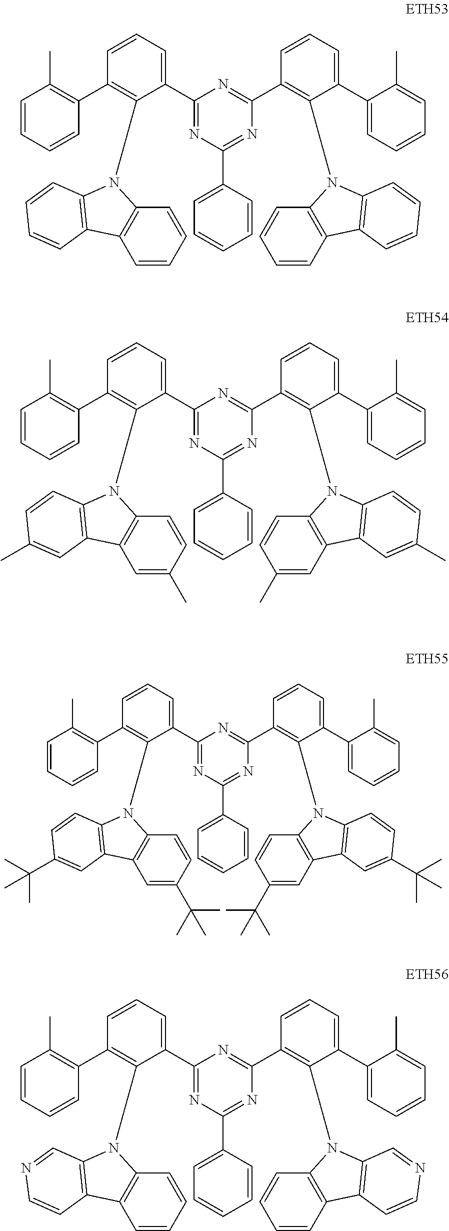

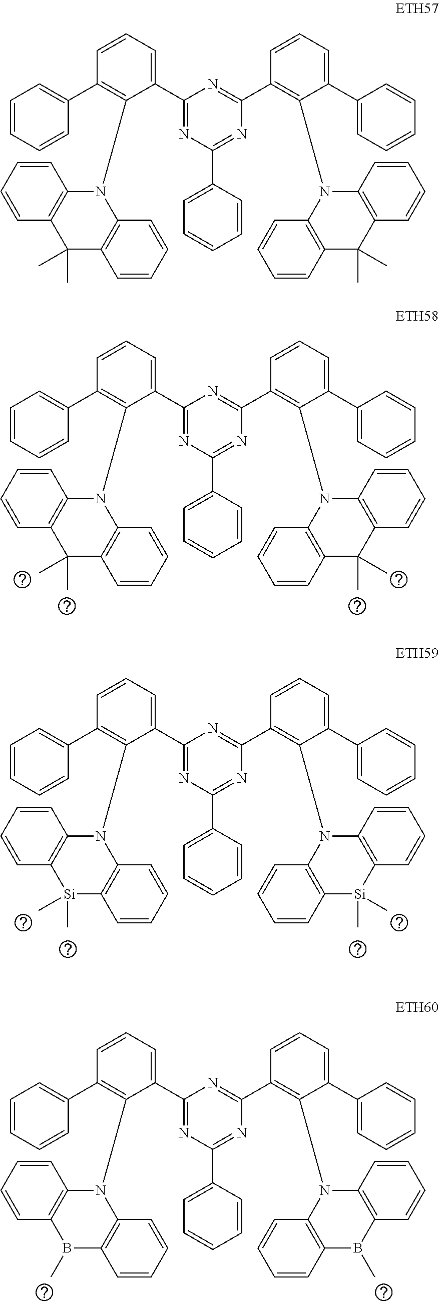 [SMILES: CC1(C)c2ccccc2N(c2c(-c3ccccc3)cccc2-c2nc(-c3ccccc3)nc(-c3cccc(-c4ccccc4)c3N3c4ccccc4C(C)(C)c4ccccc43)n2)c2ccccc21 ; CC1(C)c2ccccc2N(c2c(-c3ccccc3)cccc2-c2nc(-c3ccccc3)nc(-c3cccc(-c4ccccc4)c3N3c4ccccc4C(C)(C)c4ccccc43)n2)c2ccccc21 ; C[Si]1(C)c2ccccc2N(c2c(-c3ccccc3)cccc2-c2nc(-c3ccccc3)nc(-c3cccc(-c4ccccc4)c3N3c4ccccc4[Si](C)(C)c4ccccc43)n2)c2ccccc21 ; CB1c2ccccc2N(c2c(-c3ccccc3)cccc2-c2nc(-c3ccccc3)nc(-c3cccc(-c4ccccc4)c3N3c4ccccc4B(C)c4ccccc43)n2)c2ccccc21]

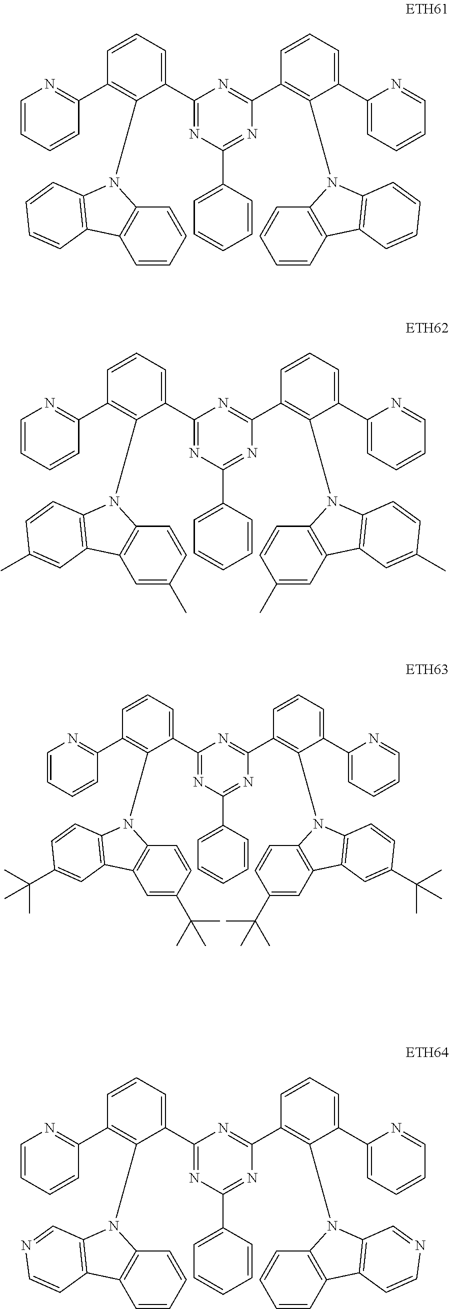

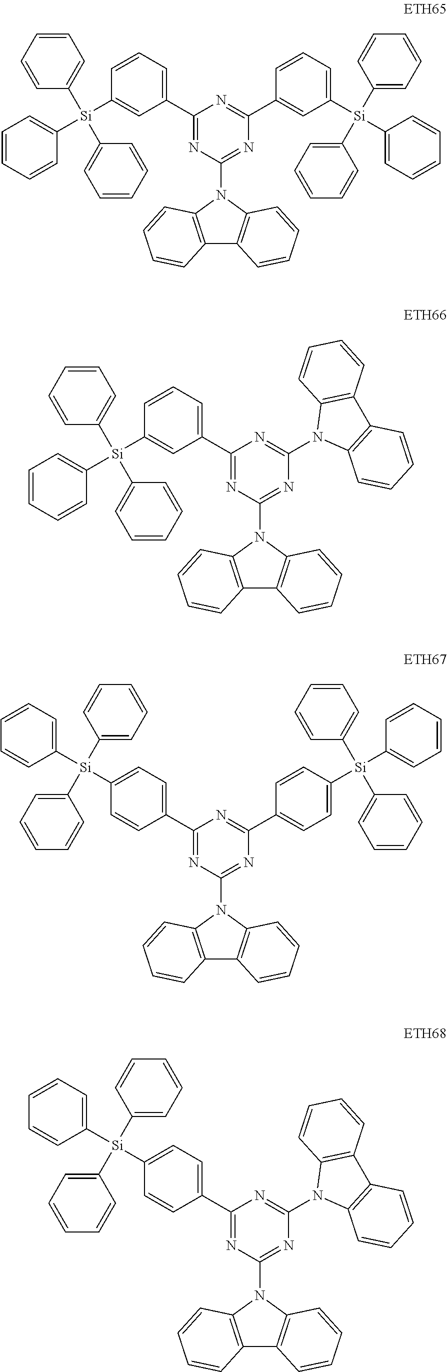 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5ccccc54)n3)c2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5ccccc54)n3)cc2)cc1 ; c1ccc([Si](c2ccccc2)(c2ccccc2)c2ccc(-c3nc(-n4c5ccccc5c5ccccc54)nc(-n4c5ccccc5c5ccccc54)n3)cc2)cc1]

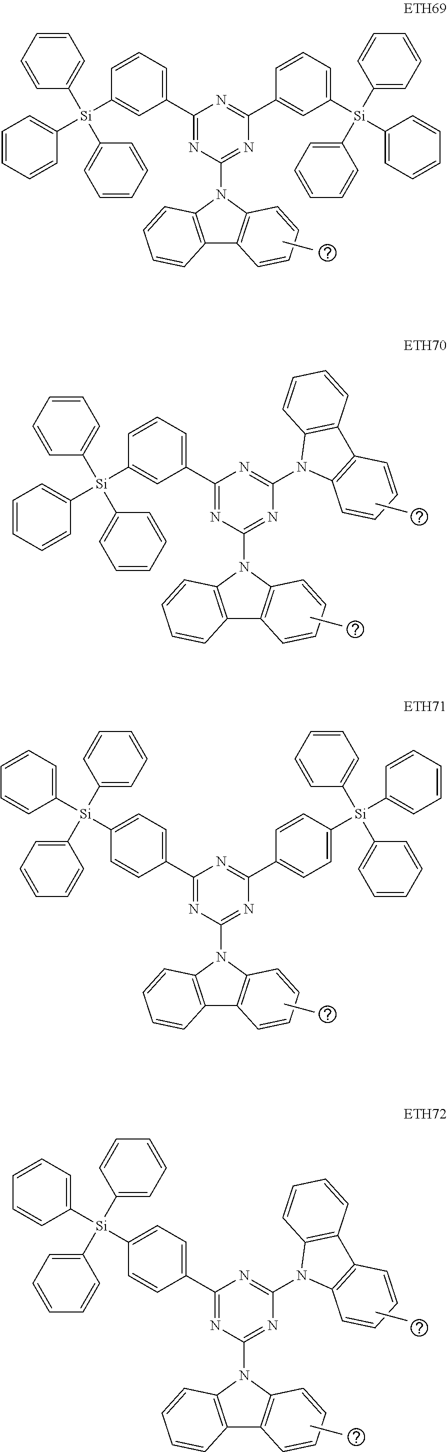

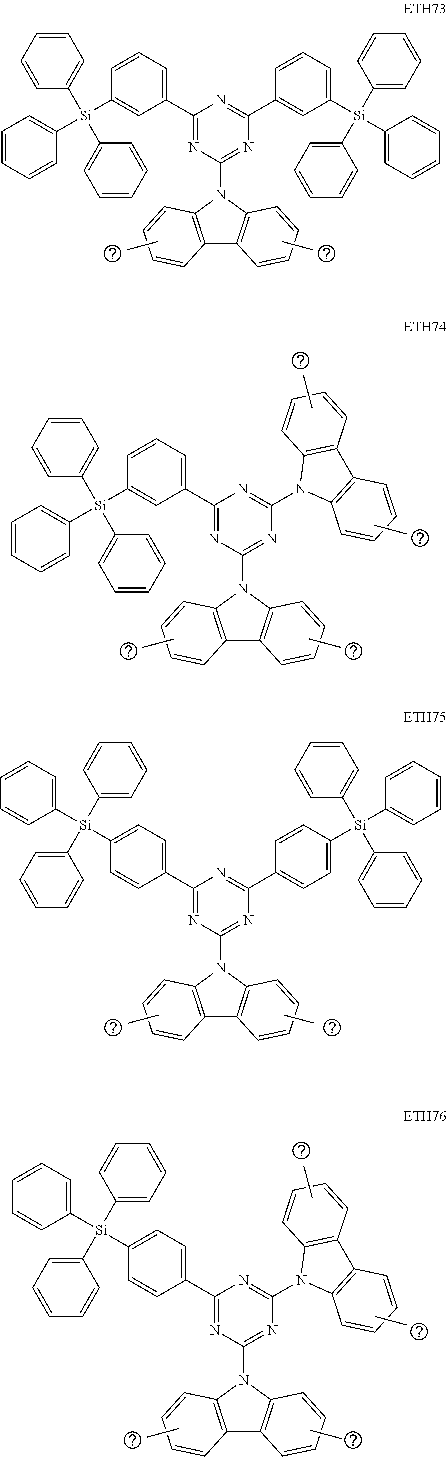

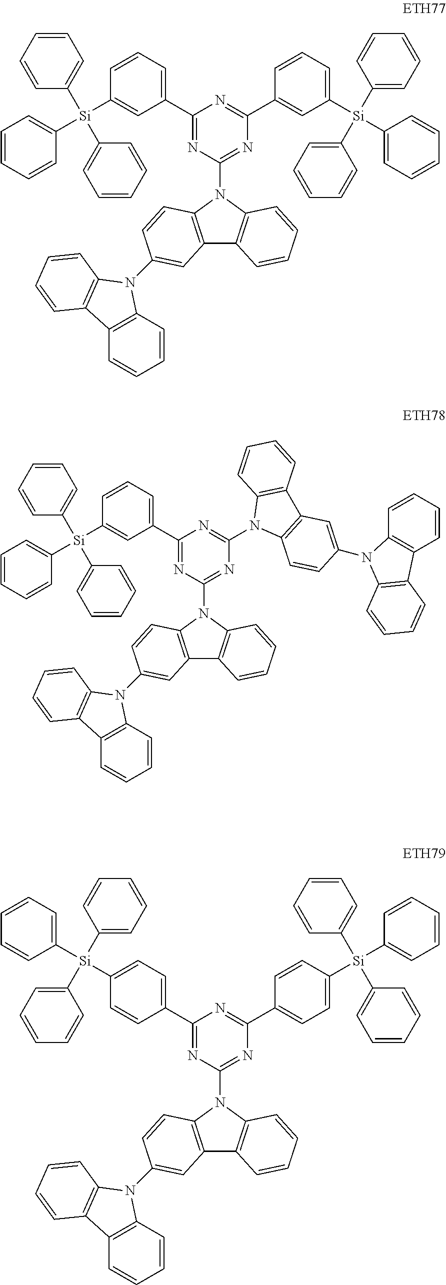 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5cc(-n6c7ccccc7c7ccccc76)ccc54)n3)c2)cc1 ; c1ccc([Si](c2ccccc2)(c2ccccc2)c2cccc(-c3nc(-n4c5ccccc5c5cc(-n6c7ccccc7c7ccccc76)ccc54)nc(-n4c5ccccc5c5cc(-n6c7ccccc7c7ccccc76)ccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5cc(-n6c7ccccc7c7ccccc76)ccc54)n3)cc2)cc1]

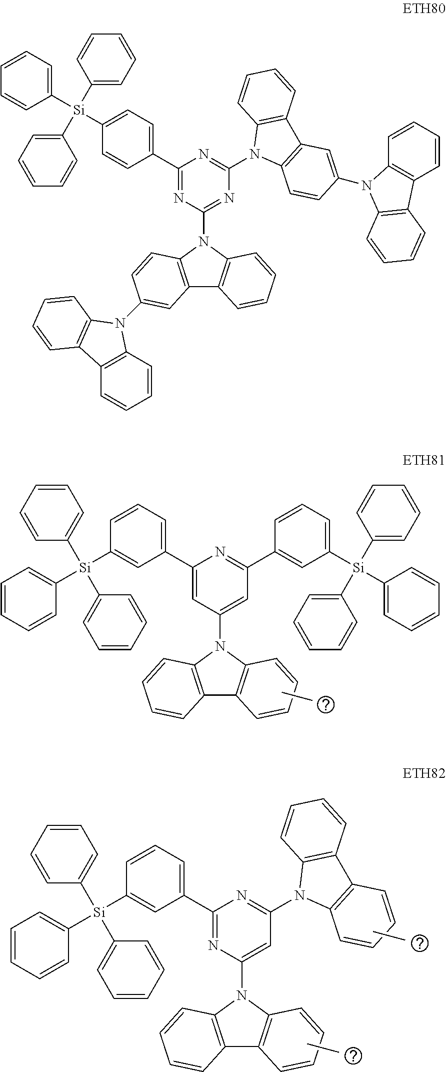

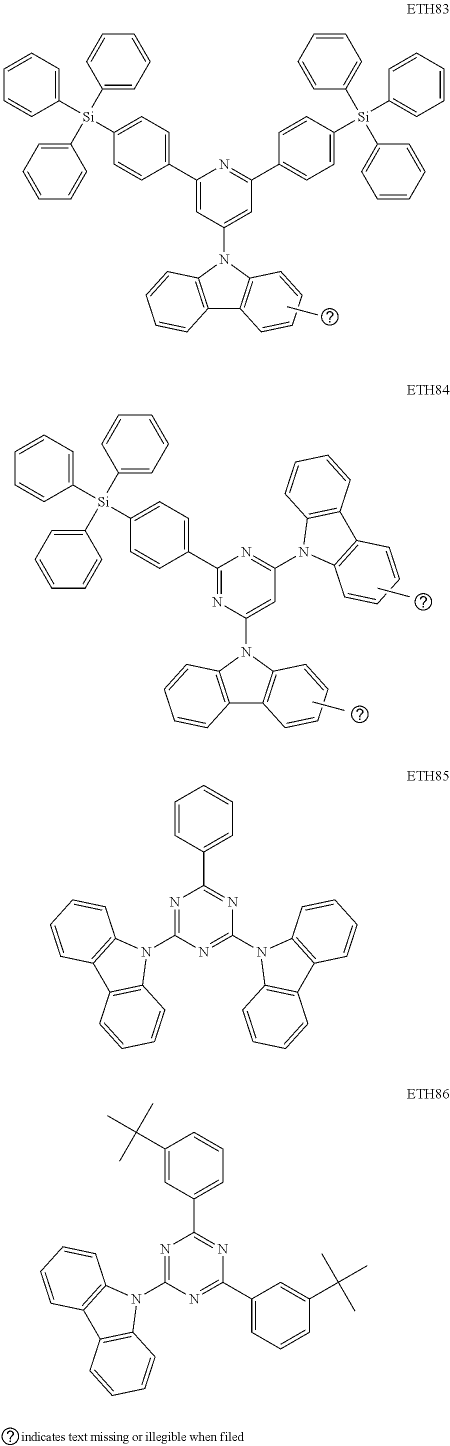

In Compound Group 3, D represents a deuterium atom, and Ph represents an unsubstituted phenyl group.

In an embodiment, the emission layer EML may include the second compound and the third compound, and the second compound and the third compound may form an exciplex. In the emission layer EML, an exciplex may be formed by a hole transport host and an electron transport host. A triplet energy level of the exciplex formed by a hole transport host and an electron transport host may correspond to a difference between a lowest unoccupied molecular orbital (LUMO) energy level of the electron transport host and a highest occupied molecular orbital (HOMO) energy level of the hole transport host.

For example, an absolute value of a triplet energy level (Ti) of the exciplex formed by the hole transport host and the electron transport host may be in a range of about 2.4 eV to about 3.0 eV. The triplet energy level of the exciplex may have a value that is smaller than an energy gap of each host material. The exciplex may have a triplet energy level equal to or less than about 3.0 eV, which is an energy gap between the hole transport host and the electron transport host.

In an embodiment, the emission layer EML may include the fourth compound, in addition to the first compound, the second compound, and the third compound. The fourth compound may be used as a phosphorescence sensitizer in the emission layer EML. Energy may be transferred from the fourth compound to the first compound, thereby emitting light.

The emission layer EML may include, as a fourth compound, an organometallic complex that includes platinum (Pt) as a central metal atom and ligands bonded to the central metal atom. In an embodiment, the emission layer EML may further include a fourth compound represented by Formula D-1. In the specification, the fourth compound may be referred to as an “organometallic compound.”

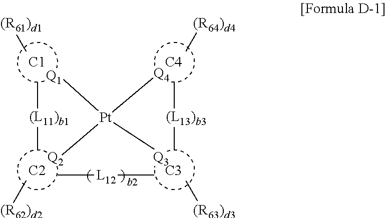

In Formula D-1, Q₁ to Q₄ may each independently be C or N.

In Formula D-1, C1 to C4 may each independently be a substituted or unsubstituted hydrocarbon ring of 5 to 30 ring-forming carbon atoms or a substituted or unsubstituted heterocycle of 2 to 30 ring-forming carbon atoms.

In Formula D-1, L₁₁ to L₁₃ may each independently be a direct linkage,

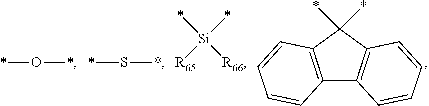

a substituted or unsubstituted alkylene group of 1 to 20 carbon atoms, a substituted or unsubstituted arylene group of 6 to 30 ring-forming carbon atoms, or a substituted or unsubstituted heteroarylene group of 2 to 30 ring-forming carbon atoms. In L₁₁ to L₁₃,

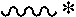 [SMILES: *C]

represents a bond to one of C1 to C4.

In Formula D-1, b1 to b3 may each independently be 0 or 1. If b1 is 0, C1 and C2 may not be directly connected to each other. If b2 is 0, C2 and C3 may not be directly connected to each other. If b3 is 0, C3 and C4 may not be directly connected to each other.

In Formula D-1, R₆₁ to R₆₆ may each independently be a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group of 1 to 20 carbon atoms, a substituted or unsubstituted alkenyl group of 2 to 20 ring-forming carbon atoms, a substituted or unsubstituted aryl group of 6 to 60 ring-forming carbon atoms, a substituted or unsubstituted heteroaryl group of 2 to 60 ring-forming carbon atoms, or bonded to an adjacent group to form a ring. For example, R₆₁ to R₆₆ may each independently be a substituted or unsubstituted methyl group or a substituted or unsubstituted t-butyl group.

In Formula D-1, d1 to d4 may each independently be an integer from 0 to 4. In Formula D-1, if d1 to d4 are each 0, the fourth compound may not be substituted with R₆₁ to R₆₄, respectively. A case where d1 to d4 are each 4 and all groups of each of R₆₁ to R₆₄ are hydrogen atoms may be the same as a case where d1 to d4 are each 0. If d1 to d4 are each 2 or more, multiple groups of each of R₆₁ to R₆₄ may all be the same, or at least one thereof may be different from the remainder.

In an embodiment, in Formula D-1, C1 to C4 may each independently be a substituted or unsubstituted hydrocarbon ring or a substituted or unsubstituted heterocycle that is represented by one of Formula C-1 to Formula C-4:

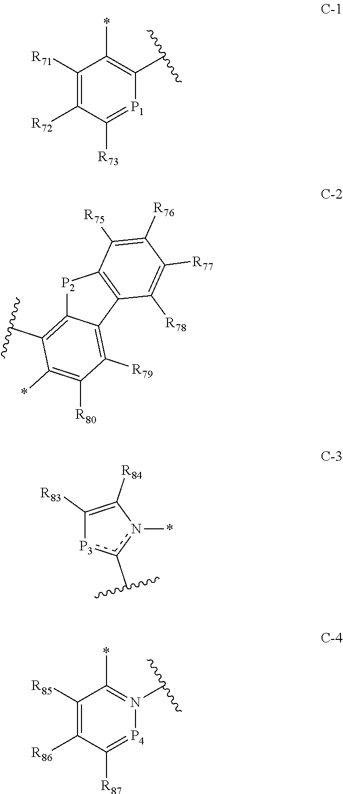

In Formula C-1 to Formula C-4, P₁ may be

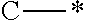 [SMILES: *C]

or C(R₇₄), P₂ may be

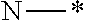 [SMILES: *N]

or N(R₈₁), P₃ may be

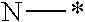 [SMILES: *N]

or N(R₈₂), and P₄ may be

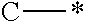 [SMILES: *C]

### or C(R₈₈).

In Formula C-1 to Formula C-4, R₇₁ to R₈₈ may each independently be a substituted or unsubstituted alkyl group of 1 to 20 carbon atoms, a substituted or unsubstituted aryl group of 6 to 30 ring-forming carbon atoms, a substituted or unsubstituted heteroaryl group of 2 to 30 ring-forming carbon atoms, or bonded to an adjacent group to form a ring.

In Formula C-1 to Formula C-4,

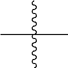

represents a bond to Pt, and

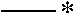 [SMILES: *C]

represents a bond to an adjacent ring group (C1 to C4) or to a linker (L₁₁ to L₁₃).

In an embodiment, the emission layer EML may include: the first compound, which is a fused polycyclic compound; and at least one of the second compound, the third compound, and the fourth compound. In an embodiment, the emission layer EML may include the first compound, the second compound, and the third compound. In the emission layer EML, the second compound and the third compound may form an exciplex, and energy may be transferred from the exciplex to the first compound, thereby emitting light.

In another embodiment, the emission layer EML may include the first compound, the second compound, the third compound, and the fourth compound. In the emission layer EML, the second compound and the third compound may form an exciplex, and energy may be transferred from the exciplex to the fourth compound and the first compound, thereby emitting light. In an embodiment, the fourth compound may be a sensitizer. In the light emitting element ED, the fourth compound included in the emission layer EML may serve as a sensitizer that transfers energy from the host to the first compound, which is a light emitting dopant. For example, the fourth compound, which serves as an auxiliary dopant, may accelerate energy transfer to the first compound, which serves as a light emitting dopant, thereby increasing an emission ratio of the first compound. Accordingly, the emission layer EML may exhibit improved emission efficiency. If energy transfer to the first compound increases, excitons formed in the emission layer EML may not accumulate and may rapidly emit light, so that deterioration of the light emitting element ED may be reduced. Accordingly, the lifetime of the light emitting element ED may increase.

The light emitting element ED may include the first compound, the second compound, the third compound and the fourth compound, and the emission layer EML may include a combination of two host materials and two dopant materials. In the light emitting element ED, the emission layer EML may include the second compound and the third compound, which are two different hosts, the first compound which emits delayed fluorescence, and the fourth compound which includes an organometallic complex, and thus the light emitting element ED may exhibit excellent emission efficiency properties.

In an embodiment, the fourth compound represented by Formula D-1 may be selected from Compound Group 4. In an embodiment, in the light emitting element ED, the fourth compound may include at least one compound selected from Compound Group 4:

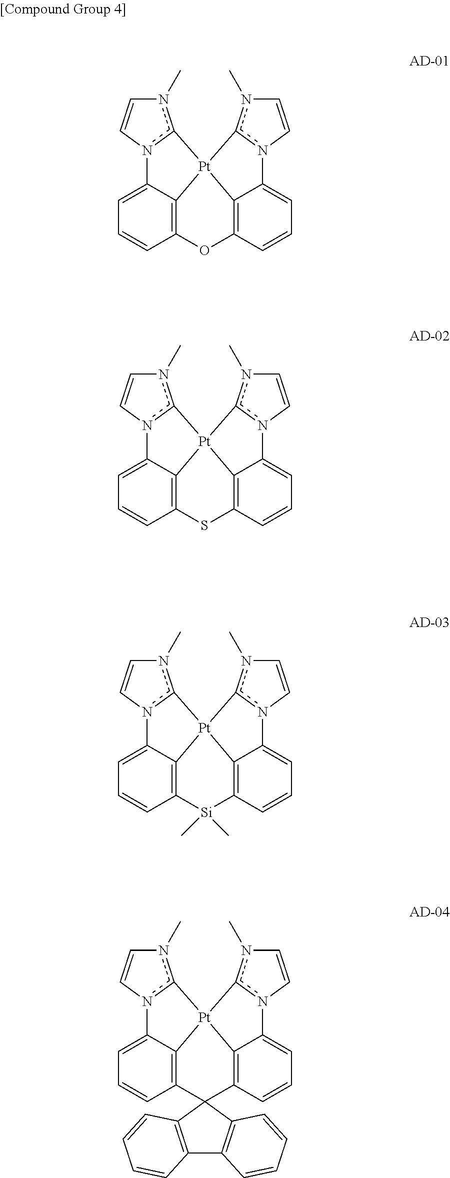

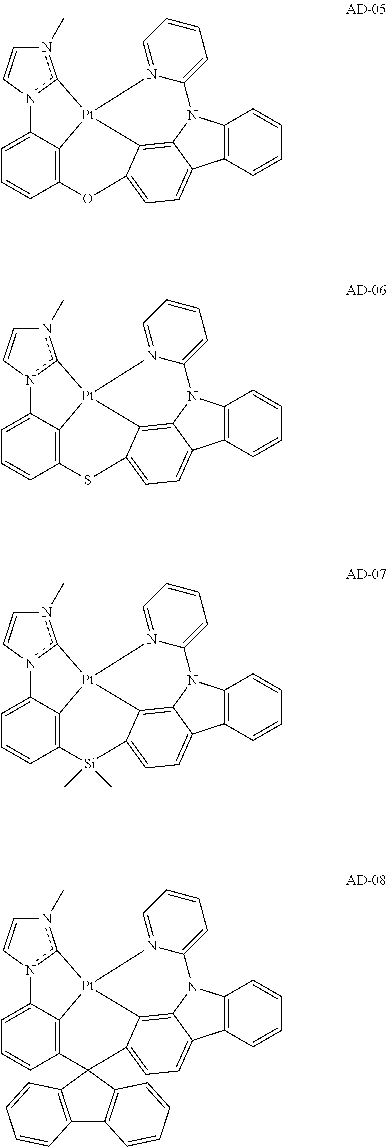

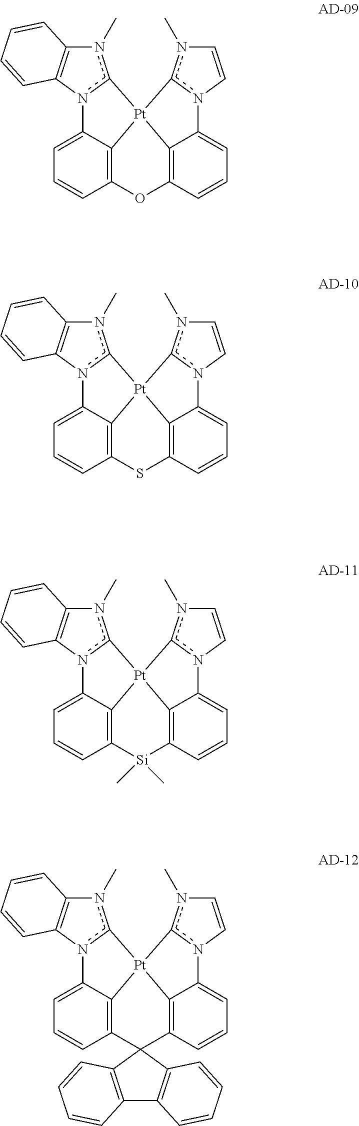

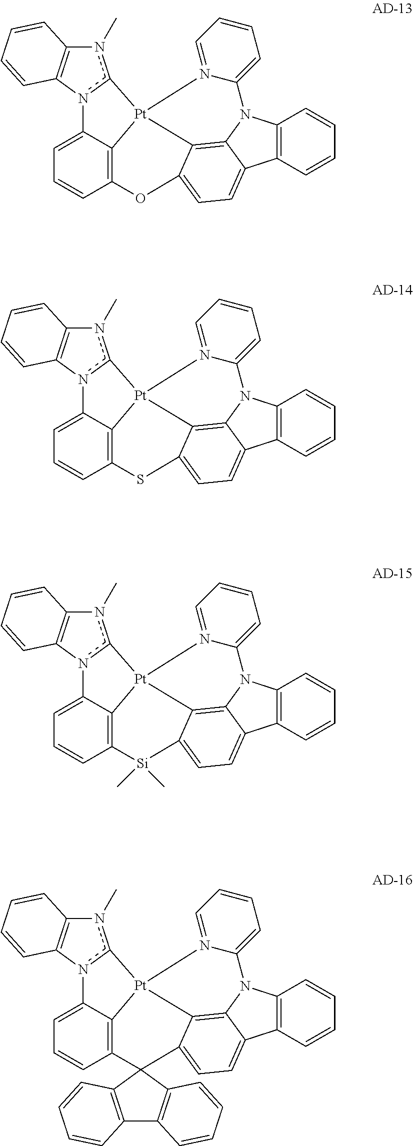

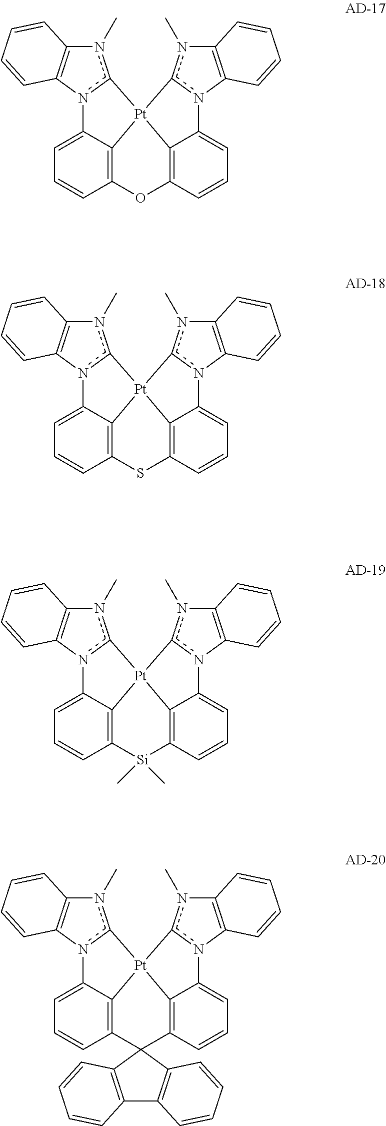

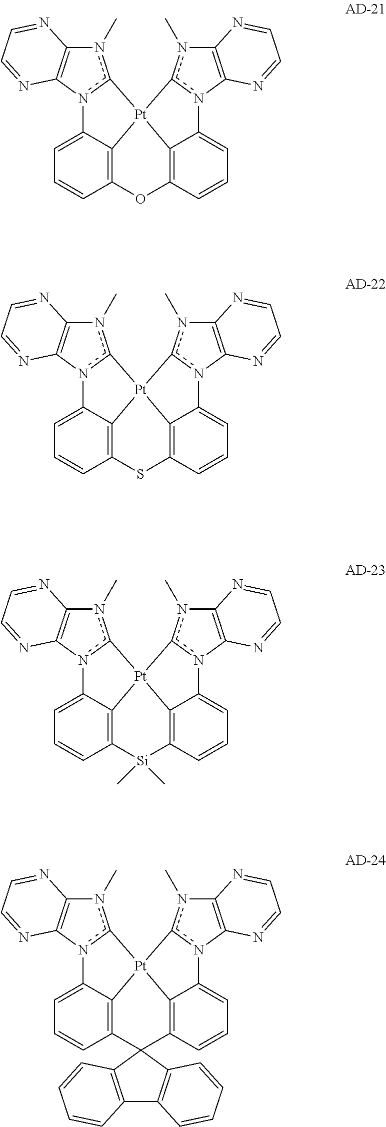

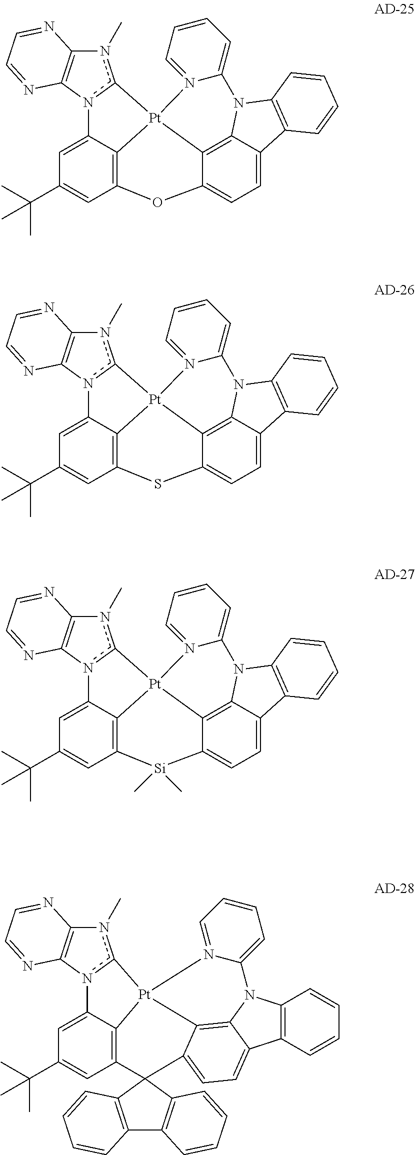

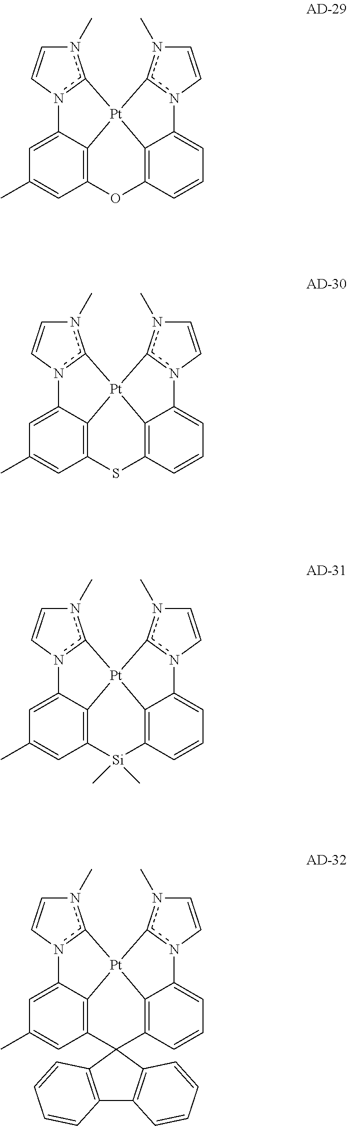

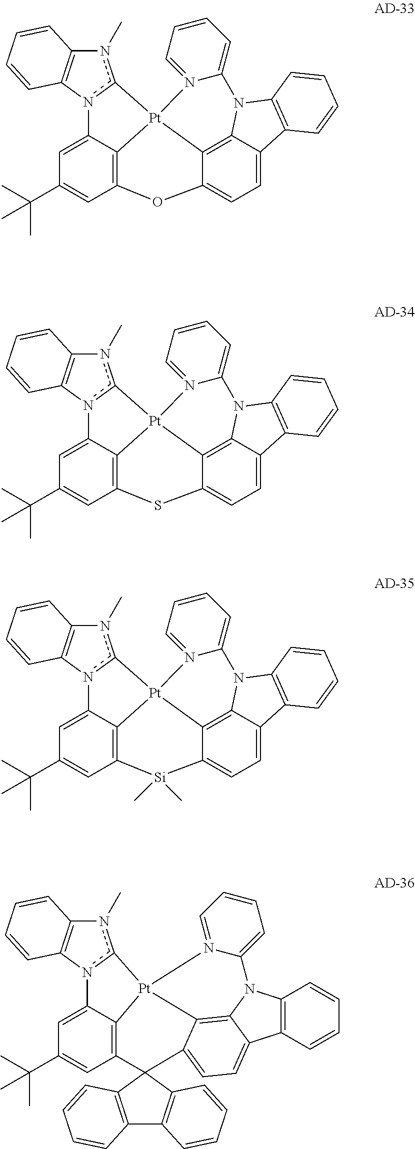

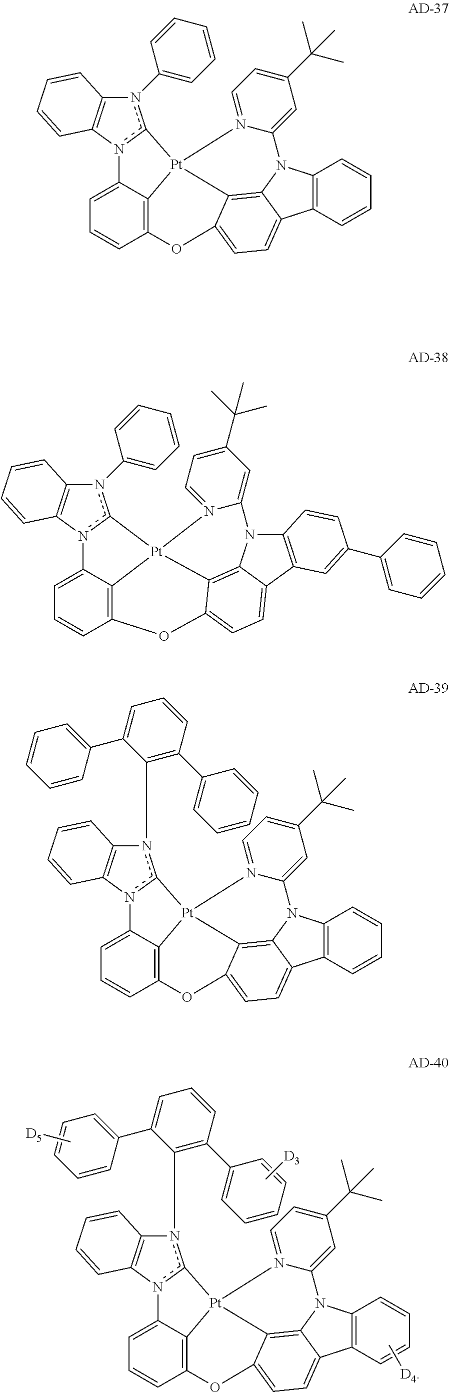

In Compound Group 4, D represents a deuterium atom.

In the light emitting element ED, if the emission layer EML includes the first compound, the second compound and the third compound, an amount of the first compound may be in a range of about 0.1 wt % to about 5 wt %, based on a total weight of the first compound, the second compound, and the third compound. However, embodiments are not limited thereto. If an amount of the first compound satisfies the above-described range, energy transfer from the second compound and the third compound to the first compound may increase, and accordingly, emission efficiency and element lifetime may increase.

In the emission layer EML, a total amount of the second compound and the third compound may be the remainder of the total weight of the first compound, the second compound, and the third compound, excluding the weight of the first compound. For example, in the emission layer EML, a combined amount of the second compound and the third compound may be in a range of about 65 wt % to about 99 wt %, based on a total weight of the first compound, the second compound, and the third compound.

Within the combined amount of the second compound and the third compound in the emission layer EML, a weight ratio of the second compound to the third compound may be in a range of about 3:7 to about 7:3.

If the amounts of the second compound and the third compound satisfy the above-described ranges and ratios, charge balance properties in the emission layer EML may be improved, and emission efficiency and element lifetime may increase. If the amounts of the second compound and the third compound deviate from the above-described ranges and ratios, charge balance in the emission layer EML may not be achieved, so that emission efficiency may be reduced, and the element may readily deteriorate.

If the emission layer EML includes the fourth compound, an amount of the fourth compound in the emission layer EML may be in a range of about 4 wt % to about 30 wt %, based on a total weight of the first compound, the second compound, the third compound, and the fourth compound. However, embodiments are not limited thereto. If an amount of the fourth compound satisfies the above-described range, energy transfer from a host to the first compound, which is a light emitting dopant, may increase so that an emission ratio may increase. Accordingly, emission efficiency of the emission layer EML may be improved. If the amounts of the first compound, the second compound, the third compound, and the fourth compound included in the emission layer EML satisfy the above-described ranges and ratios, excellent emission efficiency and long lifetime may be achieved.

In an embodiment, the emission layer EML may include a compound represented by Formula E-2a or Formula E-2b. The compound represented by Formula E-2a or Formula E-2b may be used as a phosphorescence host material.

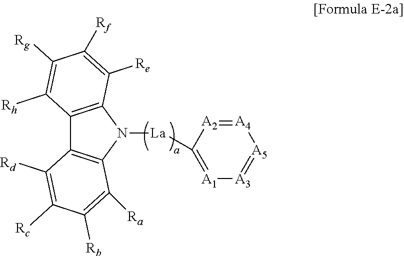

In Formula E-2a, a may be an integer from 0 to 10; and Lₐ may be a direct linkage, a substituted or unsubstituted arylene group of 6 to 30 ring-forming carbon atoms, or a substituted or unsubstituted heteroarylene group of 2 to 30 ring-forming carbon atoms. If a is 2 or more, multiple Lₐ groups may each independently be a substituted or unsubstituted arylene group of 6 to 30 ring-forming carbon atoms or a substituted or unsubstituted heteroarylene group of 2 to 30 ring-forming carbon atoms.

In Formula E-2a, A₁ to A₅ may each independently be N or C(Rᵢ). In Formula E-2a, Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, a substituted or unsubstituted heteroaryl group of 2 to 30 ring-forming carbon atoms, or bonded to an adjacent group to form a ring. For example, each of Rₐ to Rᵢ may be combined with an adjacent group to form a hydrocarbon ring or a heterocycle including N, O, S, etc. as a ring-forming atom.

In Formula E-2a, two or three of A₁ to A₅ may each be N, and the remainder of A₁ to A₅ may each independently be C(Rᵢ).

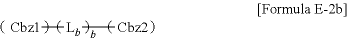

In Formula E-2b, Cbz1 and Cbz2 may each independently be an unsubstituted carbazole group or a carbazole group substituted with an aryl group of 6 to 30 ring-forming carbon atoms. In Formula E-2b, L_{b} may be a direct linkage, a substituted or unsubstituted arylene group of 6 to 30 ring-forming carbon atoms, or a substituted or unsubstituted heteroarylene group of 2 to 30 ring-forming carbon atoms. In Formula E-2b, b may be an integer from 0 to 10. If b is 2 or more, multiple L_{b} groups may each independently be a substituted or unsubstituted arylene group of 6 to 30 ring-forming carbon atoms or a substituted or unsubstituted heteroarylene group of 2 to 30 ring-forming carbon atoms.

The compound represented by Formula E-2a or Formula E-2b may be any compound selected from Compound Group E-2. However, the compounds listed in Compound Group E-2 are only examples, and the compound represented by Formula E-2a or Formula E-2b is not limited to Compound Group E-2:

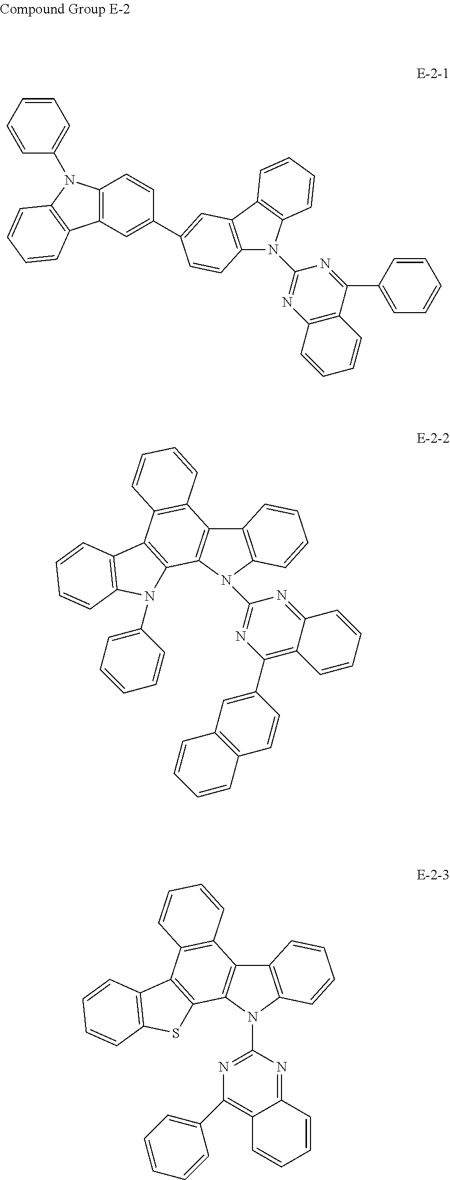 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1]

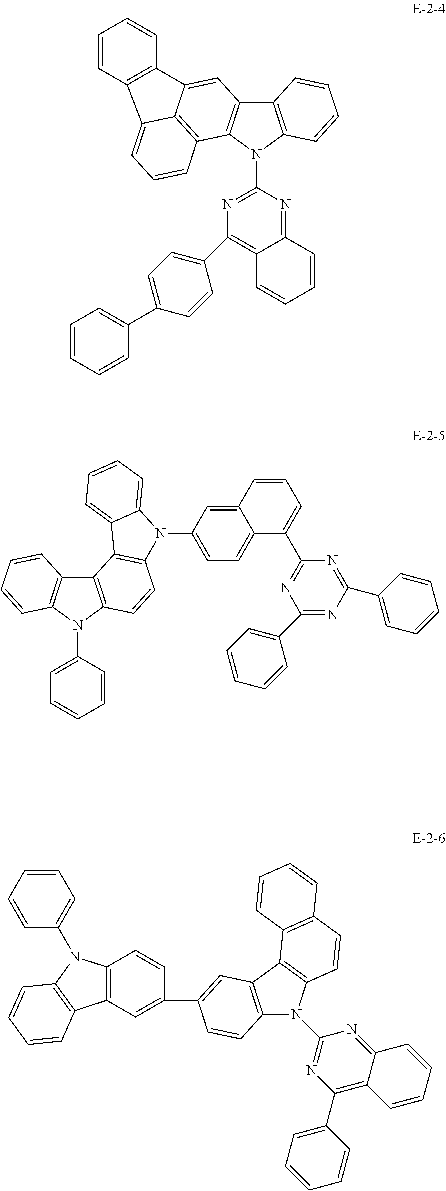 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

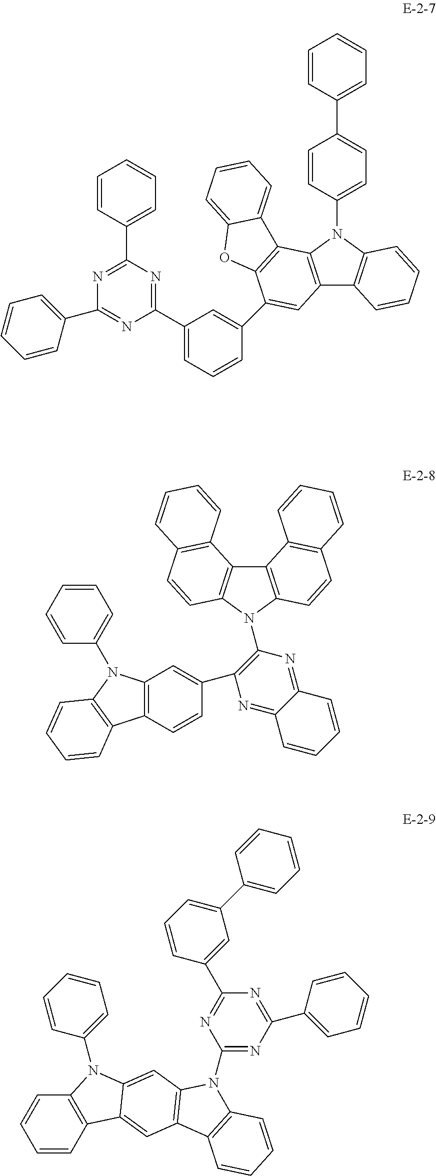 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1]

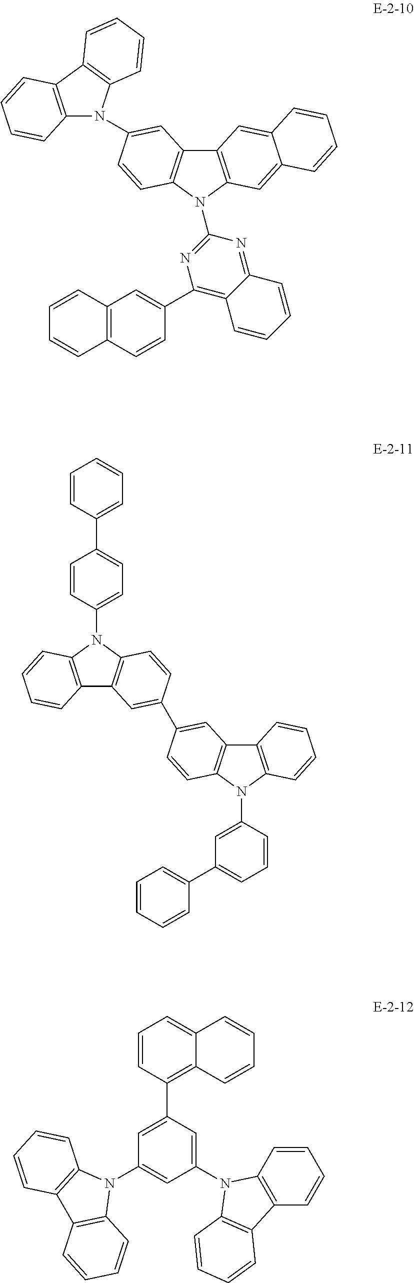 [SMILES: c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1]

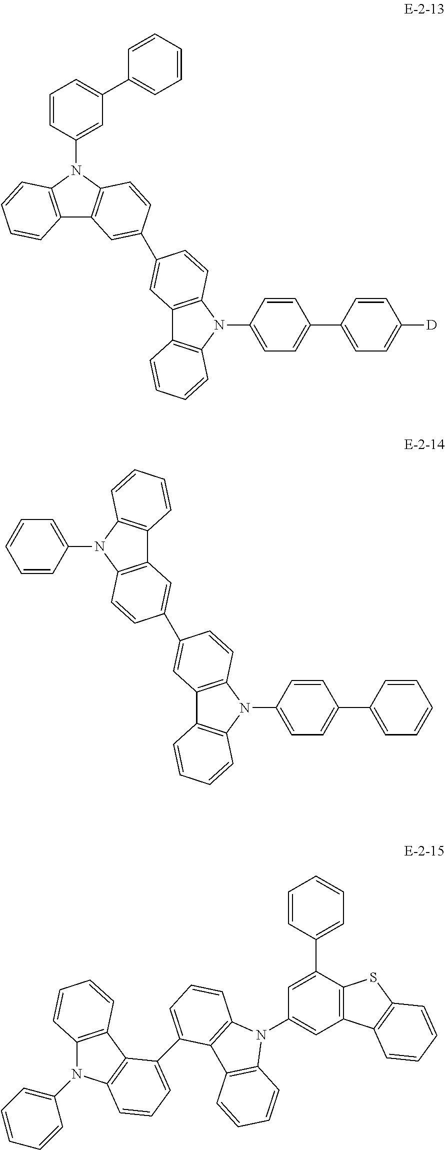 [SMILES: [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

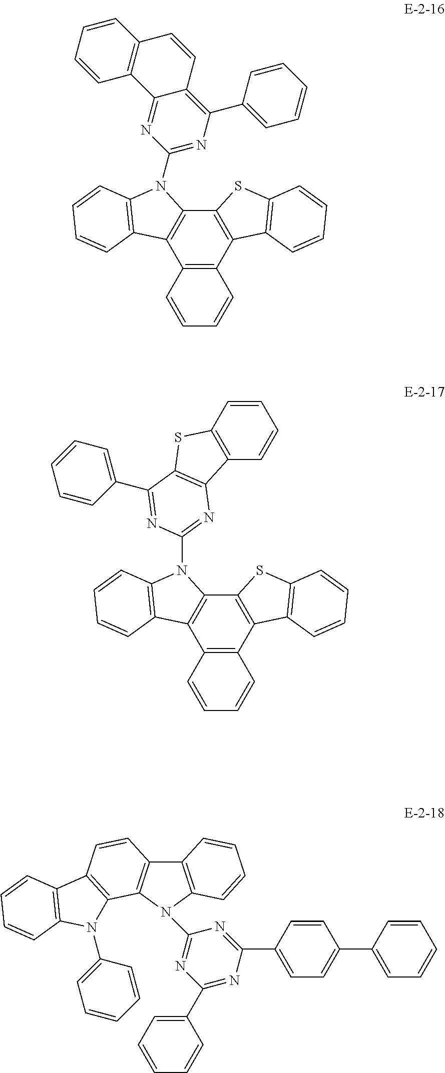 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1]

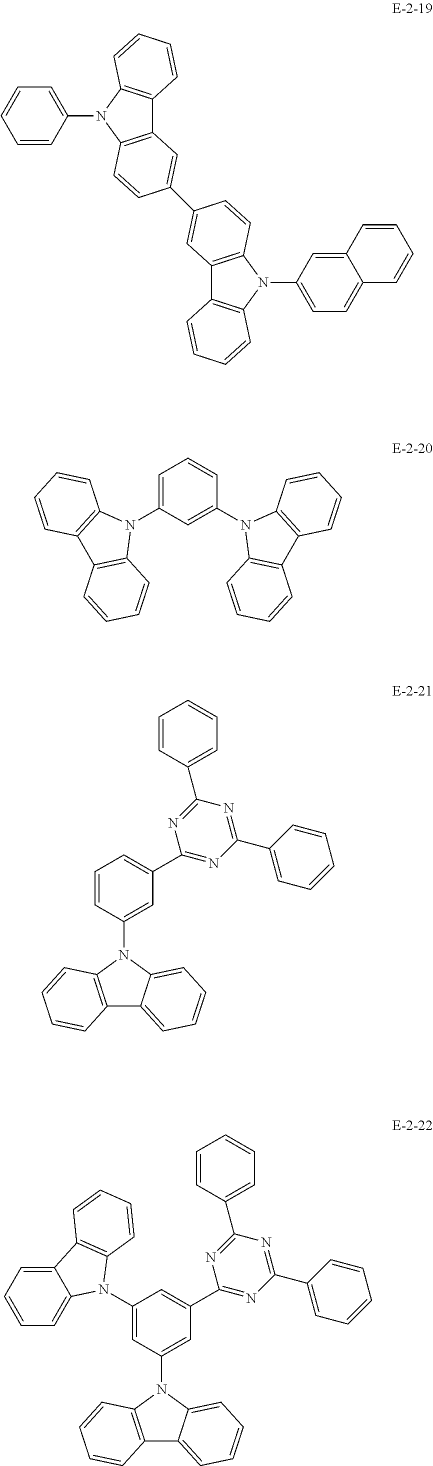 [SMILES: c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1]

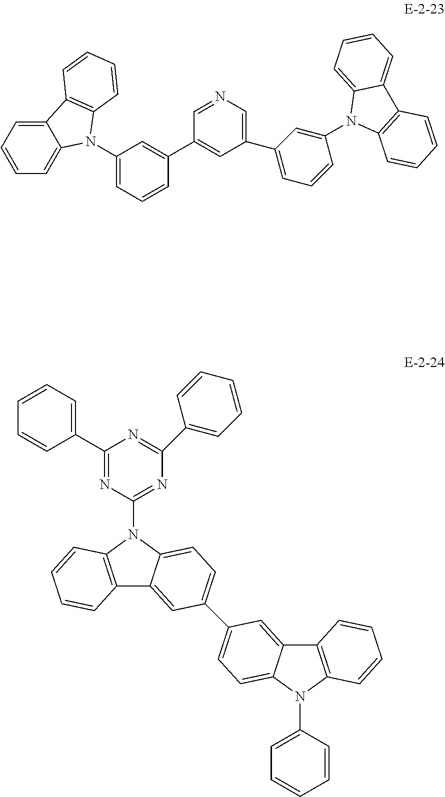 [SMILES: c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a material of the related art as a host material. For example, the emission layer EML may include as a host material, at least one of bis (4-(9H-carbazol-9-yl) phenyl) diphenylsilane (BCPDS), (4-(1-(4-(diphenylamino) phenyl) cyclohexyl) phenyl) diphenyl-phosphine oxide (POPCPA), bis [2-(diphenylphosphino) phenyl] ether oxide (DPEPO), 4,4′-bis(N-carbazolyl)-1,1′-biphenyl (CBP), 1,3-bis (carbazol-9-yl)benzene (mCP), 2,8-bis(diphenylphosphoryl)dibenzo[b,d]furan(PPF), 4,4′,4″-tris(carbazol-9-yl)-triphenylamine (TCTA), and 1,3,5-tris(1-phenyl-1H-benzo[d]imidazole-2-yl)benzene (TPBi). However, embodiments are not limited thereto. For example, tris(8-hydroxyquinolino)aluminum (Alq₃), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may be used as a host material.

In an embodiment, the emission layer EML may include a compound represented by Formula M-a. The compound represented by Formula M-a may be used as a phosphorescence dopant material.

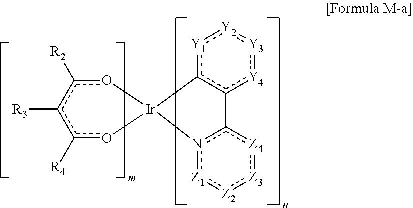

In Formula M-a, Y₁ to Y₄ and Z₁ to Z₄ may each independently be C(R₁) or N; and R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, a substituted or unsubstituted heteroaryl group of 2 to 30 ring-forming carbon atoms, or bonded to an adjacent group to form a ring. In Formula M-a, m may be 0 or 1, and n may be 2 or 3. In Formula M-a, if m is 0, n may be 3, and if m is 1, n may be 2.

The compound represented by Formula M-a may be any compound selected from Compounds M-a1 to M-a25. However, Compounds M-a1 to M-a25 are only examples, and the compound represented by Formula M-a is not limited to Compounds M-a1 to M-a25:

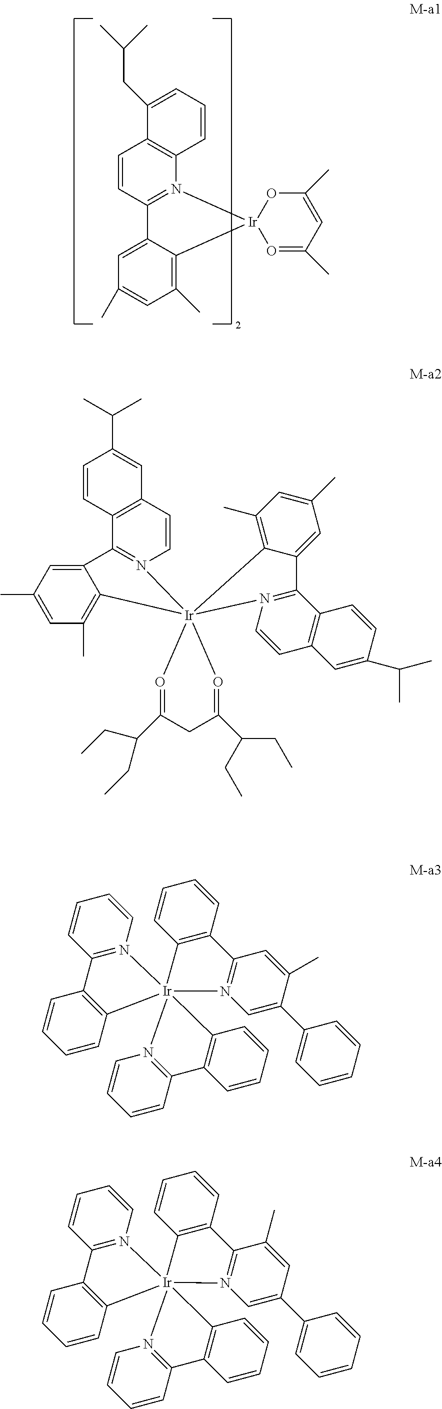

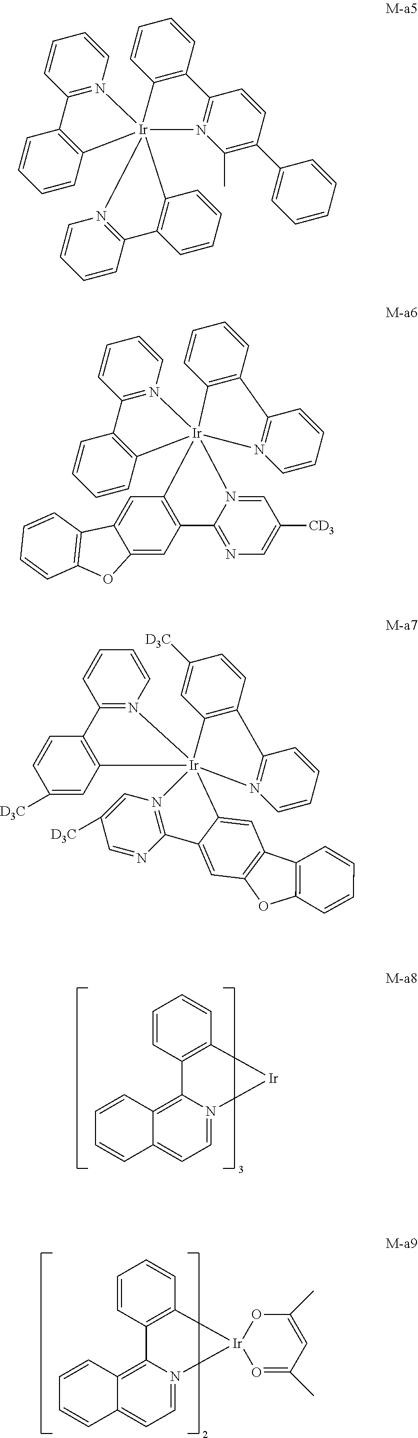

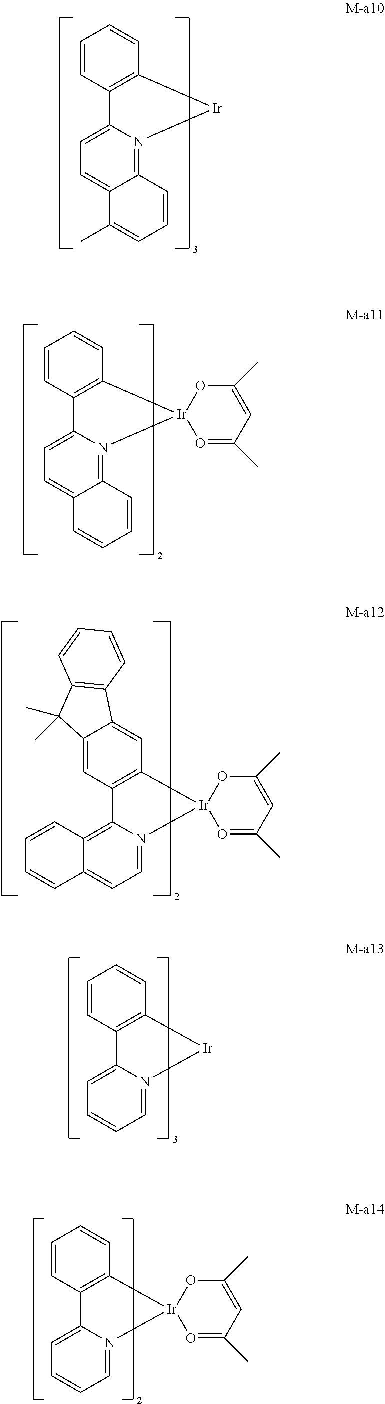

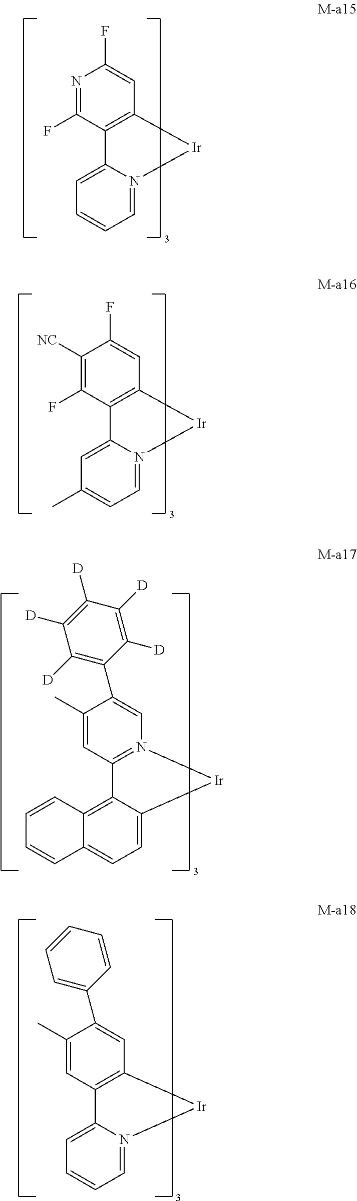

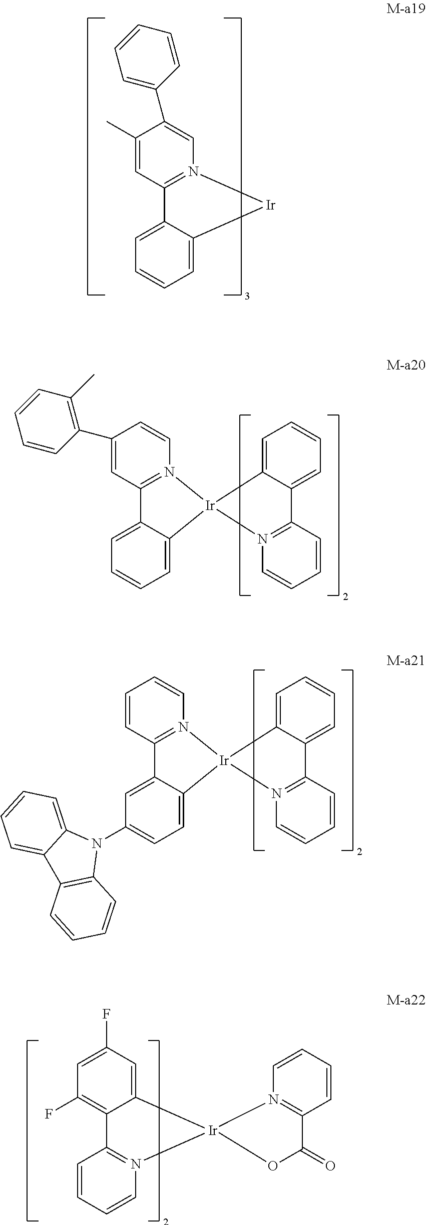

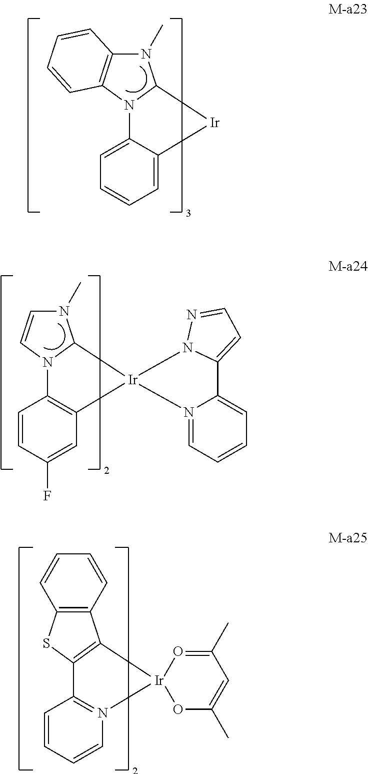

In an embodiment, the emission layer EML may include, as a dopant material of the related art, a styryl derivative (for example, 1,4-bis[2-(3-N-ethylcarbazoryl)vinyl] benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), and 4,4′-bis[2-(4-(N,N-diphenylamino)phenyl)vinyl]biphenyl (DPAVBi)), perylene or a derivative thereof (for example, 2,5,8,11-tetra-t-butylperylene (TBP)), pyrene or a derivative thereof (for example, 1,1-dipyrene, 1,4-dipyrenylbenzene, and 1,4-bis(N,N-diphenylamino)pyrene), etc.

The emission layer EML may include a phosphorescence dopant material of the related art. For example, the phosphorescence dopant may use a metal complex including iridium (Ir), platinum (Pt), osmium (Os), gold (Au), titanium (Ti), zirconium (Zr), hafnium (Hf), europium (Eu), terbium (Tb), or thulium (Tm). For example, iridium(III) bis(4,6-difluorophenylpyridinato-N,C2′)picolinate (FIrpic), bis(2,4-difluorophenylpyridinato)-tetrakis(1-pyrazolyl)borate iridium(III) (Fir6), or platinum octaethyl porphyrin (PtOEP) may be used as a phosphorescence dopant. However, embodiments are not limited thereto.

In an embodiment, the emission layer EML may include a quantum dot material. The quantum dot may be a Group II-VI compound, a Group III-VI compound, a Group 1-II-VI compound, a Group III-V compound, a Group III-II-V compound, a Group IV-VI compound, a Group IV group element, a Group IV compound, or any combination thereof.

Examples of a Group II-VI compound may include: a binary compound selected from the group consisting of CdSe, CdTe, CdS, ZnS, ZnSe, ZnTe, ZnO, HgS, HgSe, HgTe, MgSe, MgS, and mixtures thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and mixtures thereof; a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, HgZnSTe, and mixtures thereof; or any combination thereof.

Examples of a Group III-VI compound may include: a binary compound such as In₂S₃, and In₂Se₃; a ternary compound such as InGaS₃, and InGaSe₃, or any combination thereof.

Examples of a Group 1-III-VI compound may include: a ternary compound selected from the group consisting of AgInS, AgInS₂, CuInS, CuInS₂, AgGaS₂, CuGaS₂, CuGaO₂, AgGaO₂, AgAlO₂ and mixtures thereof; a quaternary compound such as AgInGaS₂, and CuInGaS₂; or any combination thereof.

Examples of a Group III-V compound may include: a binary compound selected from the group consisting of GaN, GaP, GaAs, GaSb, AlN, AlP, AlAs, AlSb, InN, InP, InAs, InSb, and mixtures thereof; a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and mixtures thereof; a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and mixtures thereof; or any combination thereof. In an embodiment, a Group III-V compound may further include a Group II metal. Examples of a Group III-II-V compound may include InZnP, etc.

Examples of a Group IV-VI compound may include: a binary compound selected from the group consisting of SnS, SnSe, SnTe, PbS, PbSe, PbTe, and mixtures thereof; a ternary compound selected from the group consisting of SnSeS, SnSeTe, SnSTe, PbSeS, PbSeTe, PbSTe, SnPbS, SnPbSe, SnPbTe, and mixtures thereof; a quaternary compound selected from the group consisting of SnPbSSe, SnPbSeTe, SnPbSTe, and mixtures thereof; or any combination thereof.

Examples of a Group IV element may include Si, Ge, and a mixture thereof. Examples of a Group IV compound may include a binary compound selected from the group consisting of SiC, SiGe, and a mixture thereof.

Each element included in a polynary compound, such as a binary compound, a ternary compound, or a quaternary compound, may be present in a particle at uniform concentration or at a non-uniform concentration. For example, a chemical formula may indicate the elements that are included in a compound, but a ratio of elements in the compound may vary. For example, AgInGaS₂ may mean AgInₓGa₁₋ₓS₂ (where x is a real number between 0 and 1).

In embodiments, a quantum dot may have a single structure in which the concentration of each element included in the quantum dot is uniform, or a quantum dot may have a core-shell structure. For example, a material included in the core and a material included in the shell may be different from each other.

The shell of the quantum dot may serve as a protection layer for preventing the chemical deformation of the core to maintain semiconductor properties and/or may serve as a charging layer for imparting the quantum dot with electrophoretic properties. The shell may be a single layer or a multilayer. An interface between the core and the shell may have a concentration gradient in which the concentration of an element that is present in the shell decreases toward the core.

In embodiments, the quantum dot may have the above-described core-shell structure including a core that includes a nanocrystal and a shell surrounding the core. Examples of a shell of a quantum dot may include a metal oxide, a non-metal oxide, a semiconductor compound, or any combination thereof.

Examples of a metal oxide or a non-metal oxide may include: a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄ and NiO; or a ternary compound such as MgAl₂O₄, CoFe₂O₄, NiFe₂O₄ and CoMn₂O₄. However, embodiments are not limited thereto.

Examples of a semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but embodiments are not limited thereto.

The quantum dot may have a full width at half maximum (FWHM) of an emission spectrum equal to or less than about 45 nm. For example, the quantum dot may have a FWHM of an emission spectrum equal to or less than about 40 nm. For example, the quantum dot may have a FWHM of an emission spectrum equal to or less than about 30 nm. Within any of the above ranges, color purity or color reproducibility may be improved. Light emitted through a quantum dot may be emitted in all directions, so that light viewing angle properties may be improved.

The shape of a quantum dot may be any shape that is used in the related art. For example, a quantum dot may have a spherical shape, a pyramidal shape, a multi-arm shape, or a cubic shape, or the quantum dot may be in the form of a nanoparticle, a nanotube, a nanowire, a nanofiber, a nanoplate particle, etc.

By controlling the size of a quantum dot or by controlling the ratio of elements in a quantum dot, an energy band gap may be controlled, and various wavelength bands of light may be obtained from a quantum dot emission layer. Accordingly, by using quantum dots (for example, using quantum dots having different sizes or controlling ratio of elements in a quantum dot compound), a light emitting element that emits various wavelengths of light may be achieved. For example, the size of the quantum dot or the ratio of elements in a quantum dot compound may be adjusted to emit red light, green light, and/or blue light. For example, the quantum dots may be configured to emit white light by combining light of various colors.

In the light emitting elements ED according to an embodiment as shown in each of FIGS. 3 to 6, the electron transport region ETR may be provided on the emission layer EML. The electron transport region ETR may include at least one of a hole blocking layer HBL, an electron transport layer ETL, and an electron injection layer EIL, but embodiments are not limited thereto.

The electron transport region ETR may be a layer consisting of a single material, a layer including different materials, or a structure including multiple layers including different materials.

For example, the electron transport region ETR may have a single layer structure of the electron injection layer EIL or the electron transport layer ETL, or may have a single layer structure formed of an electron injection material and an electron transport material. In other embodiments, the electron transport region ETR may have a single-layered structure formed of different materials, or may have a structure in which an electron transport layer ETL/electron injection layer EIL, or a hole blocking layer HBL/electron transport layer ETL/electron injection layer EIL are stacked in its respective stated order from the emission layer EML, but embodiments are not limited thereto. The electron transport region ETR may have a thickness, for example, in a range of about 1,000 Å to about 1,500 Å.

The electron transport region ETR may be formed by using various methods such as a vacuum deposition method, a spin coating method, a cast method, a Langmuir-Blodgett (LB) method, an inkjet printing method, a laser printing method, and a laser induced thermal imaging (LITI) method.

The light emitting element ED according to an embodiment may include a nitrogen-containing compound represented by Formula 1 in at least one functional layer disposed between the first electrode EL1 and the second electrode EL2. In the light emitting element ED, the electron transport region ETR may include a nitrogen-containing compound according to an embodiment. In an embodiment, the electron transport region ETR may include an electron transport layer ETL, and the electron transport layer ETL may include the nitrogen-containing compound according to an embodiment.

The nitrogen-containing compound according to an embodiment includes a triazine ring, and a first substituent and a second substituent that are each linked to a carbon atom of the triazine ring. The first substituent and the second substituent are each linked to a carbon atom of the triazine ring via an arylene linker or a heteroarylene linker, or are directly linked to a carbon atom of the triazine ring without a linker.

The first substituent may include a naphthalene moiety, and a benzene moiety that is linked to the naphthalene moiety. In the first substituent, a carbon at position-2 of the naphthalene moiety may be linked to the triazine ring, and a carbon at position-1 or a carbon at position-3 may be linked to the benzene moiety. For example, in the naphthalene moiety of the first substituent, the benzene moiety and the triazine ring may be linked at an ortho-position to each other. The numbered positions of carbon atoms constituting the naphthalene moiety of the first substituent are represented by Formula Si:

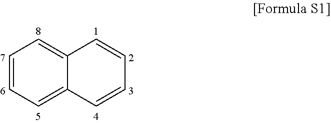

The second substituent includes a fluorene moiety. The fluorene moiety of the second substituent may be a structure substituted with a methyl group or a phenyl group. In an embodiment, the fluorene moiety of the second substituent may be a structure substituted with two phenyl groups, and the two substituted phenyl groups may be directly bonded to each other to form a spiro structure. Examples of substituted fluorene groups are as follows. However, embodiments are not limited thereto.

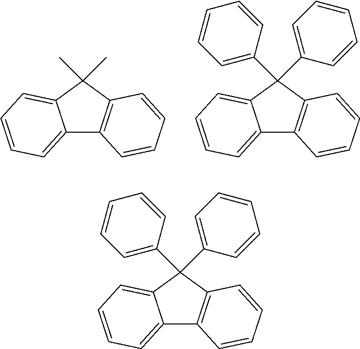

In the second substituent, a carbon atom of an aromatic ring in the fluorene moiety may be linked to the triazine ring. In an embodiment, when the second substituent includes a fluorene moiety substituted with a phenyl group, an aromatic ring of the phenyl group may be linked to the triazine ring.

The nitrogen-containing compound according to an embodiment may have excellent electrical stability and high charge transport ability, due to the introduction of the first and second substituents. Accordingly, the light emitting element according to an embodiment including the nitrogen-containing compound may have improved luminous efficiency and improved service life.

The nitrogen-containing compound according to an embodiment may be represented by Formula 1:

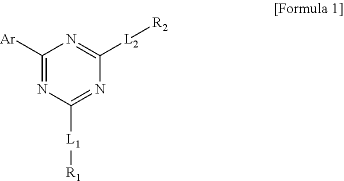

In Formula 1, Ar may be a substituted or unsubstituted aryl group having 6 to 30 ring-forming carbon atoms or a substituted or unsubstituted heteroaryl group having 2 to 30 ring-forming carbon atoms. In an embodiment, Ar may be a substituted or unsubstituted phenyl group, a substituted or unsubstituted biphenyl group, a substituted or unsubstituted terphenyl group, or a substituted or unsubstituted naphthyl group.

In Formula 1, L₁ and L₂ may each independently be a direct linkage or a substituted or unsubstituted aryl group having 6 to 30 ring-forming carbon atoms. In an embodiment, L₁ may be a direct linkage or a substituted or unsubstituted phenyl group, and L₂ may be a direct linkage.

In Formula 1, R₁ may be a group represented by Formula 2, and R₂ may be a group represented by Formula 3-1, Formula 3-2, or Formula 3-3.

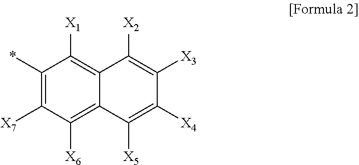

In Formula 2, X₁ to X₇ may each independently be a hydrogen atom, a deuterium atom, a halogen atom, a cyano group, a hydroxy group, a substituted or unsubstituted oxy group, a substituted or unsubstituted thio group, a substituted or unsubstituted boron group, a substituted or unsubstituted silyl group, a substituted or unsubstituted amine group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, except that X₁ or X₇ may be a substituted or unsubstituted phenyl group. For example, X₁ may be an unsubstituted phenyl group, and X₂ to X₇ may each independently be a hydrogen atom or a deuterium atom. A₈ another example, X₇ may be an unsubstituted phenyl group, and X₁ to X₆ may each independently be a hydrogen atom or a deuterium atom.

In Formula 2,

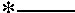 [SMILES: *C]

is a position linked to L₁ in Formula 1.

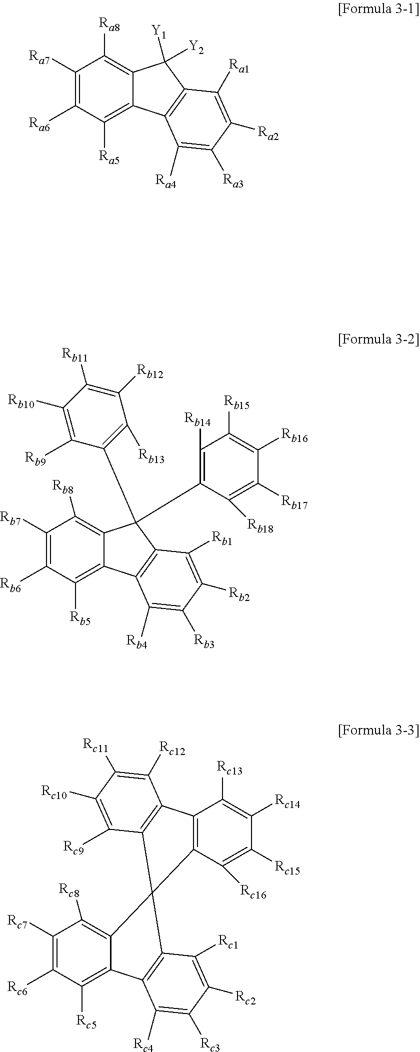

In Formula 3-1, Y₁ and Y₂ may each independently be a hydrogen atom, a deuterium atom, a halogen atom, a cyano group, a hydroxy group, or a substituted or unsubstituted alkyl group having 1 to 20 carbon atoms. For example, Y₁ and Y₂ may each independently be a substituted or unsubstituted methyl group.

In Formula 3-1 to Formula 3-3, Rₐ₁ to Rₐ₈, R_{b1} to R_{b18}, and R_{b1} to R_{c16} may each independently be a hydrogen atom, a deuterium atom, a halogen atom, a cyano group, a hydroxy group, a substituted or unsubstituted oxy group, a substituted or unsubstituted thio group, a substituted or unsubstituted boron group, a substituted or unsubstituted silyl group, a substituted or unsubstituted amine group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms. For example, Rₐ₁ to Rₐ₈, R_{b1} to R_{b18}, and R_{b1} to R_{c16} may each independently be a hydrogen atom, a deuterium atom, or a substituted or unsubstituted phenyl group.

Formula 3-1 to Formula 3-3 include the proviso that when R₂ in Formula 1 is a group represented by Formula 3-1, any one of Rₐ₁ to Rₐ₈ in Formula 3-1 may be a position linked to L₂ in Formula 1, when R₂ in Formula 1 is a group represented by Formula 3-2, any one of Rei to R_{b18} in Formula 3-2 may be a position linked to L₂ in Formula 1, and when R₂ in Formula 1 is a group represented by Formula 3-3, any one of R_{c1} to R_{c16} in Formula 3-3 may be a position linked to L₂ in Formula 1.

Formula 1 includes the triazine ring as described above. Formula 2 may correspond to the naphthalene moiety of the first substituent as described above. In Formula 2, the substituted or unsubstituted phenyl group linked to X₁ or X₇ may correspond to the benzene moiety of the first substituent as described above. Formula 3-1, Formula 3-2, and Formula 3-3 may each correspond to the fluorene moiety as described above.

In an embodiment, the nitrogen-containing compound represented by Formula 1 may be represented by any one of Formula 1-1 to Formula 1-4:

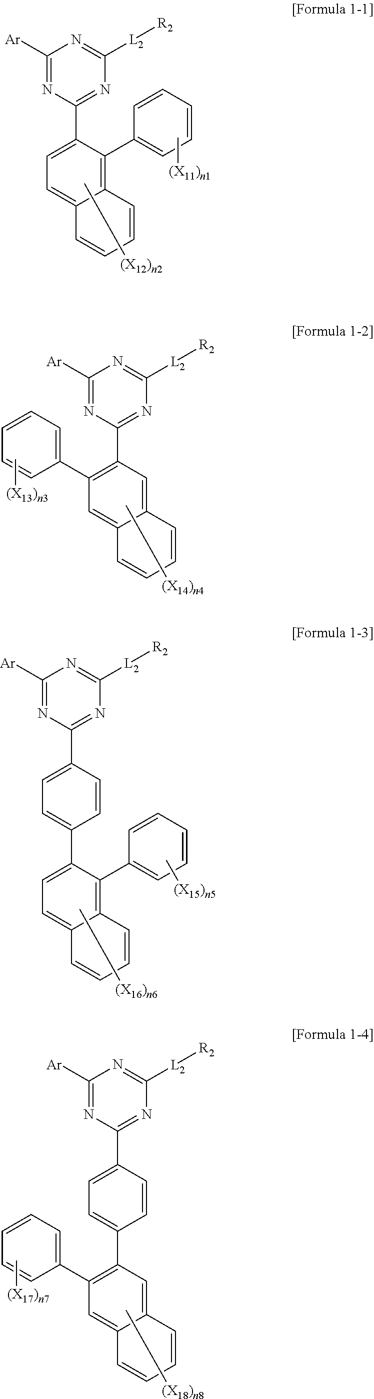

Formula 1-1 to Formula 1-4 each represent a case where in Formula 1, L₁ and R₁ are further defined. Formula 1-1 and Formula 1-2 each represent a case where L₁ is a direct linkage, and Formula 1-3 and Formula 1-4 each represent a case where L₁ is an unsubstituted phenyl group. Formula 1-1 and Formula 1-3 each represent a case where X₁ in Formula 2 is a substituted or unsubstituted phenyl group, and Formula 1-2 and Formula 1-4 each represent a case where X₇ in Formula 2 is a substituted or unsubstituted phenyl group.

In Formula 1-1 to Formula 1-4, X₁₁ to X₁₈ may each independently be a hydrogen atom or a deuterium atom. In an embodiment, X₁₁ to X₁₈ may each be a hydrogen atom.

In Formula 1-1 to Formula 1-4, n1, n3, n5, and n7 may each independently be an integer from 0 to 5. If n1, n3, n5, and n7 are each 0, the nitrogen-containing compound may not be substituted with X₁₁, X₁₃, X₁₅, and X₁₇, respectively. A case where n1, n3, n5, and n7 are each 5 and all groups of each of X₁₁, X₁₃, X₁₅, and X₁₇ are hydrogen atoms may be the same as a case where n1, n3, n5, and n7 are each 0. If n1, n3, n5, and n7 are each 2 or greater, multiple groups of each of X₁₁, X₁₃, X₁₅, and X₁₇ may all be the same or at least one thereof may be different from the remainder.

In Formula 1-1 to Formula 1-4, n2, n4, n6, and n8 may each independently be an integer from 0 to 6. If n2, n4, n6, and n8 are each 0, the nitrogen-containing compound may not be substituted with X₁₂, X₁₄, X₁₆, and X₁₈, respectively. A case where n2, n4, n6, and n8 are each 6 and all groups of each of X₁₂, X₁₄, X₁₆, and X₁₈ are hydrogen atoms may be the same as a case where n2, n4, n6, and n8 are each 0. If n2, n4, n6, and n8 are each 2 or greater, multiple groups of each of X₁₂, X₁₄, X₁₆, and X₁₈ may all be the same or at least one thereof may be different from the remainder.

In Formula 1-1 to Formula 1-4, Ar, L₂, and R₂ are the same as described in Formula 1.

In an embodiment, the nitrogen-containing compound represented by Formula 1 may be represented by any one of Formula 1-5 to Formula 1-9:

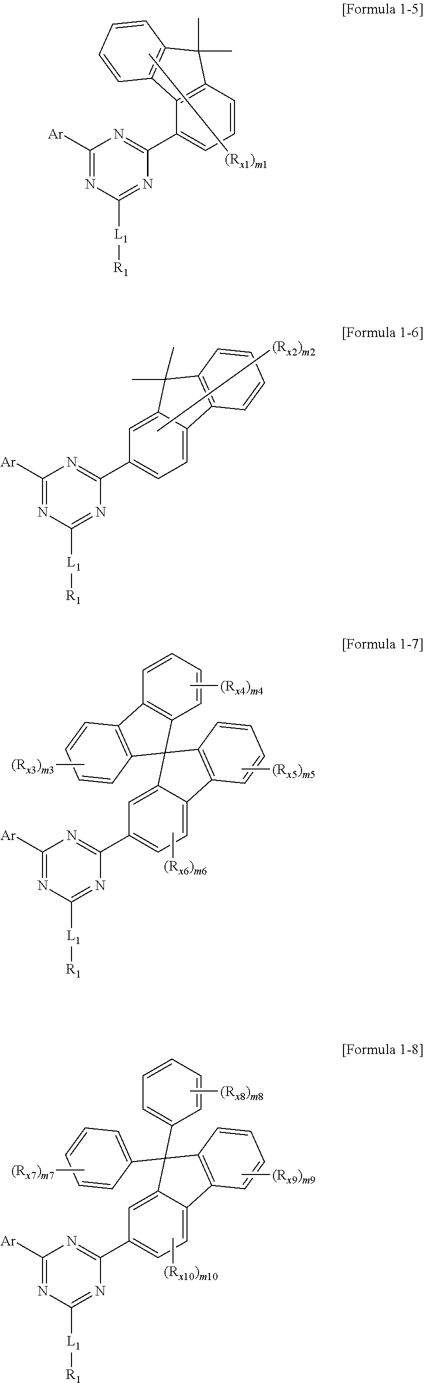

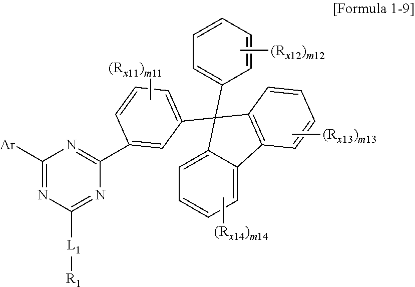

In Formula 1-5 to Formula 1-9, Rₓ₁ to Rₓ₁₄ may each independently be a hydrogen atom, a deuterium atom, a halogen atom, a cyano group, or a substituted or unsubstituted aryl group having 6 to 12 ring-forming carbon atoms. In an embodiment, Rₓ₁ to Rₓ₃ and Rₓ₅ to Rₓ₁₄ may each be a hydrogen atom, and Rₓ₄ may be a hydrogen atom or an unsubstituted phenyl group.

In Formula 1-5 and Formula 1-6, m1 and m2 may each independently be an integer from 0 to 7. If m1 and m2 are each 0, the nitrogen-containing compound may not be substituted with Rₓ₁ and Rₓ₂, respectively. A case where m1 and m2 are each 6 and all Rₓ₁ groups and all Rₓ₂ groups are hydrogen atoms may be the same as a case where m1 and m2 are each 0. If m1 and m2 are each 2 or greater, multiple Rₓ₁ groups and multiple Rₓ₂ groups may all be the same or at least one thereof may be different from the remainder.

In Formula 1-7 to Formula 1-9, m3 to m5, m9, m11, m13, and m14 may each independently be an integer from 0 to 4. If m3 to m5, m9, m11, m13, and m14 are each 0, the nitrogen-containing compound may not be substituted with Rₓ₃ to Rₓ₅, Rₓ₉, Rₓ₁₁, Rₓ₁₃, and Rₓ₁₄, respectively. A case where m3 to m5, m9, m11, m13, and m14 are each 4 and all groups of each of Rₓ₃ to Rₓ₅, Rₓ₉, Rₓ₁₁, Rₓ₁₃, and Rₓ₁₄ are hydrogen atoms may be the same as a case where m3 to m5, m9, m11, m13, and m14 are each 0. If m3 to m5, m9, m11, m13, and m14 are each 2 or greater, multiple groups of each of Rₓ₃ to Rₓ₅, Rₓ₉, Rₓ₁₁, Rₓ₁₃, and Rₓ₁₄ may all be the same or at least one thereof may be different from the remainder.

In Formula 1-7 and Formula 1-8, m6 and m10 may each independently be an integer from 0 to 3. If m6 and m10 are each 0, the nitrogen-containing compound may not be substituted with Rₓ₆ and Rₓ₁₀, respectively. A case where m6 and m10 are each 3 and all Rₓ₆ groups and all Rₓ₁₀ groups are hydrogen atoms may be the same as a case where m6 and m10 are each 0. If m6 and m10 are each 2 or greater, multiple Rₓ₆ groups and multiple Rₓ₁₀ groups may all be the same or at least one thereof may be different from the remainder.

In Formula 1-8 and Formula 1-9, m7, m8, and m12 may each independently be an integer from 0 to 5. If m7, m8, and m12 are each 0, the nitrogen-containing compound may not be substituted with Rₓ₇, Rₓ₈, and Rₓ₁₂, respectively. A case where m7, m8, and m12 are each 5 and all Rₓ₇ groups, all Rₓ₅ groups, and all Rₓ₁₂ groups are hydrogen atoms may be the same as a case where m7, m8, and m12 are each 0. If m7, m8, and m12 are each 2 or greater, multiple Rₓ₇ groups, multiple Rₓ₈ groups, and multiple Rₓ₁₂ groups may all be the same or at least one thereof may be different from the remainder.

In Formula 1-5 to Formula 1-9, Ar, L₁, and R₁ are the same as described in Formula 1.

In an embodiment, the nitrogen-containing compound represented by Formula 1 may be represented by any one of Formula 1-10 to Formula 1-14:

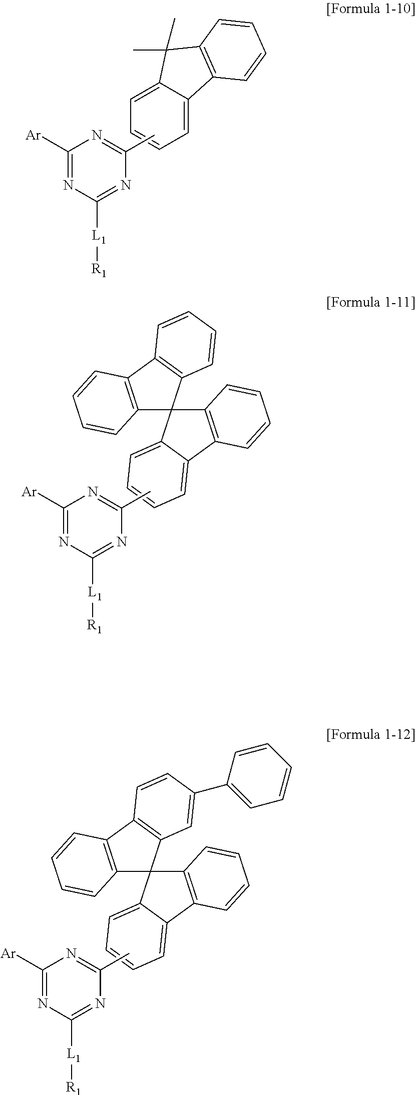

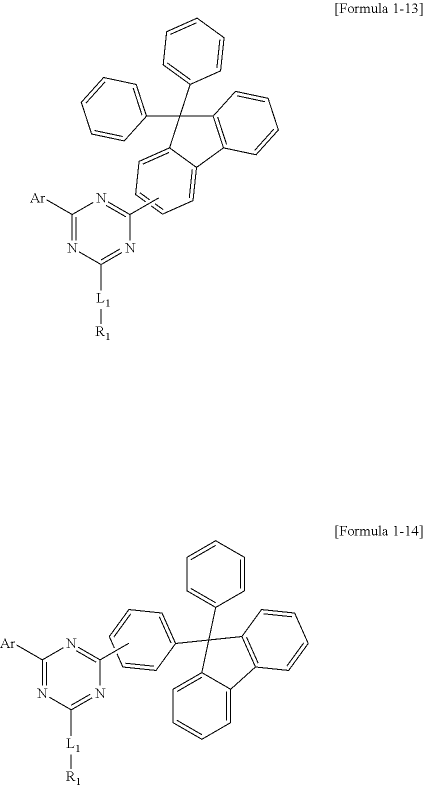

In Formula 1-10 to Formula 1-14, Ar, L₁, and R₁ are the same as described in Formula 1.

In an embodiment, the nitrogen-containing compound may be any compound selected from Compound Group 1. In an embodiment, in the light emitting element ED, the at least one functional layer (for example, an electron transport region ETR) may include at least one nitrogen-containing compound selected from Compound Group 1:

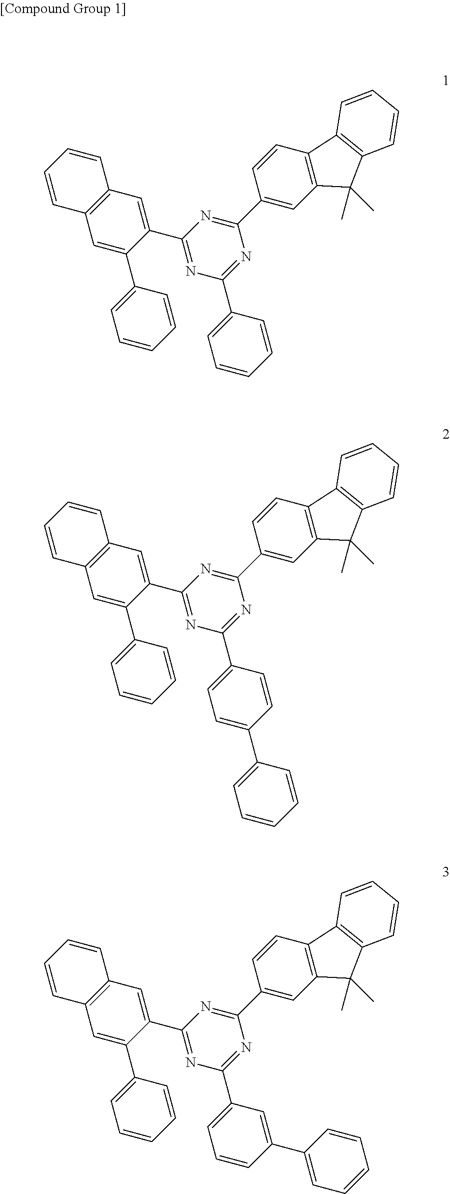 [SMILES: CC1(C)c2ccccc2-c2ccc(-c3nc(-c4ccccc4)nc(-c4cc5ccccc5cc4-c4ccccc4)n3)cc21 ; CC1(C)c2ccccc2-c2ccc(-c3nc(-c4ccc(-c5ccccc5)cc4)nc(-c4cc5ccccc5cc4-c4ccccc4)n3)cc21 ; CC1(C)c2ccccc2-c2ccc(-c3nc(-c4cccc(-c5ccccc5)c4)nc(-c4cc5ccccc5cc4-c4ccccc4)n3)cc21]

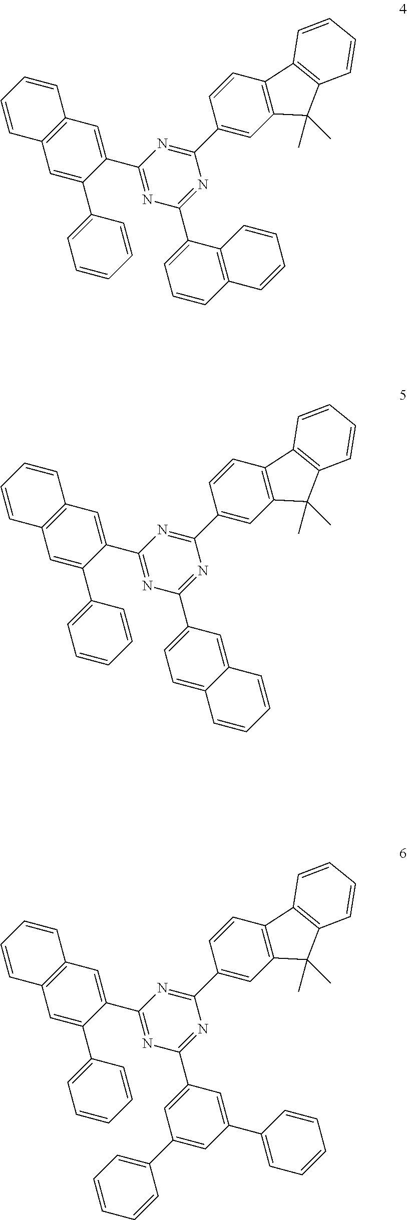 [SMILES: CC1(C)c2ccccc2-c2ccc(-c3nc(-c4cc5ccccc5cc4-c4ccccc4)nc(-c4cccc5ccccc45)n3)cc21 ; CC1(C)c2ccccc2-c2ccc(-c3nc(-c4ccc5ccccc5c4)nc(-c4cc5ccccc5cc4-c4ccccc4)n3)cc21 ; CC1(C)c2ccccc2-c2ccc(-c3nc(-c4cc(-c5ccccc5)cc(-c5ccccc5)c4)nc(-c4cc5ccccc5cc4-c4ccccc4)n3)cc21]

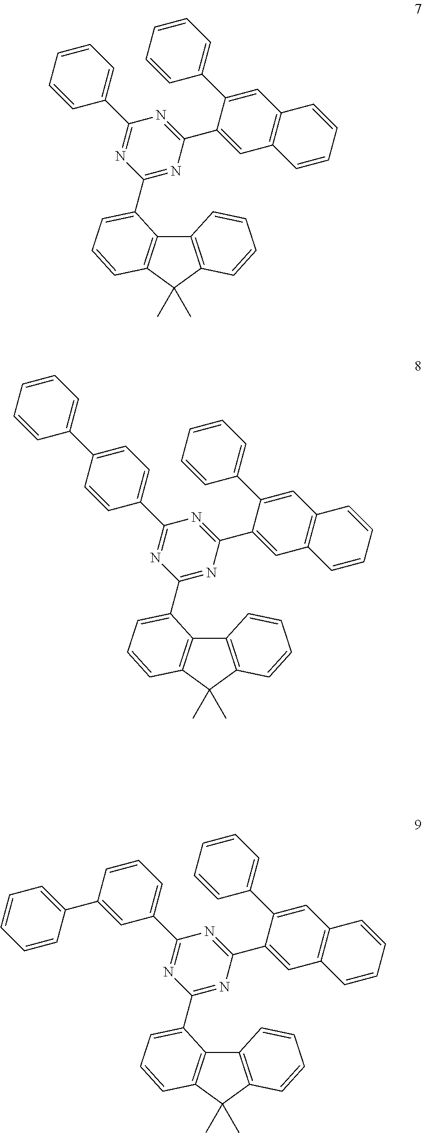 [SMILES: CC1(C)c2ccccc2-c2c(-c3nc(-c4ccccc4)nc(-c4cc5ccccc5cc4-c4ccccc4)n3)cccc21 ; CC1(C)c2ccccc2-c2c(-c3nc(-c4ccc(-c5ccccc5)cc4)nc(-c4cc5ccccc5cc4-c4ccccc4)n3)cccc21 ; CC1(C)c2ccccc2-c2c(-c3nc(-c4cccc(-c5ccccc5)c4)nc(-c4cc5ccccc5cc4-c4ccccc4)n3)cccc21]

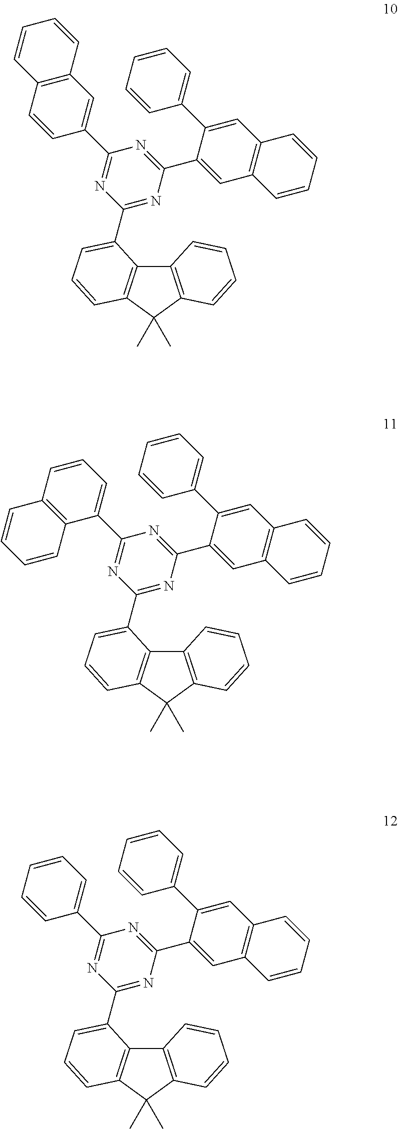 [SMILES: CC1(C)c2ccccc2-c2c(-c3nc(-c4ccc5ccccc5c4)nc(-c4cc5ccccc5cc4-c4ccccc4)n3)cccc21 ; CC1(C)c2ccccc2-c2c(-c3nc(-c4cc5ccccc5cc4-c4ccccc4)nc(-c4cccc5ccccc45)n3)cccc21 ; CC1(C)c2ccccc2-c2c(-c3nc(-c4ccccc4)nc(-c4cc5ccccc5cc4-c4ccccc4)n3)cccc21]

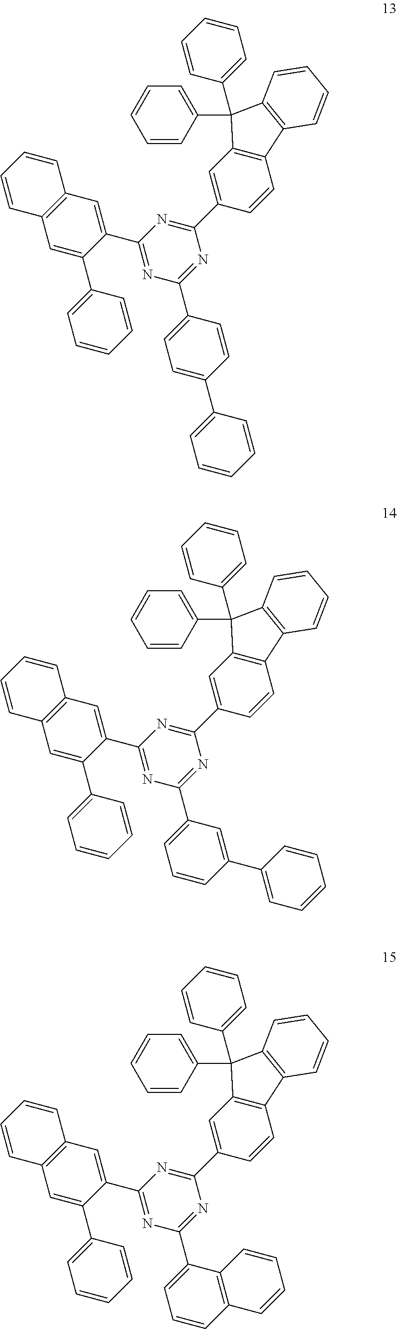 [SMILES: c1ccc(-c2ccc(-c3nc(-c4ccc5c(c4)C(c4ccccc4)(c4ccccc4)c4ccccc4-5)nc(-c4cc5ccccc5cc4-c4ccccc4)n3)cc2)cc1 ; c1ccc(-c2cccc(-c3nc(-c4ccc5c(c4)C(c4ccccc4)(c4ccccc4)c4ccccc4-5)nc(-c4cc5ccccc5cc4-c4ccccc4)n3)c2)cc1 ; c1ccc(-c2cc3ccccc3cc2-c2nc(-c3ccc4c(c3)C(c3ccccc3)(c3ccccc3)c3ccccc3-4)nc(-c3cccc4ccccc34)n2)cc1]

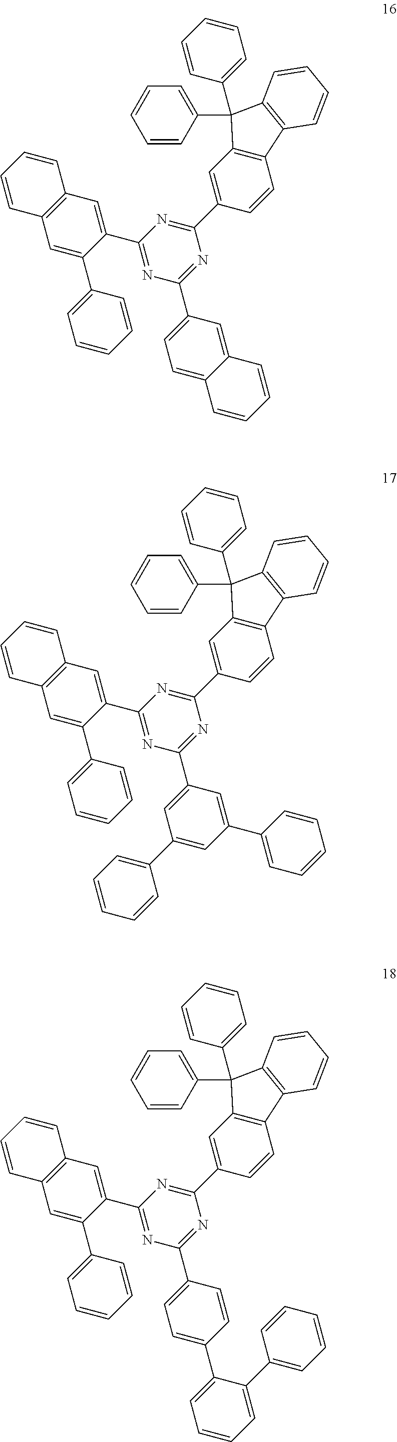 [SMILES: c1ccc(-c2cc3ccccc3cc2-c2nc(-c3ccc4c(c3)C(c3ccccc3)(c3ccccc3)c3ccccc3-4)nc(-c3ccc4ccccc4c3)n2)cc1 ; c1ccc(-c2cc(-c3ccccc3)cc(-c3nc(-c4ccc5c(c4)C(c4ccccc4)(c4ccccc4)c4ccccc4-5)nc(-c4cc5ccccc5cc4-c4ccccc4)n3)c2)cc1 ; c1ccc(-c2ccccc2-c2ccc(-c3nc(-c4ccc5c(c4)C(c4ccccc4)(c4ccccc4)c4ccccc4-5)nc(-c4cc5ccccc5cc4-c4ccccc4)n3)cc2)cc1]

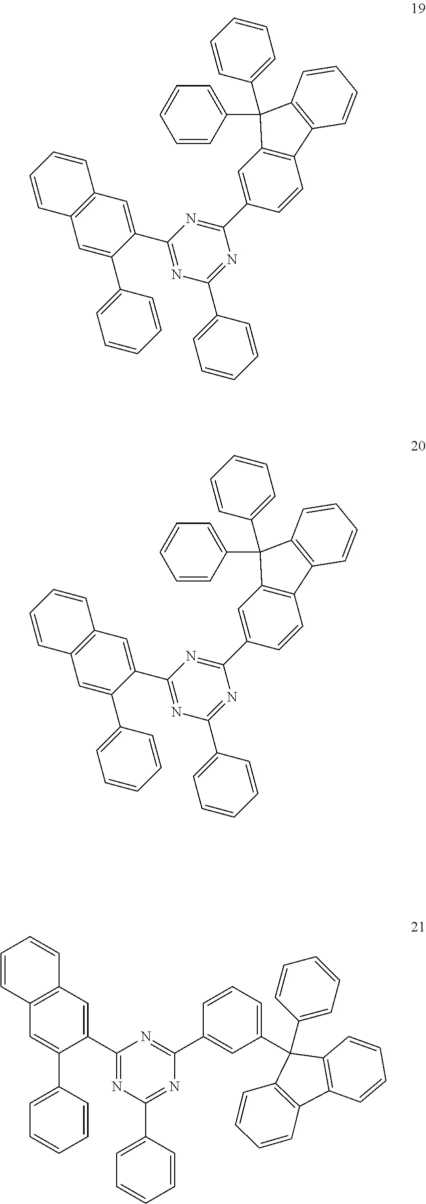 [SMILES: c1ccc(-c2nc(-c3ccc4c(c3)C(c3ccccc3)(c3ccccc3)c3ccccc3-4)nc(-c3cc4ccccc4cc3-c3ccccc3)n2)cc1 ; c1ccc(-c2nc(-c3ccc4c(c3)C(c3ccccc3)(c3ccccc3)c3ccccc3-4)nc(-c3cc4ccccc4cc3-c3ccccc3)n2)cc1 ; c1ccc(-c2nc(-c3cccc(C4(c5ccccc5)c5ccccc5-c5ccccc54)c3)nc(-c3cc4ccccc4cc3-c3ccccc3)n2)cc1]

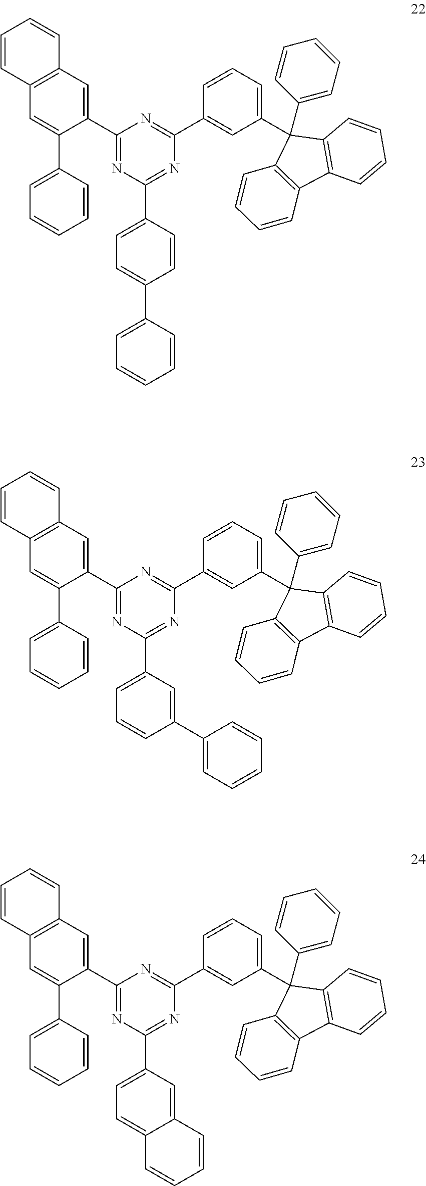 [SMILES: c1ccc(-c2ccc(-c3nc(-c4cccc(C5(c6ccccc6)c6ccccc6-c6ccccc65)c4)nc(-c4cc5ccccc5cc4-c4ccccc4)n3)cc2)cc1 ; c1ccc(-c2cccc(-c3nc(-c4cccc(C5(c6ccccc6)c6ccccc6-c6ccccc65)c4)nc(-c4cc5ccccc5cc4-c4ccccc4)n3)c2)cc1 ; c1ccc(-c2cc3ccccc3cc2-c2nc(-c3cccc(C4(c5ccccc5)c5ccccc5-c5ccccc54)c3)nc(-c3ccc4ccccc4c3)n2)cc1]

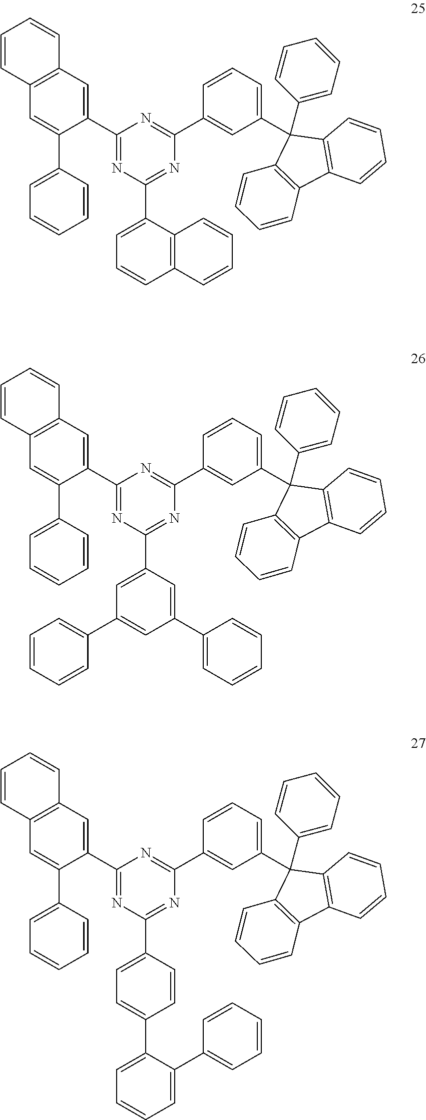 [SMILES: c1ccc(-c2cc3ccccc3cc2-c2nc(-c3cccc(C4(c5ccccc5)c5ccccc5-c5ccccc54)c3)nc(-c3cccc4ccccc34)n2)cc1 ; c1ccc(-c2cc(-c3ccccc3)cc(-c3nc(-c4cccc(C5(c6ccccc6)c6ccccc6-c6ccccc65)c4)nc(-c4cc5ccccc5cc4-c4ccccc4)n3)c2)cc1 ; c1ccc(-c2ccccc2-c2ccc(-c3nc(-c4cccc(C5(c6ccccc6)c6ccccc6-c6ccccc65)c4)nc(-c4cc5ccccc5cc4-c4ccccc4)n3)cc2)cc1]

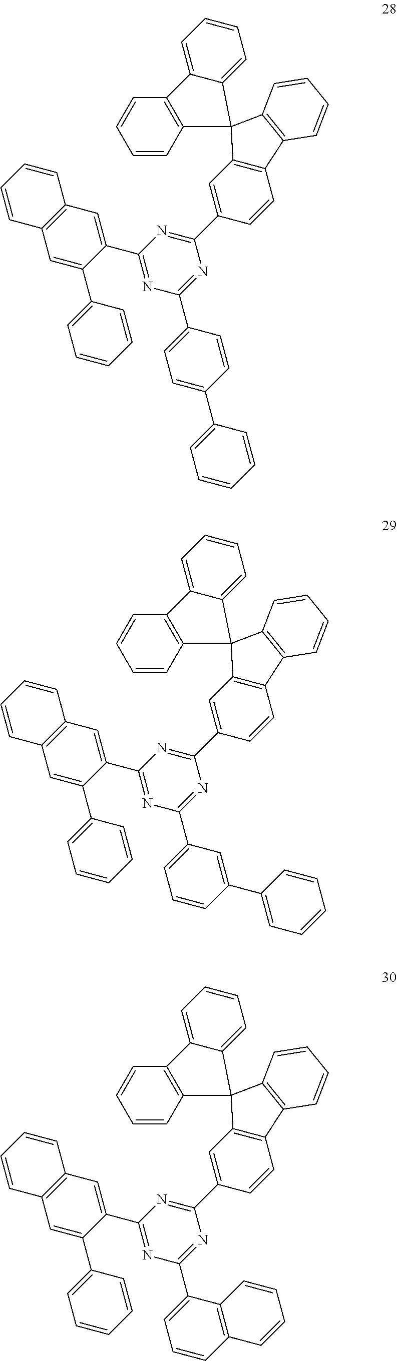 [SMILES: c1ccc(-c2ccc(-c3nc(-c4ccc5c(c4)C4(c6ccccc6-c6ccccc64)c4ccccc4-5)nc(-c4cc5ccccc5cc4-c4ccccc4)n3)cc2)cc1 ; c1ccc(-c2cccc(-c3nc(-c4ccc5c(c4)C4(c6ccccc6-c6ccccc64)c4ccccc4-5)nc(-c4cc5ccccc5cc4-c4ccccc4)n3)c2)cc1 ; c1ccc(-c2cc3ccccc3cc2-c2nc(-c3ccc4c(c3)C3(c5ccccc5-c5ccccc53)c3ccccc3-4)nc(-c3cccc4ccccc34)n2)cc1]

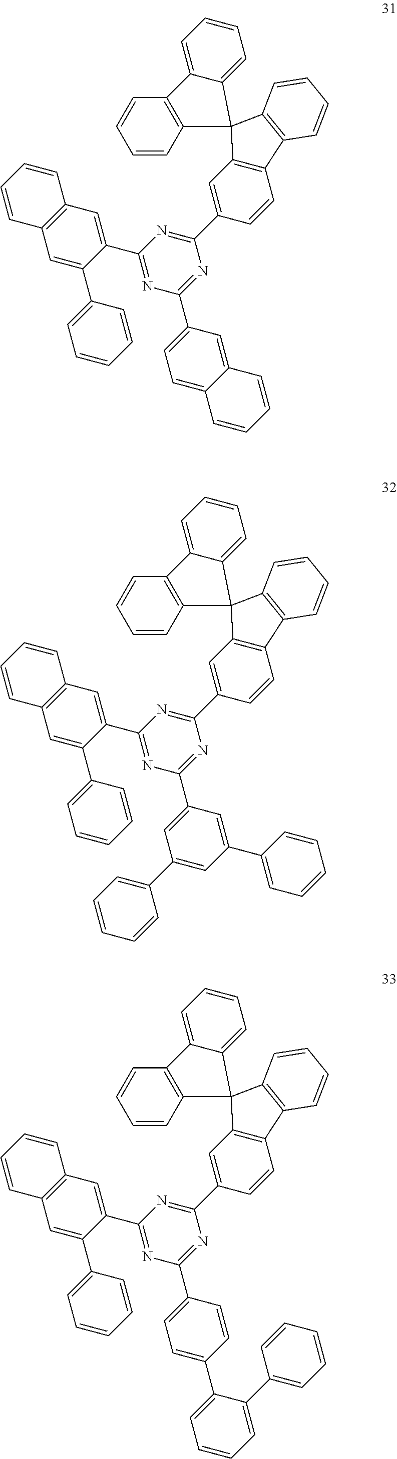 [SMILES: c1ccc(-c2cc3ccccc3cc2-c2nc(-c3ccc4c(c3)C3(c5ccccc5-c5ccccc53)c3ccccc3-4)nc(-c3ccc4ccccc4c3)n2)cc1 ; c1ccc(-c2cc(-c3ccccc3)cc(-c3nc(-c4ccc5c(c4)C4(c6ccccc6-c6ccccc64)c4ccccc4-5)nc(-c4cc5ccccc5cc4-c4ccccc4)n3)c2)cc1 ; c1ccc(-c2ccccc2-c2ccc(-c3nc(-c4ccc5c(c4)C4(c6ccccc6-c6ccccc64)c4ccccc4-5)nc(-c4cc5ccccc5cc4-c4ccccc4)n3)cc2)cc1]

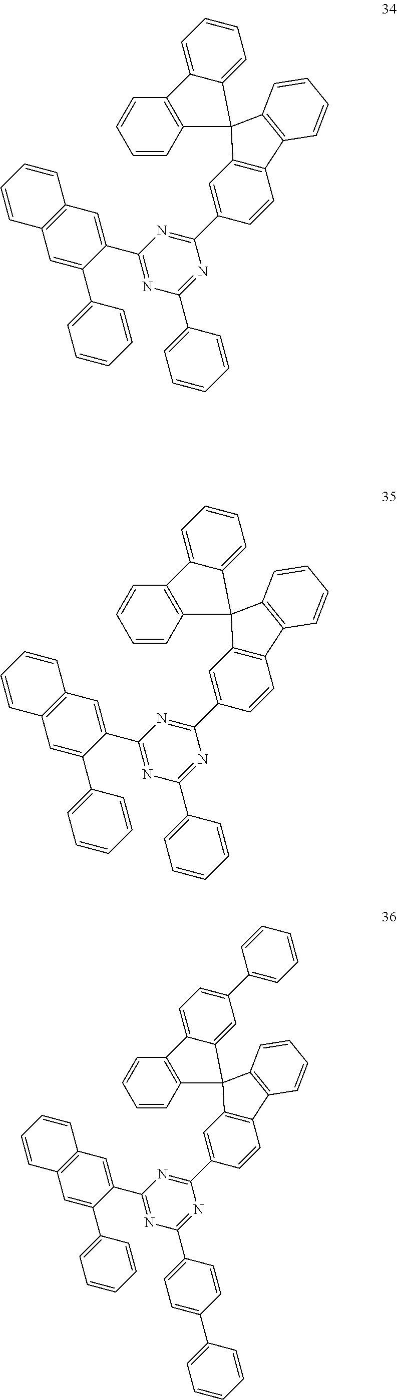 [SMILES: c1ccc(-c2nc(-c3ccc4c(c3)C3(c5ccccc5-c5ccccc53)c3ccccc3-4)nc(-c3cc4ccccc4cc3-c3ccccc3)n2)cc1 ; c1ccc(-c2nc(-c3ccc4c(c3)C3(c5ccccc5-c5ccccc53)c3ccccc3-4)nc(-c3cc4ccccc4cc3-c3ccccc3)n2)cc1 ; c1ccc(-c2ccc(-c3nc(-c4ccc5c(c4)C4(c6ccccc6-c6ccc(-c7ccccc7)cc64)c4ccccc4-5)nc(-c4cc5ccccc5cc4-c4ccccc4)n3)cc2)cc1]

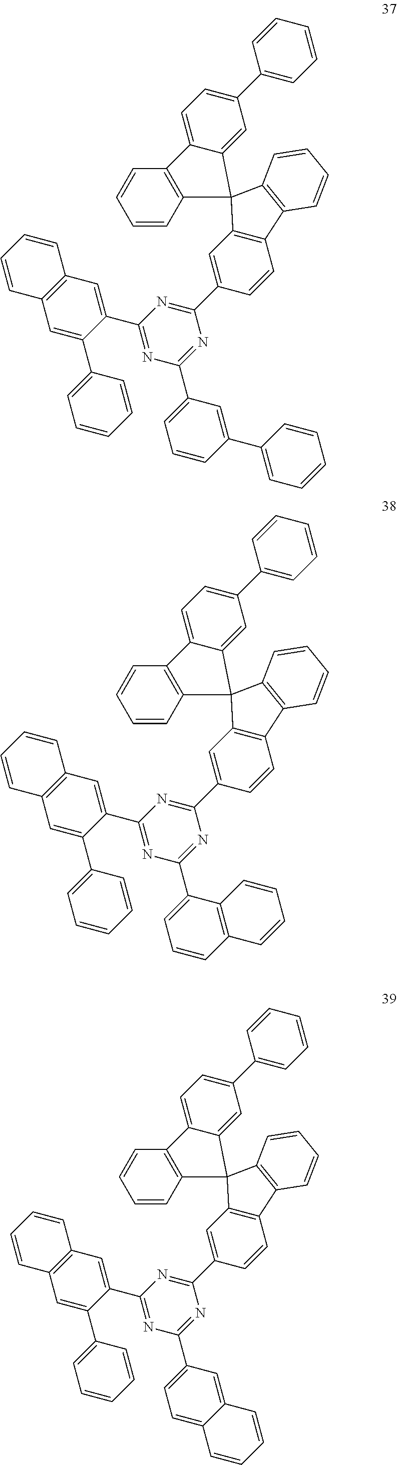

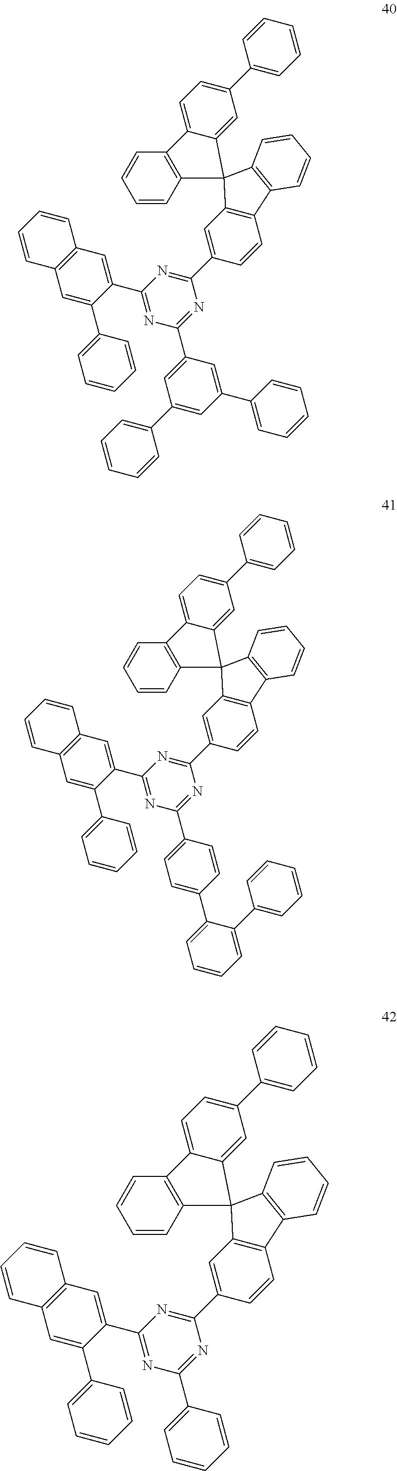 [SMILES: c1ccc(-c2cc(-c3ccccc3)cc(-c3nc(-c4ccc5c(c4)C4(c6ccccc6-c6ccc(-c7ccccc7)cc64)c4ccccc4-5)nc(-c4cc5ccccc5cc4-c4ccccc4)n3)c2)cc1 ; c1ccc(-c2ccc3c(c2)C2(c4ccccc4-3)c3ccccc3-c3ccc(-c4nc(-c5ccc(-c6ccccc6-c6ccccc6)cc5)nc(-c5cc6ccccc6cc5-c5ccccc5)n4)cc32)cc1 ; c1ccc(-c2ccc3c(c2)C2(c4ccccc4-3)c3ccccc3-c3ccc(-c4nc(-c5ccccc5)nc(-c5cc6ccccc6cc5-c5ccccc5)n4)cc32)cc1]

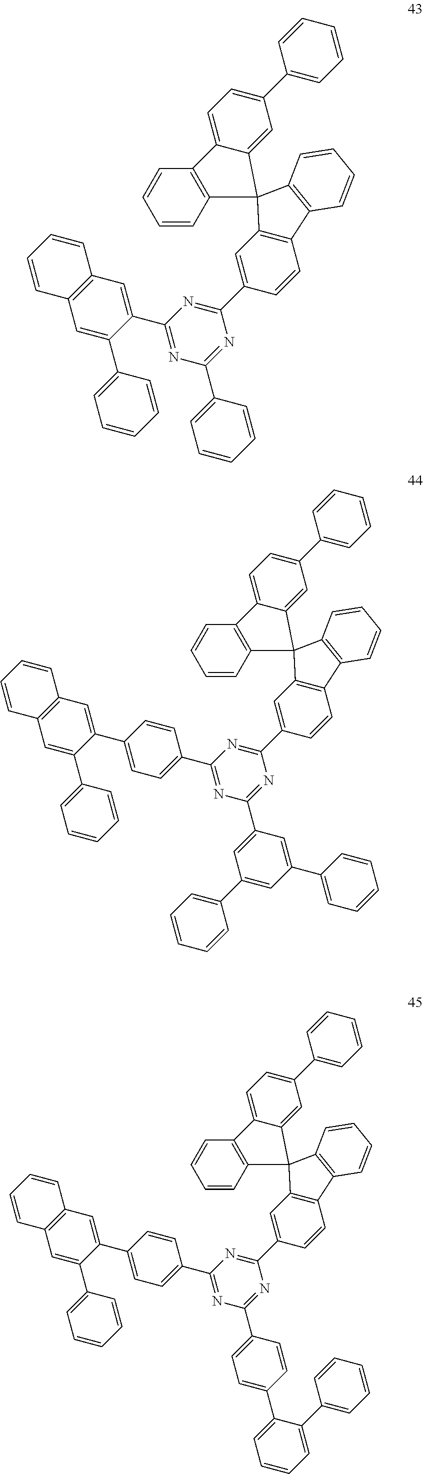 [SMILES: c1ccc(-c2ccc3c(c2)C2(c4ccccc4-3)c3ccccc3-c3ccc(-c4nc(-c5ccccc5)nc(-c5cc6ccccc6cc5-c5ccccc5)n4)cc32)cc1 ; c1ccc(-c2cc(-c3ccccc3)cc(-c3nc(-c4ccc(-c5cc6ccccc6cc5-c5ccccc5)cc4)nc(-c4ccc5c(c4)C4(c6ccccc6-c6ccc(-c7ccccc7)cc64)c4ccccc4-5)n3)c2)cc1 ; c1ccc(-c2ccc3c(c2)C2(c4ccccc4-3)c3ccccc3-c3ccc(-c4nc(-c5ccc(-c6ccccc6-c6ccccc6)cc5)nc(-c5ccc(-c6cc7ccccc7cc6-c6ccccc6)cc5)n4)cc32)cc1]

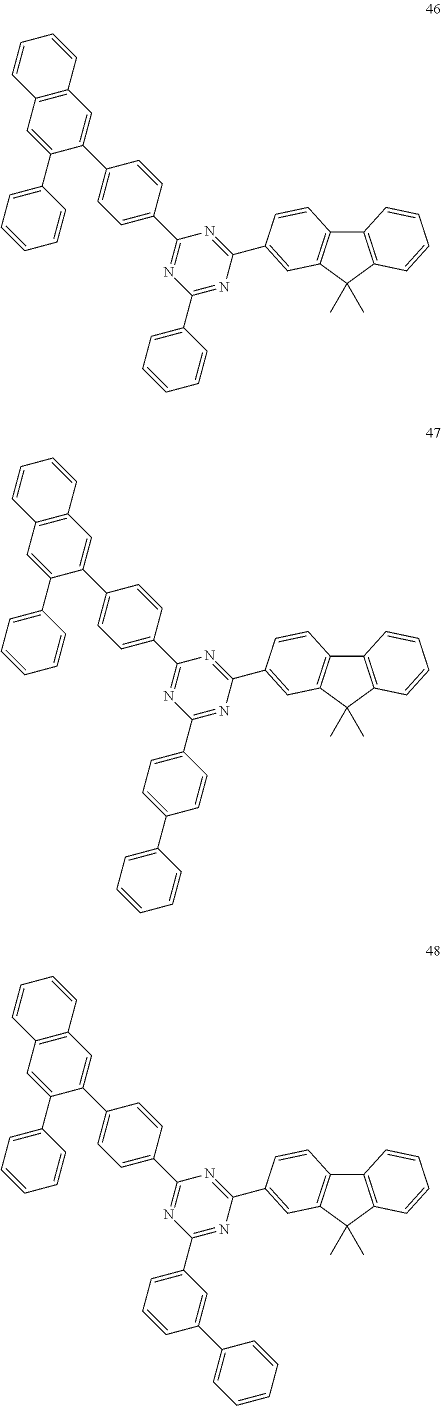 [SMILES: CC1(C)c2ccccc2-c2ccc(-c3nc(-c4ccccc4)nc(-c4ccc(-c5cc6ccccc6cc5-c5ccccc5)cc4)n3)cc21 ; CC1(C)c2ccccc2-c2ccc(-c3nc(-c4ccc(-c5ccccc5)cc4)nc(-c4ccc(-c5cc6ccccc6cc5-c5ccccc5)cc4)n3)cc21 ; CC1(C)c2ccccc2-c2ccc(-c3nc(-c4ccc(-c5cc6ccccc6cc5-c5ccccc5)cc4)nc(-c4cccc(-c5ccccc5)c4)n3)cc21]

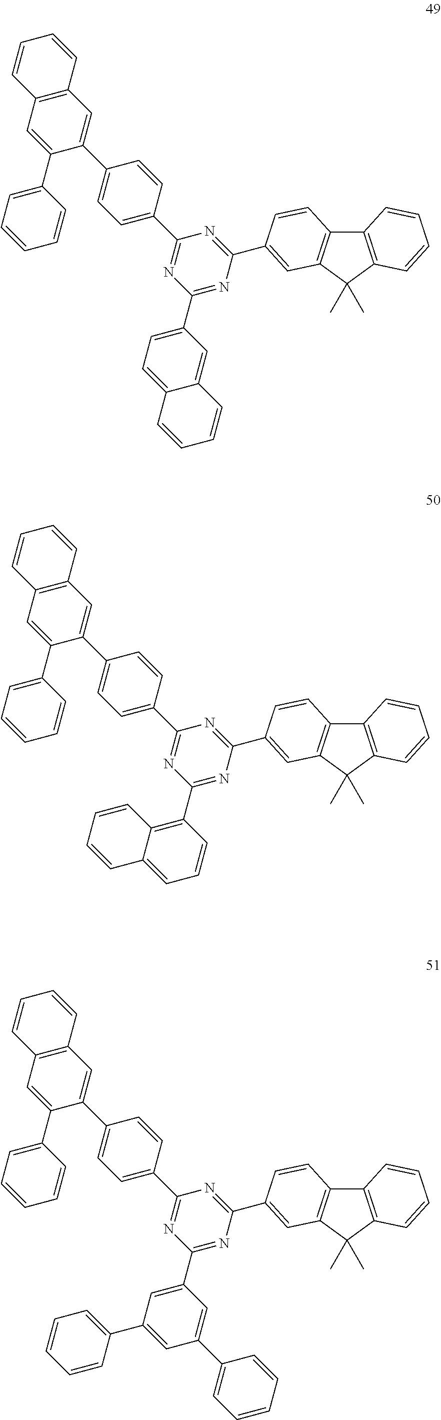 [SMILES: CC1(C)c2ccccc2-c2ccc(-c3nc(-c4ccc(-c5cc6ccccc6cc5-c5ccccc5)cc4)nc(-c4ccc5ccccc5c4)n3)cc21 ; CC1(C)c2ccccc2-c2ccc(-c3nc(-c4ccc(-c5cc6ccccc6cc5-c5ccccc5)cc4)nc(-c4cccc5ccccc45)n3)cc21 ; CC1(C)c2ccccc2-c2ccc(-c3nc(-c4ccc(-c5cc6ccccc6cc5-c5ccccc5)cc4)nc(-c4cc(-c5ccccc5)cc(-c5ccccc5)c4)n3)cc21]

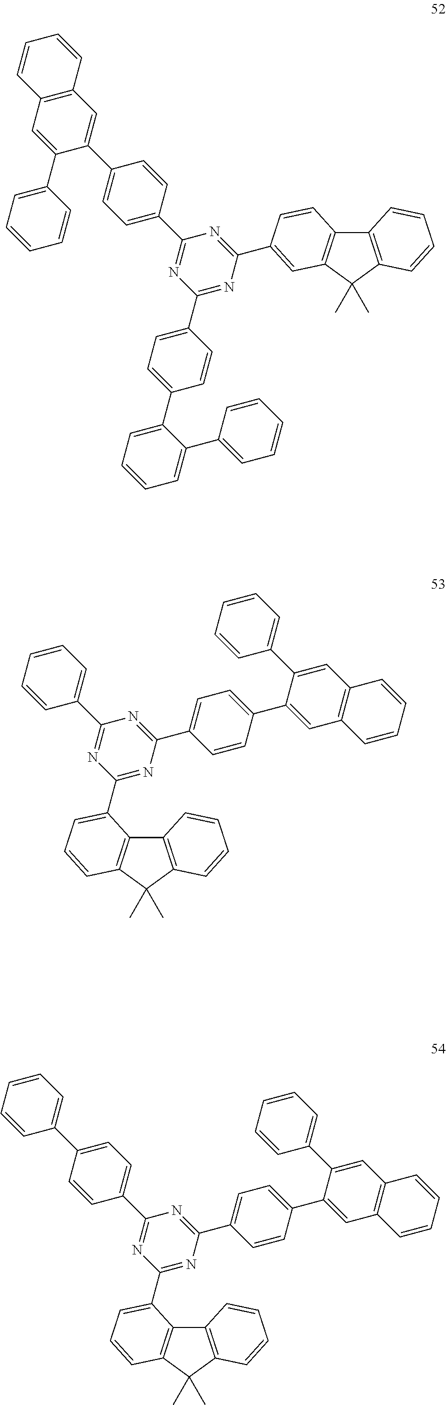 [SMILES: CC1(C)c2ccccc2-c2ccc(-c3nc(-c4ccc(-c5ccccc5-c5ccccc5)cc4)nc(-c4ccc(-c5cc6ccccc6cc5-c5ccccc5)cc4)n3)cc21 ; CC1(C)c2ccccc2-c2c(-c3nc(-c4ccccc4)nc(-c4ccc(-c5cc6ccccc6cc5-c5ccccc5)cc4)n3)cccc21 ; CC1(C)c2ccccc2-c2c(-c3nc(-c4ccc(-c5ccccc5)cc4)nc(-c4ccc(-c5cc6ccccc6cc5-c5ccccc5)cc4)n3)cccc21]

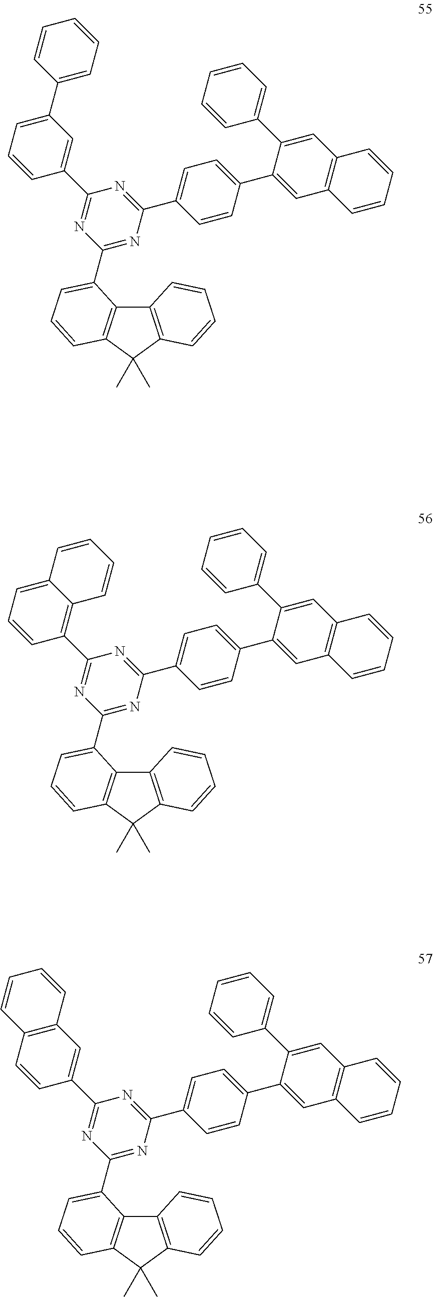 [SMILES: CC1(C)c2ccccc2-c2c(-c3nc(-c4ccc(-c5cc6ccccc6cc5-c5ccccc5)cc4)nc(-c4cccc(-c5ccccc5)c4)n3)cccc21 ; CC1(C)c2ccccc2-c2c(-c3nc(-c4ccc(-c5cc6ccccc6cc5-c5ccccc5)cc4)nc(-c4cccc5ccccc45)n3)cccc21 ; CC1(C)c2ccccc2-c2c(-c3nc(-c4ccc(-c5cc6ccccc6cc5-c5ccccc5)cc4)nc(-c4ccc5ccccc5c4)n3)cccc21]

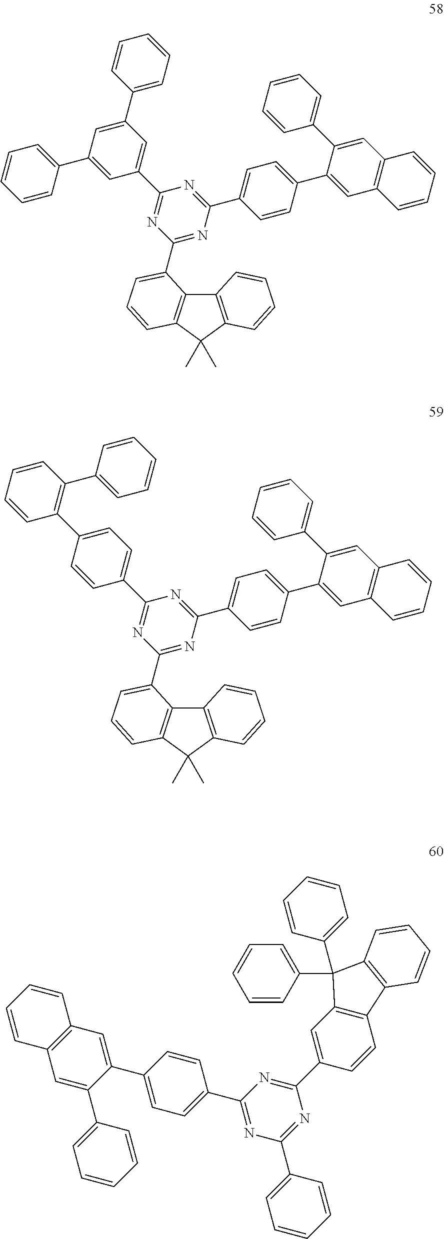 [SMILES: CC1(C)c2ccccc2-c2c(-c3nc(-c4ccc(-c5cc6ccccc6cc5-c5ccccc5)cc4)nc(-c4cc(-c5ccccc5)cc(-c5ccccc5)c4)n3)cccc21 ; CC1(C)c2ccccc2-c2c(-c3nc(-c4ccc(-c5ccccc5-c5ccccc5)cc4)nc(-c4ccc(-c5cc6ccccc6cc5-c5ccccc5)cc4)n3)cccc21 ; c1ccc(-c2nc(-c3ccc(-c4cc5ccccc5cc4-c4ccccc4)cc3)nc(-c3ccc4c(c3)C(c3ccccc3)(c3ccccc3)c3ccccc3-4)n2)cc1]

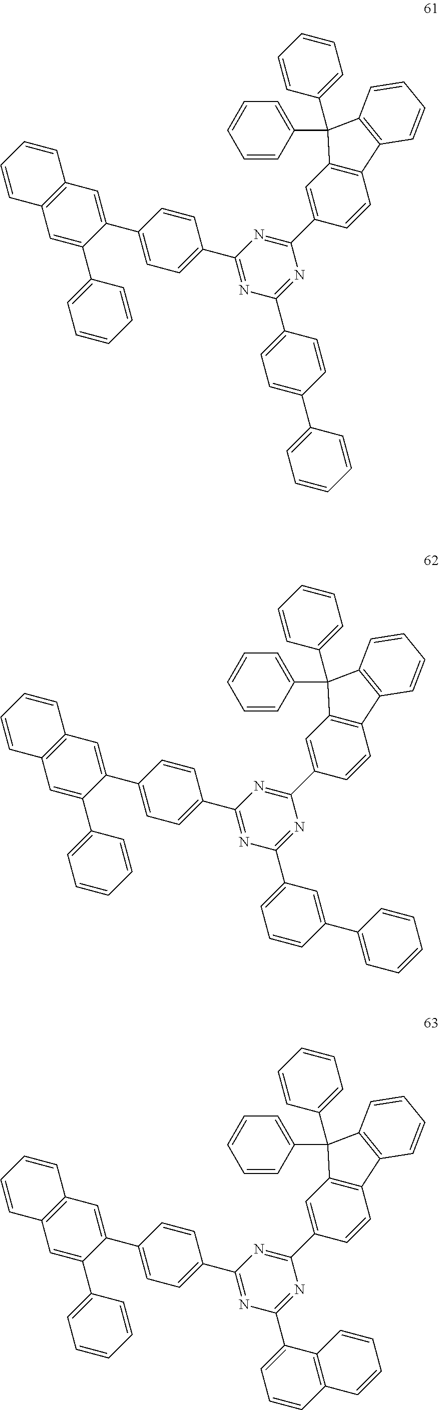 [SMILES: c1ccc(-c2ccc(-c3nc(-c4ccc(-c5cc6ccccc6cc5-c5ccccc5)cc4)nc(-c4ccc5c(c4)C(c4ccccc4)(c4ccccc4)c4ccccc4-5)n3)cc2)cc1 ; c1ccc(-c2cccc(-c3nc(-c4ccc(-c5cc6ccccc6cc5-c5ccccc5)cc4)nc(-c4ccc5c(c4)C(c4ccccc4)(c4ccccc4)c4ccccc4-5)n3)c2)cc1 ; c1ccc(-c2cc3ccccc3cc2-c2ccc(-c3nc(-c4ccc5c(c4)C(c4ccccc4)(c4ccccc4)c4ccccc4-5)nc(-c4cccc5ccccc45)n3)cc2)cc1]

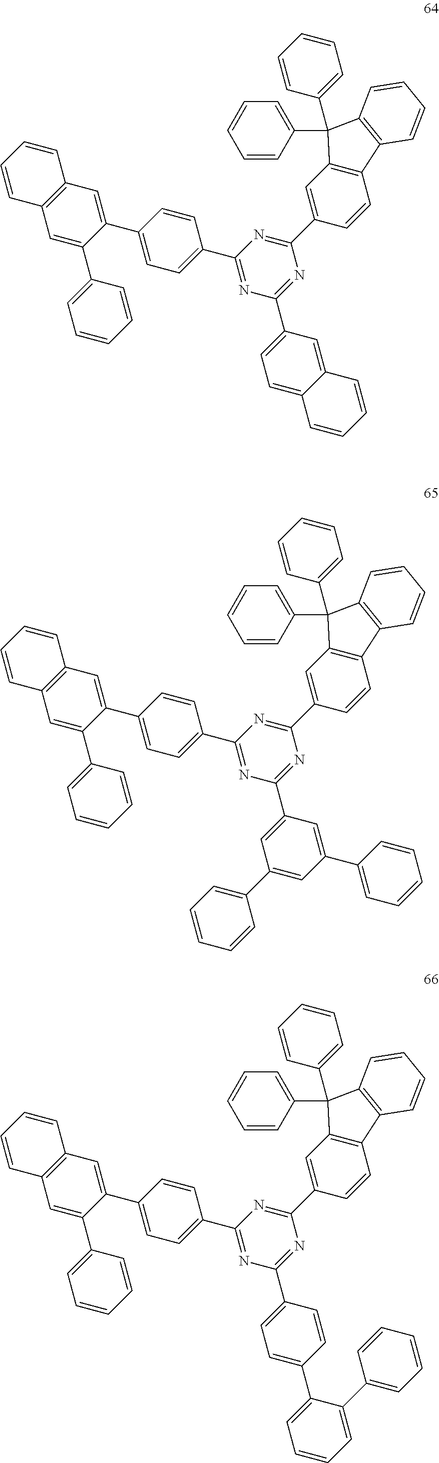 [SMILES: c1ccc(-c2cc3ccccc3cc2-c2ccc(-c3nc(-c4ccc5c(c4)C(c4ccccc4)(c4ccccc4)c4ccccc4-5)nc(-c4ccc5ccccc5c4)n3)cc2)cc1 ; c1ccc(-c2cc(-c3ccccc3)cc(-c3nc(-c4ccc(-c5cc6ccccc6cc5-c5ccccc5)cc4)nc(-c4ccc5c(c4)C(c4ccccc4)(c4ccccc4)c4ccccc4-5)n3)c2)cc1 ; c1ccc(-c2ccccc2-c2ccc(-c3nc(-c4ccc(-c5cc6ccccc6cc5-c5ccccc5)cc4)nc(-c4ccc5c(c4)C(c4ccccc4)(c4ccccc4)c4ccccc4-5)n3)cc2)cc1]

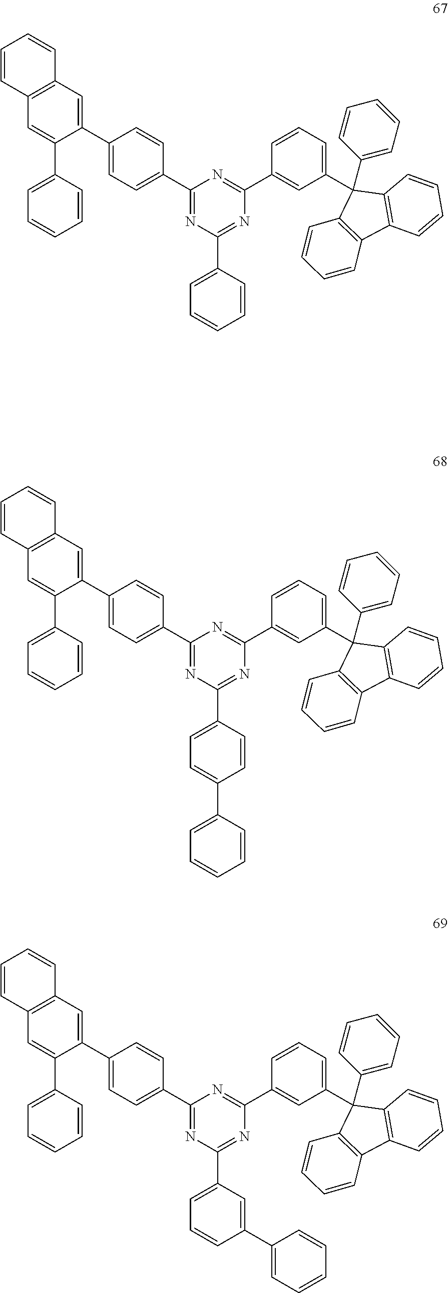 [SMILES: c1ccc(-c2nc(-c3ccc(-c4cc5ccccc5cc4-c4ccccc4)cc3)nc(-c3cccc(C4(c5ccccc5)c5ccccc5-c5ccccc54)c3)n2)cc1 ; c1ccc(-c2ccc(-c3nc(-c4ccc(-c5cc6ccccc6cc5-c5ccccc5)cc4)nc(-c4cccc(C5(c6ccccc6)c6ccccc6-c6ccccc65)c4)n3)cc2)cc1 ; c1ccc(-c2cccc(-c3nc(-c4ccc(-c5cc6ccccc6cc5-c5ccccc5)cc4)nc(-c4cccc(C5(c6ccccc6)c6ccccc6-c6ccccc65)c4)n3)c2)cc1]

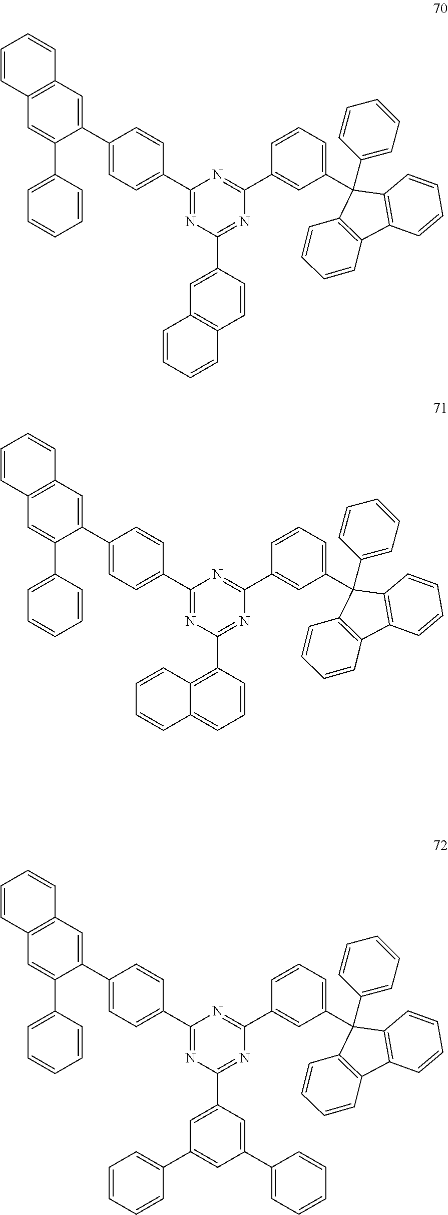 [SMILES: c1ccc(-c2cc3ccccc3cc2-c2ccc(-c3nc(-c4cccc(C5(c6ccccc6)c6ccccc6-c6ccccc65)c4)nc(-c4ccc5ccccc5c4)n3)cc2)cc1 ; c1ccc(-c2cc3ccccc3cc2-c2ccc(-c3nc(-c4cccc(C5(c6ccccc6)c6ccccc6-c6ccccc65)c4)nc(-c4cccc5ccccc45)n3)cc2)cc1 ; c1ccc(-c2cc(-c3ccccc3)cc(-c3nc(-c4ccc(-c5cc6ccccc6cc5-c5ccccc5)cc4)nc(-c4cccc(C5(c6ccccc6)c6ccccc6-c6ccccc65)c4)n3)c2)cc1]

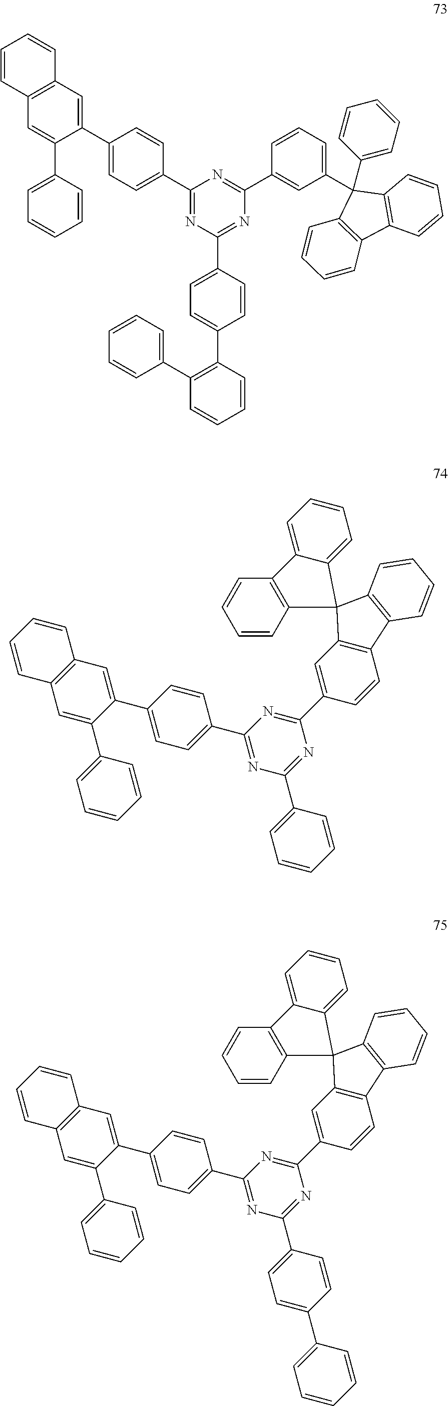 [SMILES: c1ccc(-c2ccccc2-c2ccc(-c3nc(-c4ccc(-c5cc6ccccc6cc5-c5ccccc5)cc4)nc(-c4cccc(C5(c6ccccc6)c6ccccc6-c6ccccc65)c4)n3)cc2)cc1 ; c1ccc(-c2nc(-c3ccc(-c4cc5ccccc5cc4-c4ccccc4)cc3)nc(-c3ccc4c(c3)C3(c5ccccc5-c5ccccc53)c3ccccc3-4)n2)cc1 ; c1ccc(-c2ccc(-c3nc(-c4ccc(-c5cc6ccccc6cc5-c5ccccc5)cc4)nc(-c4ccc5c(c4)C4(c6ccccc6-c6ccccc64)c4ccccc4-5)n3)cc2)cc1]

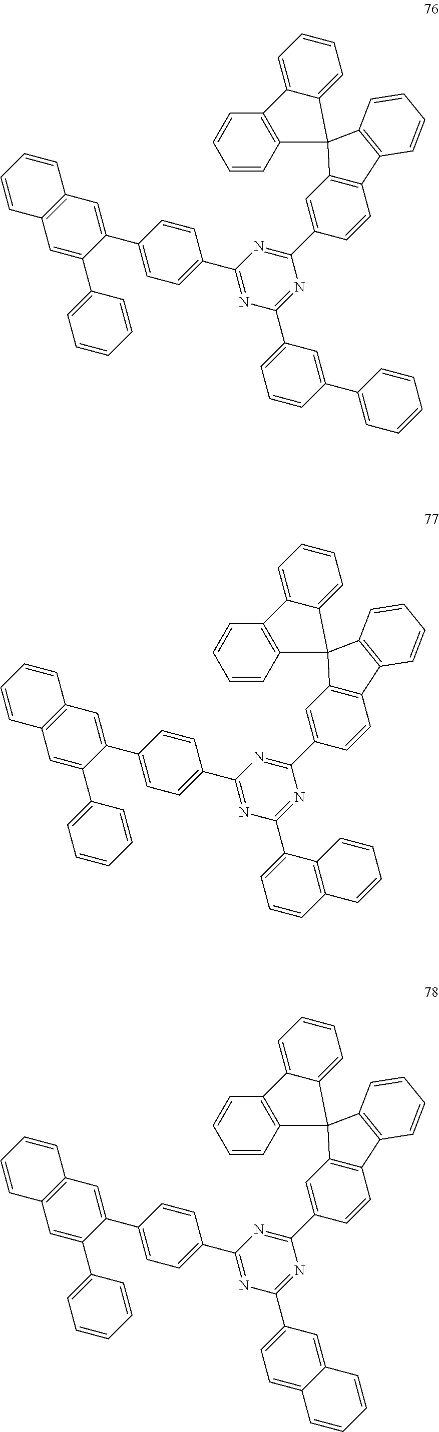 [SMILES: c1ccc(-c2cccc(-c3nc(-c4ccc(-c5cc6ccccc6cc5-c5ccccc5)cc4)nc(-c4ccc5c(c4)C4(c6ccccc6-c6ccccc64)c4ccccc4-5)n3)c2)cc1 ; c1ccc(-c2cc3ccccc3cc2-c2ccc(-c3nc(-c4ccc5c(c4)C4(c6ccccc6-c6ccccc64)c4ccccc4-5)nc(-c4cccc5ccccc45)n3)cc2)cc1 ; c1ccc(-c2cc3ccccc3cc2-c2ccc(-c3nc(-c4ccc5c(c4)C4(c6ccccc6-c6ccccc64)c4ccccc4-5)nc(-c4ccc5ccccc5c4)n3)cc2)cc1]

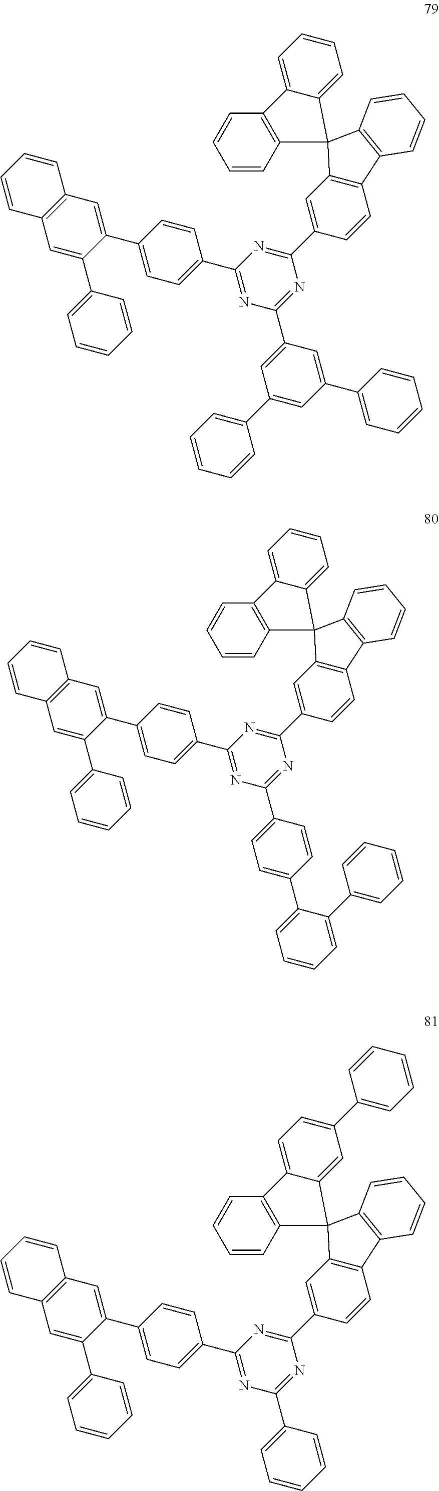 [SMILES: c1ccc(-c2cc(-c3ccccc3)cc(-c3nc(-c4ccc(-c5cc6ccccc6cc5-c5ccccc5)cc4)nc(-c4ccc5c(c4)C4(c6ccccc6-c6ccccc64)c4ccccc4-5)n3)c2)cc1 ; c1ccc(-c2ccccc2-c2ccc(-c3nc(-c4ccc(-c5cc6ccccc6cc5-c5ccccc5)cc4)nc(-c4ccc5c(c4)C4(c6ccccc6-c6ccccc64)c4ccccc4-5)n3)cc2)cc1 ; c1ccc(-c2ccc3c(c2)C2(c4ccccc4-3)c3ccccc3-c3ccc(-c4nc(-c5ccccc5)nc(-c5ccc(-c6cc7ccccc7cc6-c6ccccc6)cc5)n4)cc32)cc1]

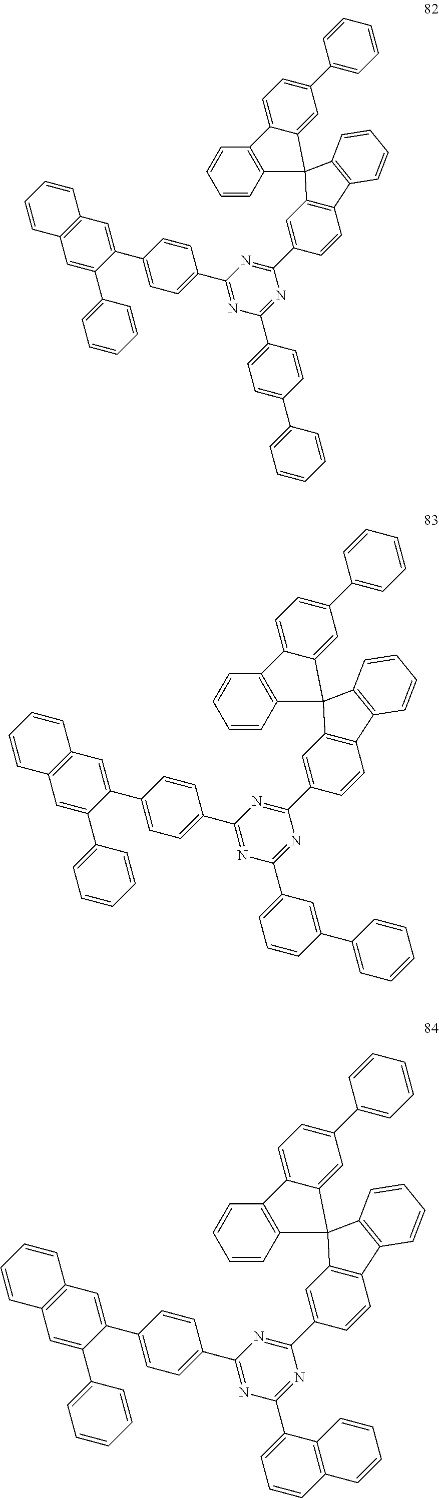 [SMILES: c1ccc(-c2ccc(-c3nc(-c4ccc(-c5cc6ccccc6cc5-c5ccccc5)cc4)nc(-c4ccc5c(c4)C4(c6ccccc6-c6ccc(-c7ccccc7)cc64)c4ccccc4-5)n3)cc2)cc1 ; c1ccc(-c2cccc(-c3nc(-c4ccc(-c5cc6ccccc6cc5-c5ccccc5)cc4)nc(-c4ccc5c(c4)C4(c6ccccc6-c6ccc(-c7ccccc7)cc64)c4ccccc4-5)n3)c2)cc1 ; c1ccc(-c2ccc3c(c2)C2(c4ccccc4-3)c3ccccc3-c3ccc(-c4nc(-c5ccc(-c6cc7ccccc7cc6-c6ccccc6)cc5)nc(-c5cccc6ccccc56)n4)cc32)cc1]

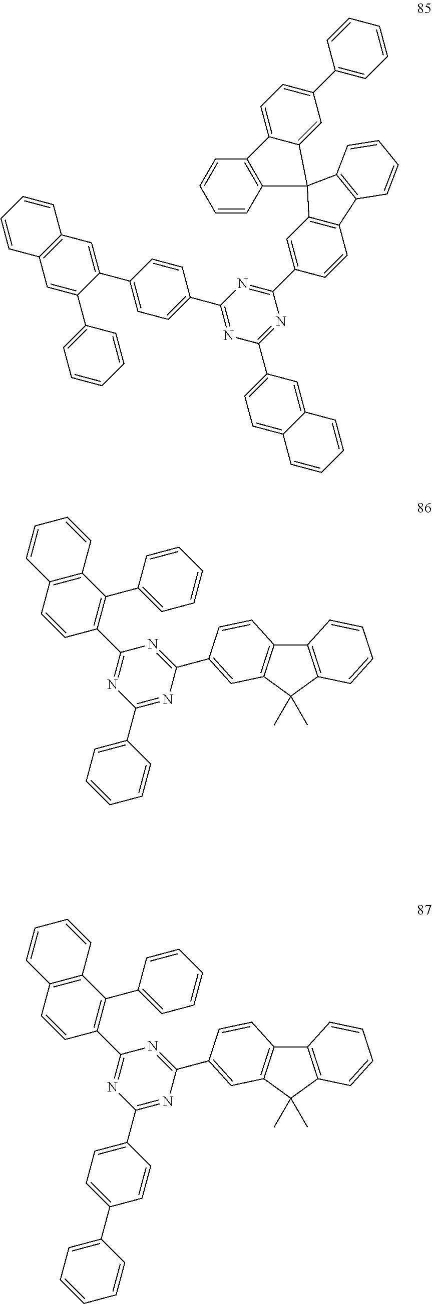 [SMILES: c1ccc(-c2ccc3c(c2)C2(c4ccccc4-3)c3ccccc3-c3ccc(-c4nc(-c5ccc(-c6cc7ccccc7cc6-c6ccccc6)cc5)nc(-c5ccc6ccccc6c5)n4)cc32)cc1 ; CC1(C)c2ccccc2-c2ccc(-c3nc(-c4ccccc4)nc(-c4ccc5ccccc5c4-c4ccccc4)n3)cc21 ; CC1(C)c2ccccc2-c2ccc(-c3nc(-c4ccc(-c5ccccc5)cc4)nc(-c4ccc5ccccc5c4-c4ccccc4)n3)cc21]

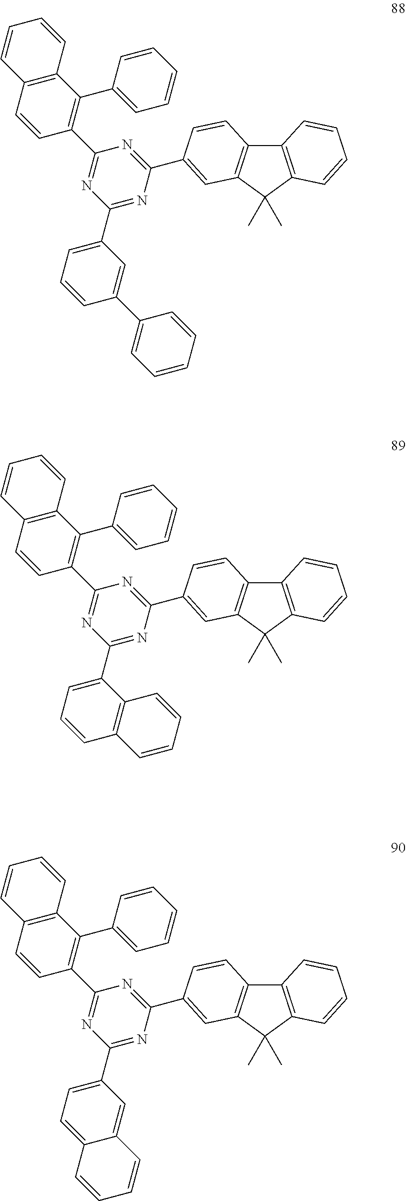 [SMILES: CC1(C)c2ccccc2-c2ccc(-c3nc(-c4cccc(-c5ccccc5)c4)nc(-c4ccc5ccccc5c4-c4ccccc4)n3)cc21 ; CC1(C)c2ccccc2-c2ccc(-c3nc(-c4ccc5ccccc5c4-c4ccccc4)nc(-c4cccc5ccccc45)n3)cc21 ; CC1(C)c2ccccc2-c2ccc(-c3nc(-c4ccc5ccccc5c4)nc(-c4ccc5ccccc5c4-c4ccccc4)n3)cc21]

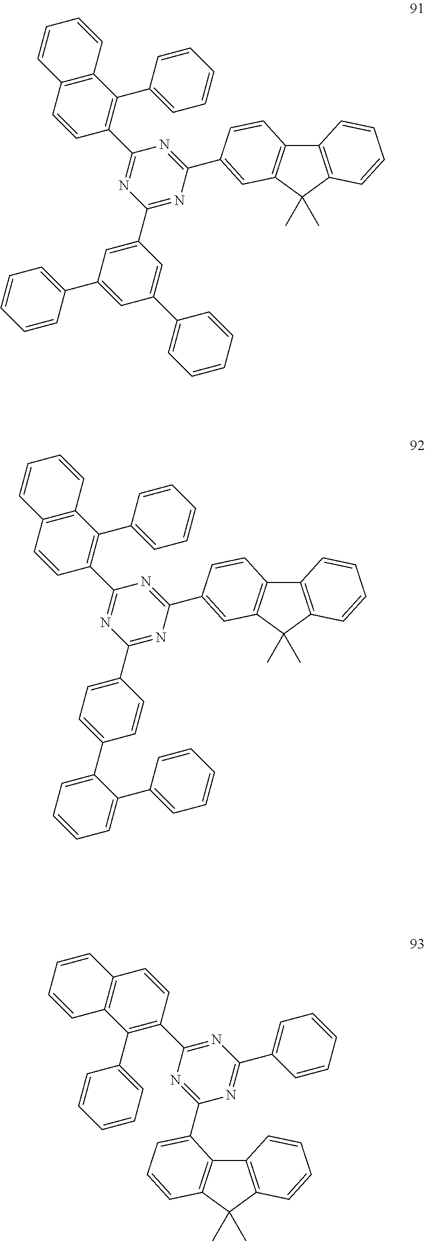 [SMILES: CC1(C)c2ccccc2-c2ccc(-c3nc(-c4cc(-c5ccccc5)cc(-c5ccccc5)c4)nc(-c4ccc5ccccc5c4-c4ccccc4)n3)cc21 ; CC1(C)c2ccccc2-c2ccc(-c3nc(-c4ccc(-c5ccccc5-c5ccccc5)cc4)nc(-c4ccc5ccccc5c4-c4ccccc4)n3)cc21 ; CC1(C)c2ccccc2-c2c(-c3nc(-c4ccccc4)nc(-c4ccc5ccccc5c4-c4ccccc4)n3)cccc21]

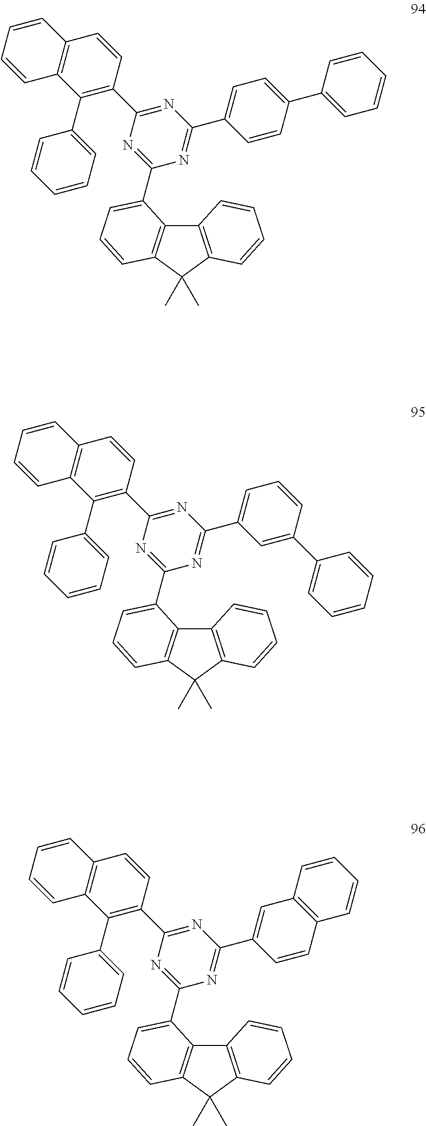 [SMILES: CC1(C)c2ccccc2-c2c(-c3nc(-c4ccc(-c5ccccc5)cc4)nc(-c4ccc5ccccc5c4-c4ccccc4)n3)cccc21 ; CC1(C)c2ccccc2-c2c(-c3nc(-c4cccc(-c5ccccc5)c4)nc(-c4ccc5ccccc5c4-c4ccccc4)n3)cccc21 ; CC1(C)c2ccccc2-c2c(-c3nc(-c4ccc5ccccc5c4)nc(-c4ccc5ccccc5c4-c4ccccc4)n3)cccc21]

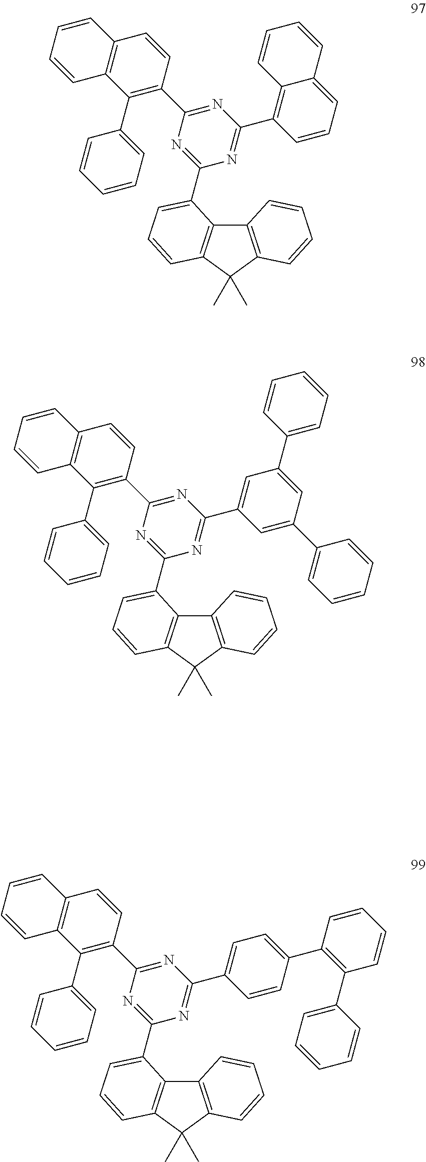 [SMILES: CC1(C)c2ccccc2-c2c(-c3nc(-c4ccc5ccccc5c4-c4ccccc4)nc(-c4cccc5ccccc45)n3)cccc21 ; CC1(C)c2ccccc2-c2c(-c3nc(-c4cc(-c5ccccc5)cc(-c5ccccc5)c4)nc(-c4ccc5ccccc5c4-c4ccccc4)n3)cccc21 ; CC1(C)c2ccccc2-c2c(-c3nc(-c4ccc(-c5ccccc5-c5ccccc5)cc4)nc(-c4ccc5ccccc5c4-c4ccccc4)n3)cccc21]

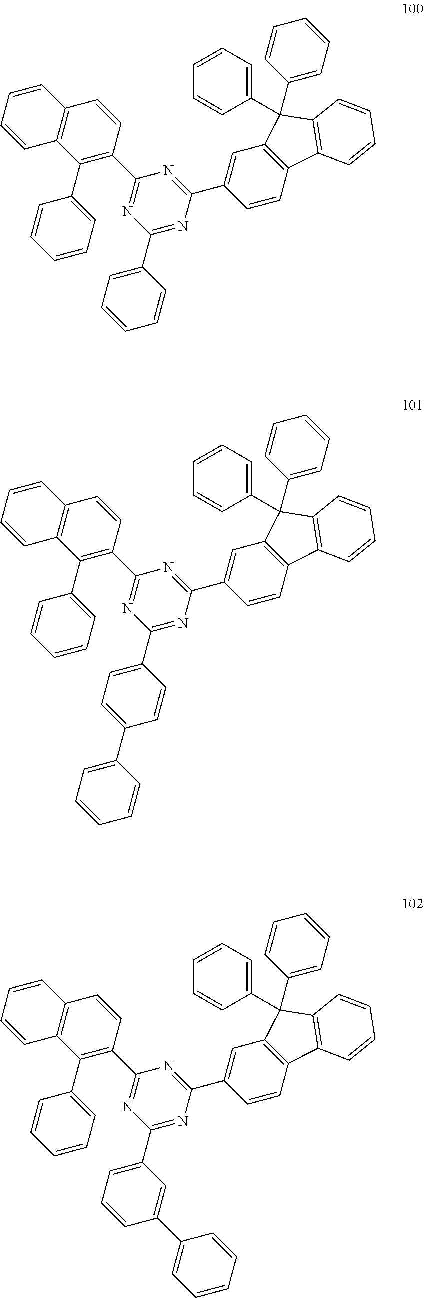 [SMILES: c1ccc(-c2nc(-c3ccc4c(c3)C(c3ccccc3)(c3ccccc3)c3ccccc3-4)nc(-c3ccc4ccccc4c3-c3ccccc3)n2)cc1 ; c1ccc(-c2ccc(-c3nc(-c4ccc5c(c4)C(c4ccccc4)(c4ccccc4)c4ccccc4-5)nc(-c4ccc5ccccc5c4-c4ccccc4)n3)cc2)cc1 ; c1ccc(-c2cccc(-c3nc(-c4ccc5c(c4)C(c4ccccc4)(c4ccccc4)c4ccccc4-5)nc(-c4ccc5ccccc5c4-c4ccccc4)n3)c2)cc1]

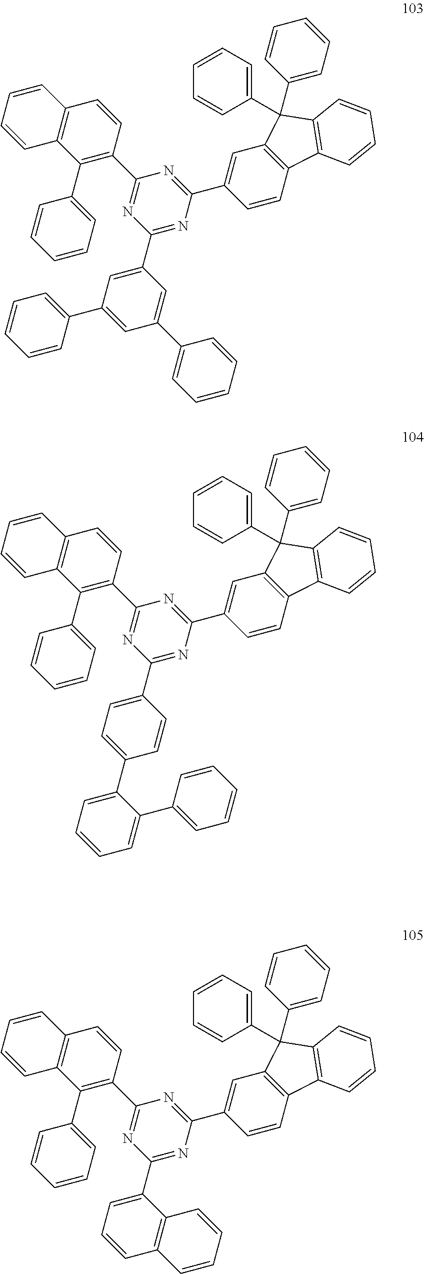 [SMILES: c1ccc(-c2cc(-c3ccccc3)cc(-c3nc(-c4ccc5c(c4)C(c4ccccc4)(c4ccccc4)c4ccccc4-5)nc(-c4ccc5ccccc5c4-c4ccccc4)n3)c2)cc1 ; c1ccc(-c2ccccc2-c2ccc(-c3nc(-c4ccc5c(c4)C(c4ccccc4)(c4ccccc4)c4ccccc4-5)nc(-c4ccc5ccccc5c4-c4ccccc4)n3)cc2)cc1 ; c1ccc(-c2c(-c3nc(-c4ccc5c(c4)C(c4ccccc4)(c4ccccc4)c4ccccc4-5)nc(-c4cccc5ccccc45)n3)ccc3ccccc23)cc1]

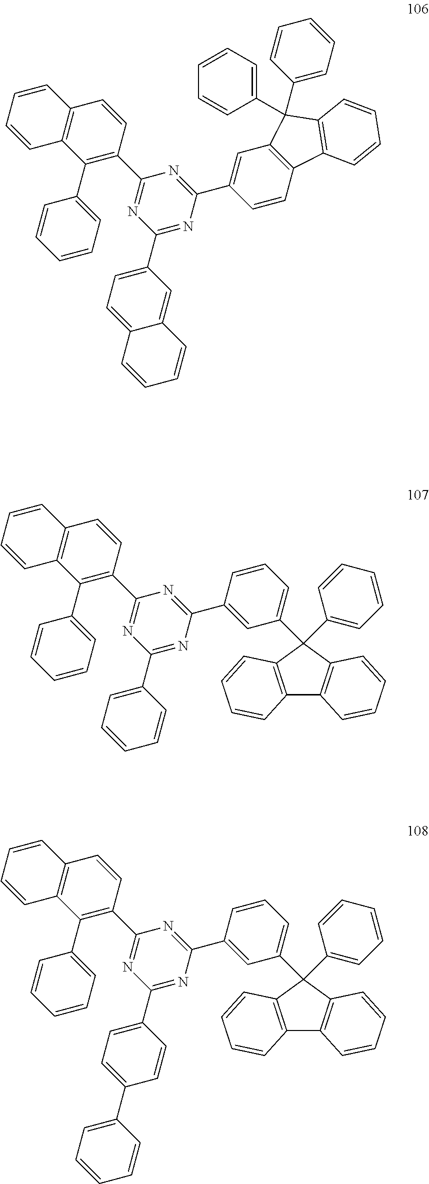 [SMILES: c1ccc(-c2c(-c3nc(-c4ccc5c(c4)C(c4ccccc4)(c4ccccc4)c4ccccc4-5)nc(-c4ccc5ccccc5c4)n3)ccc3ccccc23)cc1 ; c1ccc(-c2nc(-c3cccc(C4(c5ccccc5)c5ccccc5-c5ccccc54)c3)nc(-c3ccc4ccccc4c3-c3ccccc3)n2)cc1 ; c1ccc(-c2ccc(-c3nc(-c4cccc(C5(c6ccccc6)c6ccccc6-c6ccccc65)c4)nc(-c4ccc5ccccc5c4-c4ccccc4)n3)cc2)cc1]

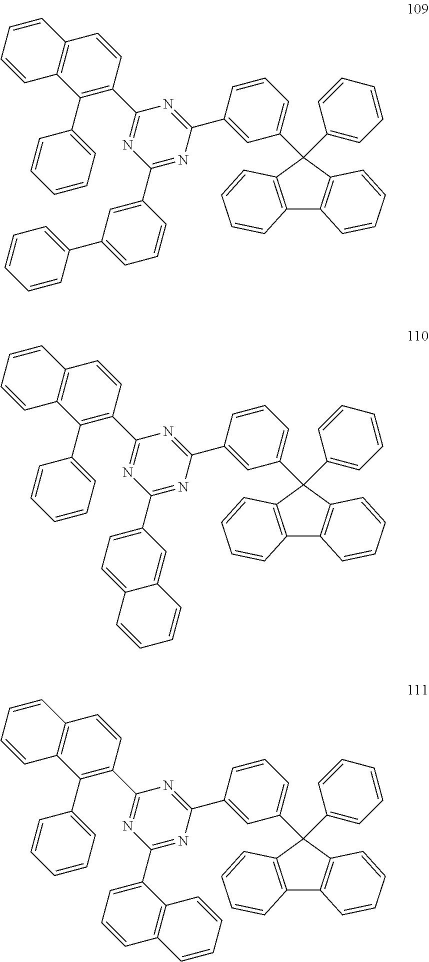 [SMILES: c1ccc(-c2cccc(-c3nc(-c4cccc(C5(c6ccccc6)c6ccccc6-c6ccccc65)c4)nc(-c4ccc5ccccc5c4-c4ccccc4)n3)c2)cc1 ; c1ccc(-c2c(-c3nc(-c4cccc(C5(c6ccccc6)c6ccccc6-c6ccccc65)c4)nc(-c4ccc5ccccc5c4)n3)ccc3ccccc23)cc1 ; c1ccc(-c2c(-c3nc(-c4cccc(C5(c6ccccc6)c6ccccc6-c6ccccc65)c4)nc(-c4cccc5ccccc45)n3)ccc3ccccc23)cc1]

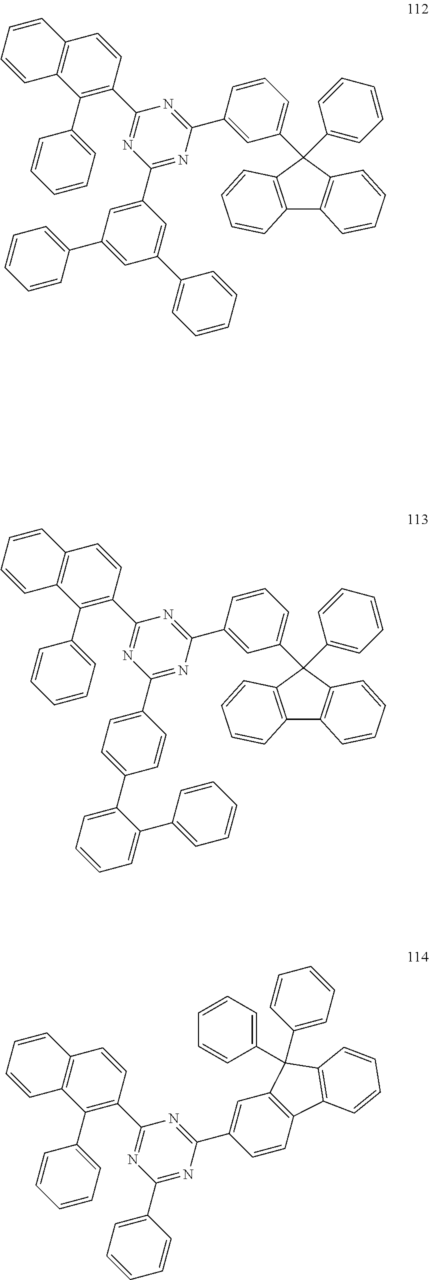 [SMILES: c1ccc(-c2cc(-c3ccccc3)cc(-c3nc(-c4cccc(C5(c6ccccc6)c6ccccc6-c6ccccc65)c4)nc(-c4ccc5ccccc5c4-c4ccccc4)n3)c2)cc1 ; c1ccc(-c2ccccc2-c2ccc(-c3nc(-c4cccc(C5(c6ccccc6)c6ccccc6-c6ccccc65)c4)nc(-c4ccc5ccccc5c4-c4ccccc4)n3)cc2)cc1 ; c1ccc(-c2nc(-c3ccc4c(c3)C(c3ccccc3)(c3ccccc3)c3ccccc3-4)nc(-c3ccc4ccccc4c3-c3ccccc3)n2)cc1]

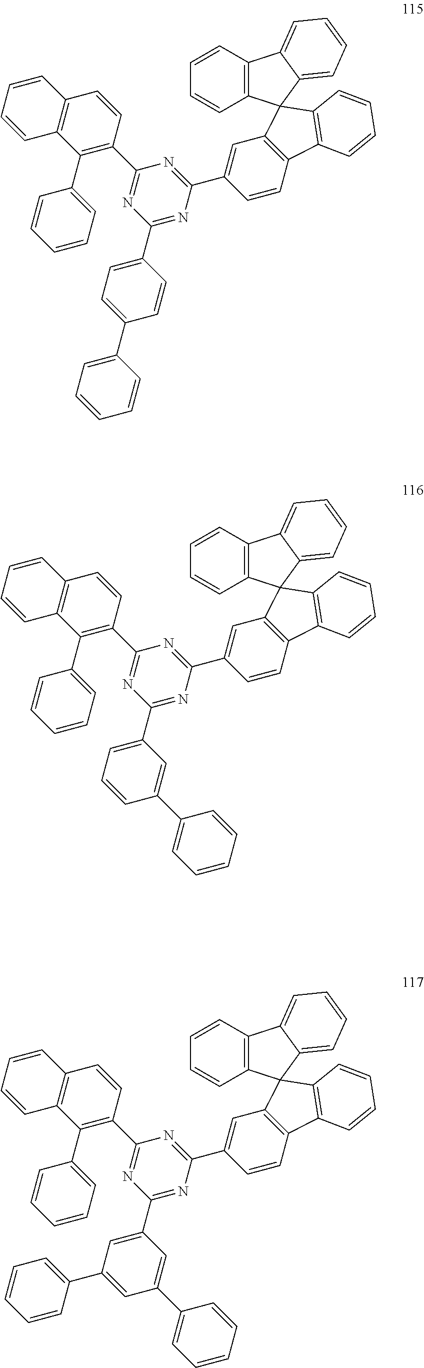 [SMILES: c1ccc(-c2ccc(-c3nc(-c4ccc5c(c4)C4(c6ccccc6-c6ccccc64)c4ccccc4-5)nc(-c4ccc5ccccc5c4-c4ccccc4)n3)cc2)cc1 ; c1ccc(-c2cccc(-c3nc(-c4ccc5c(c4)C4(c6ccccc6-c6ccccc64)c4ccccc4-5)nc(-c4ccc5ccccc5c4-c4ccccc4)n3)c2)cc1 ; c1ccc(-c2cc(-c3ccccc3)cc(-c3nc(-c4ccc5c(c4)C4(c6ccccc6-c6ccccc64)c4ccccc4-5)nc(-c4ccc5ccccc5c4-c4ccccc4)n3)c2)cc1]

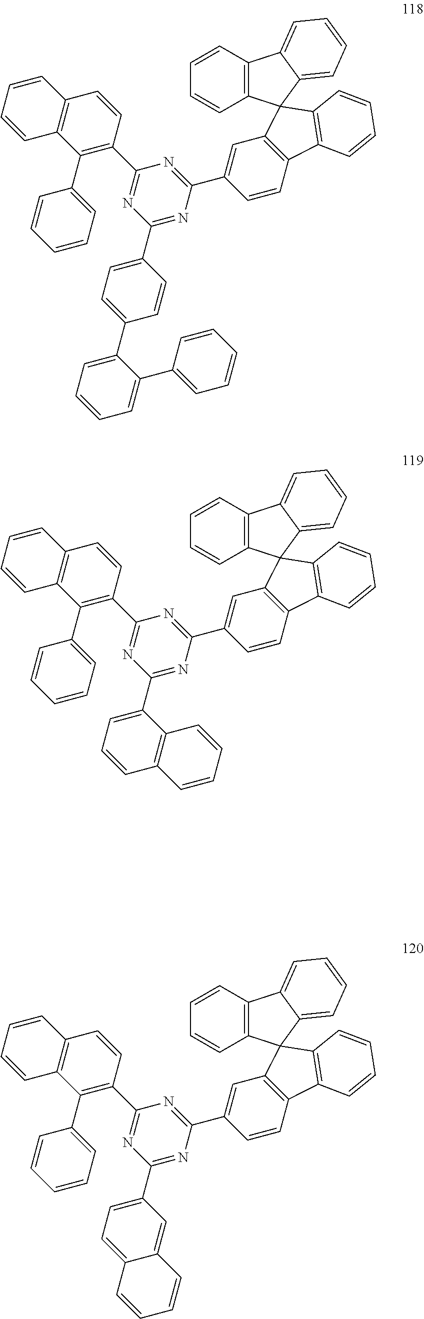 [SMILES: c1ccc(-c2ccccc2-c2ccc(-c3nc(-c4ccc5c(c4)C4(c6ccccc6-c6ccccc64)c4ccccc4-5)nc(-c4ccc5ccccc5c4-c4ccccc4)n3)cc2)cc1 ; c1ccc(-c2c(-c3nc(-c4ccc5c(c4)C4(c6ccccc6-c6ccccc64)c4ccccc4-5)nc(-c4cccc5ccccc45)n3)ccc3ccccc23)cc1 ; c1ccc(-c2c(-c3nc(-c4ccc5c(c4)C4(c6ccccc6-c6ccccc64)c4ccccc4-5)nc(-c4ccc5ccccc5c4)n3)ccc3ccccc23)cc1]

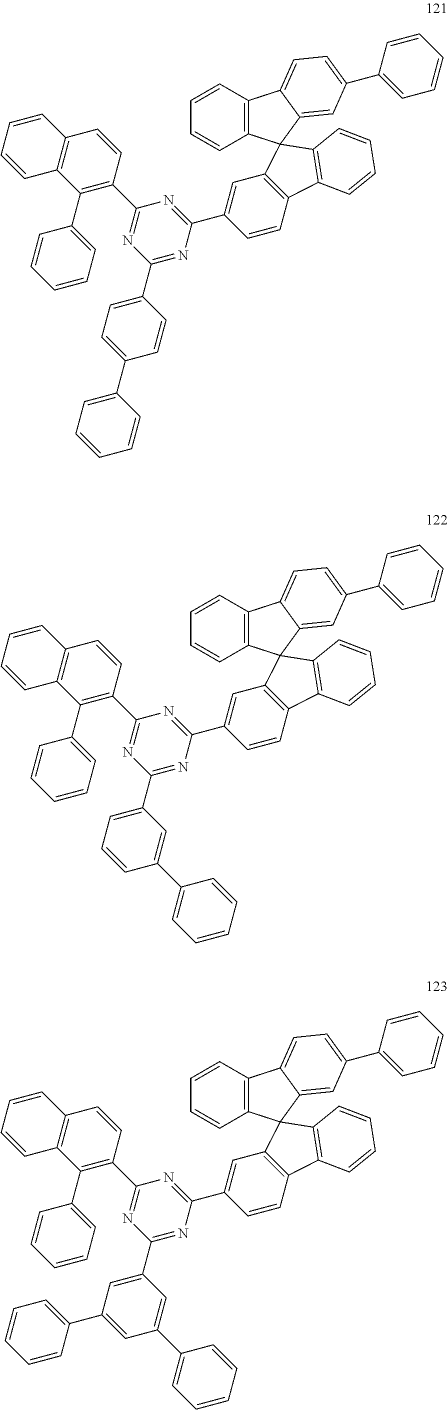 [SMILES: c1ccc(-c2ccc(-c3nc(-c4ccc5c(c4)C4(c6ccccc6-c6ccc(-c7ccccc7)cc64)c4ccccc4-5)nc(-c4ccc5ccccc5c4-c4ccccc4)n3)cc2)cc1 ; c1ccc(-c2cccc(-c3nc(-c4ccc5c(c4)C4(c6ccccc6-c6ccc(-c7ccccc7)cc64)c4ccccc4-5)nc(-c4ccc5ccccc5c4-c4ccccc4)n3)c2)cc1 ; c1ccc(-c2cc(-c3ccccc3)cc(-c3nc(-c4ccc5c(c4)C4(c6ccccc6-c6ccc(-c7ccccc7)cc64)c4ccccc4-5)nc(-c4ccc5ccccc5c4-c4ccccc4)n3)c2)cc1]

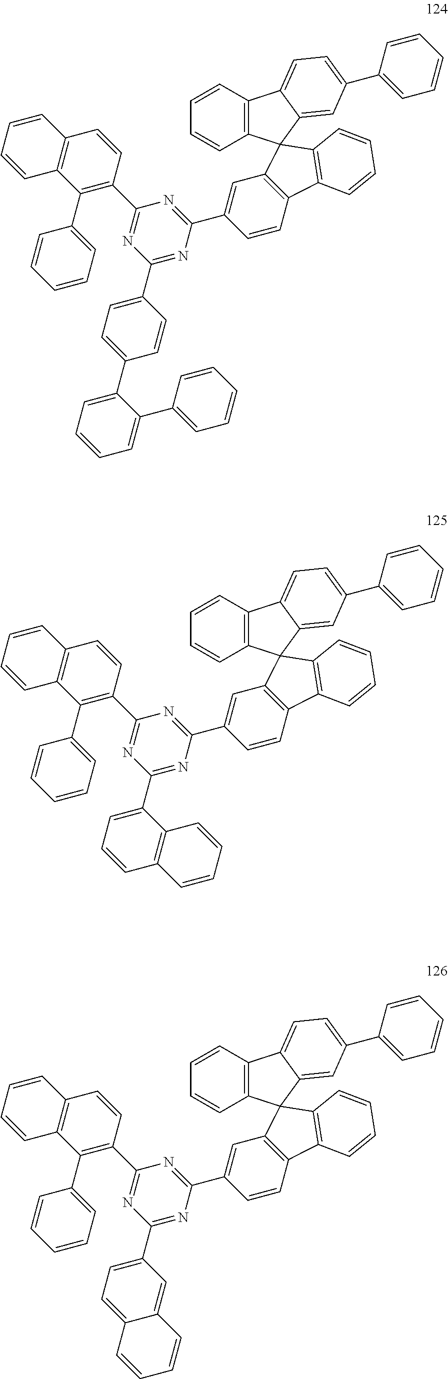 [SMILES: c1ccc(-c2ccc3c(c2)C2(c4ccccc4-3)c3ccccc3-c3ccc(-c4nc(-c5ccc(-c6ccccc6-c6ccccc6)cc5)nc(-c5ccc6ccccc6c5-c5ccccc5)n4)cc32)cc1 ; c1ccc(-c2ccc3c(c2)C2(c4ccccc4-3)c3ccccc3-c3ccc(-c4nc(-c5ccc6ccccc6c5-c5ccccc5)nc(-c5cccc6ccccc56)n4)cc32)cc1 ; c1ccc(-c2ccc3c(c2)C2(c4ccccc4-3)c3ccccc3-c3ccc(-c4nc(-c5ccc6ccccc6c5)nc(-c5ccc6ccccc6c5-c5ccccc5)n4)cc32)cc1]

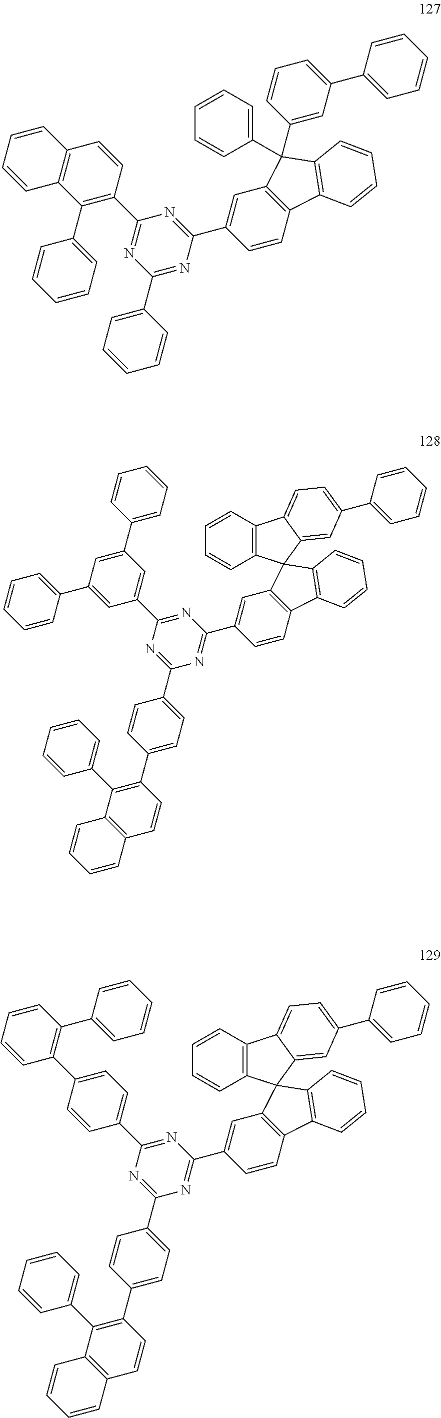 [SMILES: c1ccc(-c2cccc(C3(c4ccccc4)c4ccccc4-c4ccc(-c5nc(-c6ccccc6)nc(-c6ccc7ccccc7c6-c6ccccc6)n5)cc43)c2)cc1 ; c1ccc(-c2cc(-c3ccccc3)cc(-c3nc(-c4ccc(-c5ccc6ccccc6c5-c5ccccc5)cc4)nc(-c4ccc5c(c4)C4(c6ccccc6-c6ccc(-c7ccccc7)cc64)c4ccccc4-5)n3)c2)cc1 ; c1ccc(-c2ccc3c(c2)C2(c4ccccc4-3)c3ccccc3-c3ccc(-c4nc(-c5ccc(-c6ccccc6-c6ccccc6)cc5)nc(-c5ccc(-c6ccc7ccccc7c6-c6ccccc6)cc5)n4)cc32)cc1]

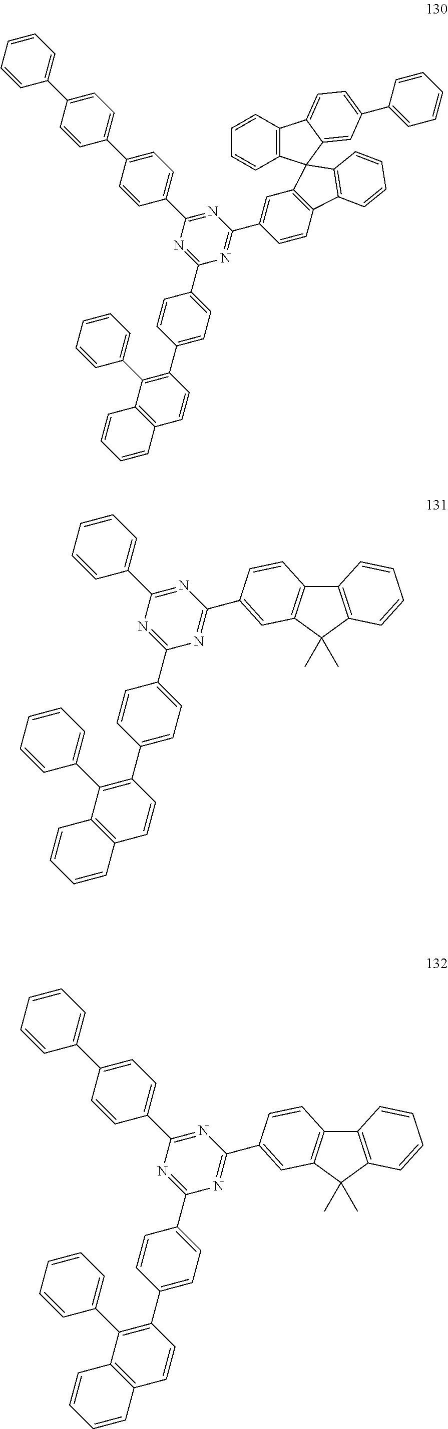 [SMILES: c1ccc(-c2ccc(-c3ccc(-c4nc(-c5ccc(-c6ccc7ccccc7c6-c6ccccc6)cc5)nc(-c5ccc6c(c5)C5(c7ccccc7-c7ccc(-c8ccccc8)cc75)c5ccccc5-6)n4)cc3)cc2)cc1 ; CC1(C)c2ccccc2-c2ccc(-c3nc(-c4ccccc4)nc(-c4ccc(-c5ccc6ccccc6c5-c5ccccc5)cc4)n3)cc21 ; CC1(C)c2ccccc2-c2ccc(-c3nc(-c4ccc(-c5ccccc5)cc4)nc(-c4ccc(-c5ccc6ccccc6c5-c5ccccc5)cc4)n3)cc21]

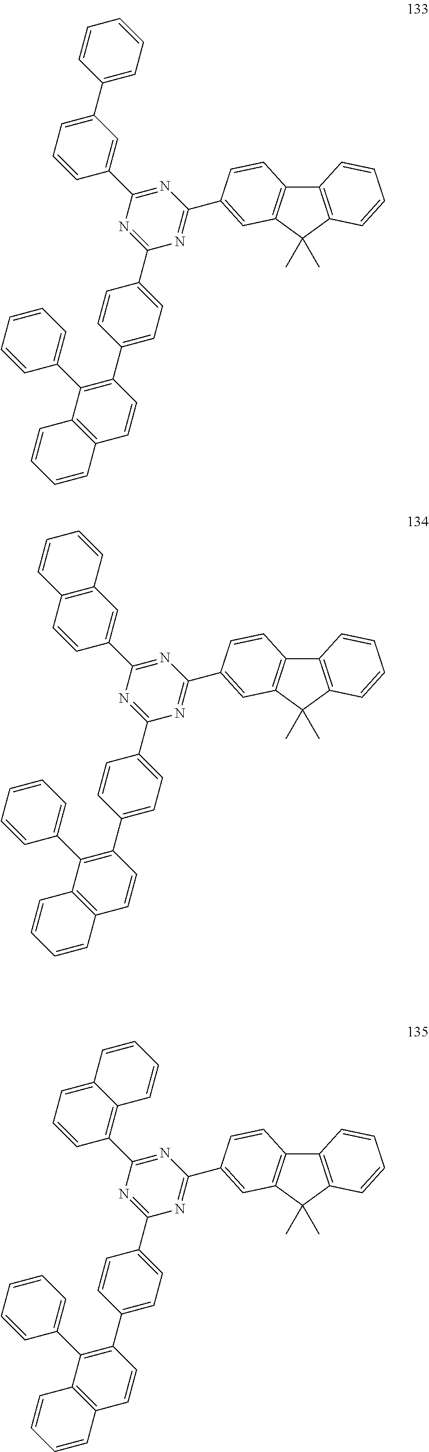 [SMILES: CC1(C)c2ccccc2-c2ccc(-c3nc(-c4ccc(-c5ccc6ccccc6c5-c5ccccc5)cc4)nc(-c4cccc(-c5ccccc5)c4)n3)cc21 ; CC1(C)c2ccccc2-c2ccc(-c3nc(-c4ccc(-c5ccc6ccccc6c5-c5ccccc5)cc4)nc(-c4ccc5ccccc5c4)n3)cc21 ; CC1(C)c2ccccc2-c2ccc(-c3nc(-c4ccc(-c5ccc6ccccc6c5-c5ccccc5)cc4)nc(-c4cccc5ccccc45)n3)cc21]

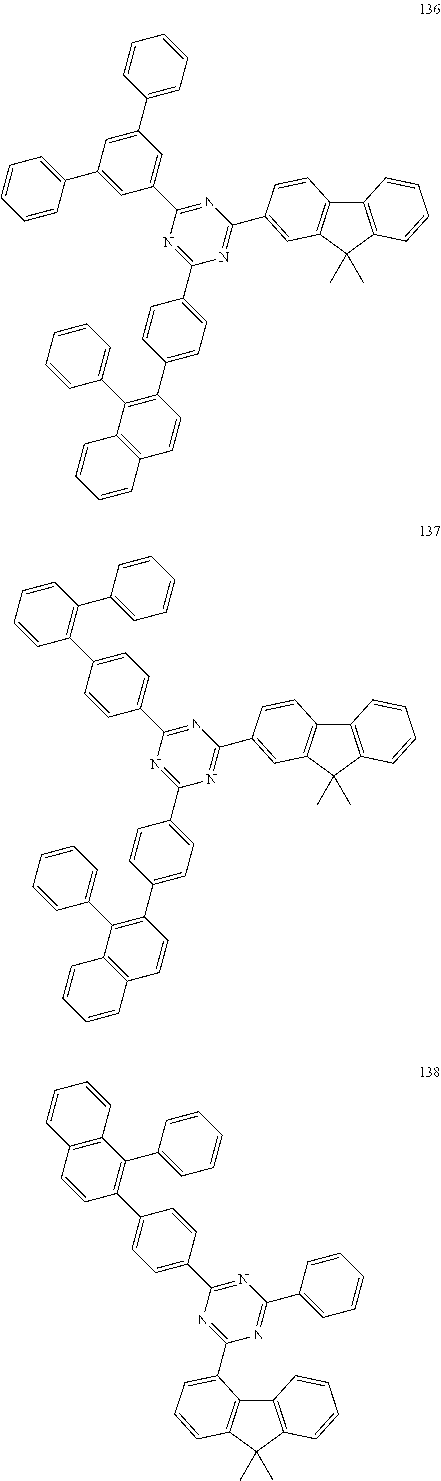 [SMILES: CC1(C)c2ccccc2-c2ccc(-c3nc(-c4ccc(-c5ccc6ccccc6c5-c5ccccc5)cc4)nc(-c4cc(-c5ccccc5)cc(-c5ccccc5)c4)n3)cc21 ; CC1(C)c2ccccc2-c2ccc(-c3nc(-c4ccc(-c5ccccc5-c5ccccc5)cc4)nc(-c4ccc(-c5ccc6ccccc6c5-c5ccccc5)cc4)n3)cc21 ; CC1(C)c2ccccc2-c2c(-c3nc(-c4ccccc4)nc(-c4ccc(-c5ccc6ccccc6c5-c5ccccc5)cc4)n3)cccc21]

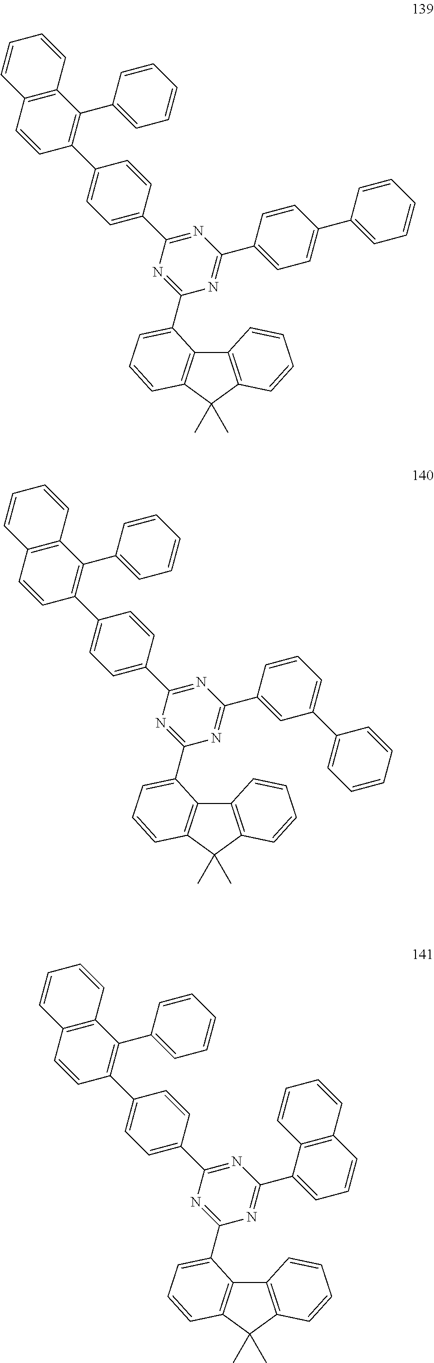 [SMILES: CC1(C)c2ccccc2-c2c(-c3nc(-c4ccc(-c5ccccc5)cc4)nc(-c4ccc(-c5ccc6ccccc6c5-c5ccccc5)cc4)n3)cccc21 ; CC1(C)c2ccccc2-c2c(-c3nc(-c4ccc(-c5ccc6ccccc6c5-c5ccccc5)cc4)nc(-c4cccc(-c5ccccc5)c4)n3)cccc21 ; CC1(C)c2ccccc2-c2c(-c3nc(-c4ccc(-c5ccc6ccccc6c5-c5ccccc5)cc4)nc(-c4cccc5ccccc45)n3)cccc21]

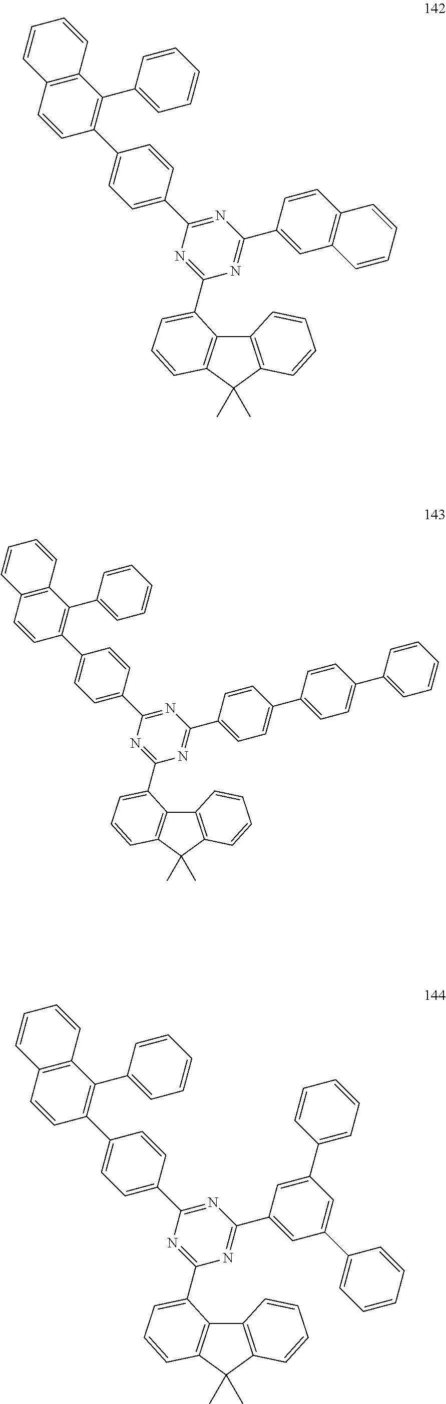 [SMILES: CC1(C)c2ccccc2-c2c(-c3nc(-c4ccc(-c5ccc6ccccc6c5-c5ccccc5)cc4)nc(-c4ccc5ccccc5c4)n3)cccc21 ; CC1(C)c2ccccc2-c2c(-c3nc(-c4ccc(-c5ccc(-c6ccccc6)cc5)cc4)nc(-c4ccc(-c5ccc6ccccc6c5-c5ccccc5)cc4)n3)cccc21 ; CC1(C)c2ccccc2-c2c(-c3nc(-c4ccc(-c5ccc6ccccc6c5-c5ccccc5)cc4)nc(-c4cc(-c5ccccc5)cc(-c5ccccc5)c4)n3)cccc21]

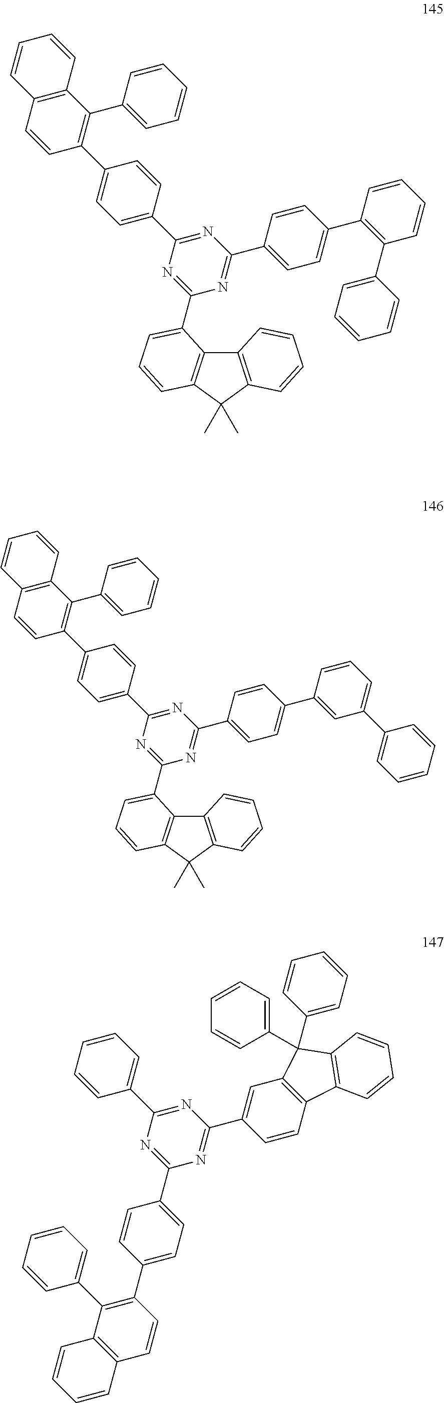 [SMILES: CC1(C)c2ccccc2-c2c(-c3nc(-c4ccc(-c5ccccc5-c5ccccc5)cc4)nc(-c4ccc(-c5ccc6ccccc6c5-c5ccccc5)cc4)n3)cccc21 ; CC1(C)c2ccccc2-c2c(-c3nc(-c4ccc(-c5cccc(-c6ccccc6)c5)cc4)nc(-c4ccc(-c5ccc6ccccc6c5-c5ccccc5)cc4)n3)cccc21 ; c1ccc(-c2nc(-c3ccc(-c4ccc5ccccc5c4-c4ccccc4)cc3)nc(-c3ccc4c(c3)C(c3ccccc3)(c3ccccc3)c3ccccc3-4)n2)cc1]

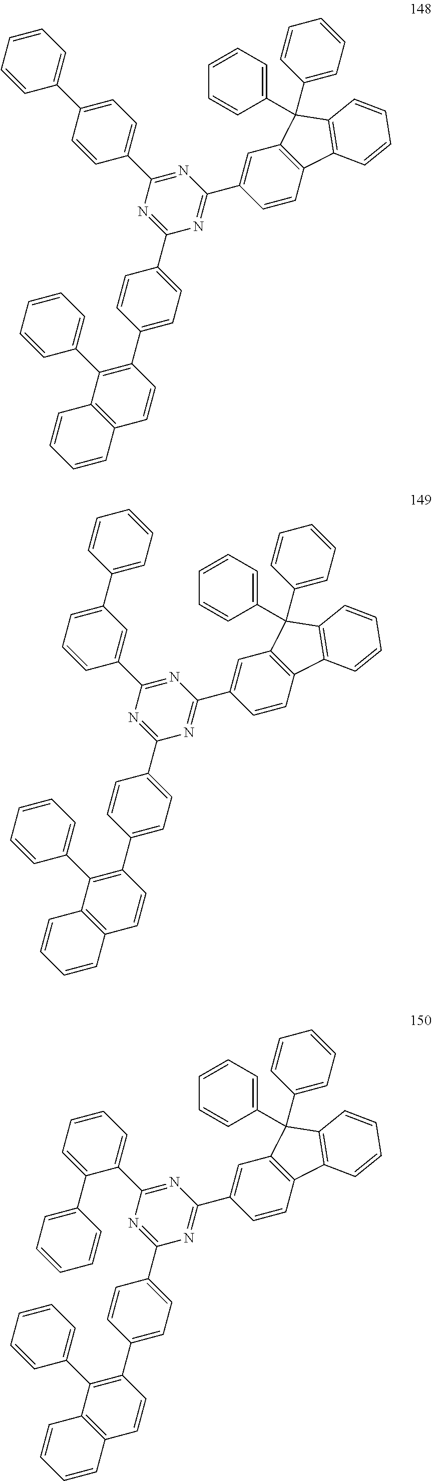 [SMILES: c1ccc(-c2ccc(-c3nc(-c4ccc(-c5ccc6ccccc6c5-c5ccccc5)cc4)nc(-c4ccc5c(c4)C(c4ccccc4)(c4ccccc4)c4ccccc4-5)n3)cc2)cc1 ; c1ccc(-c2cccc(-c3nc(-c4ccc(-c5ccc6ccccc6c5-c5ccccc5)cc4)nc(-c4ccc5c(c4)C(c4ccccc4)(c4ccccc4)c4ccccc4-5)n3)c2)cc1 ; c1ccc(-c2ccccc2-c2nc(-c3ccc(-c4ccc5ccccc5c4-c4ccccc4)cc3)nc(-c3ccc4c(c3)C(c3ccccc3)(c3ccccc3)c3ccccc3-4)n2)cc1]

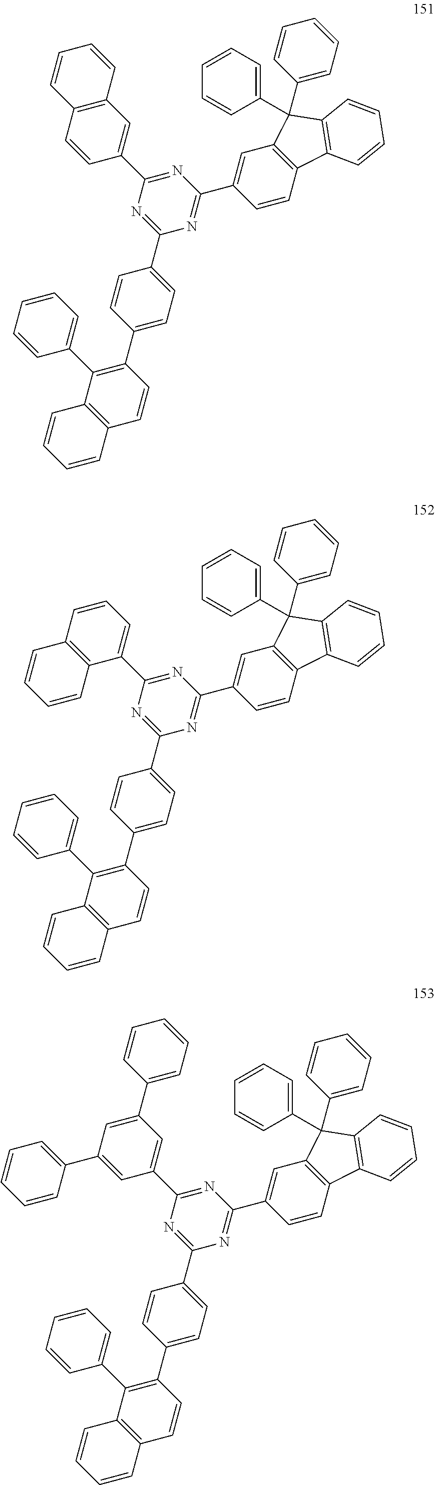 [SMILES: c1ccc(-c2c(-c3ccc(-c4nc(-c5ccc6c(c5)C(c5ccccc5)(c5ccccc5)c5ccccc5-6)nc(-c5ccc6ccccc6c5)n4)cc3)ccc3ccccc23)cc1 ; c1ccc(-c2c(-c3ccc(-c4nc(-c5ccc6c(c5)C(c5ccccc5)(c5ccccc5)c5ccccc5-6)nc(-c5cccc6ccccc56)n4)cc3)ccc3ccccc23)cc1 ; c1ccc(-c2cc(-c3ccccc3)cc(-c3nc(-c4ccc(-c5ccc6ccccc6c5-c5ccccc5)cc4)nc(-c4ccc5c(c4)C(c4ccccc4)(c4ccccc4)c4ccccc4-5)n3)c2)cc1]

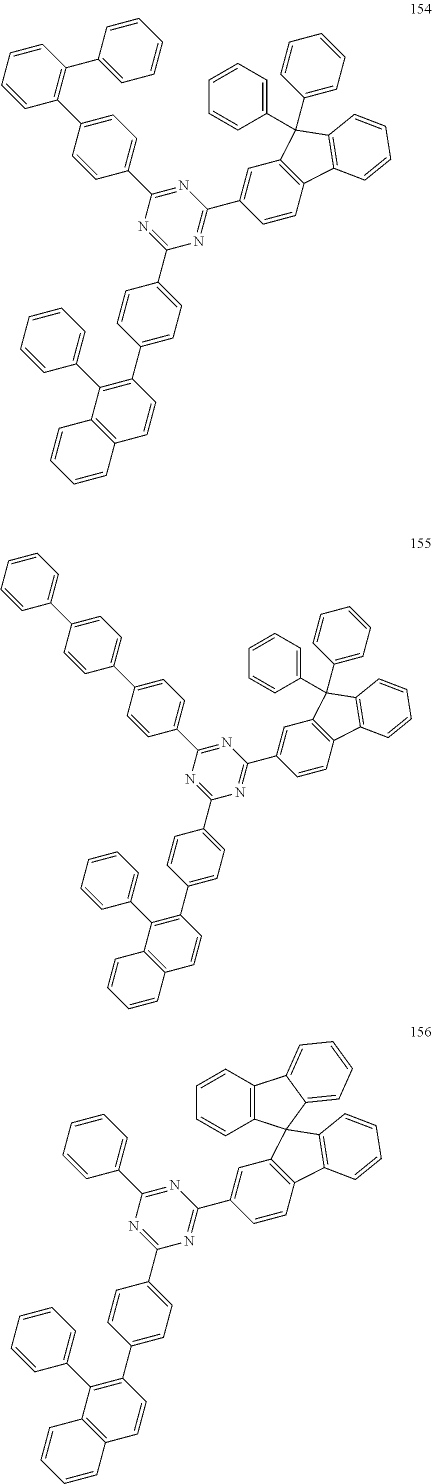 [SMILES: c1ccc(-c2ccccc2-c2ccc(-c3nc(-c4ccc(-c5ccc6ccccc6c5-c5ccccc5)cc4)nc(-c4ccc5c(c4)C(c4ccccc4)(c4ccccc4)c4ccccc4-5)n3)cc2)cc1 ; c1ccc(-c2ccc(-c3ccc(-c4nc(-c5ccc(-c6ccc7ccccc7c6-c6ccccc6)cc5)nc(-c5ccc6c(c5)C(c5ccccc5)(c5ccccc5)c5ccccc5-6)n4)cc3)cc2)cc1 ; c1ccc(-c2nc(-c3ccc(-c4ccc5ccccc5c4-c4ccccc4)cc3)nc(-c3ccc4c(c3)C3(c5ccccc5-c5ccccc53)c3ccccc3-4)n2)cc1]

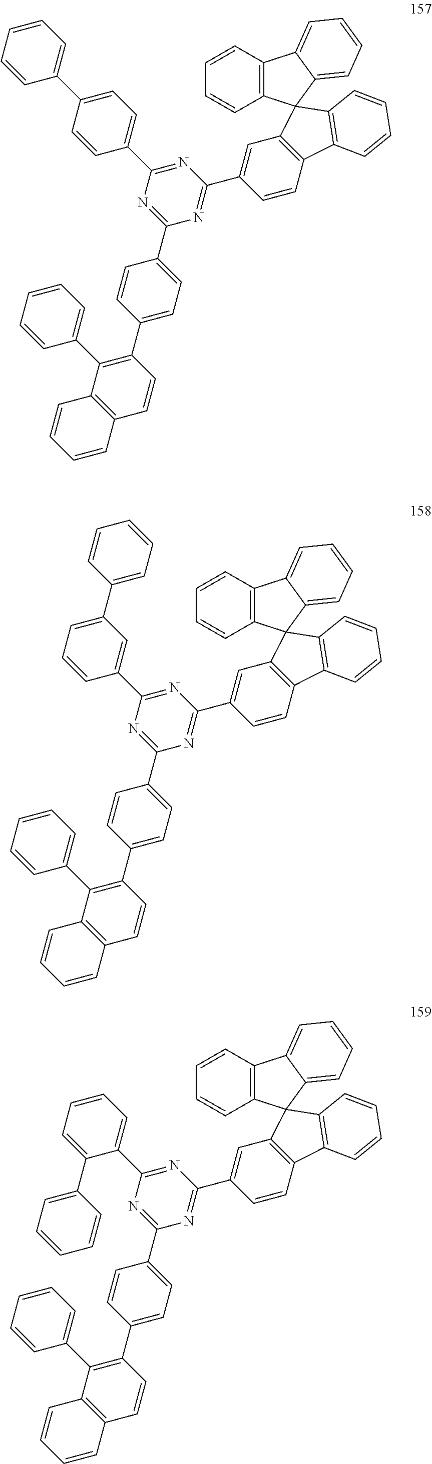 [SMILES: c1ccc(-c2ccc(-c3nc(-c4ccc(-c5ccc6ccccc6c5-c5ccccc5)cc4)nc(-c4ccc5c(c4)C4(c6ccccc6-c6ccccc64)c4ccccc4-5)n3)cc2)cc1 ; c1ccc(-c2cccc(-c3nc(-c4ccc(-c5ccc6ccccc6c5-c5ccccc5)cc4)nc(-c4ccc5c(c4)C4(c6ccccc6-c6ccccc64)c4ccccc4-5)n3)c2)cc1 ; c1ccc(-c2ccccc2-c2nc(-c3ccc(-c4ccc5ccccc5c4-c4ccccc4)cc3)nc(-c3ccc4c(c3)C3(c5ccccc5-c5ccccc53)c3ccccc3-4)n2)cc1]

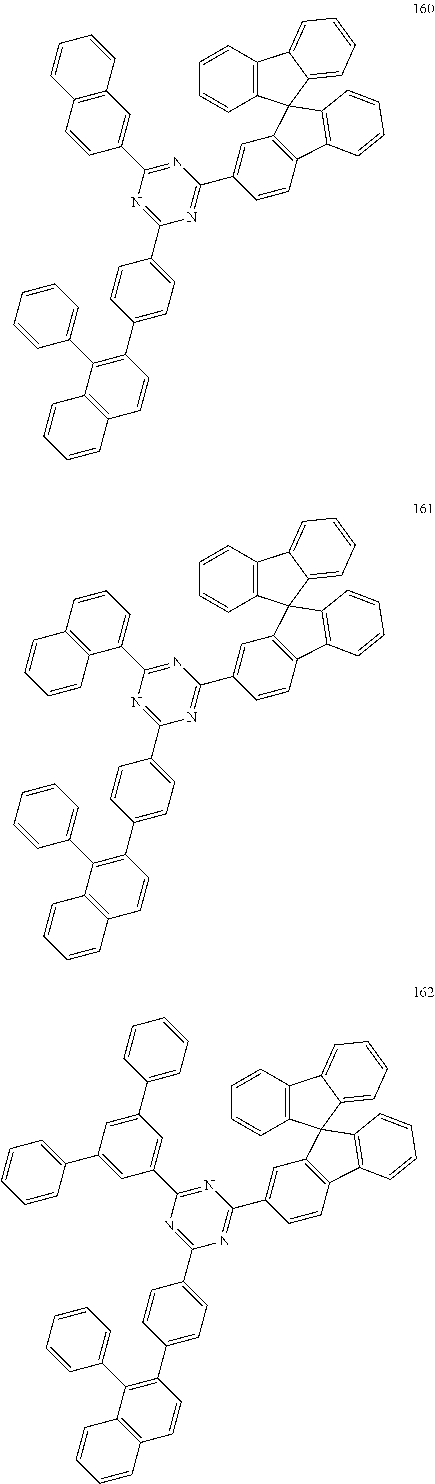 [SMILES: c1ccc(-c2c(-c3ccc(-c4nc(-c5ccc6c(c5)C5(c7ccccc7-c7ccccc75)c5ccccc5-6)nc(-c5ccc6ccccc6c5)n4)cc3)ccc3ccccc23)cc1 ; c1ccc(-c2c(-c3ccc(-c4nc(-c5ccc6c(c5)C5(c7ccccc7-c7ccccc75)c5ccccc5-6)nc(-c5cccc6ccccc56)n4)cc3)ccc3ccccc23)cc1 ; c1ccc(-c2cc(-c3ccccc3)cc(-c3nc(-c4ccc(-c5ccc6ccccc6c5-c5ccccc5)cc4)nc(-c4ccc5c(c4)C4(c6ccccc6-c6ccccc64)c4ccccc4-5)n3)c2)cc1]

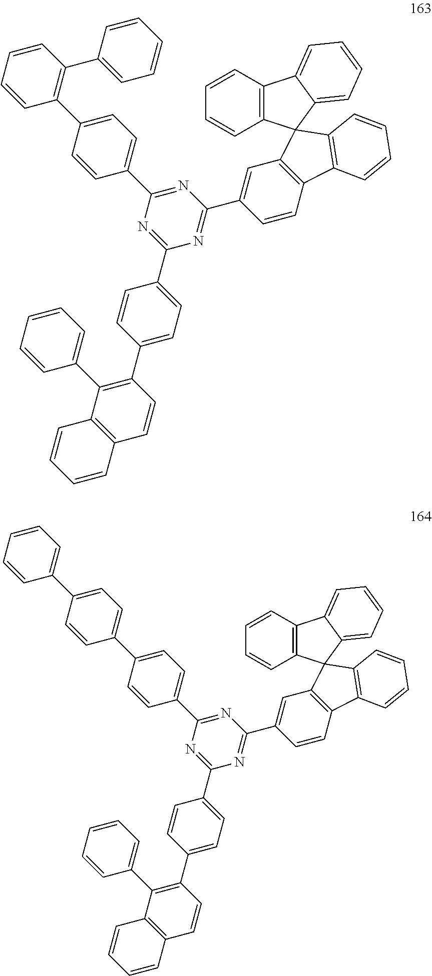 [SMILES: c1ccc(-c2ccccc2-c2ccc(-c3nc(-c4ccc(-c5ccc6ccccc6c5-c5ccccc5)cc4)nc(-c4ccc5c(c4)C4(c6ccccc6-c6ccccc64)c4ccccc4-5)n3)cc2)cc1 ; c1ccc(-c2ccc(-c3ccc(-c4nc(-c5ccc(-c6ccc7ccccc7c6-c6ccccc6)cc5)nc(-c5ccc6c(c5)C5(c7ccccc7-c7ccccc75)c5ccccc5-6)n4)cc3)cc2)cc1]

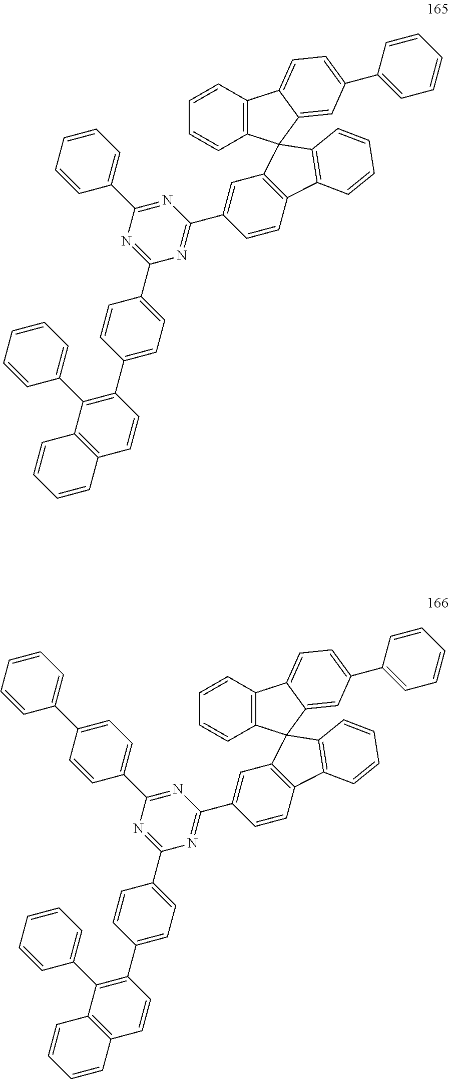 [SMILES: c1ccc(-c2ccc3c(c2)C2(c4ccccc4-3)c3ccccc3-c3ccc(-c4nc(-c5ccccc5)nc(-c5ccc(-c6ccc7ccccc7c6-c6ccccc6)cc5)n4)cc32)cc1 ; c1ccc(-c2ccc(-c3nc(-c4ccc(-c5ccc6ccccc6c5-c5ccccc5)cc4)nc(-c4ccc5c(c4)C4(c6ccccc6-c6ccc(-c7ccccc7)cc64)c4ccccc4-5)n3)cc2)cc1]

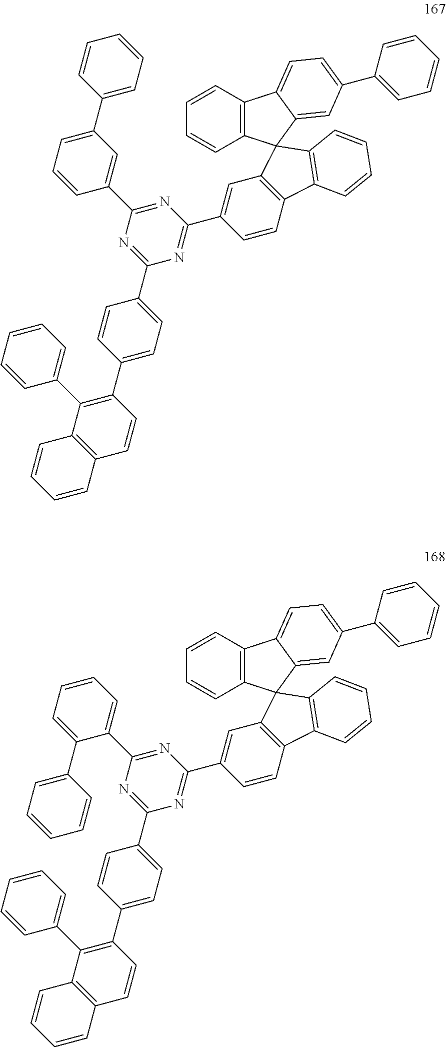 [SMILES: c1ccc(-c2cccc(-c3nc(-c4ccc(-c5ccc6ccccc6c5-c5ccccc5)cc4)nc(-c4ccc5c(c4)C4(c6ccccc6-c6ccc(-c7ccccc7)cc64)c4ccccc4-5)n3)c2)cc1 ; c1ccc(-c2ccc3c(c2)C2(c4ccccc4-3)c3ccccc3-c3ccc(-c4nc(-c5ccc(-c6ccc7ccccc7c6-c6ccccc6)cc5)nc(-c5ccccc5-c5ccccc5)n4)cc32)cc1]

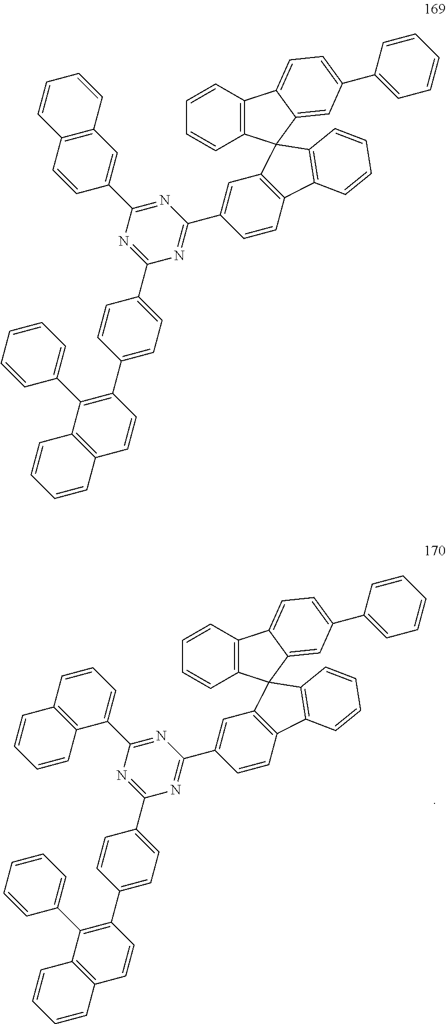 [SMILES: c1ccc(-c2ccc3c(c2)C2(c4ccccc4-3)c3ccccc3-c3ccc(-c4nc(-c5ccc(-c6ccc7ccccc7c6-c6ccccc6)cc5)nc(-c5ccc6ccccc6c5)n4)cc32)cc1 ; c1ccc(-c2ccc3c(c2)C2(c4ccccc4-3)c3ccccc3-c3ccc(-c4nc(-c5ccc(-c6ccc7ccccc7c6-c6ccccc6)cc5)nc(-c5cccc6ccccc56)n4)cc32)cc1]

The nitrogen-containing compound according to an embodiment may include a triazine ring, and a first substituent and a second substituent that are each linked to the triazine ring. The first substituent may include a phenylnaphthalene moiety, and the second substituent may include a fluorene moiety. By having such a structure, the nitrogen-containing compound according to an embodiment may thus have improved material stability and a high glass transition temperature, thereby preventing crystallization. The light emitting element ED according to an embodiment includes a nitrogen-containing compound according to an embodiment, and thus, thermal stability and chemical stability are improved, thereby exhibiting improved element characteristics. Satisfactory charge transport characteristics may be achieved without a substantial increase in driving voltage, so that increased luminous efficiency may be achieved.

In the light emitting element ED according to an embodiment, the electron transport region ETR may further include a compound represented by Formula ET-2:

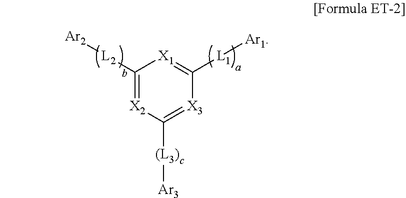

In Formula ET-2, at least one of X₁ to X₃ may each be N; and the remainder of X₁ to X₃ may each independently be C(Rₐ). In Formula ET-2, Rₐ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In Formula ET-2, Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-2, a to c may each independently be an integer from 0 to 10. In Formula ET-2, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. When a to c are each 2 or more, multiple groups of each of L₁ to L₃ may each independently be a substituted or unsubstituted arylene group having 6 to 30 ring-forming carbon atoms or a substituted or unsubstituted heteroarylene group having 2 to 30 ring-forming carbon atoms.

The electron transport region ETR may include an anthracene-based compound. However, embodiments are not limited thereto. The electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzimidazol-1-yl)phenyl)-9,10-dinaphthylanthracene, 1,3,5-tri(1-phenyl-1H-benz[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,08)-(1,1′-biphenyl-4-olato)aluminum (BAlq), beryllium bis(benzoquinolin-10-olate) (Bebg₂), 9,10-di(naphthalene-2-yl)anthracene (ADN), 1,3-bis[3,5-di(pyridin-3-yl)phenyl]benzene (BmPyPhB), or a mixture thereof.

In an embodiment, the electron transport region ETR may include at least one compound selected from Compound ET1 to Compound ET36:

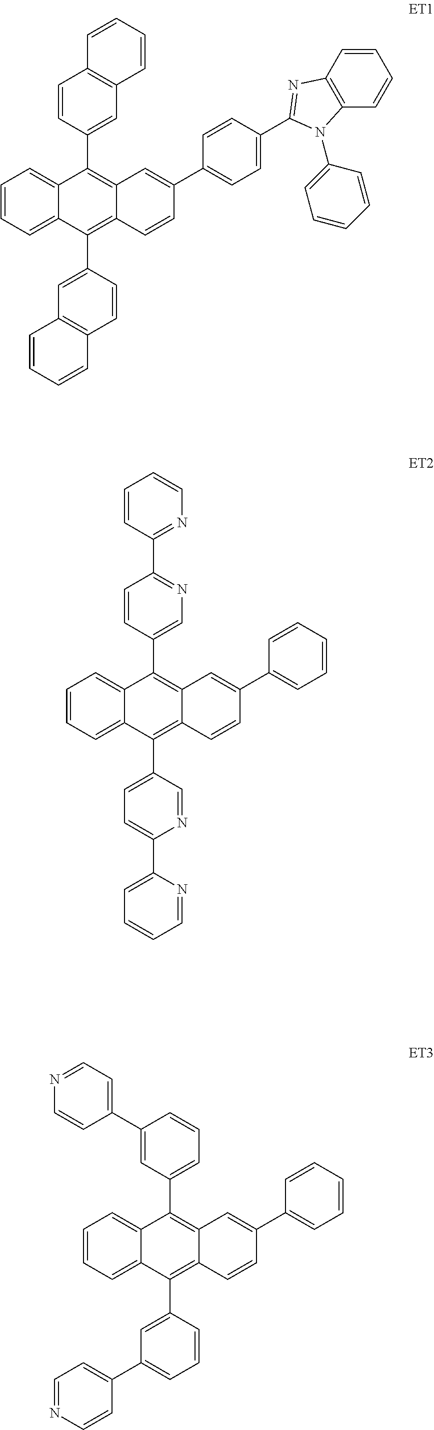 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

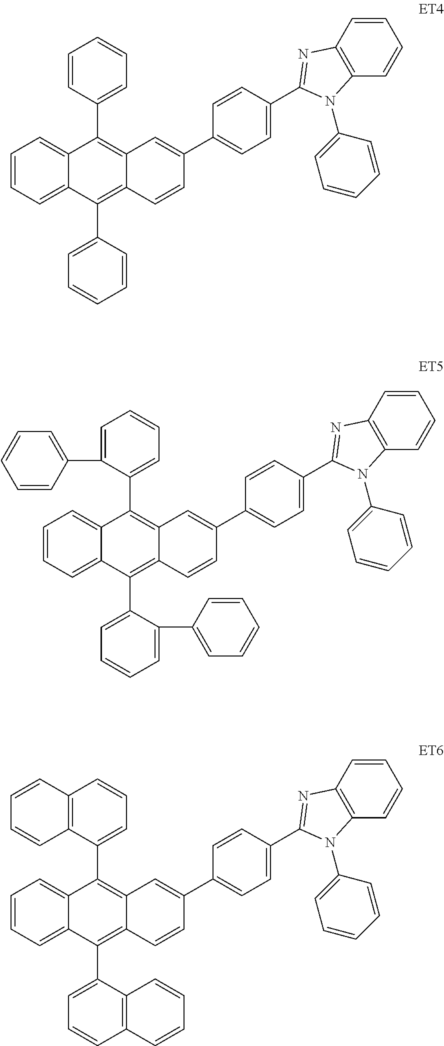 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

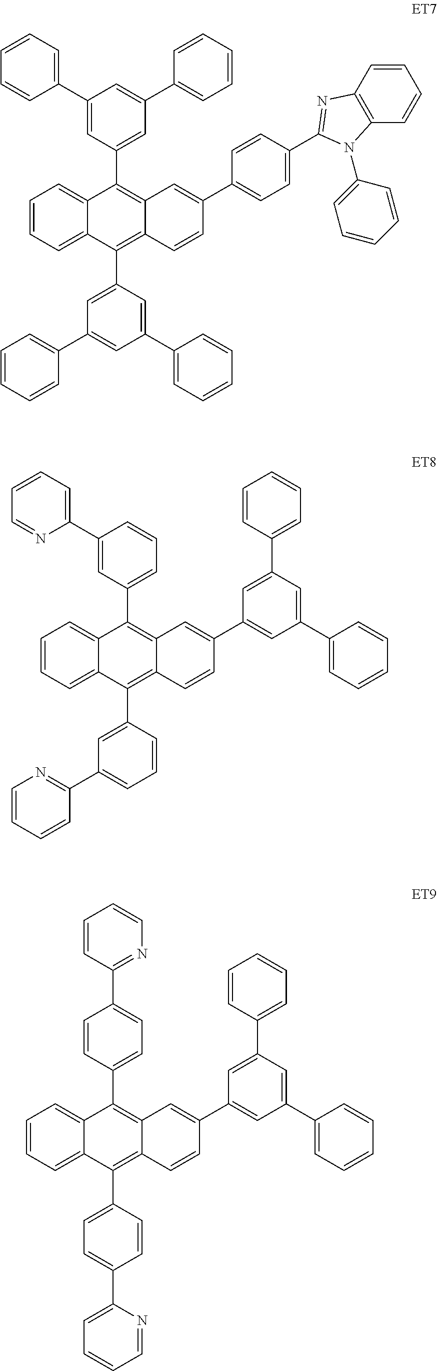 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

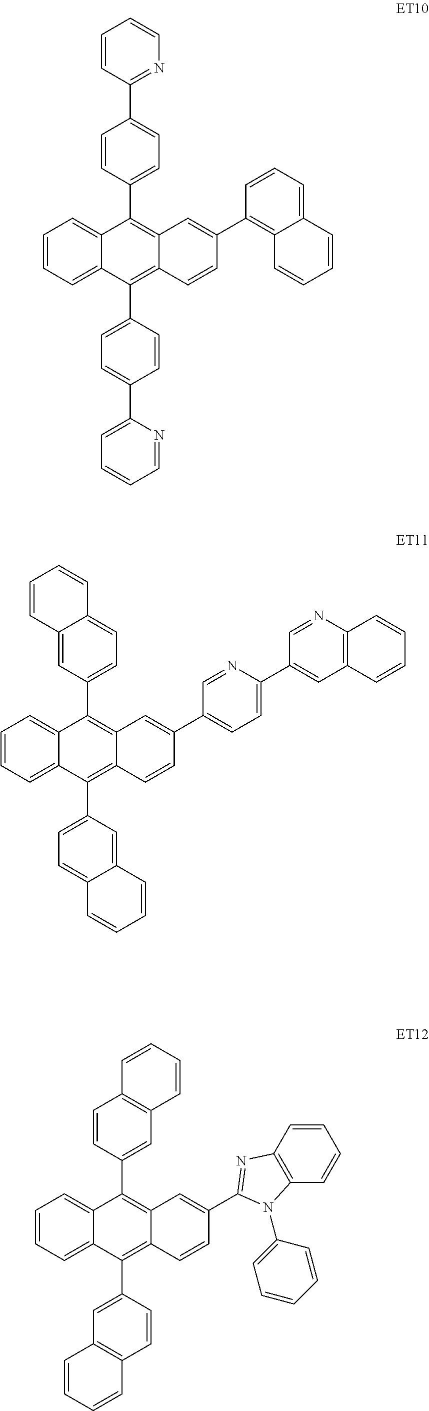 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

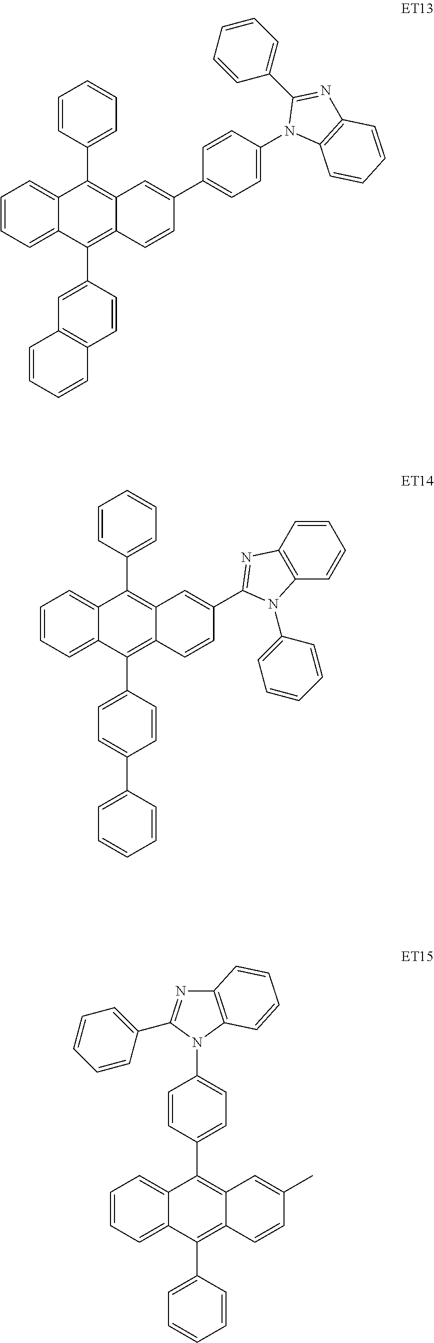 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

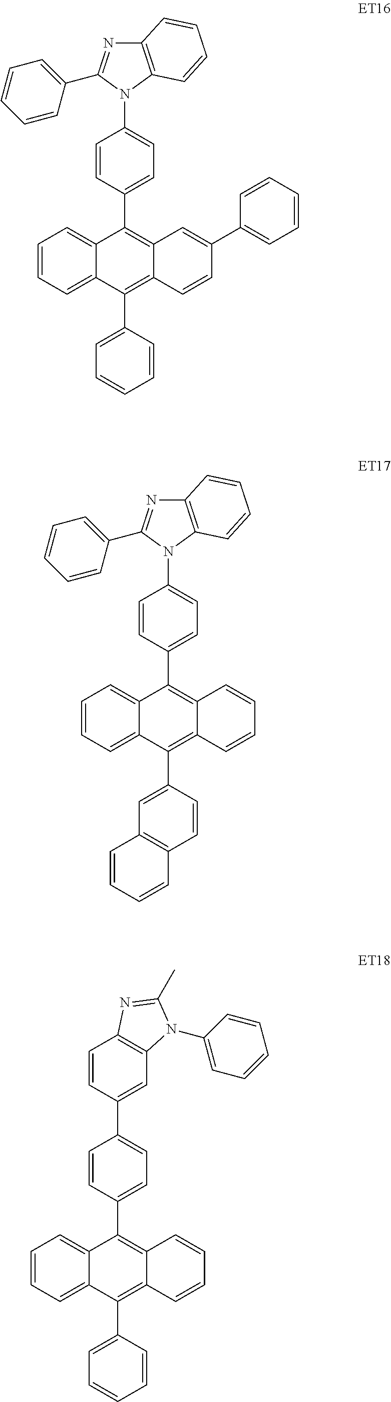 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

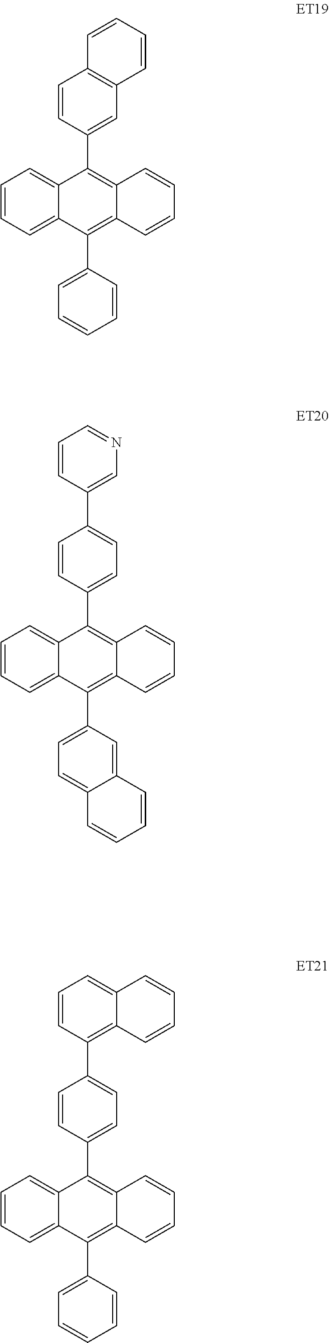

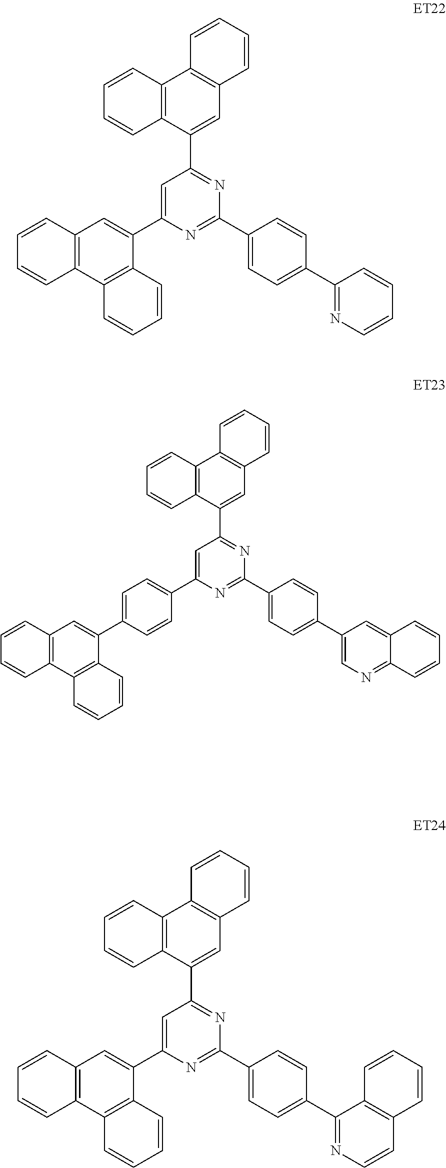 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

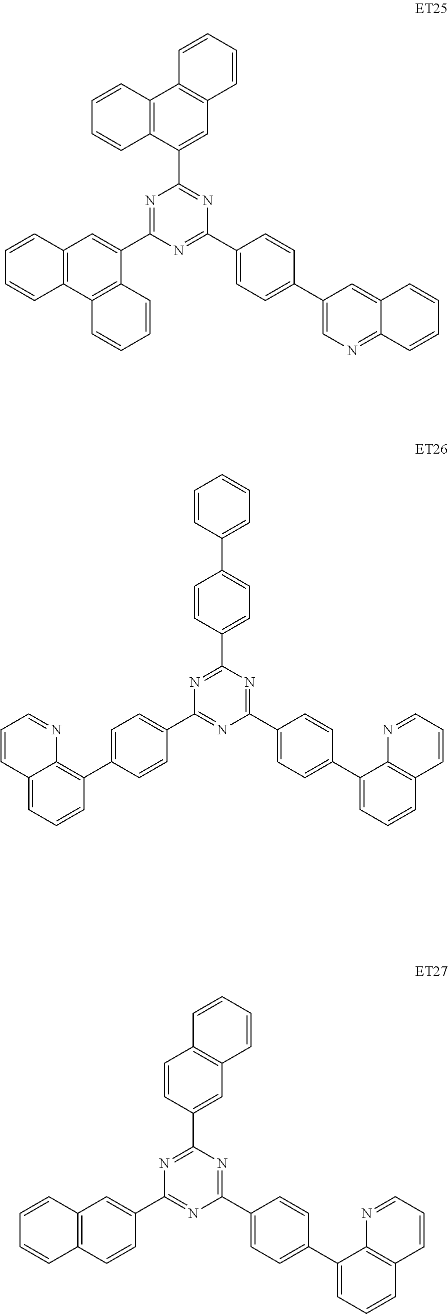 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

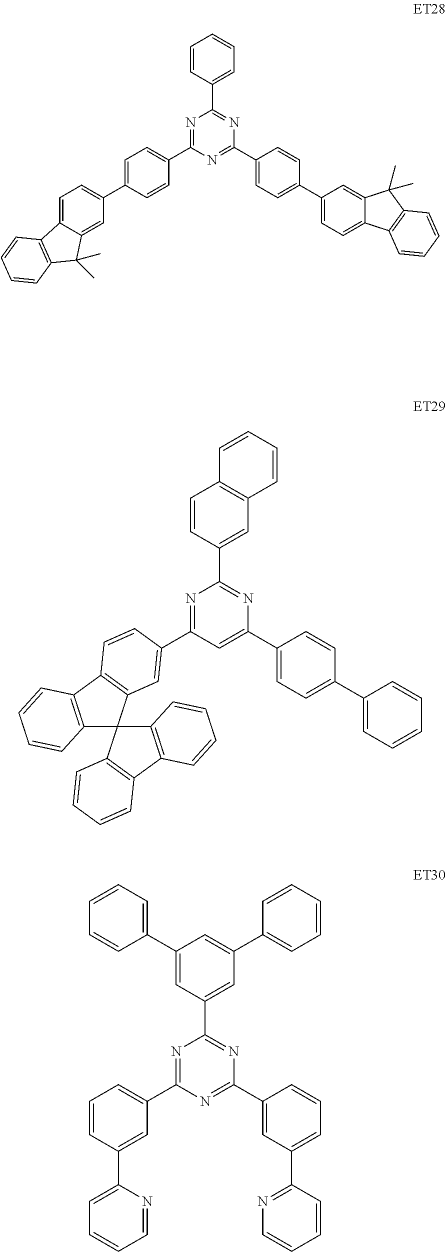 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

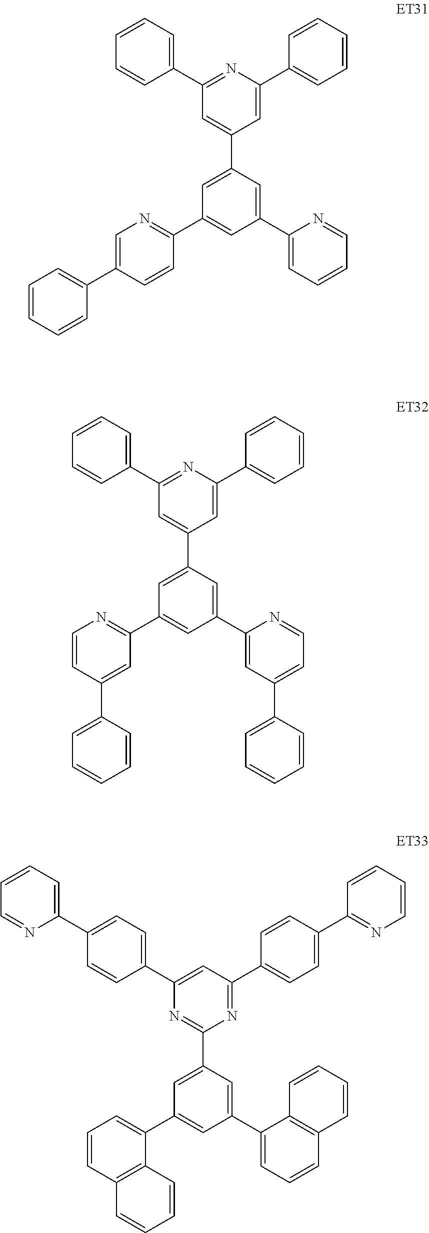 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

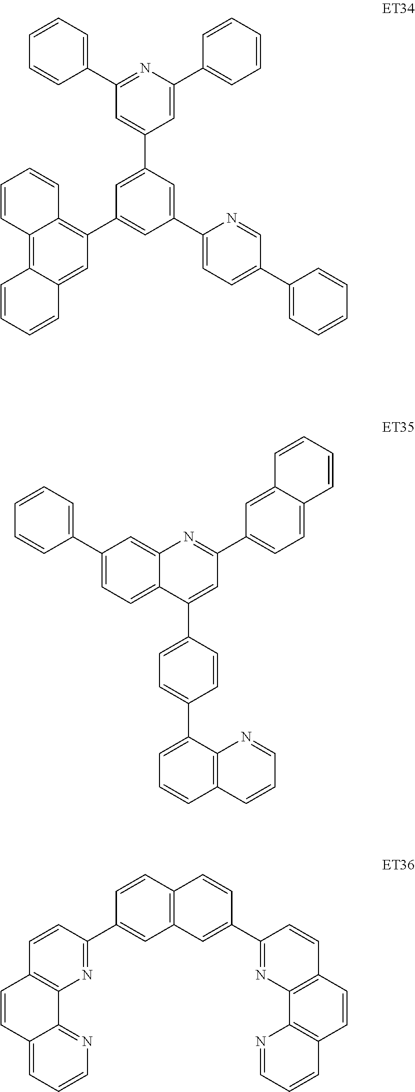 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In an embodiment, the electron transport regions ETR may include a metal halide such as LiF, NaCl, CsF, RbCl, RbI, CuI, or KI; a lanthanide metal such as Yb; or a co-deposited material of a metal halide and a lanthanide metal. For example, the electron transport region ETR may include KI:Yb, RbI:Yb, LiF:Yb, etc., as a co-deposited material. The electron transport region ETR may include metal oxide such as LizO or BaO, or 8-hydroxyl-lithium quinolate (Lig), etc., but embodiments are not limited thereto. The electron transport region ETR may also be formed of a mixture material of an electron transport material and an insulating organometallic salt. The organometallic salt may be a material having an energy band gap equal to or greater than about 4 eV. For example, the organometallic salt may include a metal acetate, a metal benzoate, a metal acetoacetate, a metal acetylacetonate, or a metal stearate.

The electron transport region ETR may further include at least one of 2,9-dimethyl-4,7-diphenyl-1,10-phenanthroline (BCP), diphenyl(4-(triphenylsilyl)phenyl)phosphine oxide (TSPO1), and 4,7-diphenyl-1,10-phenanthroline (Bphen) in addition to the above-described materials, but embodiments are not limited thereto.

The electron transport region ETR may include the above-described compounds of the electron transport region in at least one of an electron injection layer EIL, an electron transport layer ETL, and a hole blocking layer HBL.

When the electron transport region ETR includes an electron transport layer ETL, the electron transport layer ETL may have a thickness in a range of about 100 Å to about 1,000 Å. For example, the electron transport layer ETL may have a thickness in a range of about 150 Å to about 500 Å. If the thickness of the electron transport layer ETL satisfies any of the aforementioned ranges, satisfactory electron transport characteristics may be obtained without a substantial increase in driving voltage. When the electron transport region ETR includes an electron injection layer EIL, the electron injection layer EIL may have a thickness in a range of about 1 Å to about 100 Å. For example, the electron injection layer EIL may have a thickness in a range of about 3 Å to about 90 Å. If the thickness of the electron injection layer EIL satisfies any of the above-described ranges, satisfactory electron injection characteristics may be obtained without a substantial increase in driving voltage.

The second electrode EL2 may be provided on the electron transport region ETR. The second electrode EL2 may be a common electrode. The second electrode EL2 may be a cathode or an anode, but embodiments are not limited thereto. For example, when the first electrode EL1 is an anode, the second electrode EL2 may be a cathode, and when the first electrode EL1 is a cathode, the second electrode EL2 may be an anode.

The second electrode EL2 may be a transmissive electrode, a transflective electrode, or a reflective electrode. When the second electrode EL2 is a transmissive electrode, the second electrode EL2 may be formed of a transparent metal oxide, for example, indium tin oxide (ITO), indium zinc oxide (IZO), zinc oxide (ZnO), indium tin zinc oxide (ITZO), etc.

When the second electrode EL2 is a transflective electrode or a reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, a compound thereof, or a mixture thereof (e.g., AgMg, AgYb, or MgYb). In an embodiment, the second electrode EL2 may have a multilayer structure including a reflective film or a transflective film formed of the above-described materials, and a transparent conductive film formed of ITO, IZO, ZnO, ITZO, etc. For example, the second electrode EL2 may include the above-described metal materials, combinations of at least two metal materials of the above-described metal materials, oxides of the above-described metal materials, or the like.

Although not shown in the drawings, the second electrode EL2 may be electrically connected to an auxiliary electrode. If the second electrode EL2 is electrically connected to an auxiliary electrode, resistance of the second electrode EL2 may decrease.

In an embodiment, the light emitting element ED may further include a capping layer CPL disposed on the second electrode EL2. The capping layer CPL may be a multilayer or a single layer.

In an embodiment, the capping layer CPL may include an organic layer and/or an inorganic layer. For example, when the capping layer CPL includes an inorganic material, the inorganic material may include an alkaline metal compound (e.g., LiF), an alkaline earth metal compound (e.g., MgF₂), SiON, SiNₓ, SiO_{y}, etc.

For example, when the capping layer CPL includes an organic material, the organic material may include α-NPD, NPB, TPD, m-MTDATA, Alq₃, CuPc, N4,N4,N4′,N4′-tetra(biphenyl-4-yl)biphenyl-4,4′-diamine (TPD15), 4,4′,4″-tris(carbazol-9-yl)triphenylamine (TCTA), etc., or may include an epoxy resin, or an acrylate such as methacrylate. However, embodiments are not limited thereto, and the capping layer CPL may include at least one of Compounds P1 to P5:

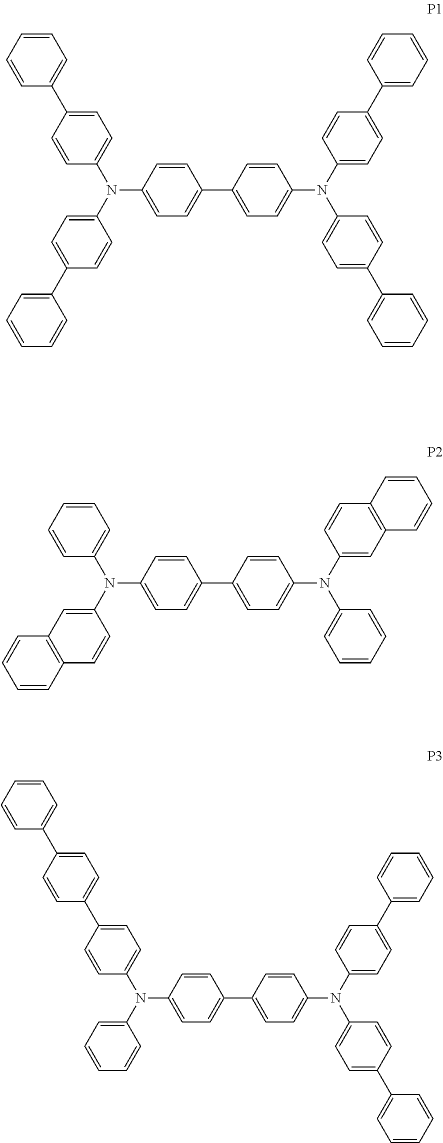 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

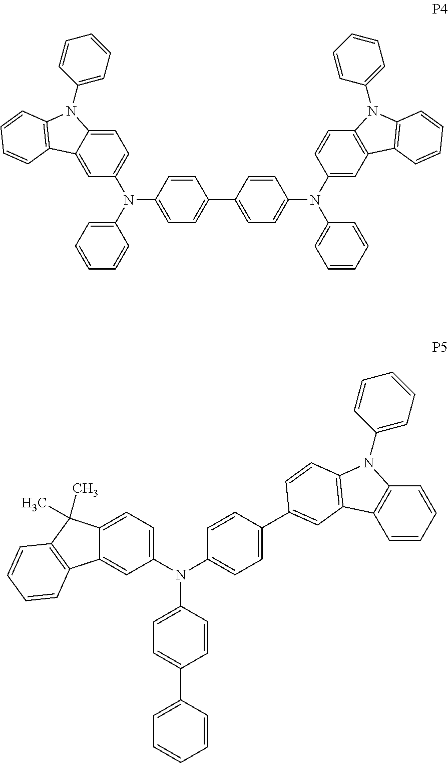 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

A refractive index of the capping layer CPL may be equal to or greater than about 1.6. For example, the refractive index of the capping layer CPL may be equal to or greater than about 1.6, with respect to light in a wavelength range of about 550 nm to about 660 nm.

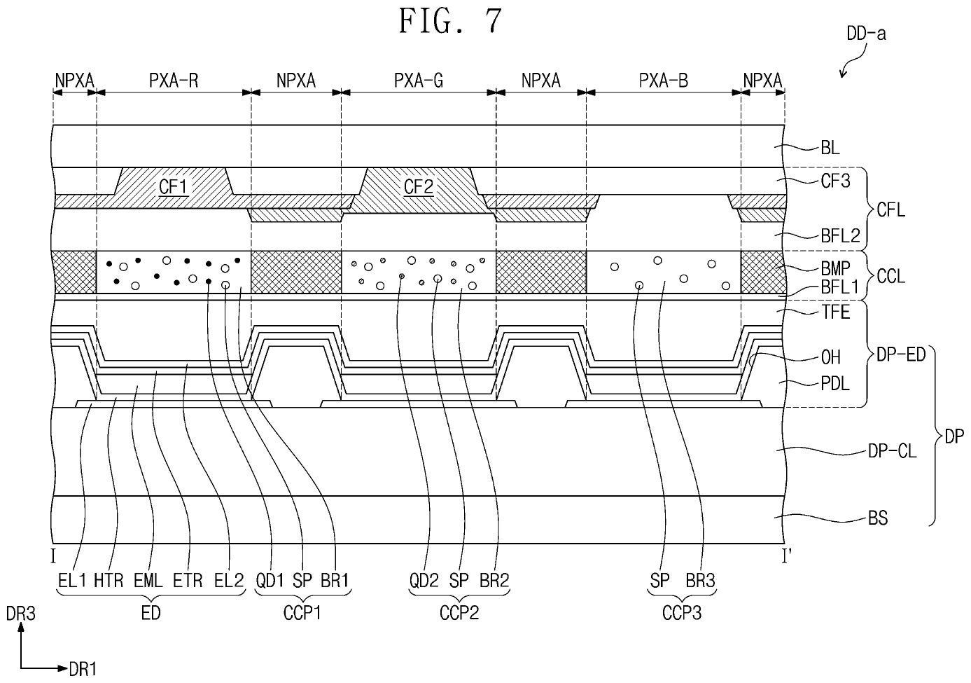

FIGS. 7 to 10 are each a schematic cross-sectional view of a display device according to an embodiment. Hereinafter, in the descriptions of the display devices according to embodiments as shown in FIGS. 7 to 10, the features which have been described above with respect to FIGS. 1 to 6 will not be explained again, and the differing features will be described.

Referring to FIG. 7, the display device DD-a according to an embodiment may include a display panel DP including a display device layer DP-ED, a light control layer CCL disposed on the display panel DP, and a color filter layer CFL.

In an embodiment illustrated in FIG. 7, the display panel DP may include a base layer BS, a circuit layer DP-CL provided on the base layer BS, and the display device layer DP-ED, and the display device layer DP-ED may include a light emitting element ED.

The light emitting element ED may include a first electrode EL1, a hole transport region HTR disposed on the first electrode EL1, an emission layer EML disposed on the hole transport region HTR, an electron transport region ETR disposed on the emission layer EML, and a second electrode EL2 disposed on the electron transport region ETR. In embodiments, a structure of the light emitting element ED shown in FIG. 7 may be the same as a structure of a light emitting element ED according to one of FIGS. 3 to 6 as described above.

The electron transport region ETR of the light emitting element ED included in the display device DD-a according to an embodiment may include the nitrogen-containing compound according to an embodiment as described above.

Referring to FIG. 7, the emission layer EML may be disposed in an opening OH defined in a pixel defining film PDL. For example, the emission layer EML, which is separated by the pixel defining film PDL and correspondingly provided to each of the light emitting regions PXA-R, PXA-G, and PXA-B, may emit light in a same wavelength range. In the display device DD-a, the emission layer EML may emit blue light. Although not shown in the drawings, in an embodiment, the emission layer EML may be provided as a common layer for each of the light emitting regions PXA-R, PXA-G, and PXA-B.

The light control layer CCL may be disposed on the display panel DP. The light control layer CCL may include a light conversion body. The light conversion body may be a quantum dot, a phosphor, or the like. The light conversion body may convert the wavelength of a provided light and emit the resulting light. For example, the light control layer CCL may be a layer containing the quantum dot or a layer containing the phosphor.

The light control layer CCL may include light control parts CCP1, CCP2, and CCP3. The light control parts CCP1, CCP2, and CCP3 may be spaced apart from each other.

Referring to FIG. 7, divided patterns BMP may be disposed between the light control parts CCP1, CCP2, and CCP3, which are spaced apart from each other, but embodiments are not limited thereto. FIG. 7 illustrates that the divided patterns BMP do not overlap the light control parts CCP1, CCP2, and CCP3, but the edges of the light control parts CCP1, CCP2, and CCP3 may overlap at least a portion of the divided patterns BMP.

The light control layer CCL may include a first light control part CCP1 containing a first quantum dot QD1 that converts first color light provided from the light emitting element ED into second color light, a second light control part CCP2 containing a second quantum dot QD2 that converts the first color light into third color light, and a third light control part CCP3 that transmits the first color light.

In an embodiment, the first light control part CCP1 may provide red light, which is the second color light, and the second light control part CCP2 may provide green light, which is the third color light. The third light control part CCP3 may provide blue light by transmitting the blue light, which is the first color light provided from the light emitting element ED. For example, the first quantum dot QD1 may be a red quantum dot, and the second quantum dot QD2 may be a green quantum dot. The quantum dots QD1 and QD2 may each be a quantum dot as described above.

The light control layer CCL may further include a scatterer SP. The first light control part CCP1 may include the first quantum dot QD1 and the scatterer SP, the second light control part CCP2 may include the second quantum dot QD2 and the scatterer SP, and the third light control part CCP3 may not include a quantum dot but may include the scatterer SP.

The scatterer SP may be inorganic particles. For example, the scatterer SP may include at least one of TiO₂, ZnO, Al₂O₃, SiO₂, and hollow silica. The scatterer SP may include any one of TiO₂, ZnO, Al₂O₃, SiO₂, and hollow silica, or may be a mixture of at least two materials selected from TiO₂, ZnO, Al₂O₃, SiO₂, and hollow silica.

The first light control part CCP1, the second light control part CCP2, and the third light control part CCP3 may include base resins BR1, BR2, and BR3, in which the quantum dots QD1 and QD2 and the scatterer SP are dispersed. In an embodiment, the first light control part CCP1 may include the first quantum dot QD1 and the scatterer SP dispersed in a first base resin BR1, the second light control part CCP2 may include the second quantum dot QD2 and the scatterer SP dispersed in a second base resin BR2, and the third light control part CCP3 may include the scatterer SP dispersed in a third base resin BR3.

The base resins BR1, BR2, and BR3 are media in which the quantum dots QD1 and QD2 and the scatterer SP are dispersed, and may be formed of various resin compositions, which may be generally referred to as a binder. For example, the base resins BR1, BR2, and BR3 may be acrylic-based resins, urethane-based resins, silicone-based resins, epoxy-based resins, etc. The base resins BR1, BR2, and BR3 may each be a transparent resin. In an embodiment, the first base resin BR1, the second base resin BR2, and the third base resin BR3 may be the same as or different from each other.

The light control layer CCL may include a barrier layer BFL1. The barrier layer BFL1 may prevent the penetration of moisture and/or oxygen (hereinafter, referred to as ‘moisture/oxygen’). The barrier layer BFL1 may block the light control parts CCP1, CCP2, and CCP3 from exposure to moisture/oxygen. The barrier layer BFL1 may cover the light control parts CCP1, CCP2, and CCP3. In an embodiment, a barrier layer BFL2 may be provided between the light control parts CCP1, CCP2, and CCP3 and the color filter layer CFL.

The barrier layers BFL1 and BFL2 may each independently include at least one inorganic layer. For example, the barrier layers BFL1 and BFL2 may each independently include an inorganic material. For example, the barrier layers BFL1 and BFL2 may each independently include a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide, a silicon oxynitride, a metal thin film which secures light transmittance, etc. The barrier layers BFL1 and BFL2 may each independently further include an organic film. The barrier layers BFL1 and BFL2 may each independently be formed of a single layer or of multiple layers.

In the display device DD-a, the color filter layer CFL may be disposed on the light control layer CCL. In an embodiment, the color filter layer CFL may be directly disposed on the light control layer CCL. For example, the barrier layer BFL2 may be omitted.

The color filter layer CFL may include filters CF1, CF2, and CF3. The color filter layer CFL may include a first filter CF1 that transmits second color light, a second filter CF2 that transmits third color light, and a third filter CF3 that transmits first color light. For example, the first filter CF1 may be a red filter, the second filter CF2 may be a green filter, and the third filter CF3 may be a blue filter. The filters CF1, CF2, and CF3 may each include a polymeric photosensitive resin and a pigment or dye. The first filter CF1 may include a red pigment or dye, the second filter CF2 may include a green pigment or dye, and the third filter CF3 may include a blue pigment or dye.

However, embodiments are not limited thereto, and the third filter CF3 may not include a pigment or dye. The third filter CF3 may include a polymeric photosensitive resin and may not include a pigment or dye. The third filter CF3 may be transparent. The third filter CF3 may be formed of a transparent photosensitive resin.

In an embodiment, the first filter CF1 and the second filter CF2 may each be a yellow filter. The first filter CF1 and the second filter CF2 may not be separated but may be provided as one filter.

Although not shown in the drawings, the color filter layer CFL may further include a light shielding part (not shown). The light shielding part (not shown) may be a black matrix. The light shielding part (not shown) may include an organic light shielding material or an inorganic light shielding material, each containing a black pigment or dye. The light shielding part (not shown) may prevent light leakage, and may separate the boundaries between adjacent filters CF1, CF2, and CF3.

The first to third filters CF1, CF2, and CF3 may be disposed to respectively correspond to the red light emitting region PXA-R, the green light emitting region PXA-G, and the blue light emitting region PXA-B.

A base substrate BL may be disposed on the color filter layer CFL. The base substrate BL may provide a base surface on which the color filter layer CFL, the light control layer CCL, and the like are disposed. The base substrate BL may be a glass substrate, a metal substrate, a plastic substrate, etc. However, embodiments are not limited thereto, and the base substrate BL may include an inorganic layer, an organic layer, or a composite material layer. Although not shown in the drawings, in an embodiment, the base substrate BL may be omitted.

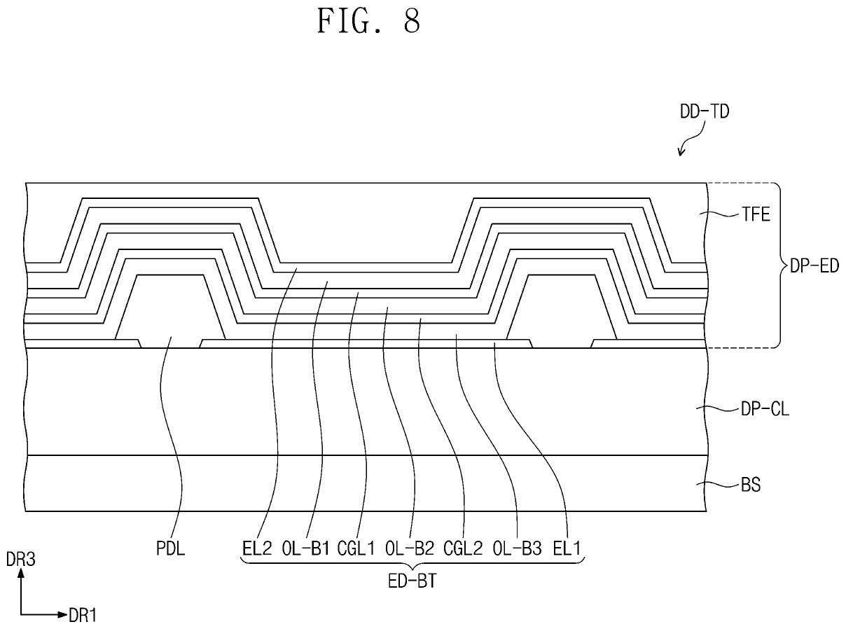

FIG. 8 is a schematic cross-sectional view of a portion of a display device according to an embodiment. In the display device DD-TD according to an embodiment, the light emitting element ED-BT may include light emitting structures OL-B1, OL-B2, and OL-B3.

The light emitting element ED-BT may include a first electrode EL1 and a second electrode EL2, which face each other, and the light emitting structures OL-B1, OL-B2, and OL-B3 stacked in a thickness direction between the first electrode EL1 and the second electrode EL2. The light emitting structures OL-B1, OL-B2, and OL-B3 may each include an emission layer EML (see FIG. 7) and a hole transport region HTR and an electron transport region ETR disposed with the emission layer EML (see FIG. 7) located therebetween.

For example, the light emitting element ED-BT included in the display device DD-TD may be a light emitting element having a tandem structure and including multiple emission layers.

In an embodiment illustrated in FIG. 8, light emitted from the light emitting structures OL-B1, OL-B2, and OL-B3 may each be blue light. However, embodiments are not limited thereto, and the light emitted from each of the light emitting structures OL-B1, OL-B2, and OL-B3 may have wavelength ranges that are different from each other. For example, the light emitting element ED-BT including the light emitting structures OL-B1, OL-B2, and OL-B3, which emit light having wavelength ranges that are different from each other, may emit white light.

Charge generation layers CGL1 and CGL2 may be respectively disposed between neighboring light emitting structures among the light emitting structures OL-B1, OL-B2, and OL-B3. Charge generation layers CGL1 and CGL2 may each independently include a p-type charge generation layer and/or an n-type charge generation layer.

At least one of the light emitting structures OL-B1, OL-B2, and OL-B3 in the display device DD-TD may include the above-described nitrogen-containing compound according to an embodiment. For example, at least one of the electron transport regions included in the light emitting element ED-BT may include the nitrogen-containing compound.

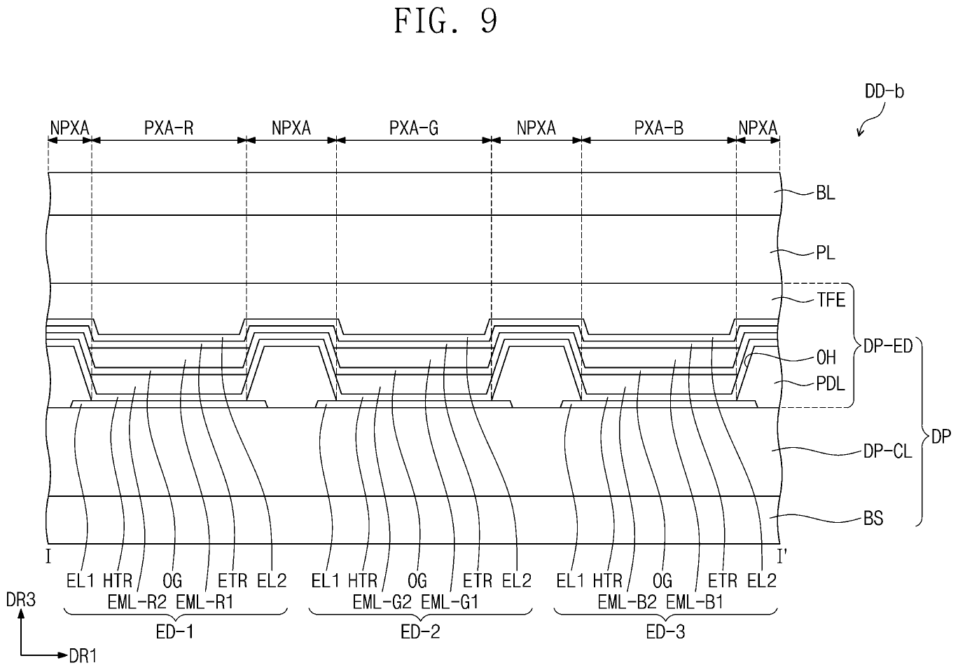

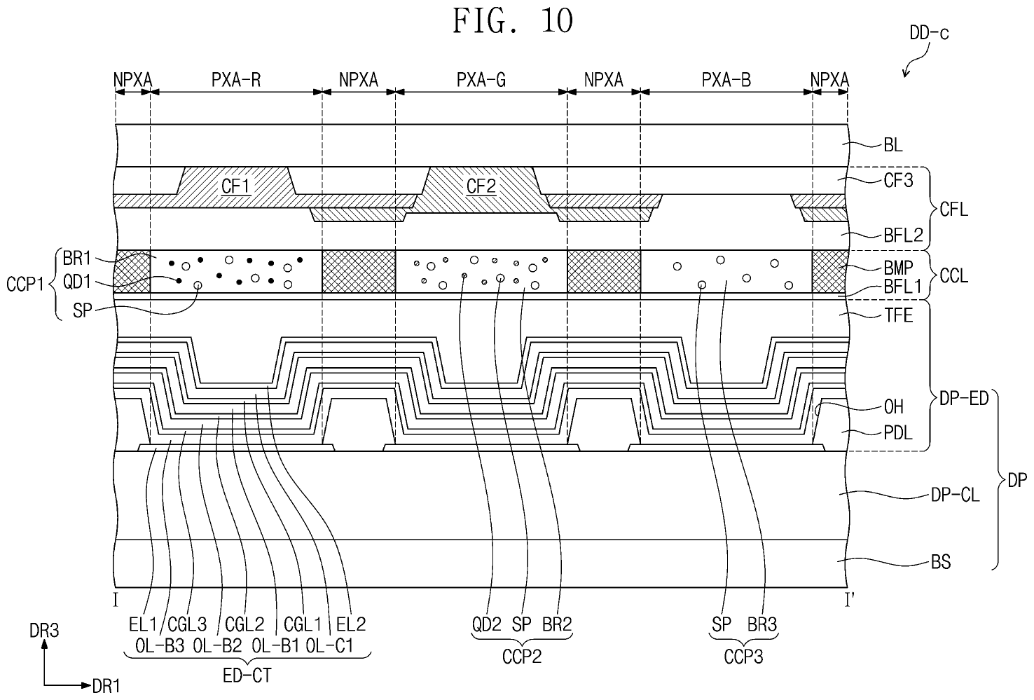

Referring to FIG. 9, a display device DD-b according to an embodiment may include light emitting elements ED-1, ED-2, and ED-3 in which two emission layers are stacked. In comparison to the display device DD illustrated in FIG. 2, the embodiment illustrated in FIG. 9 is different at least in that the first to third light emitting elements ED-1, ED-2, and ED-3 each include two emission layers that are stacked in a thickness direction. In each of the first to third light emitting elements ED-1, ED-2, and ED-3, the two emission layers may emit light in a same wavelength region.

The first light emitting element ED-1 may include a first red emission layer EML-R₁ and a second red emission layer EML-R₂. The second light emitting element ED-2 may include a first green emission layer EML-G1 and a second green emission layer EML-G2. The third light emitting element ED-3 may include a first blue emission layer EML-B1 and a second blue emission layer EML-B2. An emission auxiliary part OG may be disposed between the first red emission layer EML-R₁ and the second red emission layer EML-R₂, between the first green emission layer EML-G1 and the second green emission layer EML-G2, and between the first blue emission layer EML-B1 and the second blue emission layer EML-B2.

The emission auxiliary part OG may be a single layer or a multilayer. The emission auxiliary part OG may include a charge generation layer. For example, the emission auxiliary part OG may include an electron transport region, a charge generation layer, and a hole transport region, which may be stacked in that order. The emission auxiliary part OG may be provided as a common layer for all of the first to third light emitting elements ED-1, ED-2, and ED-3. However, embodiments are not limited thereto, and the emission auxiliary part OG may be provided by being patterned within the openings OH defined in the pixel defining film PDL.

The first red emission layer EML-R₁, the first green emission layer EML-G1, and the first blue emission layer EML-B1 may each be disposed between the emission auxiliary part OG and the electron transport region ETR. The second red emission layer EML-R₂, the second green emission layer EML-G2, and the second blue emission layer EML-B2 may each be disposed between the hole transport region HTR and the emission auxiliary part OG.

For example, the first light emitting element ED-1 may include the first electrode EL1, the hole transport region HTR, the second red emission layer EML-R₂, the emission auxiliary part OG, the first red emission layer EML-R₁, the electron transport region ETR, and the second electrode EL2, which are stacked in that order. The second light emitting element ED-2 may include the first electrode EL1, the hole transport region HTR, the second green emission layer EML-G2, the emission auxiliary part OG, the first green emission layer EML-G1, the electron transport region ETR, and the second electrode EL2, which are stacked in that order. The third light emitting element ED-3 may include the first electrode EL1, the hole transport region HTR, the second blue emission layer EML-B2, the emission auxiliary part OG, the first blue emission layer EML-B1, the electron transport region ETR, and the second electrode EL2, which are stacked in that order.

An optical auxiliary layer PL may be disposed on the display device layer DP-ED. The optical auxiliary layer PL may include a polarizing layer. The optical auxiliary layer PL may be disposed on the display panel DP and may control light that is reflected at the display panel DP from an external light. Although not shown in the drawings, in an embodiment, the optical auxiliary layer PL may be omitted from the display device DD-b.

At least one electron transport region included in the display device DD-b illustrated in FIG. 9 may include the above-described nitrogen-containing compound according to an embodiment. For example, in an embodiment, at least one of the electron transport regions ETR included in the light emitting elements ED-1, ED-2, and ED-3 may include the nitrogen-containing compound.

In contrast to FIGS. 8 and 9, FIG. 10 illustrates a display device DD-c that is different at least in that it includes four light emitting structures OL-B1, OL-B2, OL-B3, and OL-C1. A light emitting element ED-CT may include a first electrode EL1 and a second electrode EL2 which face each other, and first to fourth light emitting structures OL-B1, OL-B2, OL-B3, and OL-C1 that are stacked in a thickness direction between the first electrode EL1 and the second electrode EL2.

Charge generation layers CGL1, CGL2, and CGL3 may each be disposed between adjacent light emitting structures among the first to fourth light emitting structures OL-B1, OL-B2, OL-B3, and OL-C1.

Among the four light emitting structures, the first to third light emitting structures OL-B1, OL-B2, and OL-B3 may each emit blue light, and the fourth light emitting structure OL-C1 may emit green light. However, embodiments are not limited thereto, and the first to fourth light emitting structures OL-B1, OL-B2, OL-B3, and OL-C1 may emit light having different wavelength regions from each other.

The charge generation layers CGL1, CGL2, and CGL3, which are disposed between adjacent light emitting structures among the first to fourth light emitting structures OL-B1, OL-B2, OL-B3, and OL-C1, may each independently include a p-type charge generation layer and/or an n-type charge generation layer.

In the display device DD-c, at least one of the light emitting structures OL-B1, OL-B2, OL-B3, and OL-C1 may each independently include the nitrogen-containing compound according to an embodiment.

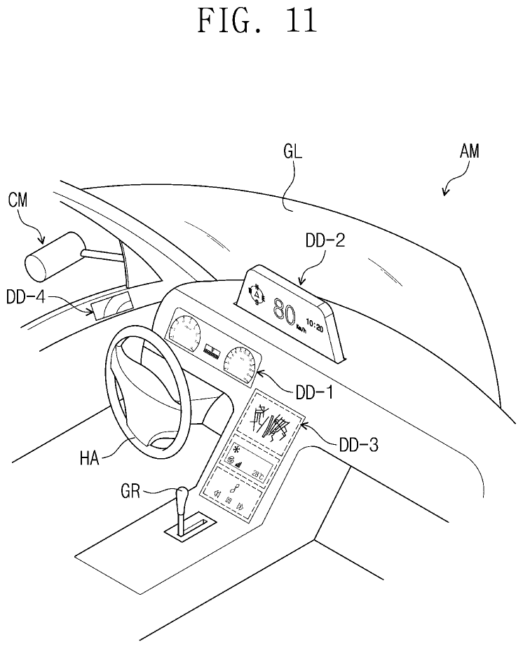

FIG. 11 is a schematic perspective view of a vehicle AM in which first to fourth display devices DD-1, DD-2, DD-3, and DD-4 are disposed. At least one of the first to fourth display devices DD-1, DD-2, DD-3, and DD-4 may have a structure according to one of display devices DD, DD-TD, DD-a, DD-b, and DD-c as described above with reference to FIGS. 1, 2, and 7 to 10.

FIG. 11 illustrates a vehicle AM, but this is only an example, and the first to fourth display devices DD-1, DD-2, DD-3, and DD-4 may be disposed in various transportation means such as bicycles, motorcycles, trains, ships, and airplanes. In an embodiment, at least one of the first to fourth display devices DD-1, DD-2, DD-3, and DD-4, having a structure according to one of the display devices DD, DD-TD, DD-a, DD-b, and DD-c as described above, may be included in a personal computer, a laptop computer, a personal digital terminal, a game console, a portable electronic device, a television, a monitor, a billboard, or the like. However, these are merely given as examples, and the display device may be included in other electronic apparatuses.

At least one of the first to fourth display devices DD-1, DD-2, DD-3, and DD-4 may each independently include a light emitting element ED according to an embodiment as described with reference to any of FIGS. 3 to 6. The light emitting element ED may include the nitrogen-containing compound according to an embodiment. At least one of the first to fourth display devices DD-1, DD-2, DD-3, and DD-4 may include a light emitting element ED that includes the nitrogen-containing compound according to an embodiment, thereby improving display service life.

Referring to FIG. 11, the vehicle AM may include a steering wheel HA and a gearshift GR for driving the vehicle AM. The vehicle AM may include a front window GL that is disposed so as to face a driver.

The first display device DD-1 may be disposed in a first region that overlaps the steering wheel HA. For example, the first display device DD-1 may be a digital cluster that displays first information of the vehicle AM. The first information may include a first scale which indicates a driving speed of the vehicle AM, a second scale which indicates an engine speed (for example, as revolutions per minute (RPM)), a fuel gauge, etc. The first scale and the second scale may be represented by digital images.

The second display device DD-2 may be disposed in a second region facing the driver's seat and overlapping the front window GL. The driver's seat may be a seat where the steering wheel HA is disposed. For example, the second display device DD-2 may be a head up display (HUD) that displays second information of the vehicle AM. The second display device DD-2 may be optically transparent. The second information may include digital numbers which indicate a driving speed, and may further include information such as the current time. Although not shown in the drawings, in an embodiment, the second information of the second display device DD-2 may be displayed by being projected onto the front window GL.

The third display device DD-3 may be disposed in a third region adjacent to the gearshift GR. For example, the third display device DD-3 may be disposed between the driver's seat and the passenger seat and may be a center information display (CID) for a vehicle that displays third information. The passenger seat may be a seat that is spaced apart from the driver's seat with the gearshift GR disposed therebetween. The third information may include information about traffic conditions (e.g., navigation information), playing music or radio or a video (or an image), temperatures inside the vehicle AM, etc.

The fourth display device DD-4 may be disposed in a fourth region that is spaced apart from the steering wheel HA and the gearshift GR and adjacent to a side of the vehicle AM. For example, the fourth display device DD-4 may be a digital side-view mirror that displays fourth information. The fourth display device DD-4 may display an image external to the vehicle AM that is taken by a camera module CM disposed outside the vehicle AM. The fourth information may include an image of the exterior of the vehicle AM.

The first to fourth information as described above are only presented as examples, and the first to fourth display devices DD-1, DD-2, DD-3, and DD-4 may further display information about the interior and exterior of the vehicle AM. The first to fourth information may include information that is different from each other. However, embodiments are not limited thereto, and a part of the first to fourth information may include the same information as one another.

Hereinafter, a nitrogen-containing compound according to an embodiment and a light emitting element according to an embodiment will be described with reference to the Examples and the Comparative Examples. The Examples described below are only provided as illustrations to assist in understanding the disclosure, and the scope thereof is not limited thereto.

### Examples

### 1. Synthesis of Nitrogen-Containing Compound

A synthesis method of the nitrogen-containing compound according to embodiments will be described in detail by describing synthesis methods for Compounds 2, 30, 40, 71, 90, 116, 120, 155, and 161. The synthesis methods for the nitrogen-containing compounds according to the following descriptions are provided only as examples, and the synthesis methods of the nitrogen-containing compounds according to embodiments are not limited to the Examples below.

### (1) Synthesis of Compound 2

### (Synthesis of Intermediate 2-2)

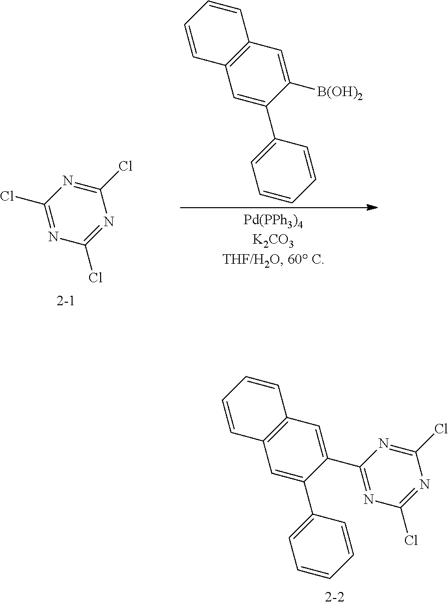 [SMILES: Clc1nc(Cl)nc(Cl)n1 ; OB(O)c1cc2ccccc2cc1-c1ccccc1 ; Clc1nc(Cl)nc(-c2cc3ccccc3cc2-c2ccccc2)n1]

Intermediate 2-1 (1.84 g), Pd(PPh₃)₄ (0.56 g), K₂CO₃ (3.45 g), and (3-phenylnaphthalen-2-yl)boronic acid (2.48 g) were dissolved in tetrahydrofuran (THF)/H₂O (100 mL/25 mL), and the reaction solution was stirred at about 60° C. for about 12 hours. This reaction solution was cooled to room temperature and the reaction was terminated with water, and the extraction was performed three times with ethyl ether to separate an organic layer. The separated organic layer was dried over anhydrous magnesium sulfate and distilled under reduced pressure to obtain residues. The obtained residues were separated and purified by column chromatography to obtain Intermediate 2-2 (2.36 g, yield: 67%).

### (Synthesis of Intermediate 2-3)

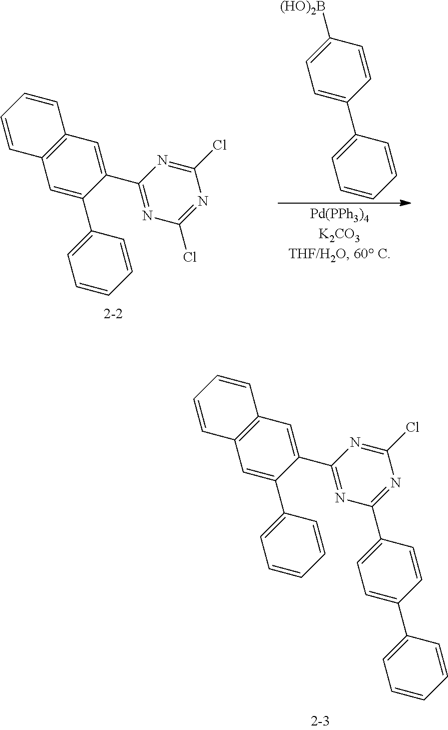 [SMILES: Clc1nc(Cl)nc(-c2cc3ccccc3cc2-c2ccccc2)n1 ; OB(O)c1ccc(-c2ccccc2)cc1 ; Clc1nc(-c2ccc(-c3ccccc3)cc2)nc(-c2cc3ccccc3cc2-c2ccccc2)n1]

Intermediate 2-2 (3.52 g), Pd(PPh₃)₄ (0.56 g), K₂CO₃ (3.45 g), and [1,1′-biphenyl]-4-ylboronic acid (1.98 g) were dissolved in THF/H₂O (100 mL/25 mL), and the reaction solution was stirred at about 60° C. for about 12 hours. This reaction solution was cooled to room temperature and the reaction was terminated with water, and the extraction was performed three times with ethyl ether to separate an organic layer. The separated organic layer was dried over anhydrous magnesium sulfate and distilled under reduced pressure to obtain residues. The obtained residues were separated and purified by column chromatography to obtain Intermediate 2-3 (3.75 g, yield: 80%).

### (Synthesis of Compound 2)

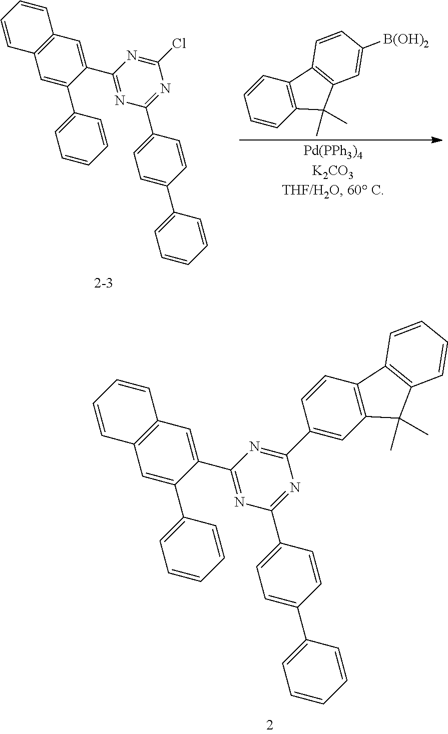 [SMILES: Clc1nc(-c2ccc(-c3ccccc3)cc2)nc(-c2cc3ccccc3cc2-c2ccccc2)n1 ; CC1(C)c2ccccc2-c2ccc(B(O)O)cc21 ; CC1(C)c2ccccc2-c2ccc(-c3nc(-c4ccc(-c5ccccc5)cc4)nc(-c4cc5ccccc5cc4-c4ccccc4)n3)cc21]

Intermediate 2-3 (4.69 g), Pd(PPh₃)₄ (0.56 g), K₂CO₃ (3.45 g), and (9,9-dimethyl-9H-fluoren-2-yl)boronic acid (2.38 g) were dissolved in THF/H₂O (100 mL/25 mL), and the reaction solution was stirred at about 60° C. for about 12 hours. This reaction solution was cooled to room temperature and the reaction was terminated with water, and the extraction was performed three times with ethyl ether to separate an organic layer. The separated organic layer was dried over anhydrous magnesium sulfate and distilled at reduced pressure to obtain residues. The obtained residues were separated and purified by column chromatography to obtain Compound 2 (4.26 g, yield: 68%).

### (2) Synthesis of Compound 30

### (Synthesis of Intermediate 30-1)

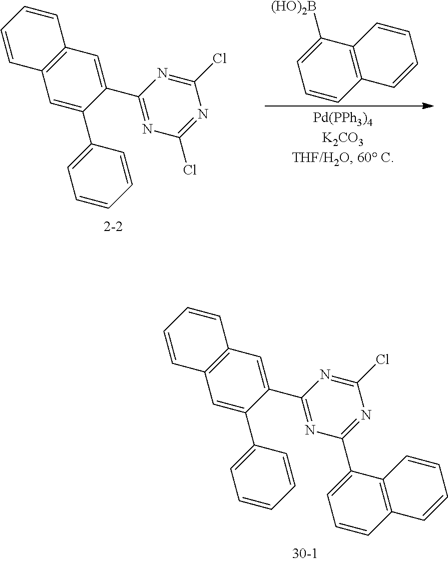 [SMILES: Clc1nc(Cl)nc(-c2cc3ccccc3cc2-c2ccccc2)n1 ; OB(O)c1cccc2ccccc12 ; Clc1nc(-c2cc3ccccc3cc2-c2ccccc2)nc(-c2cccc3ccccc23)n1]

Intermediate 2-2 (3.52 g), Pd(PPh₃)₄ (0.56 g), K₂CO₃ (3.45 g), and naphthalen-1-ylboronic acid (1.71 g) were dissolved in THF/H₂O (100 mL/25 mL), and the reaction solution was stirred at about 60° C. for about 12 hours. This reaction solution was cooled to room temperature and the reaction was terminated with water, and the extraction was performed three times with ethyl ether to separate an organic layer. The separated organic layer was dried over anhydrous magnesium sulfate and distilled under reduced pressure to obtain residues. The obtained residues were separated and purified by column chromatography to obtain Intermediate 30-1 (2.70 g, yield: 61%).

### (Synthesis of Compound 30)

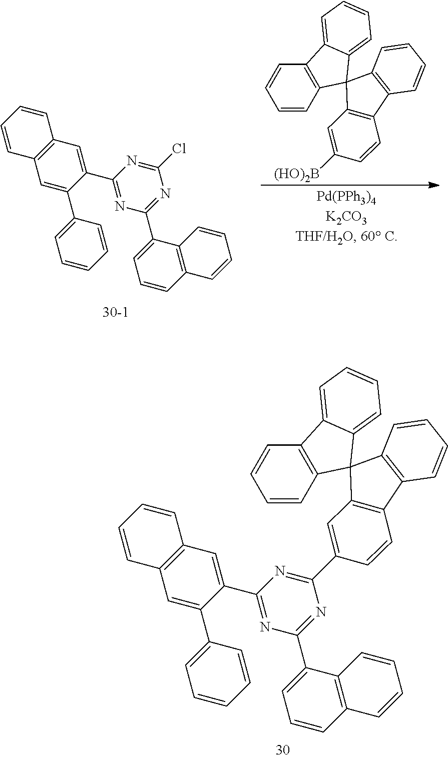 [SMILES: Clc1nc(-c2cc3ccccc3cc2-c2ccccc2)nc(-c2cccc3ccccc23)n1 ; OB(O)c1ccc2c(c1)C1(c3ccccc3-c3ccccc31)c1ccccc1-2 ; c1ccc(-c2cc3ccccc3cc2-c2nc(-c3ccc4c(c3)C3(c5ccccc5-c5ccccc53)c3ccccc3-4)nc(-c3cccc4ccccc34)n2)cc1]

Intermediate 30-1 (4.43 g), Pd(PPh₃)₄ (0.56 g), K₂CO₃ (3.45 g), and 9,9′-spirobi[fluoren]-2-ylboronic acid (3.60 g) were dissolved in THF/H₂O (100 mL/25 mL), and the reaction solution was stirred at about 60° C. for about 12 hours. This reaction solution was cooled to room temperature and the reaction was terminated with water, and the extraction was performed three times with ethyl ether to separate an organic layer. The separated organic layer was dried over anhydrous magnesium sulfate and distilled at reduced pressure to obtain residues. The obtained residues were separated and purified by column chromatography to obtain Compound 30 (5.56 g, yield: 77%).

### (3) Synthesis of Compound 40

### (Synthesis of Intermediate 40-1)

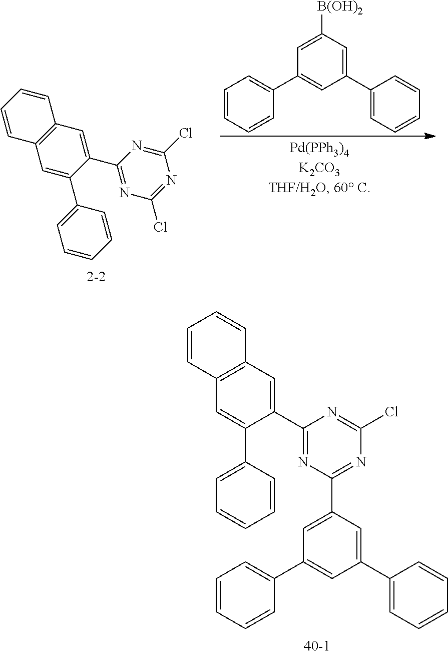 [SMILES: Clc1nc(Cl)nc(-c2cc3ccccc3cc2-c2ccccc2)n1 ; OB(O)c1cc(-c2ccccc2)cc(-c2ccccc2)c1 ; Clc1nc(-c2cc(-c3ccccc3)cc(-c3ccccc3)c2)nc(-c2cc3ccccc3cc2-c2ccccc2)n1]

Intermediate 2-2 (3.52 g), Pd(PPh₃)₄ (0.56 g), K₂CO₃ (3.45 g), and [1,1′:3′,1″-terphenyl]-5′-ylboronic acid (2.74 g) were dissolved in THF/H₂O (100 mL/25 mL), and the reaction solution was stirred at about 60° C. for about 12 hours. This reaction solution was cooled to room temperature and the reaction was terminated with water, and the extraction was performed three times with ethyl ether to separate an organic layer. The separated organic layer was dried over anhydrous magnesium sulfate and distilled under reduced pressure to obtain residues. The obtained residues were separated and purified by column chromatography to obtain Intermediate 40-1 (3.43 g, yield: 63%).

### (Synthesis of Compound 40)

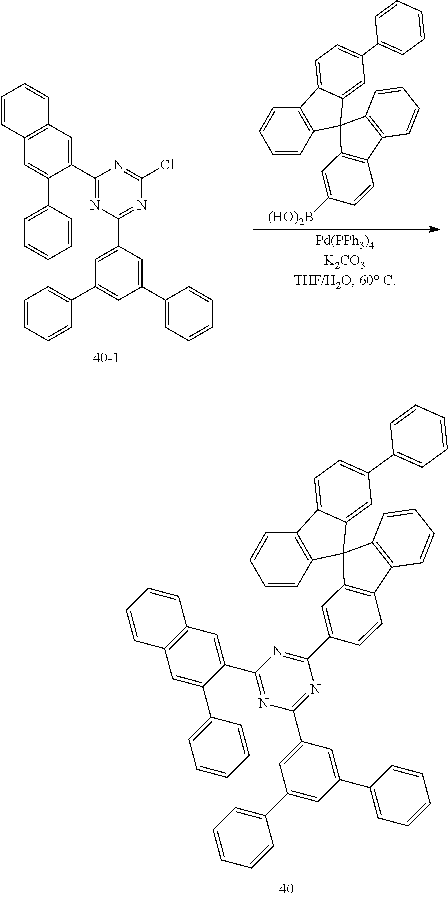 [SMILES: Clc1nc(-c2cc(-c3ccccc3)cc(-c3ccccc3)c2)nc(-c2cc3ccccc3cc2-c2ccccc2)n1 ; OB(O)c1ccc2c(c1)C1(c3ccccc3-2)c2ccccc2-c2ccc(-c3ccccc3)cc21 ; c1ccc(-c2cc(-c3ccccc3)cc(-c3nc(-c4ccc5c(c4)C4(c6ccccc6-c6ccc(-c7ccccc7)cc64)c4ccccc4-5)nc(-c4cc5ccccc5cc4-c4ccccc4)n3)c2)cc1]

Intermediate 40-1 (5.46 g), Pd(PPh₃)₄ (0.56 g), K₂CO₃ (3.45 g), and (2′-phenyl-9,9′-spirobi[fluoren]-2-yl)boronic acid (4.36 g) were dissolved in THF/H₂O (100 mL/25 mL), and the reaction solution was stirred at about 60° C. for about 12 hours. This reaction solution was cooled to room temperature and the reaction was terminated with water, and the extraction was performed three times with ethyl ether to separate an organic layer. The separated organic layer was dried over anhydrous magnesium sulfate and distilled at reduced pressure to obtain residues. The obtained residues were separated and purified by column chromatography to obtain Compound 40 (7.57 g, yield: 84%).

### (4) Synthesis of Compound 71

### (Synthesis of Intermediate 71-1)

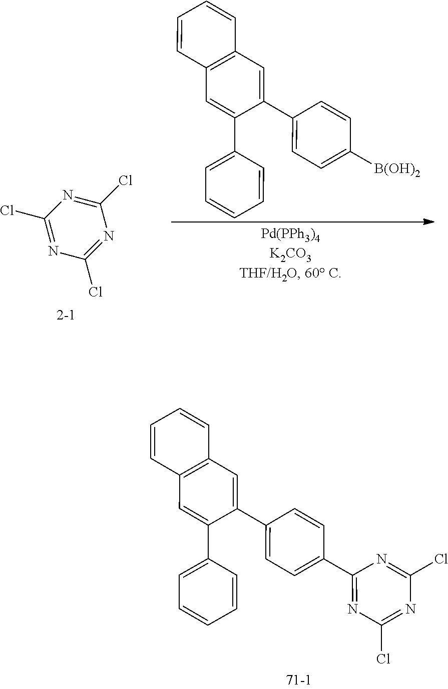 [SMILES: Clc1nc(Cl)nc(Cl)n1 ; OB(O)c1ccc(-c2cc3ccccc3cc2-c2ccccc2)cc1 ; Clc1nc(Cl)nc(-c2ccc(-c3cc4ccccc4cc3-c3ccccc3)cc2)n1]

Intermediate 2-1 (1.84 g), Pd(PPh₃)₄ (0.56 g), K₂CO₃ (3.45 g), and (4-(3-phenylnaphthalen-2-yl)phenyl)boronic acid (3.24 g) were dissolved in THF/H₂O (100 mL/25 mL), and the reaction solution was stirred at about 60° C. for about 12 hours. This reaction solution was cooled to room temperature and the reaction was terminated with water, and the extraction was performed three times with ethyl ether to separate an organic layer. The separated organic layer was dried over anhydrous magnesium sulfate and distilled under reduced pressure to obtain residues. The obtained residues were separated and purified by column chromatography to obtain Intermediate 71-1 (3.12 g, yield: 73%).

### (Synthesis of Intermediate 71-2)

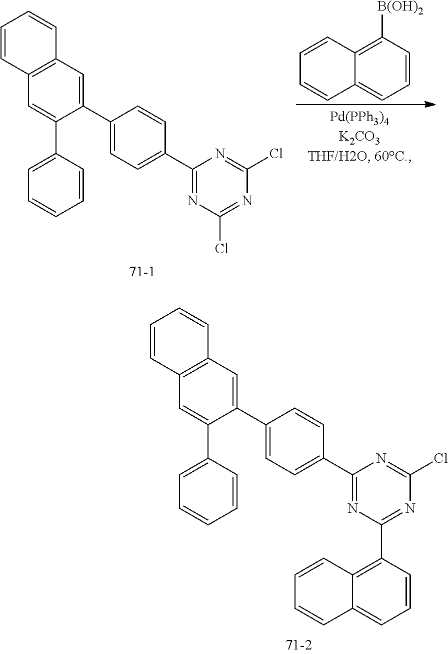 [SMILES: Clc1nc(Cl)nc(-c2ccc(-c3cc4ccccc4cc3-c3ccccc3)cc2)n1 ; OB(O)c1cccc2ccccc12 ; Clc1nc(-c2ccc(-c3cc4ccccc4cc3-c3ccccc3)cc2)nc(-c2cccc3ccccc23)n1]

Intermediate 71-1 (4.28 g), Pd(PPh₃)₄ (0.56 g), K₂CO₃ (3.45 g), and naphthalen-1-ylboronic acid (1.71 g) were dissolved in THF/H₂O (100 mL/25 mL), and the reaction solution was stirred at about 60° C. for about 12 hours. This reaction solution was cooled to room temperature and the reaction was terminated with water, and the extraction was performed three times with ethyl ether to separate an organic layer. The separated organic layer was dried over anhydrous magnesium sulfate and distilled under reduced pressure to obtain residues. The obtained residues were separated and purified by column chromatography to obtain Intermediate 71-2 (3.84 g, yield: 74%).

### (Synthesis of Compound 71)

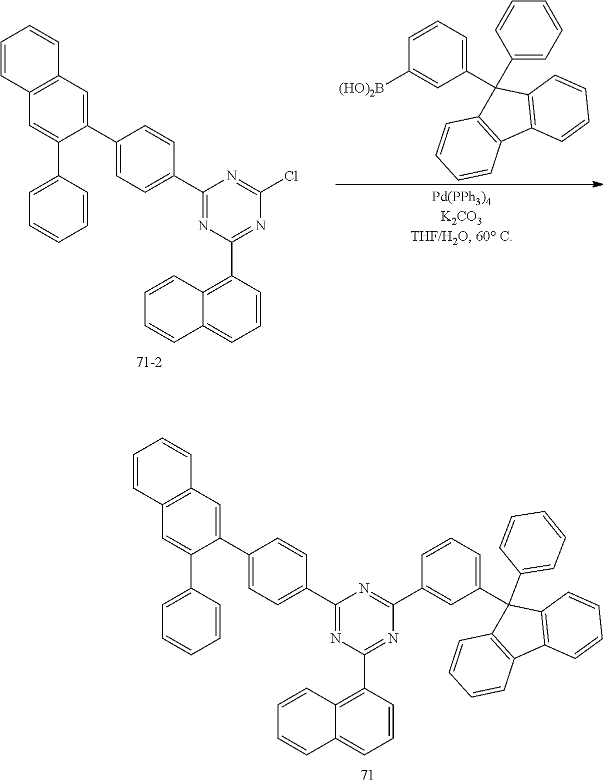 [SMILES: Clc1nc(-c2ccc(-c3cc4ccccc4cc3-c3ccccc3)cc2)nc(-c2cccc3ccccc23)n1 ; OB(O)c1cccc(C2(c3ccccc3)c3ccccc3-c3ccccc32)c1 ; c1ccc(-c2cc3ccccc3cc2-c2ccc(-c3nc(-c4cccc(C5(c6ccccc6)c6ccccc6-c6ccccc65)c4)nc(-c4cccc5ccccc45)n3)cc2)cc1]

Intermediate 71-2 (5.20 g), Pd(PPh₃)₄ (0.56 g), K₂CO₃ (3.45 g), and (3-(9-phenyl-9H-fluoren-9-yl)phenyl)boronic acid (3.62 g) were dissolved in THF/H₂O (100 mL/25 mL), and the reaction solution was stirred at about 60° C. for about 12 hours. This reaction solution was cooled to room temperature and the reaction was terminated with water, and the extraction was performed three times with ethyl ether to separate an organic layer. The separated organic layer was dried over anhydrous magnesium sulfate and distilled at reduced pressure to obtain residues. The obtained residues were separated and purified by column chromatography to obtain Compound 71 (5.04 g, yield: 63%).

### (5) Synthesis of Compound 90

### (Synthesis of Intermediate 90-1)

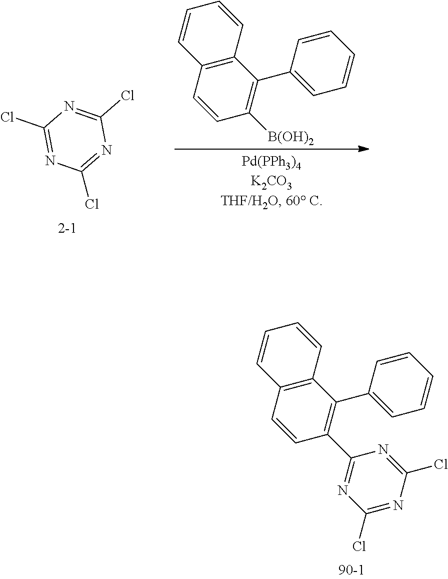 [SMILES: Clc1nc(Cl)nc(Cl)n1 ; OB(O)c1ccc2ccccc2c1-c1ccccc1 ; Clc1nc(Cl)nc(-c2ccc3ccccc3c2-c2ccccc2)n1]

Intermediate 2-1 (1.84 g), Pd(PPh₃)₄ (0.56 g), K₂CO₃ (3.45 g), and (1-phenylnaphthalen-2-yl)boronic acid (2.48 g) were dissolved in THF/H₂O (100 mL/25 mL), and the reaction solution was stirred at about 60° C. for about 12 hours. This reaction solution was cooled to room temperature and the reaction was terminated with water, and the extraction was performed three times with ethyl ether to separate an organic layer. The separated organic layer was dried over anhydrous magnesium sulfate and distilled under reduced pressure to obtain residues. The obtained residues were separated and purified by column chromatography to obtain Intermediate 90-1 (2.28 g, yield: 65%).

### (Synthesis of Intermediate 90-2)

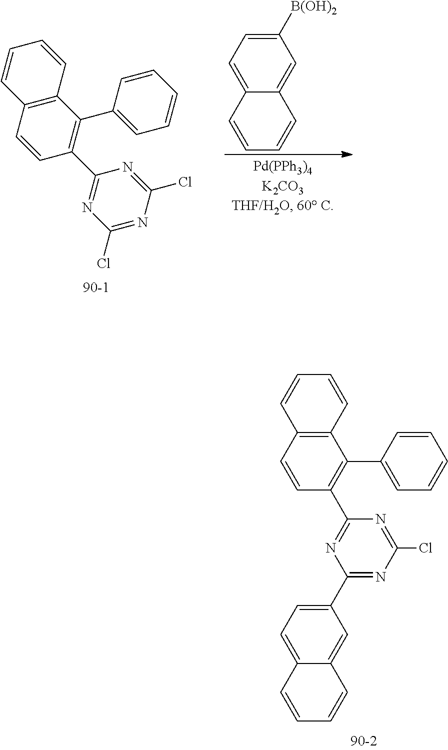 [SMILES: Clc1nc(Cl)nc(-c2ccc3ccccc3c2-c2ccccc2)n1 ; OB(O)c1ccc2ccccc2c1 ; Clc1nc(-c2ccc3ccccc3c2)nc(-c2ccc3ccccc3c2-c2ccccc2)n1]

Intermediate 90-1 (3.52 g), Pd(PPh₃)₄ (0.56 g), K₂CO₃ (3.45 g), and naphthalen-2-ylboronic acid (1.71 g) were dissolved in THF/H₂O (100 mL/25 mL), and the reaction solution was stirred at about 60° C. for about 12 hours. This reaction solution was cooled to room temperature and the reaction was terminated with water, and the extraction was performed three times with ethyl ether to separate an organic layer. The separated organic layer was dried over anhydrous magnesium sulfate and distilled under reduced pressure to obtain residues. The obtained residues were separated and purified by column chromatography to obtain Intermediate 90-2 (3.54 g, yield: 80%).

### (Synthesis of Compound 90)

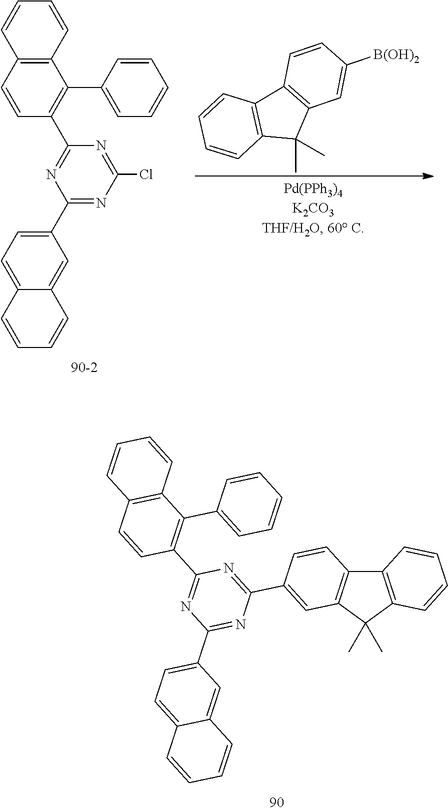

Intermediate 90-2 (4.43 g), Pd(PPh₃)₄ (0.56 g), K₂CO₃ (3.45 g), and (9,9-dimethyl-9H-fluoren-2-yl)boronic acid (2.38 g) were dissolved in THF/H₂O (100 mL/25 mL), and the reaction solution was stirred at about 60° C. for about 12 hours. This reaction solution was cooled to room temperature and the reaction was terminated with water, and the extraction was performed three times with ethyl ether to separate an organic layer. The separated organic layer was dried over anhydrous magnesium sulfate and distilled at reduced pressure to obtain residues. The obtained residues were separated and purified by column chromatography to obtain Compound 90 (3.78 g, yield: 63%).

### (6) Synthesis of Compound 116

### (Synthesis of Intermediate 116-1)

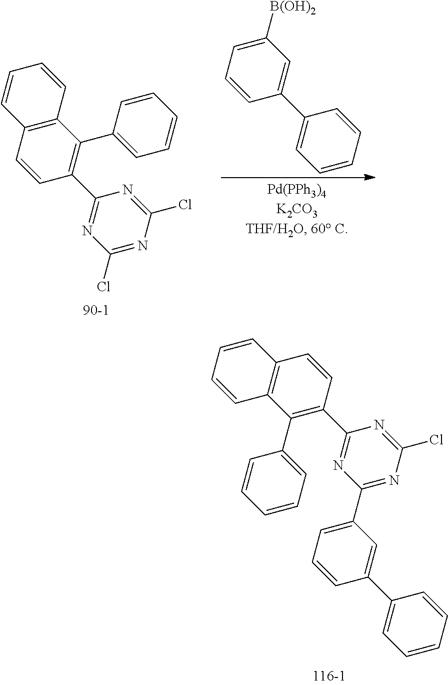 [SMILES: Clc1nc(Cl)nc(-c2ccc3ccccc3c2-c2ccccc2)n1 ; OB(O)c1cccc(-c2ccccc2)c1 ; Clc1nc(-c2cccc(-c3ccccc3)c2)nc(-c2ccc3ccccc3c2-c2ccccc2)n1]

Intermediate 90-1 (3.52 g), Pd(PPh₃)₄ (0.56 g), K₂CO₃ (3.45 g), and [1,1′-biphenyl]-3-ylboronic acid (1.98 g) were dissolved in THF/H₂O (100 mL/25 mL), and the reaction solution was stirred at about 60° C. for about 12 hours. This reaction solution was cooled to room temperature and the reaction was terminated with water, and the extraction was performed three times with ethyl ether to separate an organic layer. The separated organic layer was dried over anhydrous magnesium sulfate and distilled under reduced pressure to obtain residues. The obtained residues were separated and purified by column chromatography to obtain Intermediate 116-1 (3.33 g, yield: 71%).

### (Synthesis of Compound 116)

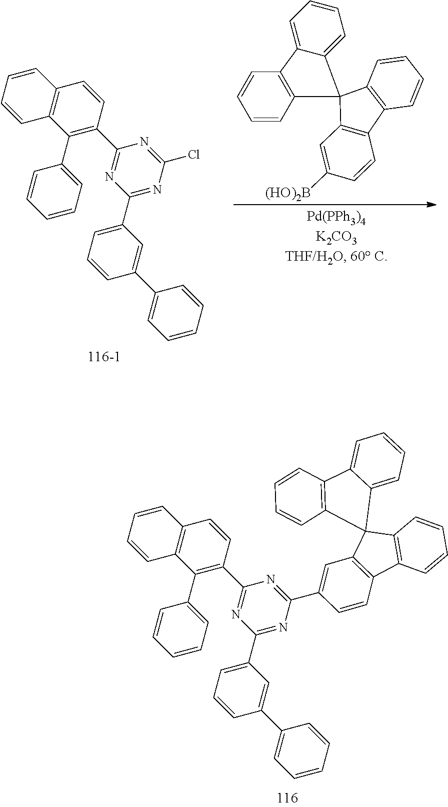 [SMILES: Clc1nc(-c2cccc(-c3ccccc3)c2)nc(-c2ccc3ccccc3c2-c2ccccc2)n1 ; OB(O)c1ccc2c(c1)C1(c3ccccc3-c3ccccc31)c1ccccc1-2 ; c1ccc(-c2cccc(-c3nc(-c4ccc5c(c4)C4(c6ccccc6-c6ccccc64)c4ccccc4-5)nc(-c4ccc5ccccc5c4-c4ccccc4)n3)c2)cc1]

Intermediate 116-1 (4.69 g), Pd(PPh₃)₄ (0.56 g), K₂CO₃ (3.45 g), and 9,9′-spirobi[fluoren]-2-ylboronic acid (3.60 g) were dissolved in THF/H-20 (100 mL/25 mL), and the reaction solution was stirred at about 60° C. for about 12 hours. This reaction solution was cooled to room temperature and the reaction was terminated with water, and the extraction was performed three times with ethyl ether to separate an organic layer. The separated organic layer was dried over anhydrous magnesium sulfate and distilled at reduced pressure to obtain residues. The obtained residues were separated and purified by column chromatography to obtain Compound 116 (5.46 g, yield: 73%).

### (7) Synthesis of Compound 120

### (Synthesis of Intermediate 120-1)

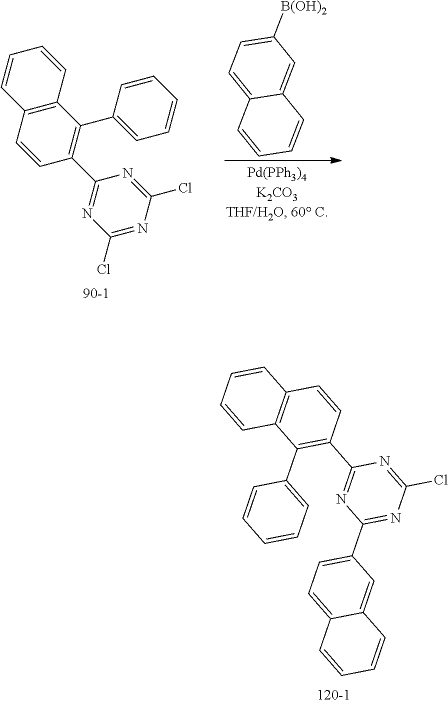 [SMILES: Clc1nc(Cl)nc(-c2ccc3ccccc3c2-c2ccccc2)n1 ; OB(O)c1ccc2ccccc2c1 ; Clc1nc(-c2ccc3ccccc3c2)nc(-c2ccc3ccccc3c2-c2ccccc2)n1]

Intermediate 90-1 (3.52 g), Pd(PPh₃)₄ (0.56 g), K₂CO₃ (3.45 g), and naphthalen-2-ylboronic acid (1.71 g) were dissolved in THF/H₂O (100 mL/25 mL), and the reaction solution was stirred at about 60° C. for about 12 hours. This reaction solution was cooled to room temperature and the reaction was terminated with water, and the extraction was performed three times with ethyl ether to separate an organic layer. The separated organic layer was dried over anhydrous magnesium sulfate and distilled under reduced pressure to obtain residues. The obtained residues were separated and purified by column chromatography to obtain Compound 120-1 (3.01 g, yield: 68%).

### (Synthesis of Compound 120)

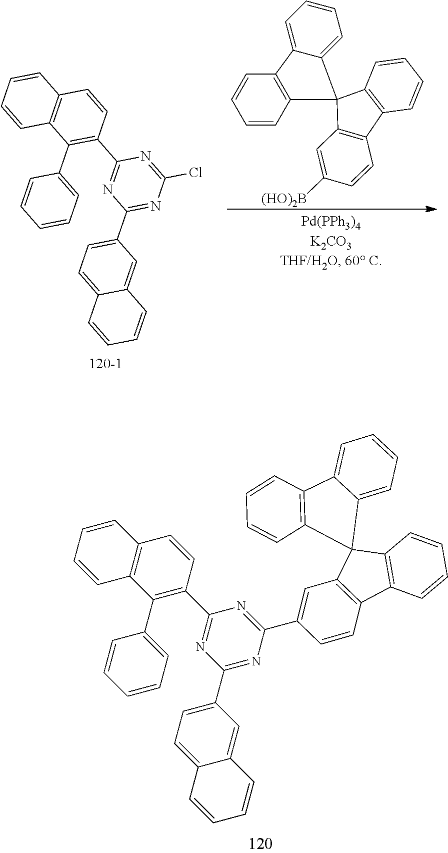 [SMILES: Clc1nc(-c2ccc3ccccc3c2)nc(-c2ccc3ccccc3c2-c2ccccc2)n1 ; OB(O)c1ccc2c(c1)C1(c3ccccc3-c3ccccc31)c1ccccc1-2 ; c1ccc(-c2c(-c3nc(-c4ccc5c(c4)C4(c6ccccc6-c6ccccc64)c4ccccc4-5)nc(-c4ccc5ccccc5c4)n3)ccc3ccccc23)cc1]

Intermediate 120-1 (4.43 g), Pd(PPh₃)₄ (0.56 g), K₂CO₃ (3.45 g), and 9,9′-spirobi[fluoren]-2-ylboronic acid (3.60 g) were dissolved in THF/H₂O (100 mL/25 mL), and the reaction solution was stirred at about 60° C. for about 12 hours. This reaction solution was cooled to room temperature and the reaction was terminated with water, and the extraction was performed three times with ethyl ether to separate an organic layer. The separated organic layer was dried over anhydrous magnesium sulfate and distilled at reduced pressure to obtain residues. The obtained residues were separated and purified by column chromatography to obtain Compound 120 (4.98 g, yield: 69%).

### (8) Synthesis of Compound 155

### (Synthesis of Intermediate 155-1)

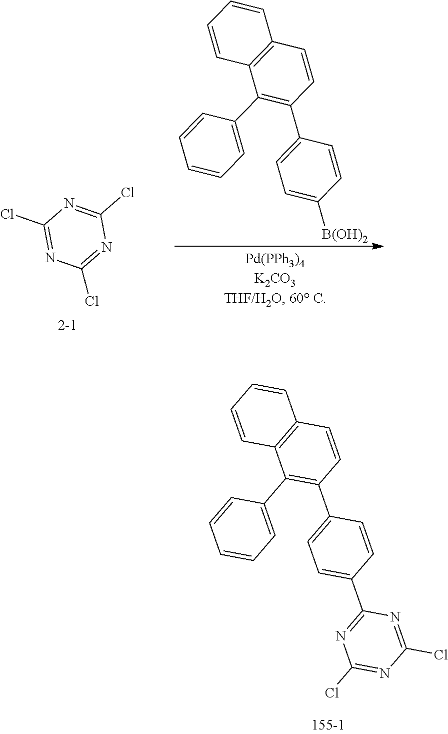 [SMILES: Clc1nc(Cl)nc(Cl)n1 ; OB(O)c1ccc(-c2ccc3ccccc3c2-c2ccccc2)cc1 ; Clc1nc(Cl)nc(-c2ccc(-c3ccc4ccccc4c3-c3ccccc3)cc2)n1]

Intermediate 2-1 (1.84 g), Pd(PPh₃)₄ (0.56 g), K₂CO₃ (3.45 g), and (4-(1-phenylnaphthalen-2-yl)phenyl)boronic acid (3.24 g) were dissolved in THF/H₂O (100 mL/25 mL), and the reaction solution was stirred at about 60° C. for about 12 hours. This reaction solution was cooled to room temperature and the reaction was terminated with water, and the extraction was performed three times with ethyl ether to separate an organic layer. The separated organic layer was dried over anhydrous magnesium sulfate and distilled under reduced pressure to obtain residues. The obtained residues were separated and purified by column chromatography to obtain Intermediate 155-1 (2.82 g, yield: 66%).

### (Synthesis of Compound 155-2)

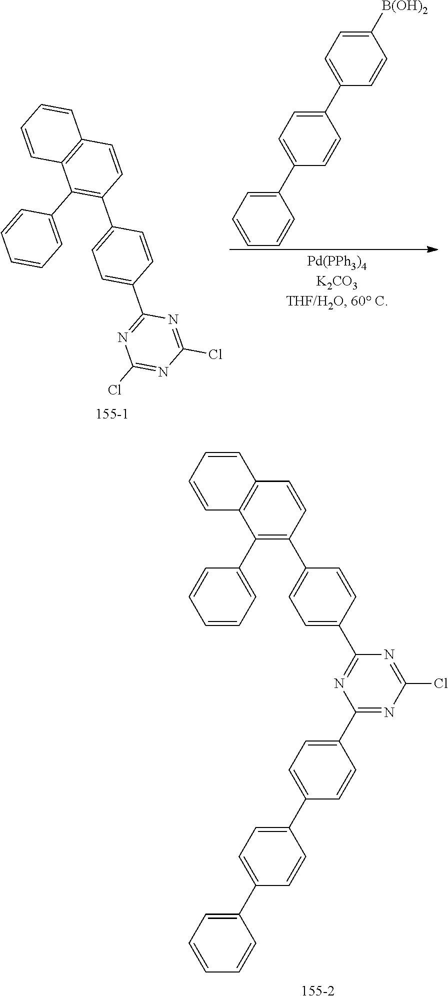 [SMILES: Clc1nc(Cl)nc(-c2ccc(-c3ccc4ccccc4c3-c3ccccc3)cc2)n1 ; OB(O)c1ccc(-c2ccc(-c3ccccc3)cc2)cc1 ; Clc1nc(-c2ccc(-c3ccc(-c4ccccc4)cc3)cc2)nc(-c2ccc(-c3ccc4ccccc4c3-c3ccccc3)cc2)n1]

Intermediate 155-1 (4.28 g), Pd(PPh₃)₄ (0.56 g), K₂CO₃ (3.45 g), and [1,1′:4′,1″-terphenyl]-4-ylboronic acid (2.74 g) were dissolved in THF/H₂O (100 mL/25 mL), and the reaction solution was stirred at about 60° C. for about 12 hours. This reaction solution was cooled to room temperature and the reaction was terminated with water, and the extraction was performed three times with ethyl ether to separate an organic layer. The separated organic layer was dried over anhydrous magnesium sulfate and distilled under reduced pressure to obtain residues. The obtained residues were separated and purified by column chromatography to obtain Intermediate 155-2 (4.16 g, yield: 67%).

### (Synthesis of Compound 155)

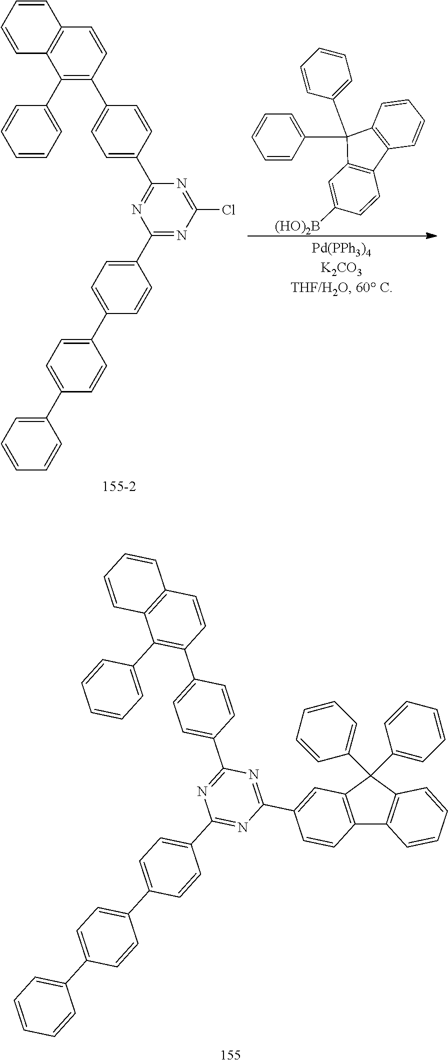 [SMILES: Clc1nc(-c2ccc(-c3ccc(-c4ccccc4)cc3)cc2)nc(-c2ccc(-c3ccc4ccccc4c3-c3ccccc3)cc2)n1 ; OB(O)c1ccc2c(c1)C(c1ccccc1)(c1ccccc1)c1ccccc1-2 ; c1ccc(-c2ccc(-c3ccc(-c4nc(-c5ccc(-c6ccc7ccccc7c6-c6ccccc6)cc5)nc(-c5ccc6c(c5)C(c5ccccc5)(c5ccccc5)c5ccccc5-6)n4)cc3)cc2)cc1]

Intermediate 155-2 (6.22 g), Pd(PPh₃)₄ (0.56 g), K₂CO₃ (3.45 g), and (9,9-diphenyl-9H-fluoren-2-yl)boronic acid (3.60 g) were dissolved in THF/H₂O (100 mL/25 mL), and the reaction solution was stirred at about 60° C. for about 12 hours. This reaction solution was cooled to room temperature and the reaction was terminated with water, and the extraction was performed three times with ethyl ether to separate an organic layer. The separated organic layer was dried over anhydrous magnesium sulfate and distilled at reduced pressure to obtain residues. The obtained residues were separated and purified by column chromatography to obtain Compound 155 (6.32 g, yield: 70%).

### (9) Synthesis of Compound 161

### (Synthesis of Compound 161-1)

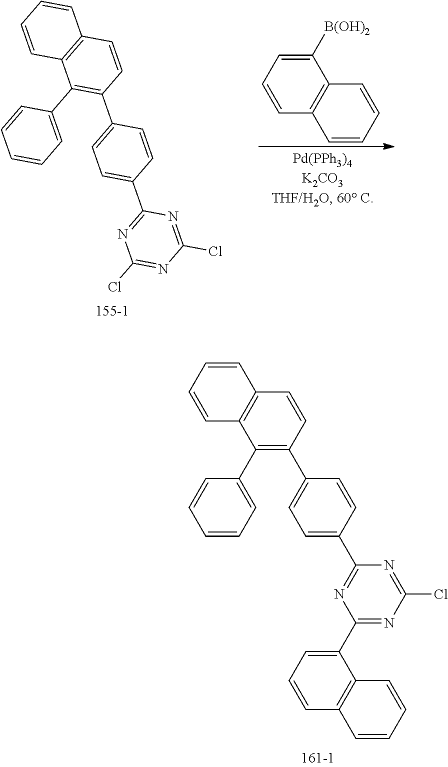 [SMILES: Clc1nc(Cl)nc(-c2ccc(-c3ccc4ccccc4c3-c3ccccc3)cc2)n1 ; OB(O)c1cccc2ccccc12 ; Clc1nc(-c2ccc(-c3ccc4ccccc4c3-c3ccccc3)cc2)nc(-c2cccc3ccccc23)n1]

Intermediate 155-1 (4.28 g), Pd(PPh₃)₄ (0.56 g), K₂CO₃ (3.45 g), and naphthalen-1-ylboronic acid (1.71 g) were dissolved in THF/H₂O (100 mL/25 mL), and the reaction solution was stirred at about 60° C. for about 12 hours. This reaction solution was cooled to room temperature and the reaction was terminated with water, and the extraction was performed three times with ethyl ether to separate an organic layer. The separated organic layer was dried over anhydrous magnesium sulfate and distilled under reduced pressure to obtain residues. The obtained residues were separated and purified by column chromatography to obtain Intermediate 161-1 (3.64 g, yield: 70%).

### (Synthesis of Compound 161)

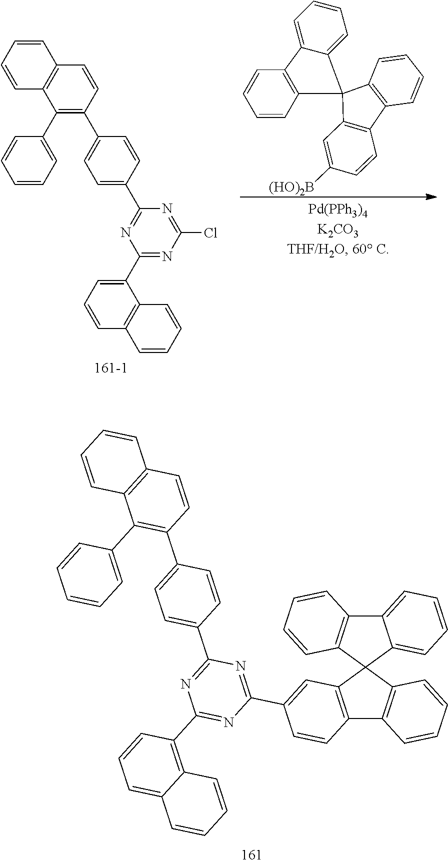 [SMILES: Clc1nc(-c2ccc(-c3ccc4ccccc4c3-c3ccccc3)cc2)nc(-c2cccc3ccccc23)n1 ; OB(O)c1ccc2c(c1)C1(c3ccccc3-c3ccccc31)c1ccccc1-2 ; c1ccc(-c2c(-c3ccc(-c4nc(-c5ccc6c(c5)C5(c7ccccc7-c7ccccc75)c5ccccc5-6)nc(-c5cccc6ccccc56)n4)cc3)ccc3ccccc23)cc1]

Intermediate 161-1 (5.20 g), Pd(PPh₃)₄ (0.56 g), K₂CO₃ (3.45 g), and 9,9′-spirobi[fluoren]-2-ylboronic acid (3.60 g) were dissolved in THF/H₂O (100 mL/25 mL), and the reaction solution was stirred at about 60° C. for about 12 hours. This reaction solution was cooled to room temperature and the reaction was terminated with water, and the extraction was performed three times with ethyl ether to separate an organic layer. The separated organic layer was dried over anhydrous magnesium sulfate and distilled at reduced pressure to obtain residues. The obtained residues were separated and purified by column chromatography to obtain Compound 161 (6.39 g, yield: 80%).

### 2. Manufacture and Evaluation of Light Emitting Elements

### (1) Manufacture of Light Emitting Elements

A light emitting element according to an embodiment including the nitrogen-containing compound according to an embodiment in the electron transport region was manufactured as follows. Compounds 2, 30, 40, 71, 90, 116, 120, 155, and 161, which are Example Compounds as described above, were used as materials for the electron transport layers to manufacture the light emitting elements of Examples 1 to 9, respectively. Comparative Examples 1 to 5 correspond to the light emitting elements manufactured by using Comparative Example Compounds C1 to C4 as materials for the electron transport region.

### Example Compounds

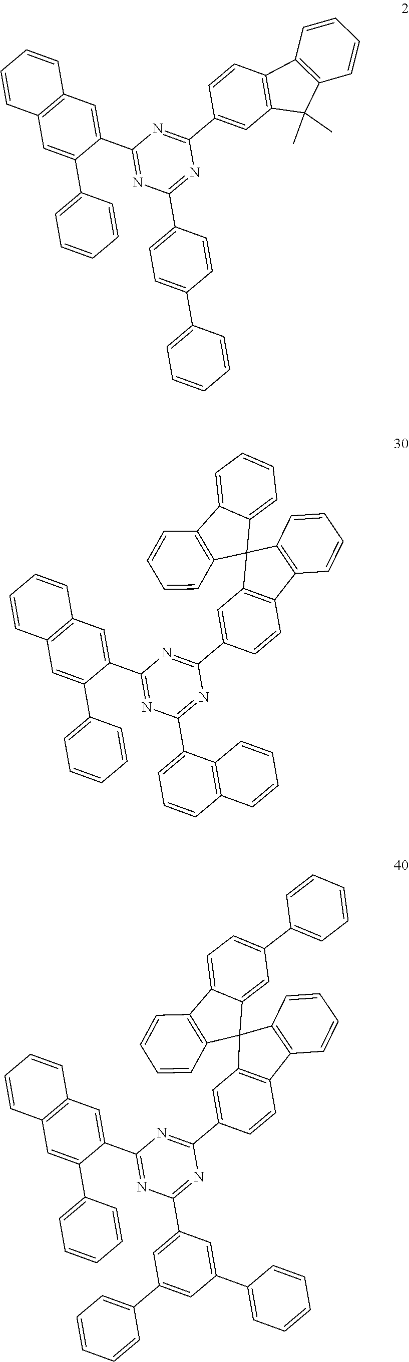 [SMILES: CC1(C)c2ccccc2-c2ccc(-c3nc(-c4ccc(-c5ccccc5)cc4)nc(-c4cc5ccccc5cc4-c4ccccc4)n3)cc21 ; c1ccc(-c2cc3ccccc3cc2-c2nc(-c3ccc4c(c3)C3(c5ccccc5-c5ccccc53)c3ccccc3-4)nc(-c3cccc4ccccc34)n2)cc1 ; c1ccc(-c2cc(-c3ccccc3)cc(-c3nc(-c4ccc5c(c4)C4(c6ccccc6-c6ccc(-c7ccccc7)cc64)c4ccccc4-5)nc(-c4cc5ccccc5cc4-c4ccccc4)n3)c2)cc1]

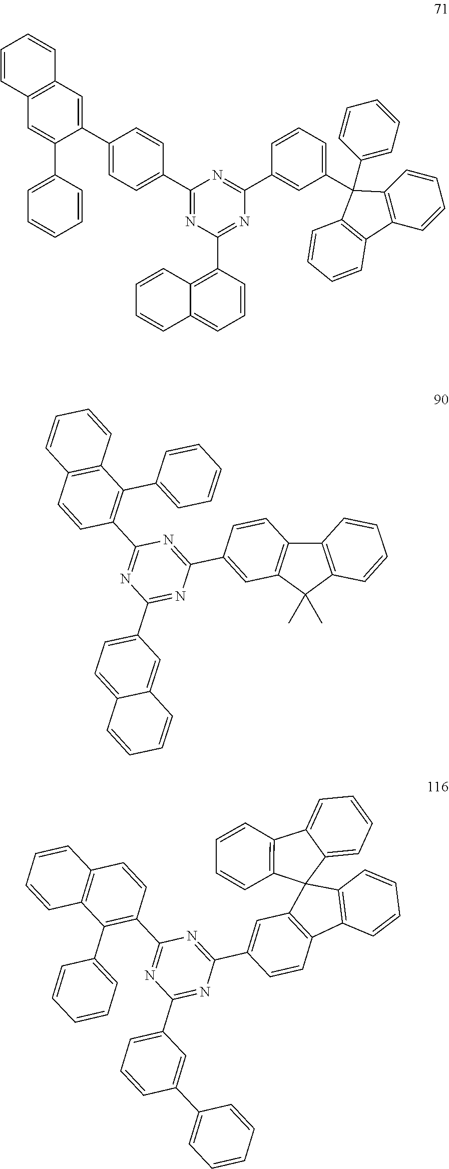 [SMILES: c1ccc(-c2cc3ccccc3cc2-c2ccc(-c3nc(-c4cccc(C5(c6ccccc6)c6ccccc6-c6ccccc65)c4)nc(-c4cccc5ccccc45)n3)cc2)cc1 ; CC1(C)c2ccccc2-c2ccc(-c3nc(-c4ccc5ccccc5c4)nc(-c4ccc5ccccc5c4-c4ccccc4)n3)cc21 ; c1ccc(-c2cccc(-c3nc(-c4ccc5c(c4)C4(c6ccccc6-c6ccccc64)c4ccccc4-5)nc(-c4ccc5ccccc5c4-c4ccccc4)n3)c2)cc1]

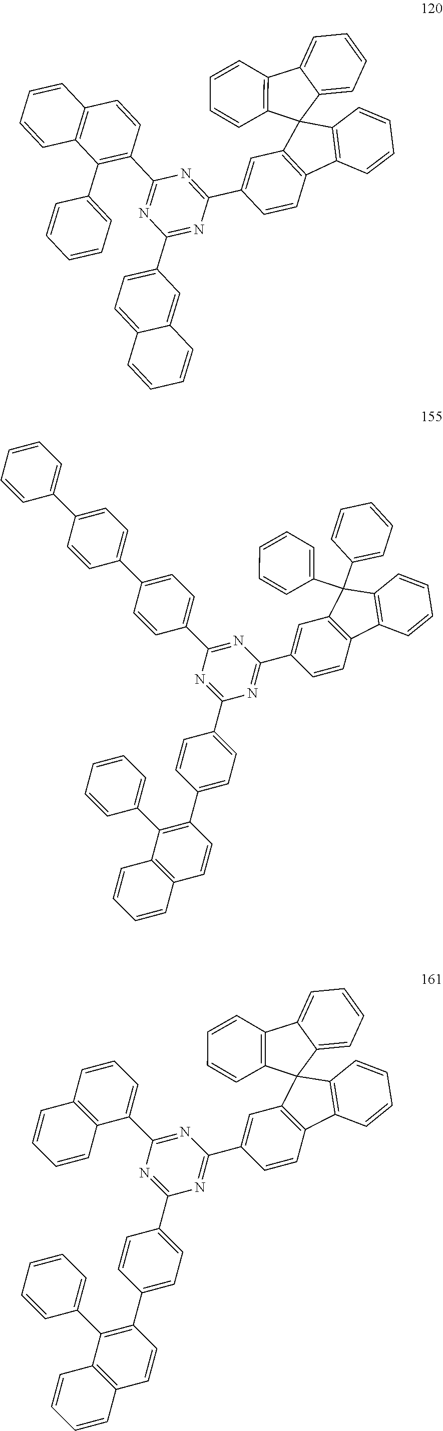 [SMILES: c1ccc(-c2c(-c3nc(-c4ccc5c(c4)C4(c6ccccc6-c6ccccc64)c4ccccc4-5)nc(-c4ccc5ccccc5c4)n3)ccc3ccccc23)cc1 ; c1ccc(-c2ccc(-c3ccc(-c4nc(-c5ccc(-c6ccc7ccccc7c6-c6ccccc6)cc5)nc(-c5ccc6c(c5)C(c5ccccc5)(c5ccccc5)c5ccccc5-6)n4)cc3)cc2)cc1 ; c1ccc(-c2c(-c3ccc(-c4nc(-c5ccc6c(c5)C5(c7ccccc7-c7ccccc75)c5ccccc5-6)nc(-c5cccc6ccccc56)n4)cc3)ccc3ccccc23)cc1]

### Comparative Example Compounds

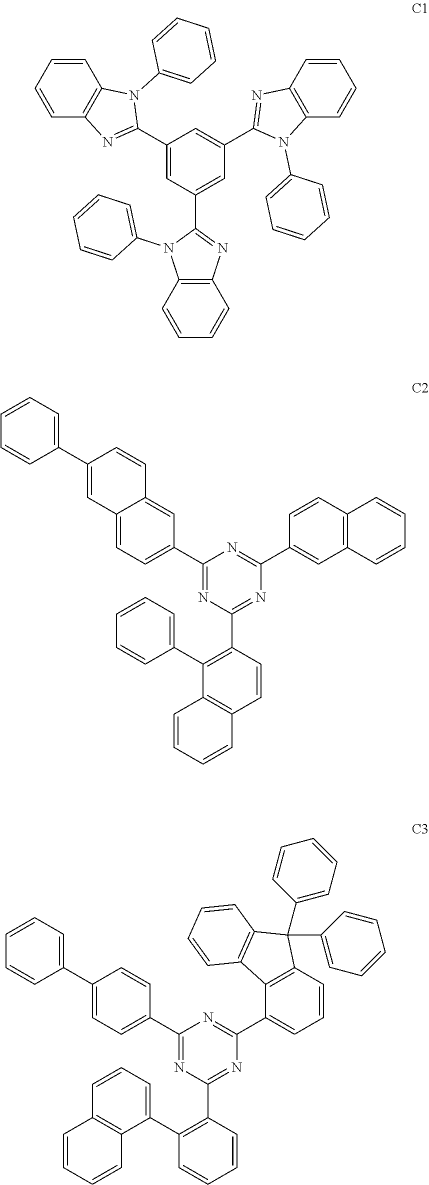 [SMILES: c1ccc(-n2c(-c3cc(-c4nc5ccccc5n4-c4ccccc4)cc(-c4nc5ccccc5n4-c4ccccc4)c3)nc3ccccc32)cc1 ; c1ccc(-c2ccc3cc(-c4nc(-c5ccc6ccccc6c5)nc(-c5ccc6ccccc6c5-c5ccccc5)n4)ccc3c2)cc1 ; c1ccc(-c2ccc(-c3nc(-c4ccccc4-c4cccc5ccccc45)nc(-c4cccc5c4-c4ccccc4C5(c4ccccc4)c4ccccc4)n3)cc2)cc1]

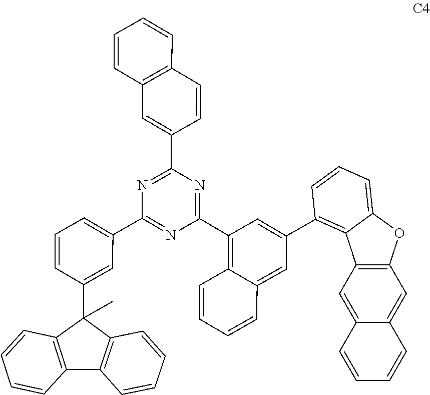 [SMILES: CC1(c2cccc(-c3nc(-c4ccc5ccccc5c4)nc(-c4cc(-c5cccc6oc7cc8ccccc8cc7c56)cc5ccccc45)n3)c2)c2ccccc2-c2ccccc21]

### (Manufacture of Light Emitting Elements)

In the light emitting elements of the Examples and the Comparative Examples, a glass substrate (made by Corning Co.), on which an ITO electrode of about 15 Ω/cm² (about 1,200 Å) is formed as a first electrode, was cut to a size of about 50 mm×50 mm×0.7 mm, cleansed by ultrasonic waves by using isopropyl alcohol and pure water for about five minutes each, and irradiated with ultraviolet rays for about 30 minutes and exposed to ozone and cleansed. The glass substrate was installed on a vacuum deposition apparatus.

NPD was deposited on the upper portion of the first electrode to form a 300 Å-thick hole injection layer, Compound H-1-1 was deposited on the upper portion of the hole injection layer to form a 200 Å-thick hole transport layer, and CzSi was deposited on the upper portion of the hole transport layer to form a 100 Å-thick emission-auxiliary layer.

A host material, in which the second compound and the third compound were mixed at a weight ratio of about 1:1, the fourth compound as a sensitizer, and the first compound as a dopant material were co-deposited at a weight ratio of about 84:15:1 to form a 200 Å-thick emission layer. TSPO1 was deposited on the upper portion of the emission layer to form a 200 Å-thick hole blocking layer, an Example Compound or a Comparative Example Compound was deposited on the upper portion of the hole blocking layer to form a 300 Å-thick electron transport layer, LiF was deposited on the upper portion of the electron transport layer to form a 10 Å-thick electron injection layer, and Al was deposited on the upper portion of the electron injection layer to form a 3,000 Å-thick second electrode, thereby manufacturing a light emitting element. Each layer was formed by a vacuum deposition method.

Compound HT2 or Compound HT3, each from Compound Group 2 as described above, were used as the second compound; Compound ETH66 or Compound ETH86, each from Compound Group 3 as described above, were used as the third compound; Compound AD-37 or Compound AD-38, each from Compound Group 4 as described above, or Compound AD-C below, was used as the fourth compound; and t-DABNA was used as the first compound. Comparative Example 5 does not use a host material in which the second compound and the third compound are mixed at a weight ratio of about 1:1, but uses Compound E-C below as a host material.

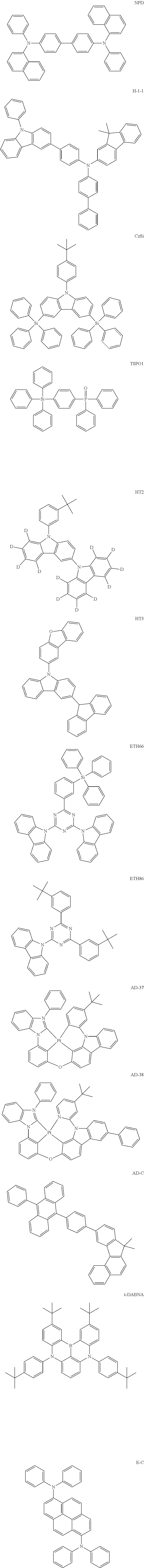

### (2) Evaluation of Light Emitting Element Characteristics

### (Evaluation of Light Emitting Element Characteristics)

Element efficiencies and element service lives of the light emitting elements manufactured with Example Compounds 2, 30, 40, 71, 90, 116, 120, 155, and 161 and Comparative Example Compounds C1 to C4 as described above were evaluated. Evaluation results of the light emitting elements in Examples 1 to 9 and Comparative Examples 1 to 5 are listed in Table 1. To evaluate the characteristics of the light emitting elements manufactured in Examples 1 to 9 and Comparative Examples 1 to 5 above, driving voltages (V) and luminous efficiencies (Cd/A) at a current density of 1,000 cd/m² were each measured by using Keithley MU 236 and a luminance meter PR650, and the time taken to reach 95% brightness relative to an initial brightness was measured as a service life (T95), and a relative service life was calculated on the basis of the element of Comparative Example 1, and the results are listed in Table 1.

TABLE 1

| | Host | | | | | |
|---|---|---|---|---|---|---|
| | (second | | | | | Relative |
| | compound:third | | Material for | Driving | | service |
| | compound = | Fourth | electron | voltage | Efficiency | life |
| | 5:5) | compound | transport layer | (V) | (Cd/A) | (T95) |
| Example 1 | HT3/ETH66 | AD-38 | Compound 2 | 4.2 | 25.7 | 4.1 |
| Example 2 | HT3/ETH66 | AD-38 | Compound 30 | 4.6 | 26.5 | 4.7 |
| Example 3 | HT3/ETH66 | AD-38 | Compound 40 | 4.4 | 26.1 | 5.1 |
| Example 4 | HT3/ETH66 | AD-38 | Compound 71 | 4.3 | 25.9 | 5.3 |
| Example 5 | HT3/ETH66 | AD-38 | Compound 90 | 4.2 | 25.1 | 4.4 |
| Example 6 | HT3/ETH66 | AD-38 | Compound 116 | 4.1 | 25.4 | 3.9 |
| Example 7 | HT3/ETH66 | AD-38 | Compound 120 | 4.6 | 26.3 | 6.1 |
| Example 8 | HT2/ETH86 | AD-37 | Compound 155 | 4.7 | 25.5 | 4.7 |
| Example 9 | HT2/ETH86 | AD-37 | Compound 161 | 4.8 | 25.8 | 4.6 |
| Comparative | HT3/ETH66 | AD-38 | Comparative | 5.6 | 18.8 | 1 |
| Example 1 | | | Example | | | |
| | | | Compound C1 | | | |
| Comparative | HT3/ETH66 | AD-38 | Comparative | 4.9 | 22.3 | 3.1 |
| Example 2 | | | Example | | | |
| | | | Compound C2 | | | |
| Comparative | HT3/ETH66 | AD-38 | Comparative | 5.2 | 21.1 | 3.4 |
| Example 3 | | | Example | | | |
| | | | Compound C3 | | | |
| Comparative | HT3/ETH66 | AD-38 | Comparative | 5.7 | 20.3 | 2.3 |
| Example 4 | | | Example | | | |
| | | | Compound C4 | | | |
| Comparative | E-C | AD-C | Comparative | 5.0 | 13.7 | 3.9 |
| Example 5 | | | Example | | | |
| | | | Compound C2 | | | |

Referring to the results of Table 1, it may be seen that the light emitting elements of the Examples, in which the nitrogen-containing compounds according to embodiments are used as a material for the hole transport layer, exhibit relatively low driving voltages, high luminous efficiencies, and long element service lives, as compared to the light emitting elements of the Comparative Examples. The Example Compounds have a structure that includes a triazine ring, and a first substituent and a second substituent that are each linked to the carbon atoms of the triazine ring, and thus may achieve high luminous efficiency and long service life. The first substituent may include a phenylnaphthalene moiety. The phenylnaphthalene moiety of the first substituent may include a naphthalene moiety, and a benzene moiety linked to the naphthalene moiety. The benzene moiety and the triazine ring may be linked to the naphthalene moiety of the first substituent at an ortho-position to each other. The second substituent may include a fluorene moiety. The fluorene moiety of the second substituent may be a structure substituted with a methyl group or a phenyl group, or two phenyl substituents of the fluorene moiety may be bonded to each other to form a spiro structure. In the second substituent, a carbon atom of an aromatic ring in the fluorene moiety may be linked to the triazine ring. The nitrogen-containing compound of an Example may have a triazine ring to which the first substituent and the second substituent are linked, and thus have charge transport ability. Therefore, it may be expected that the elements of the Examples including the Example Compounds as a material for the electron transport layer exhibit relatively high luminous efficiency and long element service life, as compared to the elements in the Comparative Examples.

Comparative Example Compound C1 includes a heterocycle containing a nitrogen atom, but does not include a triazine ring, or the first and second substituents according to embodiments. Accordingly, it is considered that the light emitting elements of Comparative Example 1 including Comparative Example Compound C1 exhibits deterioration in luminous efficiency and element service life.

Comparative Example Compound C2 includes a triazine ring and a first substituent linked to the triazine ring according to embodiments, but does not include a second substituent. Accordingly, it may be confirmed that the light emitting elements of Comparative Examples 2 and 5 each including Comparative Example Compound C2 has a relatively high driving voltage value as compared to the elements including the Example Compounds, and exhibits deterioration in luminous efficiency and element service life. It is considered that Comparative Example Compound C2 includes a naphthyl group or a phenylnaphthyl group rather than the second substituent, and thus the characteristics of the elements of Comparative Examples 2 and 5 including Comparative Example Compound C2 are deteriorated.

Comparative Example Compound C3 includes a triazine ring and a second substituent linked to the triazine ring according to embodiments, but does not include a first substituent. Accordingly, it may be confirmed that the light emitting element of Comparative Example 3 including Comparative Example Compound C3 has a relatively high driving voltage value as compared to the elements including the Example Compounds, and exhibits deterioration in luminous efficiency and element service life. It is considered that Comparative Example Compound C3 includes an unsubstituted naphthyl group or an unsubstituted phenyl group rather than the first substituent, and thus the characteristics of the element of Comparative Example 3 including Comparative Example Compound C3 are deteriorated.

Comparative Example Compound C4 includes a triazine ring, but does not include the first and second substituents according to embodiments. Accordingly, it may be confirmed that the light emitting element of Comparative Example 4 including Comparative Example Compound C4 has a relatively high driving voltage value as compared to the elements including the Example Compounds, and exhibits deterioration in luminous efficiency and element service life. It is considered that Comparative Example Compound C4 includes a naphthyl group to which naphthobenzofuran is linked rather than the first substituent, and a methylfluorenyl group to which the triazine ring and the carbon atom constituting the pentagonal ring in the fluorene moiety are linked, rather than the second substituent, and thus the characteristics of the element of Comparative Example 4 including Comparative Example Compound C4 are deteriorated.

The light emitting element according to an embodiment may exhibit improved element characteristics with high efficiency and a long service life.

The nitrogen-containing compound according to an embodiment may be included in an electron transport region of a light emitting element to contribute to high efficiency and a long service life of the light emitting element.

The display device according to an embodiment may include the light emitting element having excellent efficiency and service life.

Embodiments have been disclosed herein, and although terms are employed, they are used and are to be interpreted in a generic and descriptive sense only and not for the purposes of limitation. In some instances, as would be apparent to one of ordinary skill in the art, features, characteristics, and/or elements described in connection with an embodiment may be used singly or in combination with features, characteristics, and/or elements described in connection with other embodiments unless otherwise specifically indicated. Accordingly, it will be understood by those of ordinary skill in the art that various changes in form and details may be made without departing from the spirit and scope of the disclosure as set forth in the claims.

What is claimed is:

1. A light emitting element comprising:
a first electrode;
a hole transport region disposed on the first electrode;
an emission layer disposed on the hole transport region;
an electron transport region disposed on the emission layer; and
a second electrode disposed on the electron transport region, wherein the electron transport region includes a nitrogen-containing compound represented by Formula 1, and
the emission layer includes an organometallic compound represented by Formula D-1: 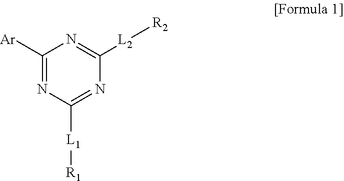
wherein in Formula 1,
Ar is a substituted or unsubstituted aryl group having 6 to 30 ring-forming carbon atoms or a substituted or unsubstituted heteroaryl group having 2 to 30 ring-forming carbon atoms,
L₁ and L₂ are each independently a direct linkage or a substituted or unsubstituted aryl group having 6 to 30 ring-forming carbon atoms,
R₁ is a group represented by Formula 2, and
R₂ is a group represented by Formula 3-1, Formula 3-2, or Formula 3-3: 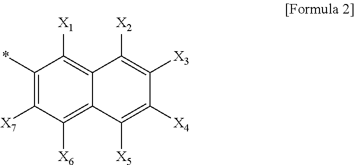
wherein in Formula 2,
X₁ to X₇ are each independently a hydrogen atom, a deuterium atom, a halogen atom, a cyano group, a hydroxy group, a substituted or unsubstituted oxy group, a substituted or unsubstituted thio group, a substituted or unsubstituted boron group, a substituted or unsubstituted silyl group, a substituted or unsubstituted amine group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms,
except that X₁ or X₇ is a substituted or unsubstituted phenyl group, and 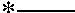 [SMILES: *C]
is a position linked to L₁ in Formula 1: 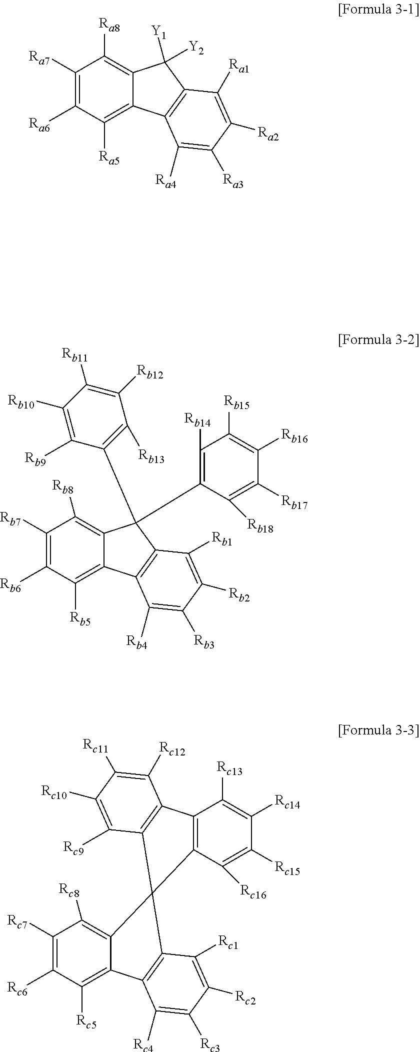
wherein in Formula 3-1,
Y₁ and Y₂ are each independently a hydrogen atom, a deuterium atom, a halogen atom, a cyano group, a hydroxy group, or a substituted or unsubstituted alkyl group having 1 to 20 carbon atoms,
wherein in Formula 3-1 to Formula 3-3,
Rₐ₁ to Rₐ₈, R_{b1} to R_{b18}, and R_{c1} to R_{c16} are each independently a hydrogen atom, a deuterium atom, a halogen atom, a cyano group, a hydroxy group, a substituted or unsubstituted oxy group, a substituted or unsubstituted thio group, a substituted or unsubstituted boron group, a substituted or unsubstituted silyl group, a substituted or unsubstituted amine group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms,
except that
when R₂ in Formula 1 is a group represented by Formula 3-1, one of Rₐ₁ to Rₐ₈ in Formula 3-1 is a position linked to L₂ in Formula 1,
when R₂ in Formula 1 is a group represented by Formula 3-2, one of R_{b1} to R_{b18} in Formula 3-2 is a position linked to L₂ in Formula 1, and
when R₂ in Formula 1 is a group represented by Formula 3-3, one of R_{c1} to R_{c16} in Formula 3-3 is a position linked to L₂ in Formula 1; 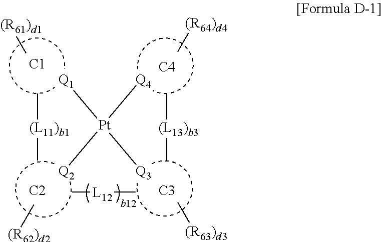
wherein in Formula D-1,
Q₁ to Q₄ are each independently C or N,
C1 to C4 are each independently a substituted or unsubstituted hydrocarbon ring having 5 to 30 ring-forming carbon atoms or a substituted or unsubstituted heterocycle having 2 to 30 ring-forming carbon atoms,
L₁₁ to L₁₃ are each independently a direct linkage, 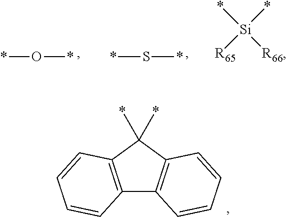
a substituted or unsubstituted alkylene group having 1 to 20 carbon atoms, a substituted or unsubstituted arylene group having 6 to 30 ring-forming carbon atoms, or a substituted or unsubstituted heteroarylene group having 2 to 30 ring-forming carbon atoms,
b1 to b3 are each independently 0 or 1,
R₆₁ to R₆₆ are each independently a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 carbon atoms, a substituted or unsubstituted aryl group having 6 to 60 ring-forming carbon atoms, or a substituted or unsubstituted heteroaryl group having 2 to 60 ring-forming carbon atoms, and
d1 to d4 are each independently an integer from 0 to 4.

2. The light emitting element of claim 1, wherein
the electron transport region comprises:
an electron transport layer disposed on the emission layer; and
an electron injection layer disposed on the electron transport layer, and
the electron transport layer comprises the nitrogen-containing compound.

3. The light emitting element of claim 1, wherein
L₁ is a direct linkage or a substituted or unsubstituted phenyl group, and
L₂ is a direct linkage.

4. The light emitting element of claim 1, wherein Ar is a substituted or unsubstituted phenyl group, a substituted or unsubstituted biphenyl group, a substituted or unsubstituted terphenyl group, or a substituted or unsubstituted naphthyl group.

5. The light emitting element of claim 1, wherein the nitrogen-containing compound is represented by one of Formula 1-1 to Formula 1-4: 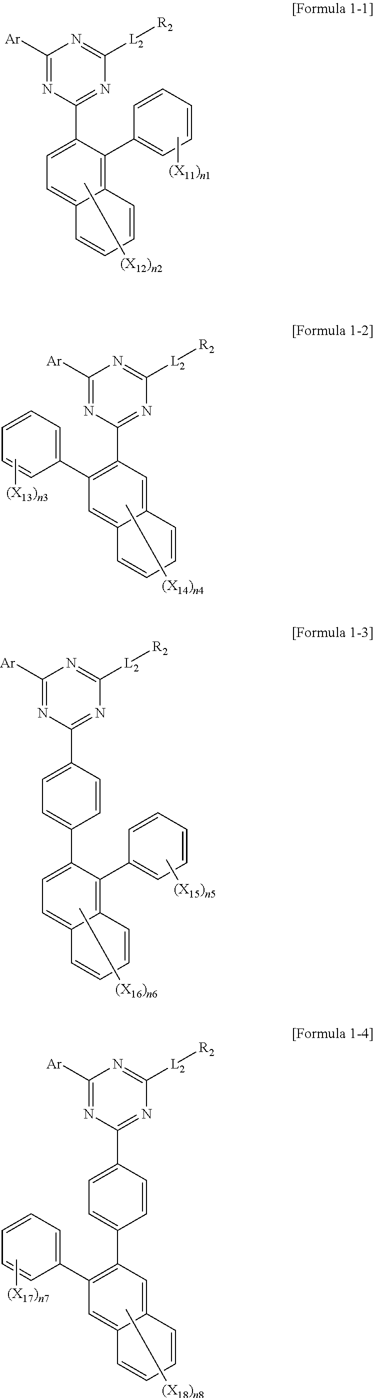
wherein in Formula 1-1 to Formula 1-4,
X₁₁ to X₁₈ are each independently a hydrogen atom or a deuterium atom,
n1, n3, n5, and n7 are each independently an integer from 0 to 5,
n2, n4, n6, and n8 are each independently an integer from 0 to 6, and
Ar, L₂, and R₂ are the same as defined in Formula 1.

6. The light emitting element of claim 5, wherein X₁₁ to X₁₈ are each a hydrogen atom.

7. The light emitting element of claim 1, wherein the nitrogen-containing compound is represented by one of Formula 1-5 to Formula 1-9: 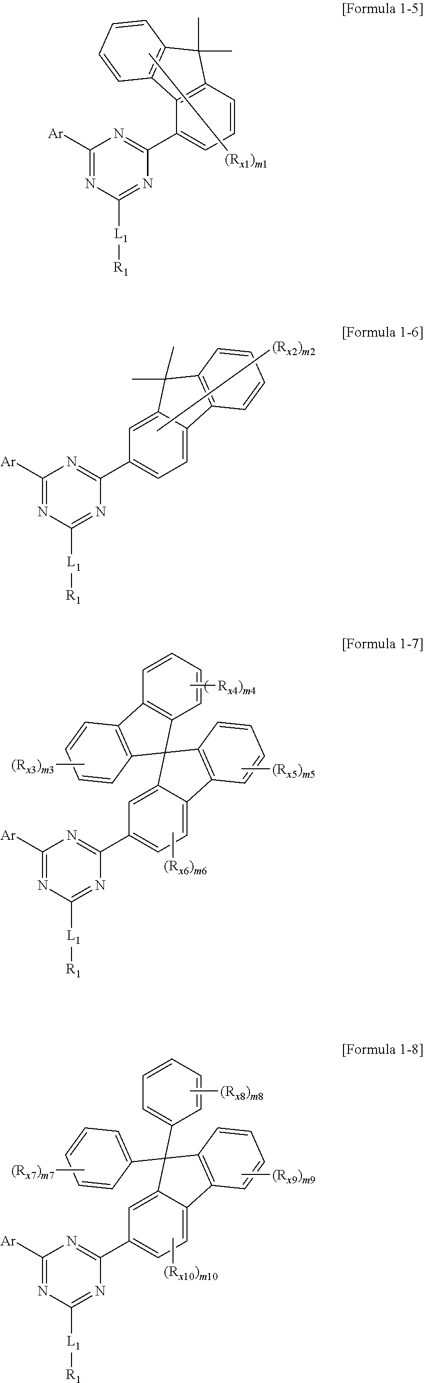 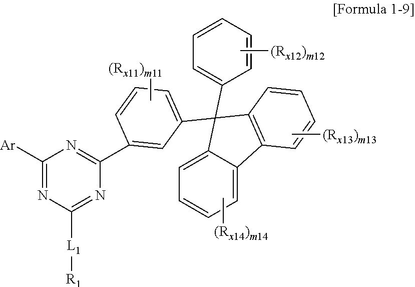
wherein in Formula 1-5 to Formula 1-9,
Rₓ₁ to Rₓ₁₄ are each independently a hydrogen atom, a deuterium atom, a halogen atom, a cyano group, or a substituted or unsubstituted aryl group having 6 to 12 ring-forming carbon atoms,
m1 and m2 are each independently an integer from 0 to 7,
m3 to m5, m9, m11, m13, and m14 are each independently an integer from 0 to 4,
m6 and m10 are each independently an integer from 0 to 3,
m7, m8, and m12 are each independently an integer from 0 to 5, and
Ar, L₁, and R₁ are the same as defined in Formula 1.

8. The light emitting element of claim 7, wherein
Rₓ₁ to Rₓ₃ and Rₓ₅ to Rₓ₁₄ are each a hydrogen atom, and
Rₓ₄ is a hydrogen atom or an unsubstituted phenyl group.

9. The light emitting element of claim 1, wherein the nitrogen-containing compound is represented by one of Formula 1-10 to Formula 1-14: 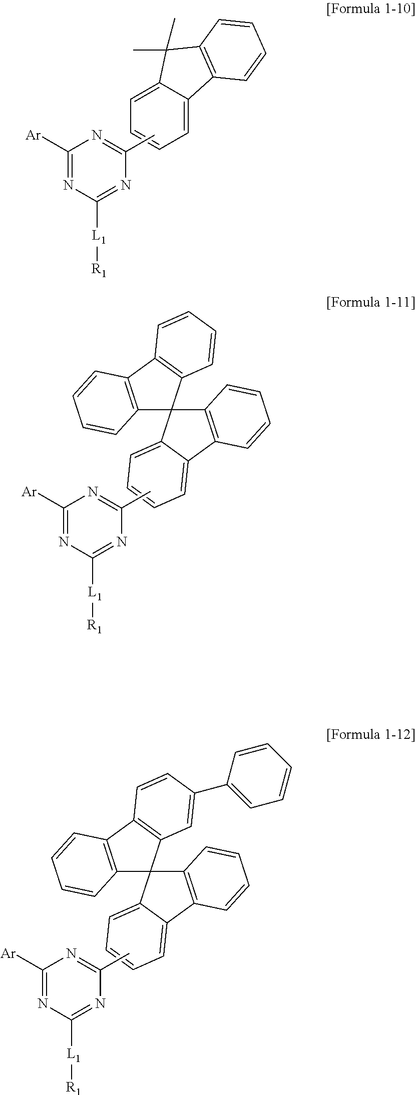 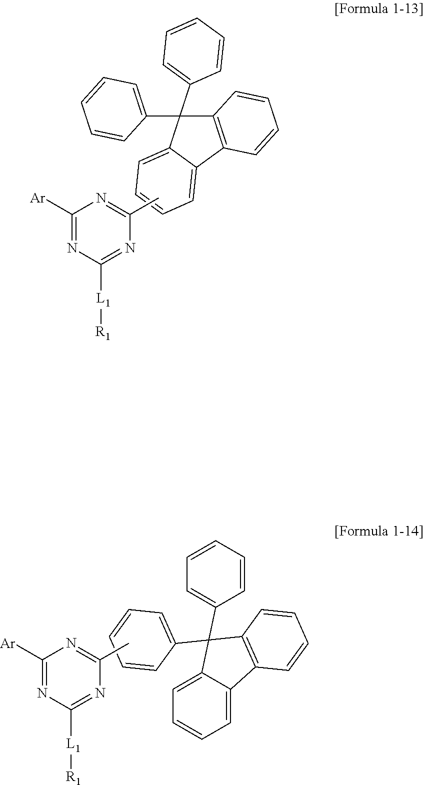
wherein in Formula 1-10 to Formula 1-14,
Ar, L₁, and R₁ are the same as defined in Formula 1.

10. The light emitting element of claim 1, wherein the nitrogen-containing compound comprises at least one compound selected from Compound Group 1:

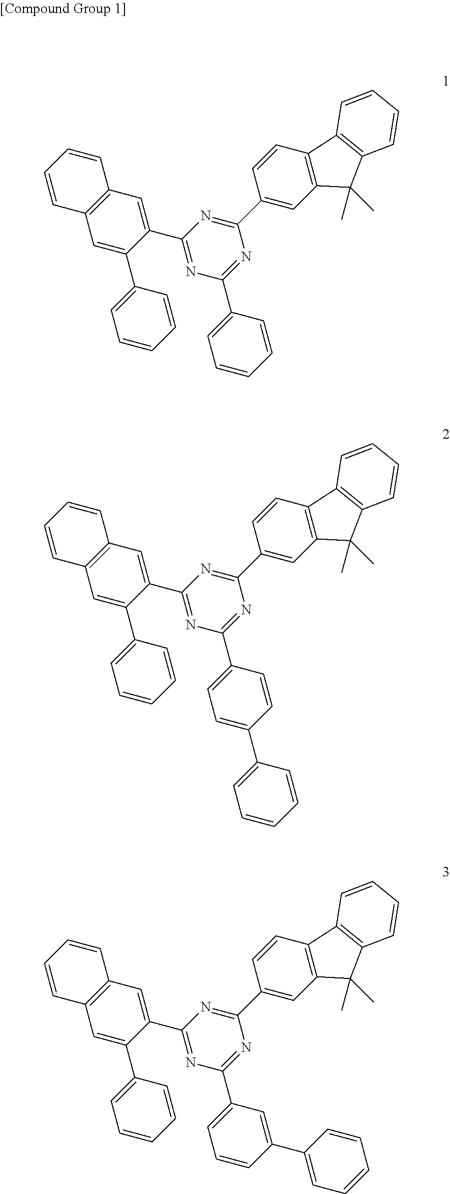 [SMILES: CC1(C)c2ccccc2-c2ccc(-c3nc(-c4ccccc4)nc(-c4cc5ccccc5cc4-c4ccccc4)n3)cc21 ; CC1(C)c2ccccc2-c2ccc(-c3nc(-c4ccc(-c5ccccc5)cc4)nc(-c4cc5ccccc5cc4-c4ccccc4)n3)cc21 ; CC1(C)c2ccccc2-c2ccc(-c3nc(-c4cccc(-c5ccccc5)c4)nc(-c4cc5ccccc5cc4-c4ccccc4)n3)cc21]

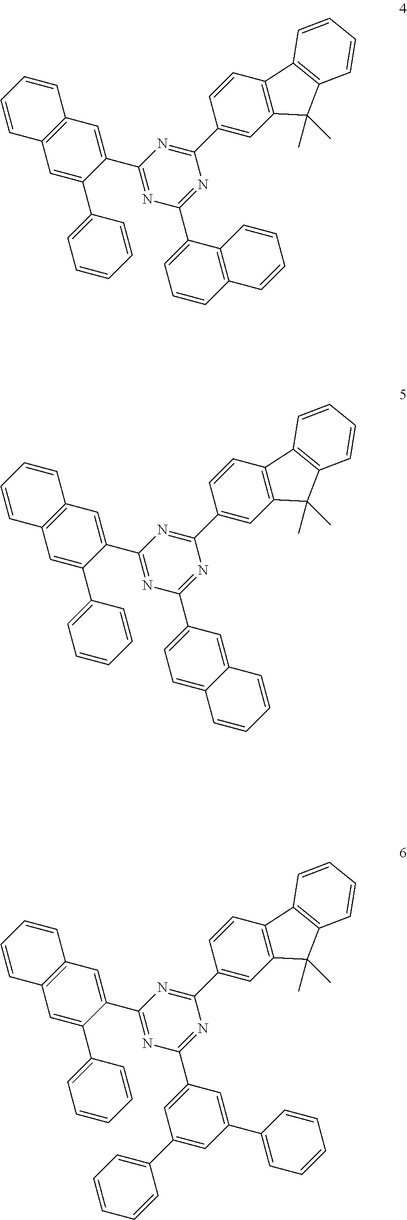 [SMILES: CC1(C)c2ccccc2-c2ccc(-c3nc(-c4cc5ccccc5cc4-c4ccccc4)nc(-c4cccc5ccccc45)n3)cc21 ; CC1(C)c2ccccc2-c2ccc(-c3nc(-c4ccc5ccccc5c4)nc(-c4cc5ccccc5cc4-c4ccccc4)n3)cc21 ; CC1(C)c2ccccc2-c2ccc(-c3nc(-c4cc(-c5ccccc5)cc(-c5ccccc5)c4)nc(-c4cc5ccccc5cc4-c4ccccc4)n3)cc21]

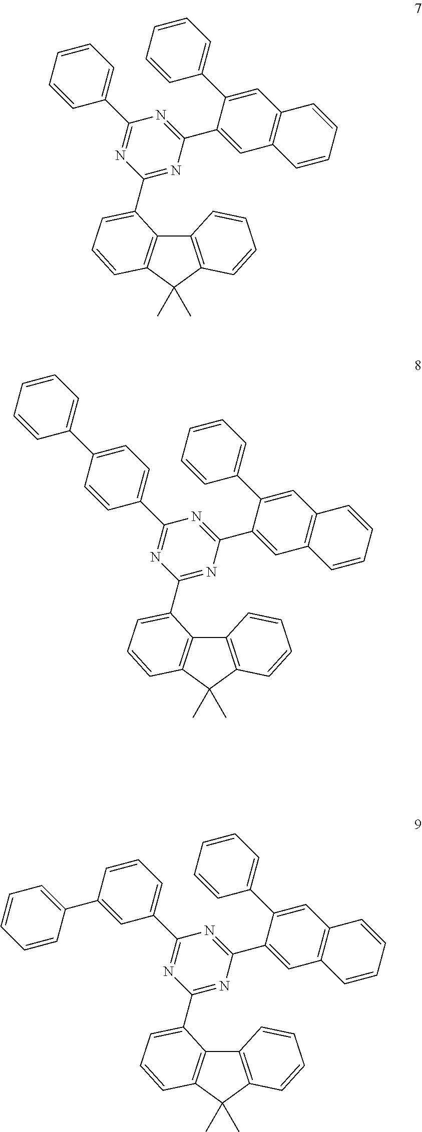 [SMILES: CC1(C)c2ccccc2-c2c(-c3nc(-c4ccccc4)nc(-c4cc5ccccc5cc4-c4ccccc4)n3)cccc21 ; CC1(C)c2ccccc2-c2c(-c3nc(-c4ccc(-c5ccccc5)cc4)nc(-c4cc5ccccc5cc4-c4ccccc4)n3)cccc21 ; CC1(C)c2ccccc2-c2c(-c3nc(-c4cccc(-c5ccccc5)c4)nc(-c4cc5ccccc5cc4-c4ccccc4)n3)cccc21]

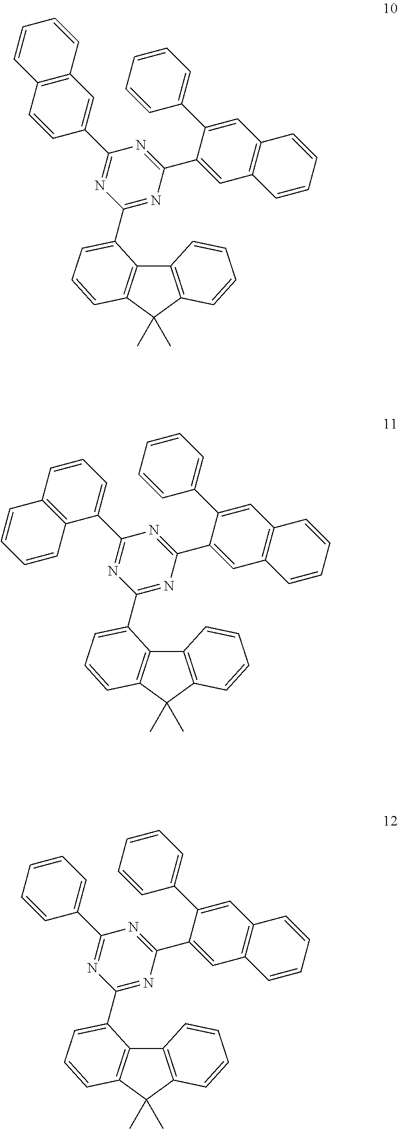 [SMILES: CC1(C)c2ccccc2-c2c(-c3nc(-c4ccc5ccccc5c4)nc(-c4cc5ccccc5cc4-c4ccccc4)n3)cccc21 ; CC1(C)c2ccccc2-c2c(-c3nc(-c4cc5ccccc5cc4-c4ccccc4)nc(-c4cccc5ccccc45)n3)cccc21 ; CC1(C)c2ccccc2-c2c(-c3nc(-c4ccccc4)nc(-c4cc5ccccc5cc4-c4ccccc4)n3)cccc21]

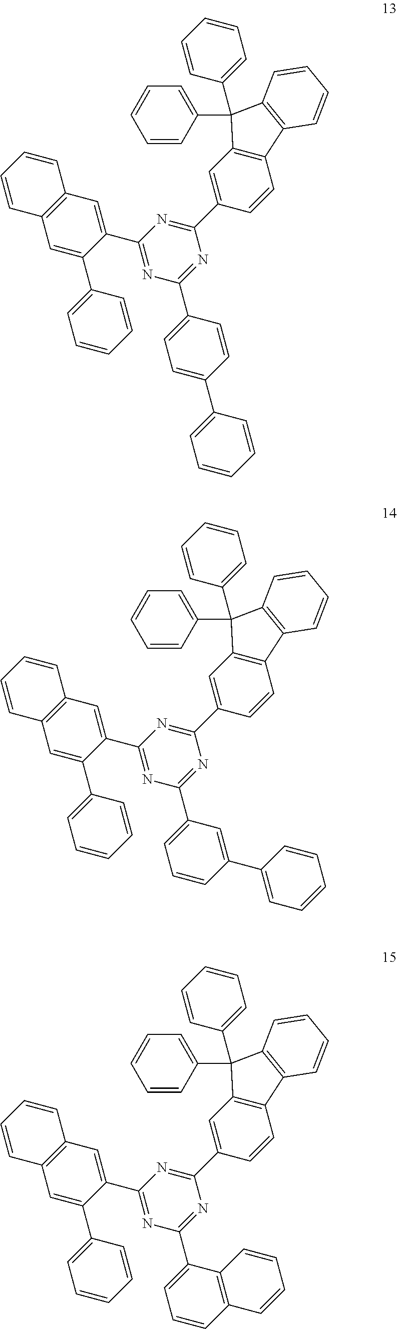 [SMILES: c1ccc(-c2ccc(-c3nc(-c4ccc5c(c4)C(c4ccccc4)(c4ccccc4)c4ccccc4-5)nc(-c4cc5ccccc5cc4-c4ccccc4)n3)cc2)cc1 ; c1ccc(-c2cccc(-c3nc(-c4ccc5c(c4)C(c4ccccc4)(c4ccccc4)c4ccccc4-5)nc(-c4cc5ccccc5cc4-c4ccccc4)n3)c2)cc1 ; c1ccc(-c2cc3ccccc3cc2-c2nc(-c3ccc4c(c3)C(c3ccccc3)(c3ccccc3)c3ccccc3-4)nc(-c3cccc4ccccc34)n2)cc1]

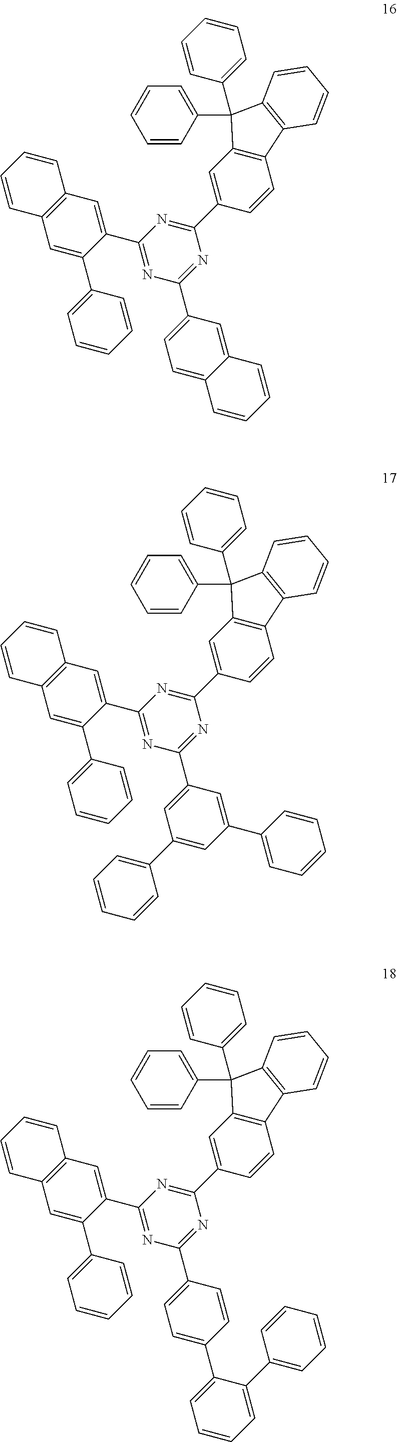 [SMILES: c1ccc(-c2cc3ccccc3cc2-c2nc(-c3ccc4c(c3)C(c3ccccc3)(c3ccccc3)c3ccccc3-4)nc(-c3ccc4ccccc4c3)n2)cc1 ; c1ccc(-c2cc(-c3ccccc3)cc(-c3nc(-c4ccc5c(c4)C(c4ccccc4)(c4ccccc4)c4ccccc4-5)nc(-c4cc5ccccc5cc4-c4ccccc4)n3)c2)cc1 ; c1ccc(-c2ccccc2-c2ccc(-c3nc(-c4ccc5c(c4)C(c4ccccc4)(c4ccccc4)c4ccccc4-5)nc(-c4cc5ccccc5cc4-c4ccccc4)n3)cc2)cc1]

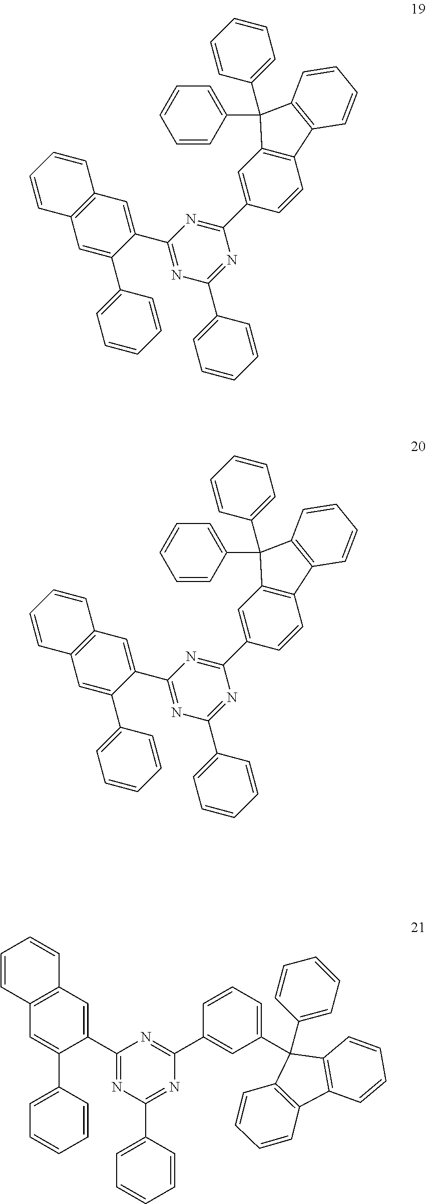 [SMILES: c1ccc(-c2nc(-c3ccc4c(c3)C(c3ccccc3)(c3ccccc3)c3ccccc3-4)nc(-c3cc4ccccc4cc3-c3ccccc3)n2)cc1 ; c1ccc(-c2nc(-c3ccc4c(c3)C(c3ccccc3)(c3ccccc3)c3ccccc3-4)nc(-c3cc4ccccc4cc3-c3ccccc3)n2)cc1 ; c1ccc(-c2nc(-c3cccc(C4(c5ccccc5)c5ccccc5-c5ccccc54)c3)nc(-c3cc4ccccc4cc3-c3ccccc3)n2)cc1]

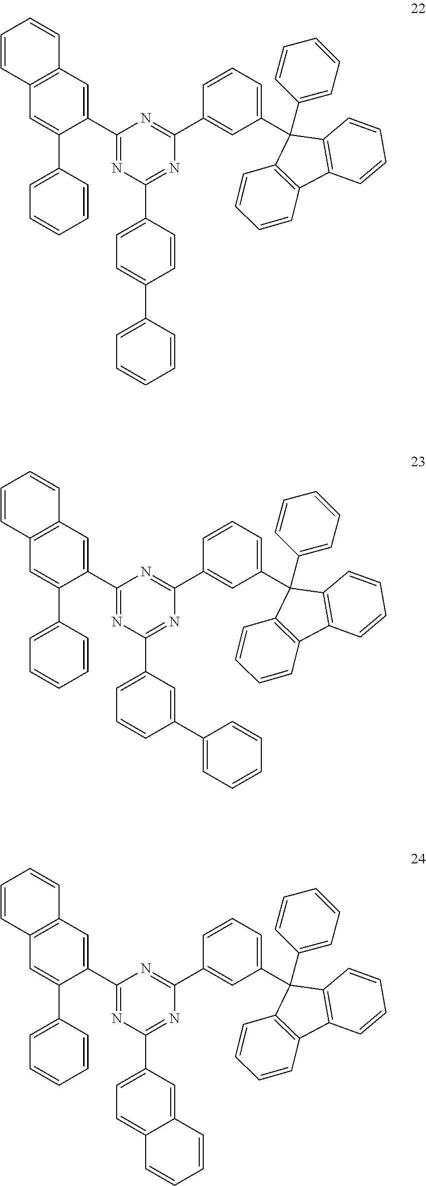 [SMILES: c1ccc(-c2ccc(-c3nc(-c4cccc(C5(c6ccccc6)c6ccccc6-c6ccccc65)c4)nc(-c4cc5ccccc5cc4-c4ccccc4)n3)cc2)cc1 ; c1ccc(-c2cccc(-c3nc(-c4cccc(C5(c6ccccc6)c6ccccc6-c6ccccc65)c4)nc(-c4cc5ccccc5cc4-c4ccccc4)n3)c2)cc1 ; c1ccc(-c2cc3ccccc3cc2-c2nc(-c3cccc(C4(c5ccccc5)c5ccccc5-c5ccccc54)c3)nc(-c3ccc4ccccc4c3)n2)cc1]

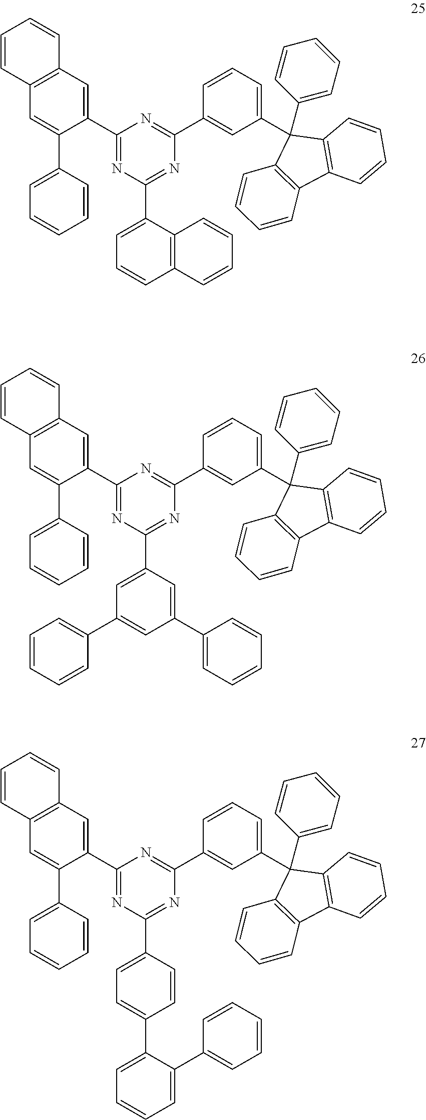 [SMILES: c1ccc(-c2cc3ccccc3cc2-c2nc(-c3cccc(C4(c5ccccc5)c5ccccc5-c5ccccc54)c3)nc(-c3cccc4ccccc34)n2)cc1 ; c1ccc(-c2cc(-c3ccccc3)cc(-c3nc(-c4cccc(C5(c6ccccc6)c6ccccc6-c6ccccc65)c4)nc(-c4cc5ccccc5cc4-c4ccccc4)n3)c2)cc1 ; c1ccc(-c2ccccc2-c2ccc(-c3nc(-c4cccc(C5(c6ccccc6)c6ccccc6-c6ccccc65)c4)nc(-c4cc5ccccc5cc4-c4ccccc4)n3)cc2)cc1]

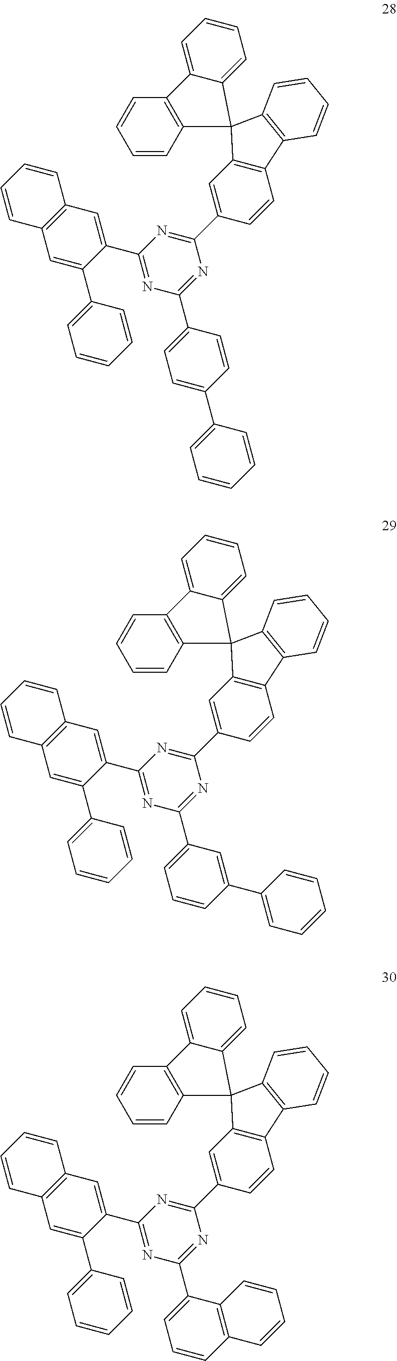 [SMILES: c1ccc(-c2ccc(-c3nc(-c4ccc5c(c4)C4(c6ccccc6-c6ccccc64)c4ccccc4-5)nc(-c4cc5ccccc5cc4-c4ccccc4)n3)cc2)cc1 ; c1ccc(-c2cccc(-c3nc(-c4ccc5c(c4)C4(c6ccccc6-c6ccccc64)c4ccccc4-5)nc(-c4cc5ccccc5cc4-c4ccccc4)n3)c2)cc1 ; c1ccc(-c2cc3ccccc3cc2-c2nc(-c3ccc4c(c3)C3(c5ccccc5-c5ccccc53)c3ccccc3-4)nc(-c3cccc4ccccc34)n2)cc1]

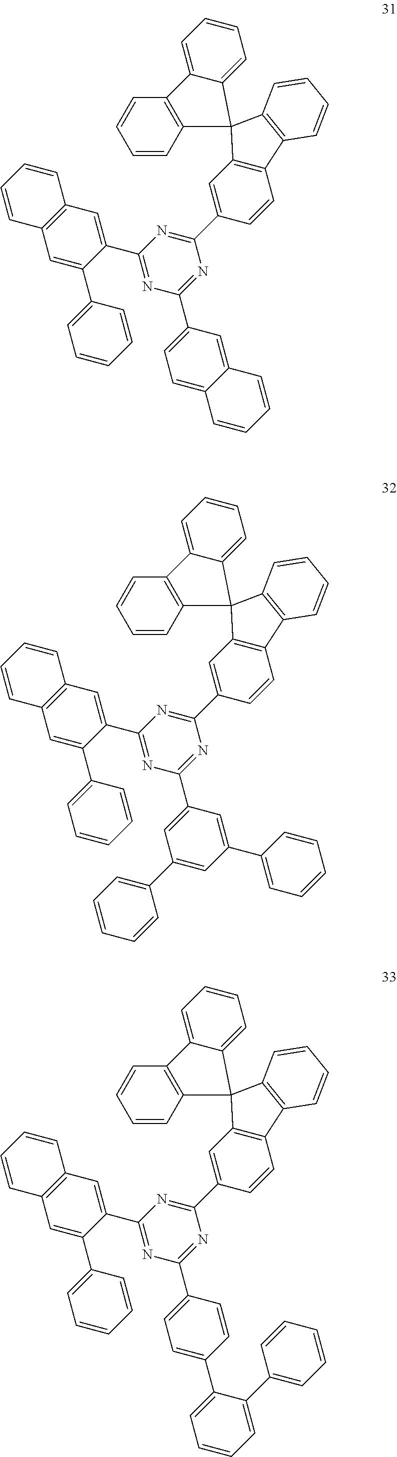 [SMILES: c1ccc(-c2cc3ccccc3cc2-c2nc(-c3ccc4c(c3)C3(c5ccccc5-c5ccccc53)c3ccccc3-4)nc(-c3ccc4ccccc4c3)n2)cc1 ; c1ccc(-c2cc(-c3ccccc3)cc(-c3nc(-c4ccc5c(c4)C4(c6ccccc6-c6ccccc64)c4ccccc4-5)nc(-c4cc5ccccc5cc4-c4ccccc4)n3)c2)cc1 ; c1ccc(-c2ccccc2-c2ccc(-c3nc(-c4ccc5c(c4)C4(c6ccccc6-c6ccccc64)c4ccccc4-5)nc(-c4cc5ccccc5cc4-c4ccccc4)n3)cc2)cc1]

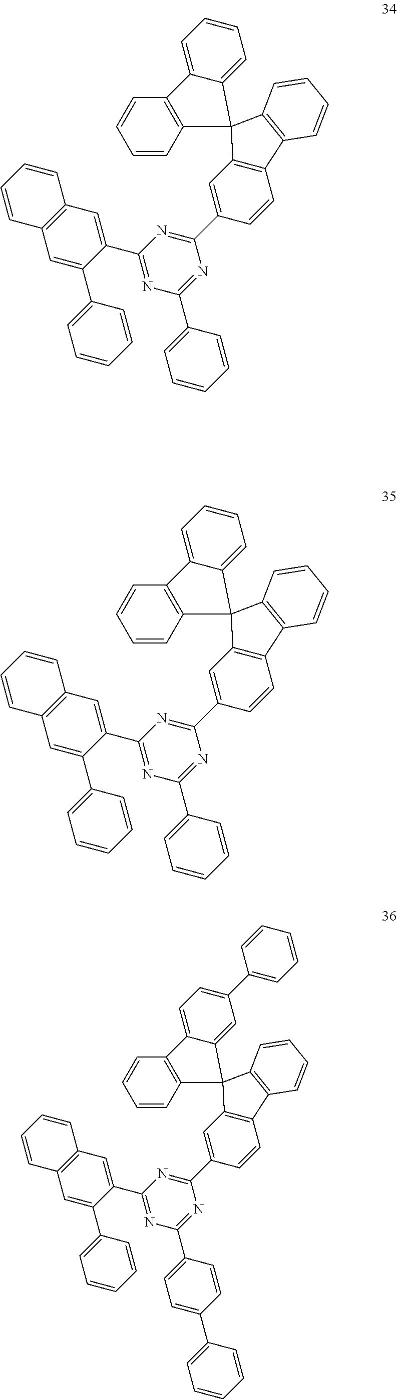 [SMILES: c1ccc(-c2nc(-c3ccc4c(c3)C3(c5ccccc5-c5ccccc53)c3ccccc3-4)nc(-c3cc4ccccc4cc3-c3ccccc3)n2)cc1 ; c1ccc(-c2nc(-c3ccc4c(c3)C3(c5ccccc5-c5ccccc53)c3ccccc3-4)nc(-c3cc4ccccc4cc3-c3ccccc3)n2)cc1 ; c1ccc(-c2ccc(-c3nc(-c4ccc5c(c4)C4(c6ccccc6-c6ccc(-c7ccccc7)cc64)c4ccccc4-5)nc(-c4cc5ccccc5cc4-c4ccccc4)n3)cc2)cc1]

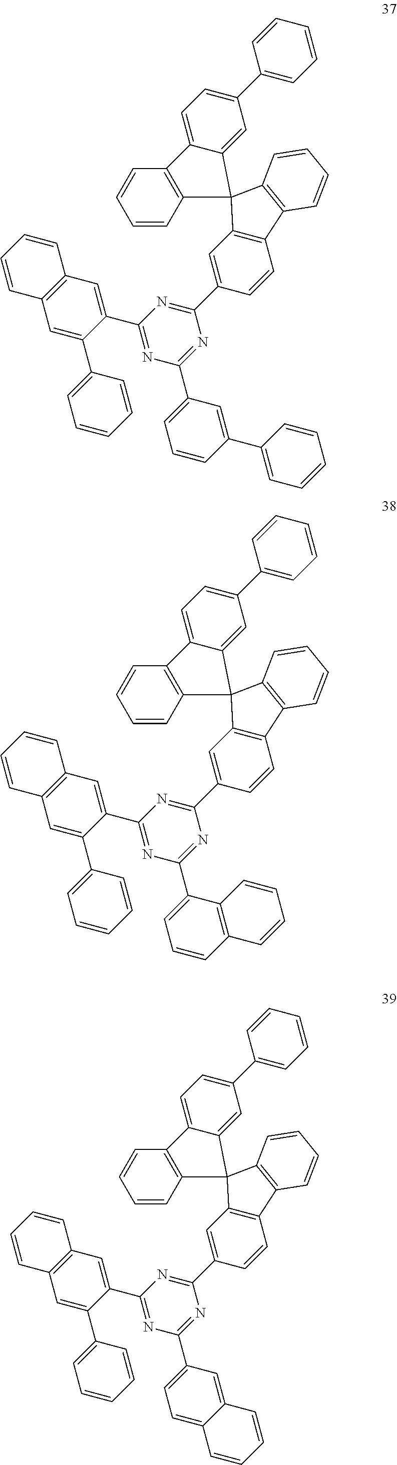

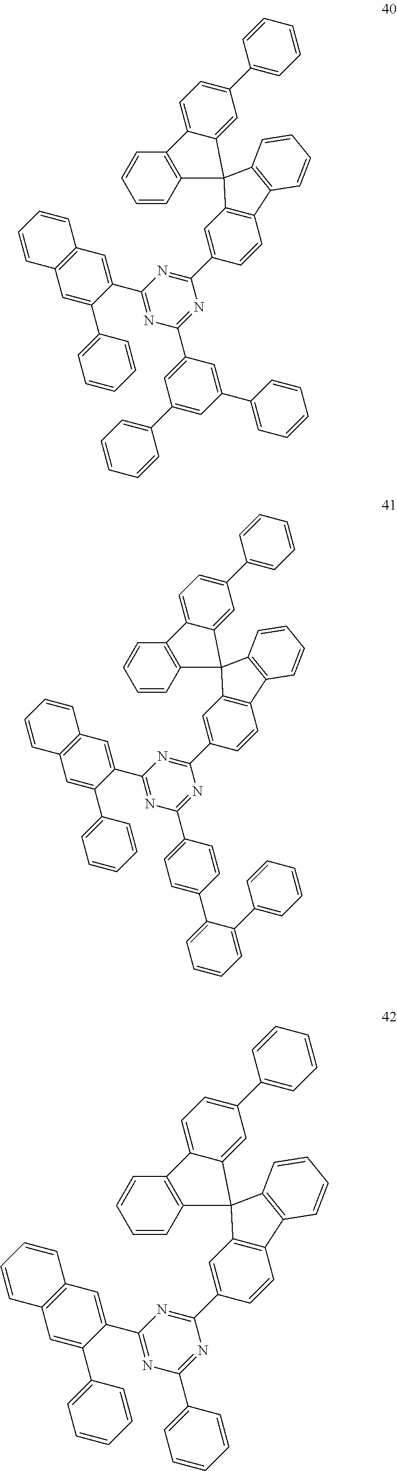 [SMILES: c1ccc(-c2cc(-c3ccccc3)cc(-c3nc(-c4ccc5c(c4)C4(c6ccccc6-c6ccc(-c7ccccc7)cc64)c4ccccc4-5)nc(-c4cc5ccccc5cc4-c4ccccc4)n3)c2)cc1 ; c1ccc(-c2ccc3c(c2)C2(c4ccccc4-3)c3ccccc3-c3ccc(-c4nc(-c5ccc(-c6ccccc6-c6ccccc6)cc5)nc(-c5cc6ccccc6cc5-c5ccccc5)n4)cc32)cc1 ; c1ccc(-c2ccc3c(c2)C2(c4ccccc4-3)c3ccccc3-c3ccc(-c4nc(-c5ccccc5)nc(-c5cc6ccccc6cc5-c5ccccc5)n4)cc32)cc1]

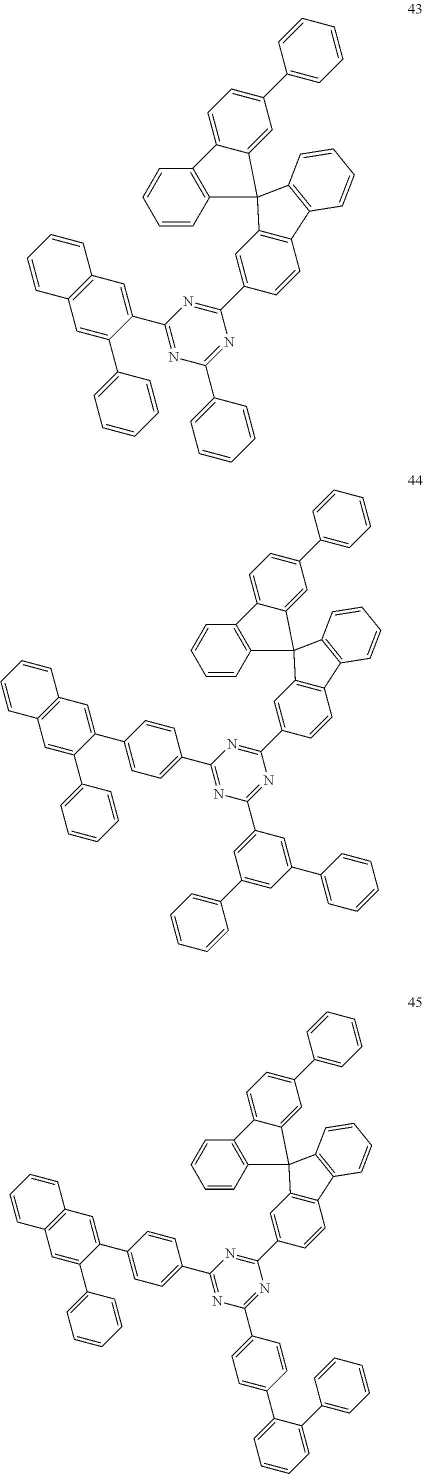 [SMILES: c1ccc(-c2ccc3c(c2)C2(c4ccccc4-3)c3ccccc3-c3ccc(-c4nc(-c5ccccc5)nc(-c5cc6ccccc6cc5-c5ccccc5)n4)cc32)cc1 ; c1ccc(-c2cc(-c3ccccc3)cc(-c3nc(-c4ccc(-c5cc6ccccc6cc5-c5ccccc5)cc4)nc(-c4ccc5c(c4)C4(c6ccccc6-c6ccc(-c7ccccc7)cc64)c4ccccc4-5)n3)c2)cc1 ; c1ccc(-c2ccc3c(c2)C2(c4ccccc4-3)c3ccccc3-c3ccc(-c4nc(-c5ccc(-c6ccccc6-c6ccccc6)cc5)nc(-c5ccc(-c6cc7ccccc7cc6-c6ccccc6)cc5)n4)cc32)cc1]

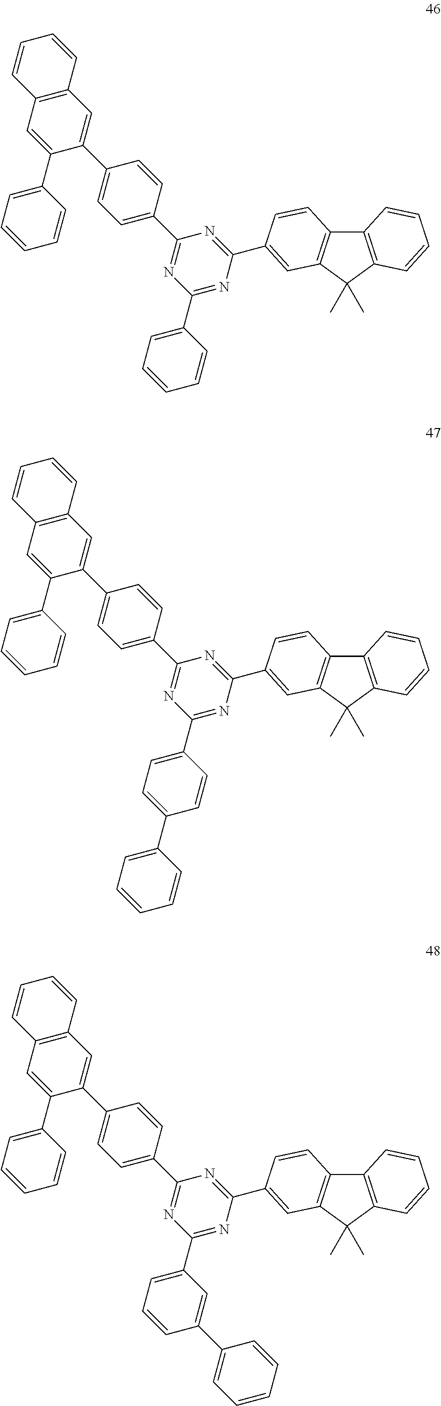 [SMILES: CC1(C)c2ccccc2-c2ccc(-c3nc(-c4ccccc4)nc(-c4ccc(-c5cc6ccccc6cc5-c5ccccc5)cc4)n3)cc21 ; CC1(C)c2ccccc2-c2ccc(-c3nc(-c4ccc(-c5ccccc5)cc4)nc(-c4ccc(-c5cc6ccccc6cc5-c5ccccc5)cc4)n3)cc21 ; CC1(C)c2ccccc2-c2ccc(-c3nc(-c4ccc(-c5cc6ccccc6cc5-c5ccccc5)cc4)nc(-c4cccc(-c5ccccc5)c4)n3)cc21]

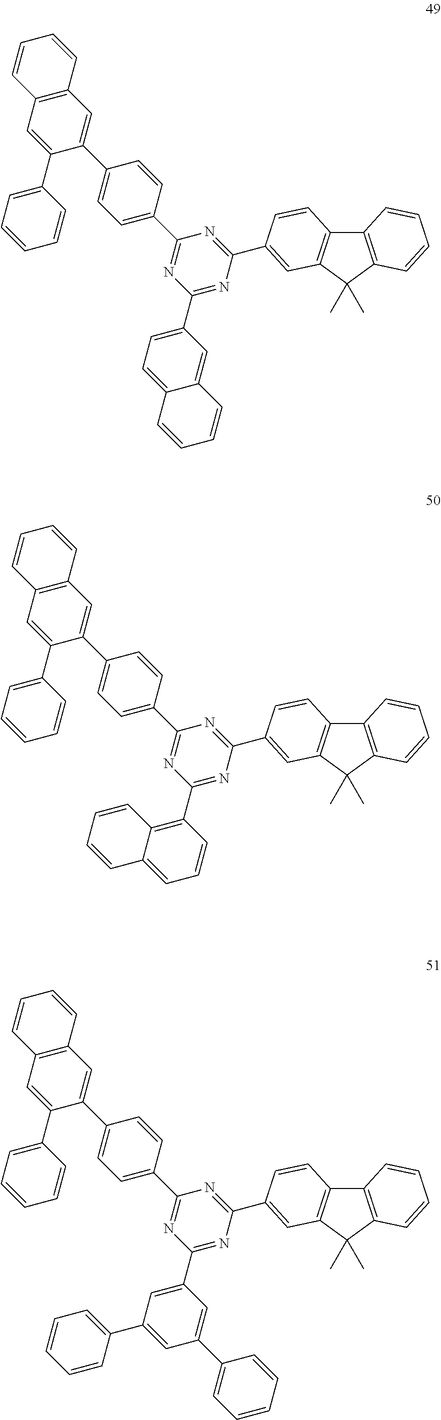 [SMILES: CC1(C)c2ccccc2-c2ccc(-c3nc(-c4ccc(-c5cc6ccccc6cc5-c5ccccc5)cc4)nc(-c4ccc5ccccc5c4)n3)cc21 ; CC1(C)c2ccccc2-c2ccc(-c3nc(-c4ccc(-c5cc6ccccc6cc5-c5ccccc5)cc4)nc(-c4cccc5ccccc45)n3)cc21 ; CC1(C)c2ccccc2-c2ccc(-c3nc(-c4ccc(-c5cc6ccccc6cc5-c5ccccc5)cc4)nc(-c4cc(-c5ccccc5)cc(-c5ccccc5)c4)n3)cc21]

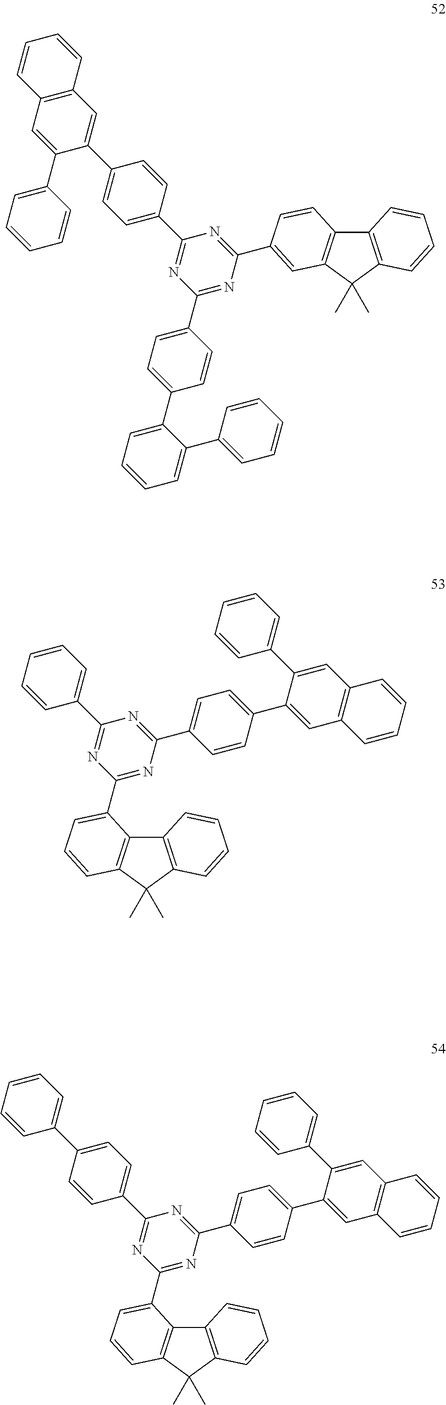 [SMILES: CC1(C)c2ccccc2-c2ccc(-c3nc(-c4ccc(-c5ccccc5-c5ccccc5)cc4)nc(-c4ccc(-c5cc6ccccc6cc5-c5ccccc5)cc4)n3)cc21 ; CC1(C)c2ccccc2-c2c(-c3nc(-c4ccccc4)nc(-c4ccc(-c5cc6ccccc6cc5-c5ccccc5)cc4)n3)cccc21 ; CC1(C)c2ccccc2-c2c(-c3nc(-c4ccc(-c5ccccc5)cc4)nc(-c4ccc(-c5cc6ccccc6cc5-c5ccccc5)cc4)n3)cccc21]

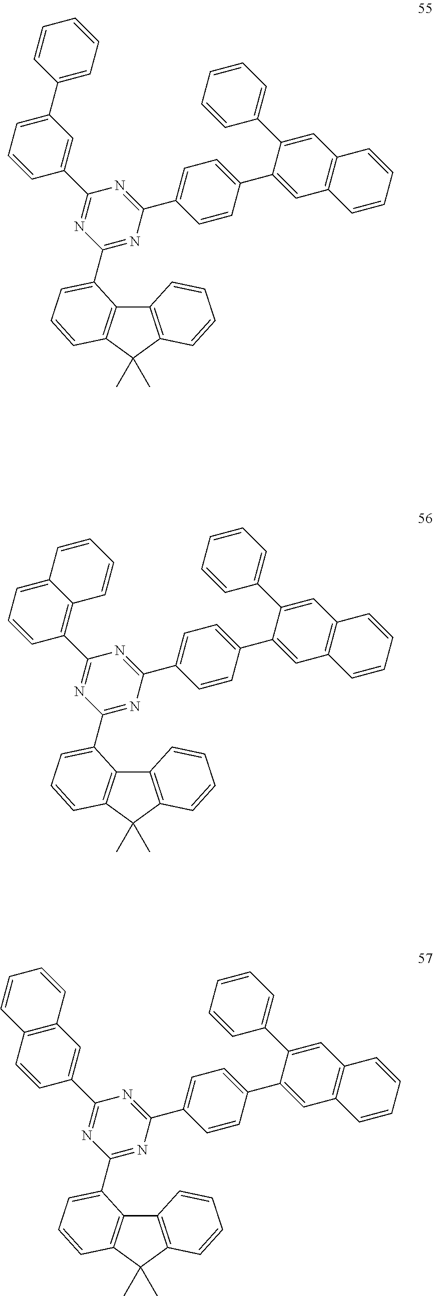 [SMILES: CC1(C)c2ccccc2-c2c(-c3nc(-c4ccc(-c5cc6ccccc6cc5-c5ccccc5)cc4)nc(-c4cccc(-c5ccccc5)c4)n3)cccc21 ; CC1(C)c2ccccc2-c2c(-c3nc(-c4ccc(-c5cc6ccccc6cc5-c5ccccc5)cc4)nc(-c4cccc5ccccc45)n3)cccc21 ; CC1(C)c2ccccc2-c2c(-c3nc(-c4ccc(-c5cc6ccccc6cc5-c5ccccc5)cc4)nc(-c4ccc5ccccc5c4)n3)cccc21]

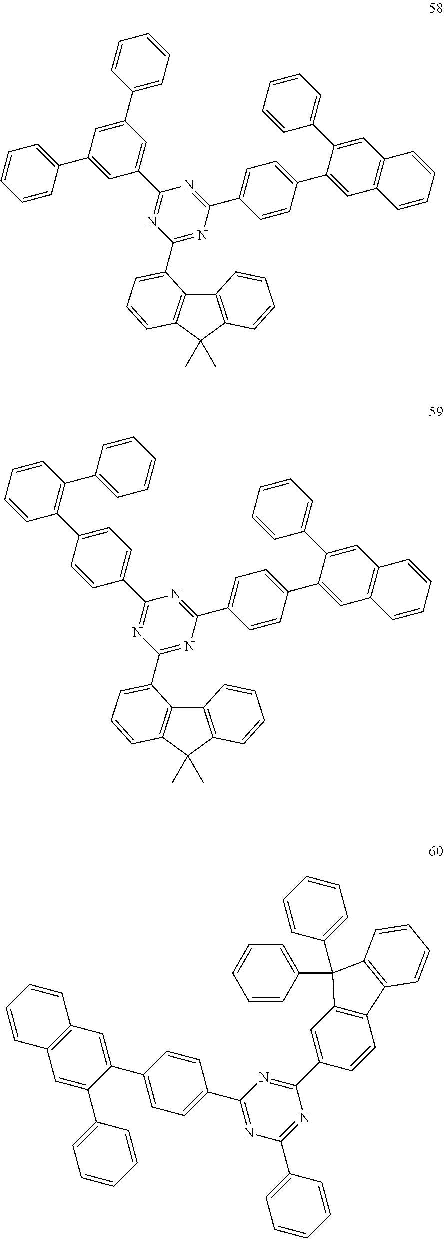 [SMILES: CC1(C)c2ccccc2-c2c(-c3nc(-c4ccc(-c5cc6ccccc6cc5-c5ccccc5)cc4)nc(-c4cc(-c5ccccc5)cc(-c5ccccc5)c4)n3)cccc21 ; CC1(C)c2ccccc2-c2c(-c3nc(-c4ccc(-c5ccccc5-c5ccccc5)cc4)nc(-c4ccc(-c5cc6ccccc6cc5-c5ccccc5)cc4)n3)cccc21 ; c1ccc(-c2nc(-c3ccc(-c4cc5ccccc5cc4-c4ccccc4)cc3)nc(-c3ccc4c(c3)C(c3ccccc3)(c3ccccc3)c3ccccc3-4)n2)cc1]

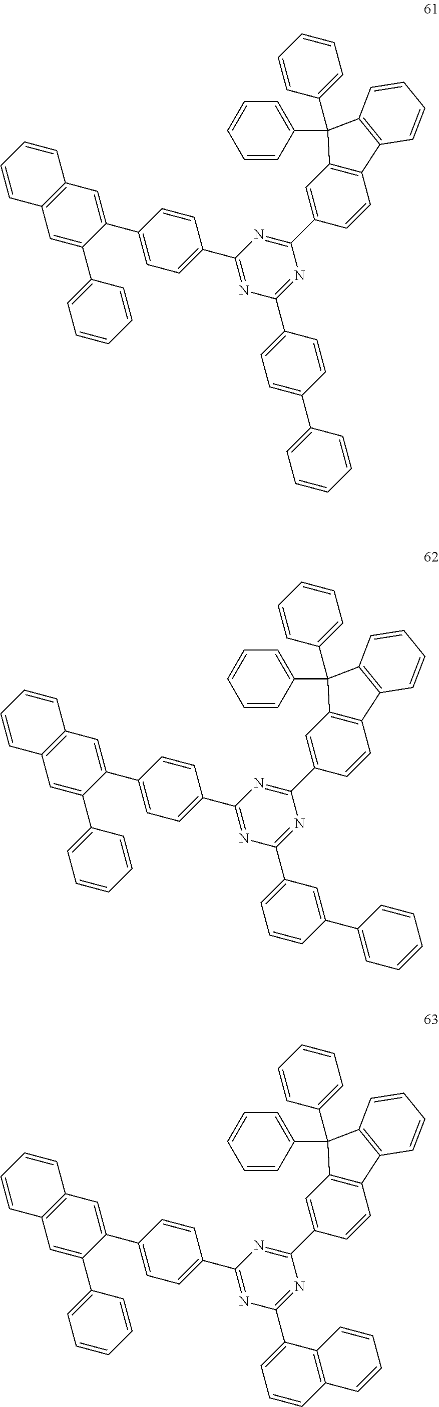 [SMILES: c1ccc(-c2ccc(-c3nc(-c4ccc(-c5cc6ccccc6cc5-c5ccccc5)cc4)nc(-c4ccc5c(c4)C(c4ccccc4)(c4ccccc4)c4ccccc4-5)n3)cc2)cc1 ; c1ccc(-c2cccc(-c3nc(-c4ccc(-c5cc6ccccc6cc5-c5ccccc5)cc4)nc(-c4ccc5c(c4)C(c4ccccc4)(c4ccccc4)c4ccccc4-5)n3)c2)cc1 ; c1ccc(-c2cc3ccccc3cc2-c2ccc(-c3nc(-c4ccc5c(c4)C(c4ccccc4)(c4ccccc4)c4ccccc4-5)nc(-c4cccc5ccccc45)n3)cc2)cc1]

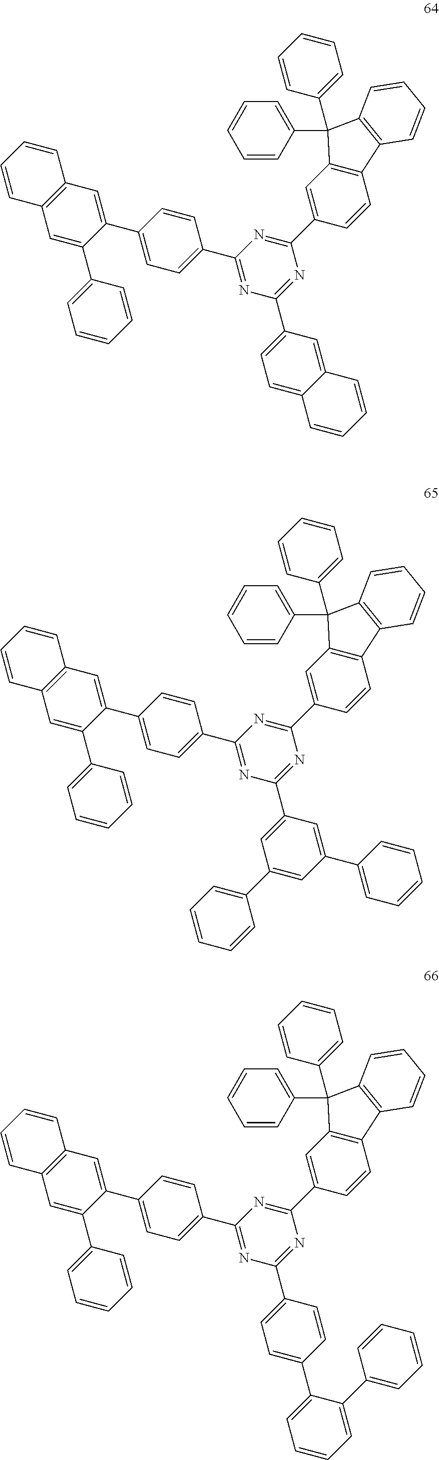 [SMILES: c1ccc(-c2cc3ccccc3cc2-c2ccc(-c3nc(-c4ccc5c(c4)C(c4ccccc4)(c4ccccc4)c4ccccc4-5)nc(-c4ccc5ccccc5c4)n3)cc2)cc1 ; c1ccc(-c2cc(-c3ccccc3)cc(-c3nc(-c4ccc(-c5cc6ccccc6cc5-c5ccccc5)cc4)nc(-c4ccc5c(c4)C(c4ccccc4)(c4ccccc4)c4ccccc4-5)n3)c2)cc1 ; c1ccc(-c2ccccc2-c2ccc(-c3nc(-c4ccc(-c5cc6ccccc6cc5-c5ccccc5)cc4)nc(-c4ccc5c(c4)C(c4ccccc4)(c4ccccc4)c4ccccc4-5)n3)cc2)cc1]

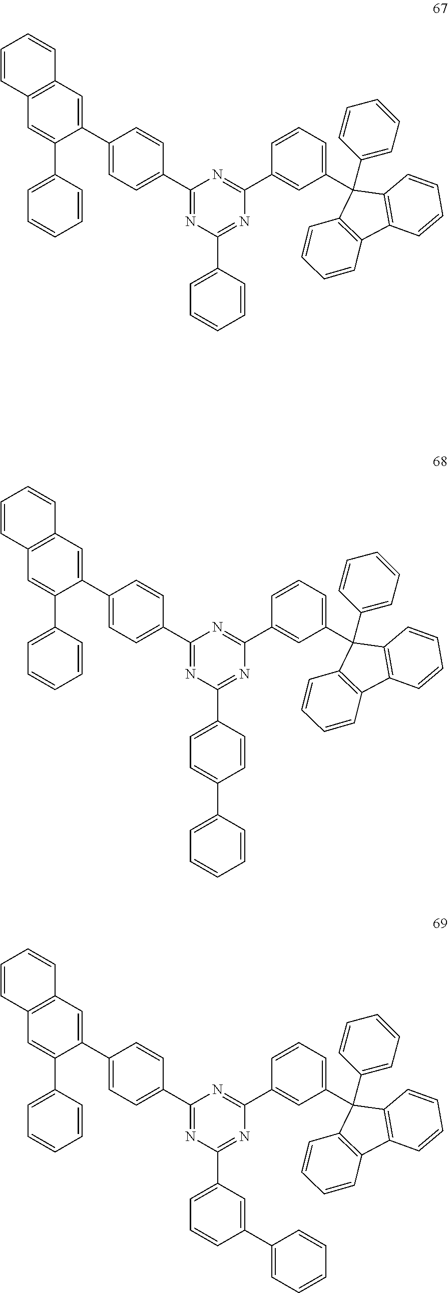 [SMILES: c1ccc(-c2nc(-c3ccc(-c4cc5ccccc5cc4-c4ccccc4)cc3)nc(-c3cccc(C4(c5ccccc5)c5ccccc5-c5ccccc54)c3)n2)cc1 ; c1ccc(-c2ccc(-c3nc(-c4ccc(-c5cc6ccccc6cc5-c5ccccc5)cc4)nc(-c4cccc(C5(c6ccccc6)c6ccccc6-c6ccccc65)c4)n3)cc2)cc1 ; c1ccc(-c2cccc(-c3nc(-c4ccc(-c5cc6ccccc6cc5-c5ccccc5)cc4)nc(-c4cccc(C5(c6ccccc6)c6ccccc6-c6ccccc65)c4)n3)c2)cc1]

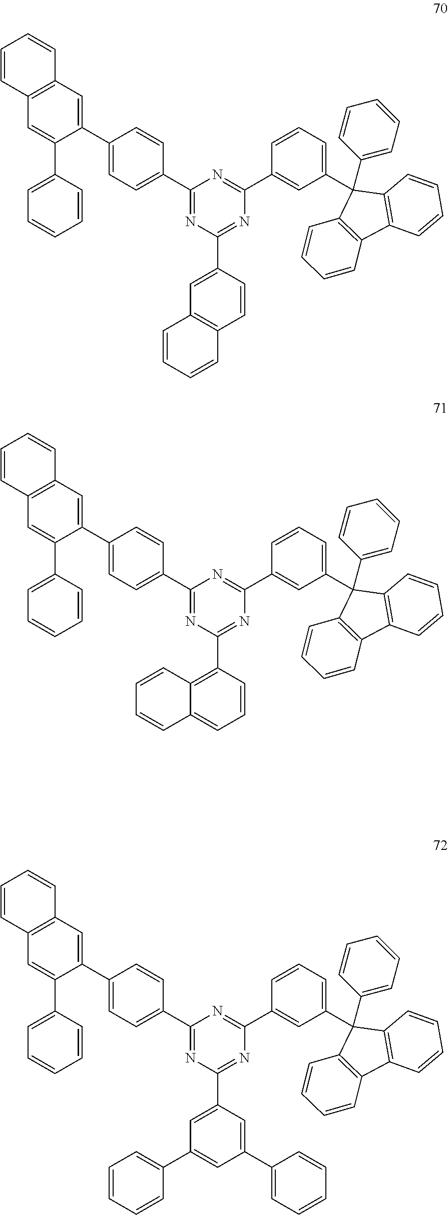 [SMILES: c1ccc(-c2cc3ccccc3cc2-c2ccc(-c3nc(-c4cccc(C5(c6ccccc6)c6ccccc6-c6ccccc65)c4)nc(-c4ccc5ccccc5c4)n3)cc2)cc1 ; c1ccc(-c2cc3ccccc3cc2-c2ccc(-c3nc(-c4cccc(C5(c6ccccc6)c6ccccc6-c6ccccc65)c4)nc(-c4cccc5ccccc45)n3)cc2)cc1 ; c1ccc(-c2cc(-c3ccccc3)cc(-c3nc(-c4ccc(-c5cc6ccccc6cc5-c5ccccc5)cc4)nc(-c4cccc(C5(c6ccccc6)c6ccccc6-c6ccccc65)c4)n3)c2)cc1]

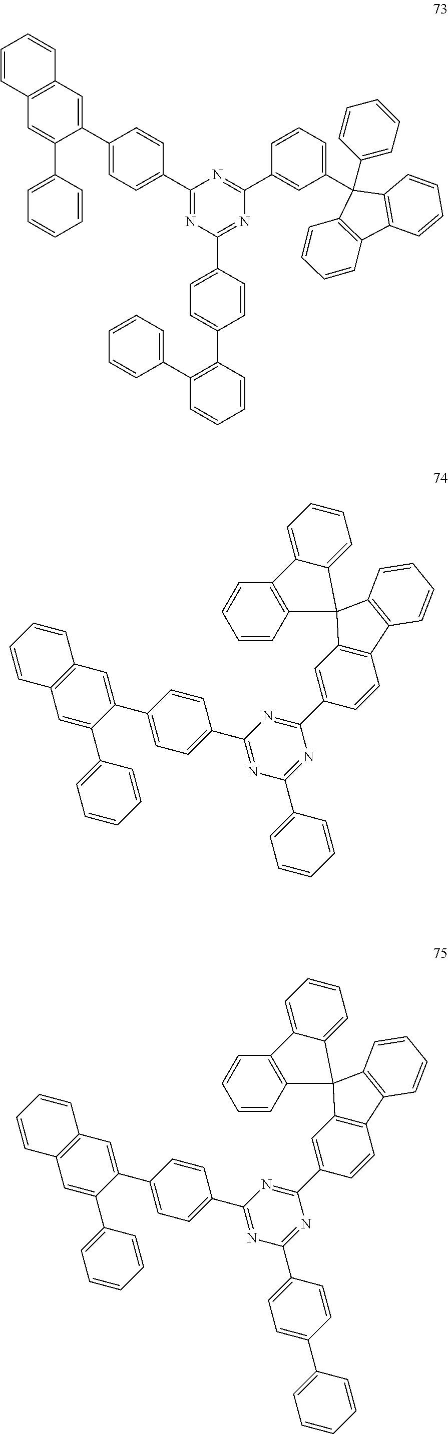 [SMILES: c1ccc(-c2ccccc2-c2ccc(-c3nc(-c4ccc(-c5cc6ccccc6cc5-c5ccccc5)cc4)nc(-c4cccc(C5(c6ccccc6)c6ccccc6-c6ccccc65)c4)n3)cc2)cc1 ; c1ccc(-c2nc(-c3ccc(-c4cc5ccccc5cc4-c4ccccc4)cc3)nc(-c3ccc4c(c3)C3(c5ccccc5-c5ccccc53)c3ccccc3-4)n2)cc1 ; c1ccc(-c2ccc(-c3nc(-c4ccc(-c5cc6ccccc6cc5-c5ccccc5)cc4)nc(-c4ccc5c(c4)C4(c6ccccc6-c6ccccc64)c4ccccc4-5)n3)cc2)cc1]

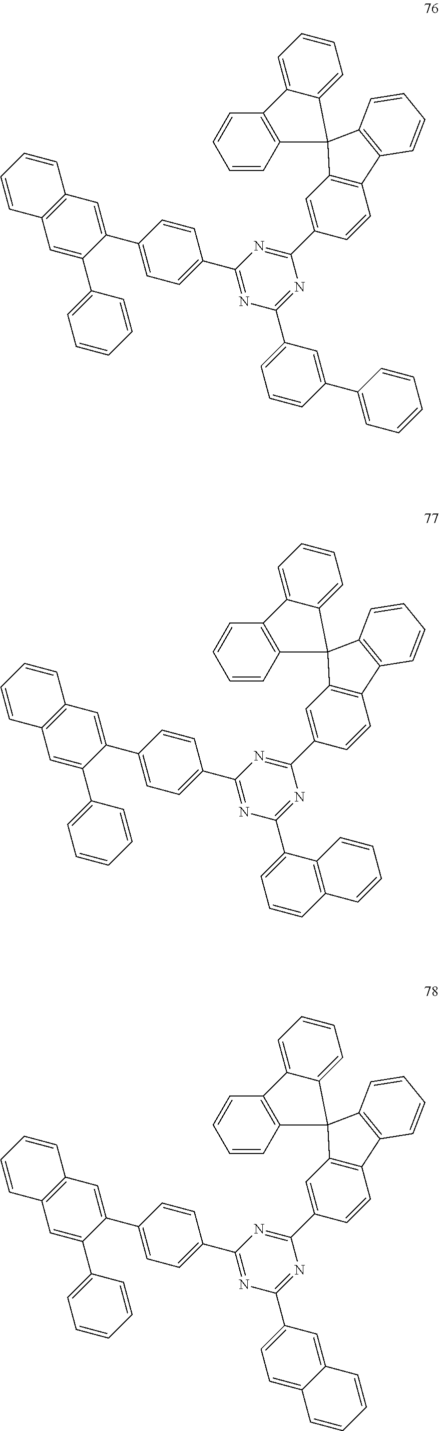 [SMILES: c1ccc(-c2cccc(-c3nc(-c4ccc(-c5cc6ccccc6cc5-c5ccccc5)cc4)nc(-c4ccc5c(c4)C4(c6ccccc6-c6ccccc64)c4ccccc4-5)n3)c2)cc1 ; c1ccc(-c2cc3ccccc3cc2-c2ccc(-c3nc(-c4ccc5c(c4)C4(c6ccccc6-c6ccccc64)c4ccccc4-5)nc(-c4cccc5ccccc45)n3)cc2)cc1 ; c1ccc(-c2cc3ccccc3cc2-c2ccc(-c3nc(-c4ccc5c(c4)C4(c6ccccc6-c6ccccc64)c4ccccc4-5)nc(-c4ccc5ccccc5c4)n3)cc2)cc1]

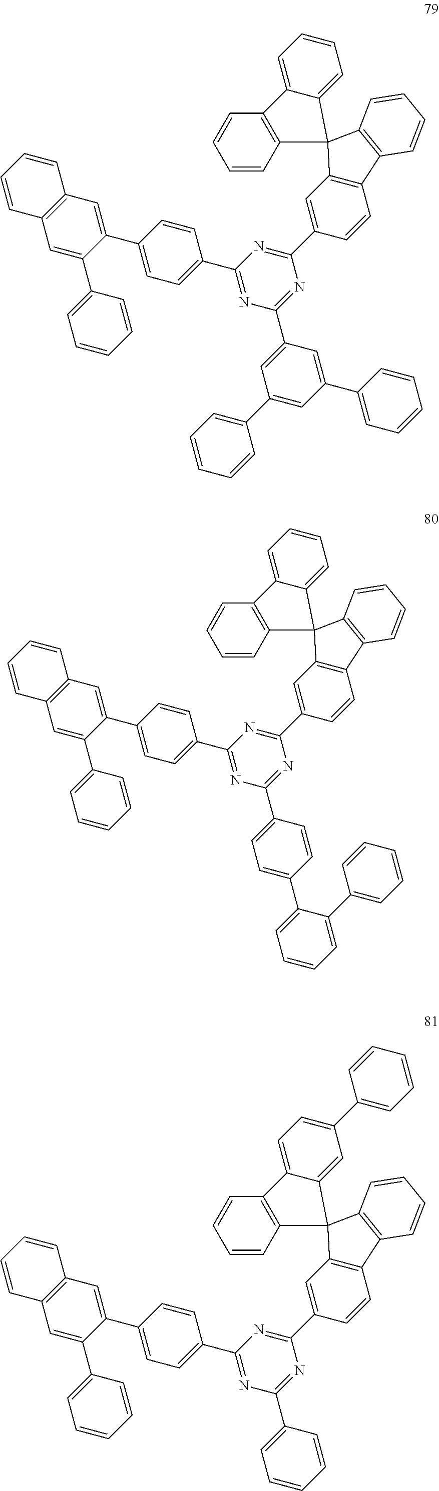 [SMILES: c1ccc(-c2cc(-c3ccccc3)cc(-c3nc(-c4ccc(-c5cc6ccccc6cc5-c5ccccc5)cc4)nc(-c4ccc5c(c4)C4(c6ccccc6-c6ccccc64)c4ccccc4-5)n3)c2)cc1 ; c1ccc(-c2ccccc2-c2ccc(-c3nc(-c4ccc(-c5cc6ccccc6cc5-c5ccccc5)cc4)nc(-c4ccc5c(c4)C4(c6ccccc6-c6ccccc64)c4ccccc4-5)n3)cc2)cc1 ; c1ccc(-c2ccc3c(c2)C2(c4ccccc4-3)c3ccccc3-c3ccc(-c4nc(-c5ccccc5)nc(-c5ccc(-c6cc7ccccc7cc6-c6ccccc6)cc5)n4)cc32)cc1]

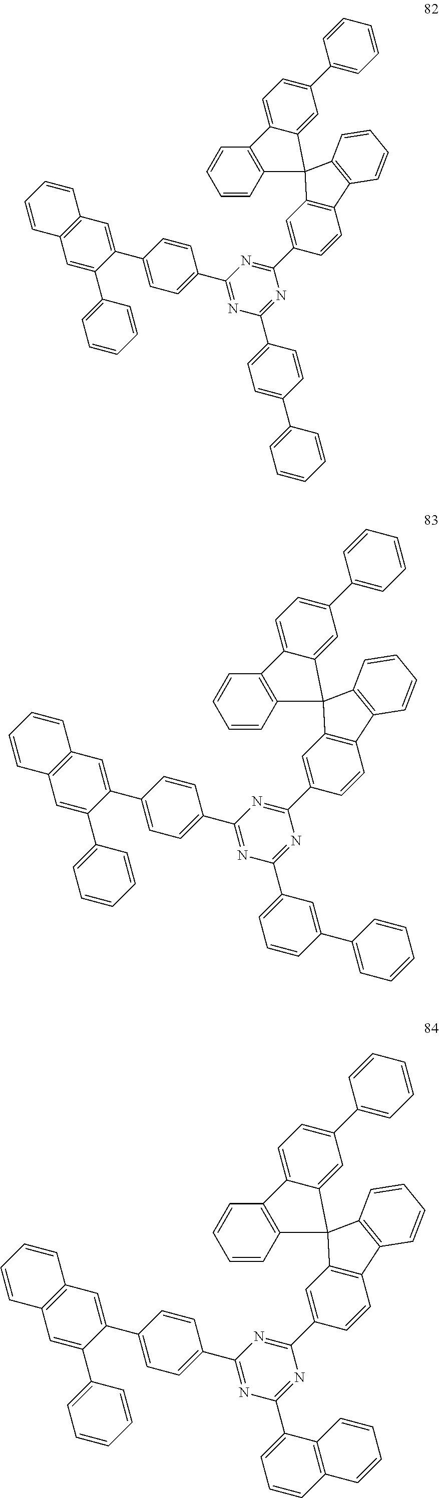 [SMILES: c1ccc(-c2ccc(-c3nc(-c4ccc(-c5cc6ccccc6cc5-c5ccccc5)cc4)nc(-c4ccc5c(c4)C4(c6ccccc6-c6ccc(-c7ccccc7)cc64)c4ccccc4-5)n3)cc2)cc1 ; c1ccc(-c2cccc(-c3nc(-c4ccc(-c5cc6ccccc6cc5-c5ccccc5)cc4)nc(-c4ccc5c(c4)C4(c6ccccc6-c6ccc(-c7ccccc7)cc64)c4ccccc4-5)n3)c2)cc1 ; c1ccc(-c2ccc3c(c2)C2(c4ccccc4-3)c3ccccc3-c3ccc(-c4nc(-c5ccc(-c6cc7ccccc7cc6-c6ccccc6)cc5)nc(-c5cccc6ccccc56)n4)cc32)cc1]

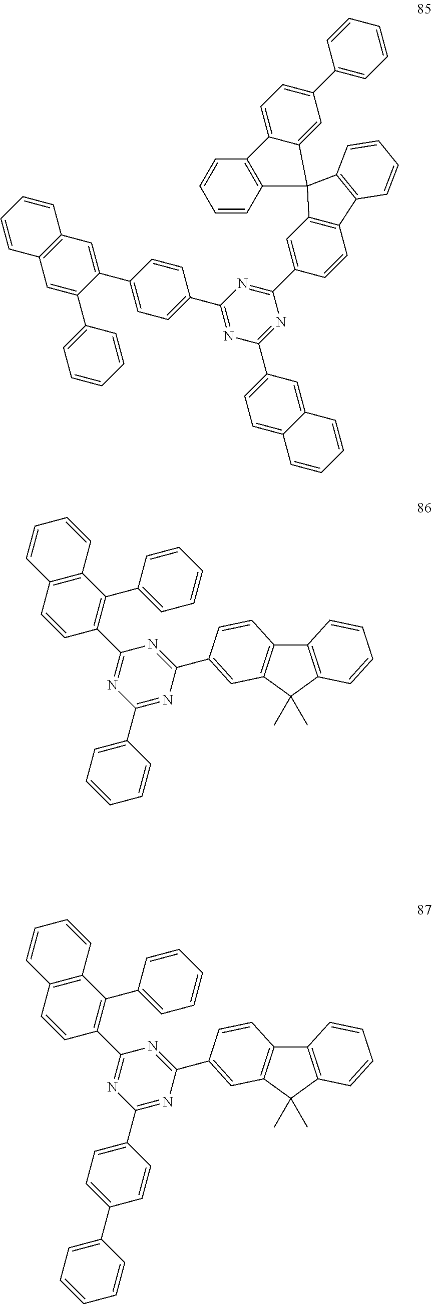 [SMILES: c1ccc(-c2ccc3c(c2)C2(c4ccccc4-3)c3ccccc3-c3ccc(-c4nc(-c5ccc(-c6cc7ccccc7cc6-c6ccccc6)cc5)nc(-c5ccc6ccccc6c5)n4)cc32)cc1 ; CC1(C)c2ccccc2-c2ccc(-c3nc(-c4ccccc4)nc(-c4ccc5ccccc5c4-c4ccccc4)n3)cc21 ; CC1(C)c2ccccc2-c2ccc(-c3nc(-c4ccc(-c5ccccc5)cc4)nc(-c4ccc5ccccc5c4-c4ccccc4)n3)cc21]

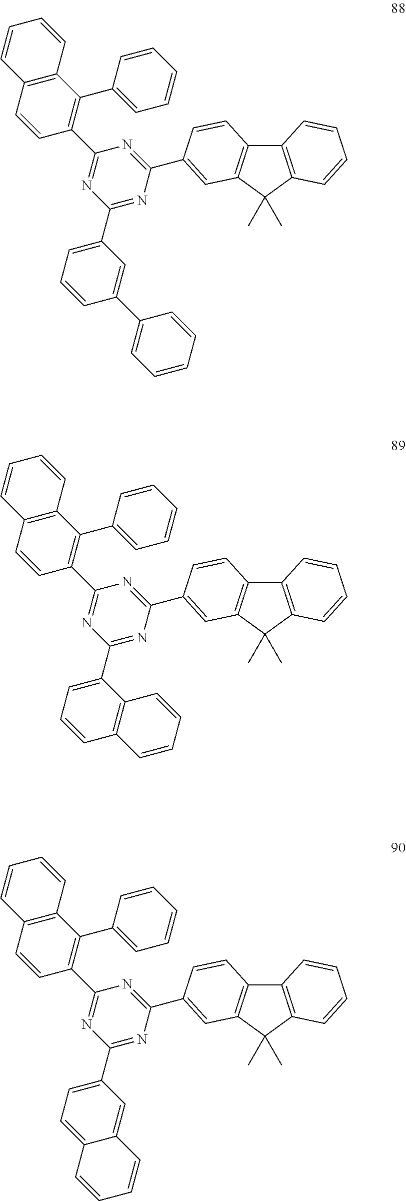 [SMILES: CC1(C)c2ccccc2-c2ccc(-c3nc(-c4cccc(-c5ccccc5)c4)nc(-c4ccc5ccccc5c4-c4ccccc4)n3)cc21 ; CC1(C)c2ccccc2-c2ccc(-c3nc(-c4ccc5ccccc5c4-c4ccccc4)nc(-c4cccc5ccccc45)n3)cc21 ; CC1(C)c2ccccc2-c2ccc(-c3nc(-c4ccc5ccccc5c4)nc(-c4ccc5ccccc5c4-c4ccccc4)n3)cc21]

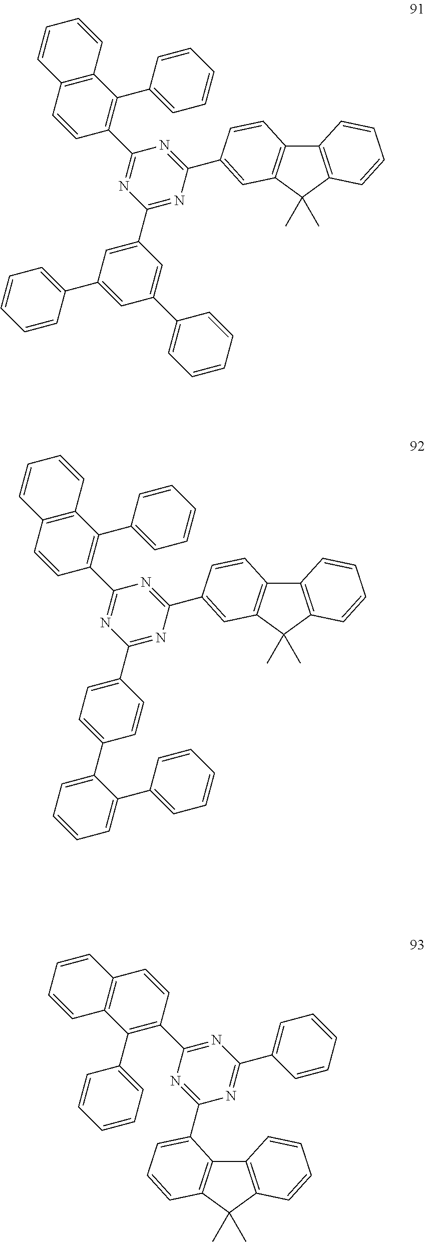 [SMILES: CC1(C)c2ccccc2-c2ccc(-c3nc(-c4cc(-c5ccccc5)cc(-c5ccccc5)c4)nc(-c4ccc5ccccc5c4-c4ccccc4)n3)cc21 ; CC1(C)c2ccccc2-c2ccc(-c3nc(-c4ccc(-c5ccccc5-c5ccccc5)cc4)nc(-c4ccc5ccccc5c4-c4ccccc4)n3)cc21 ; CC1(C)c2ccccc2-c2c(-c3nc(-c4ccccc4)nc(-c4ccc5ccccc5c4-c4ccccc4)n3)cccc21]

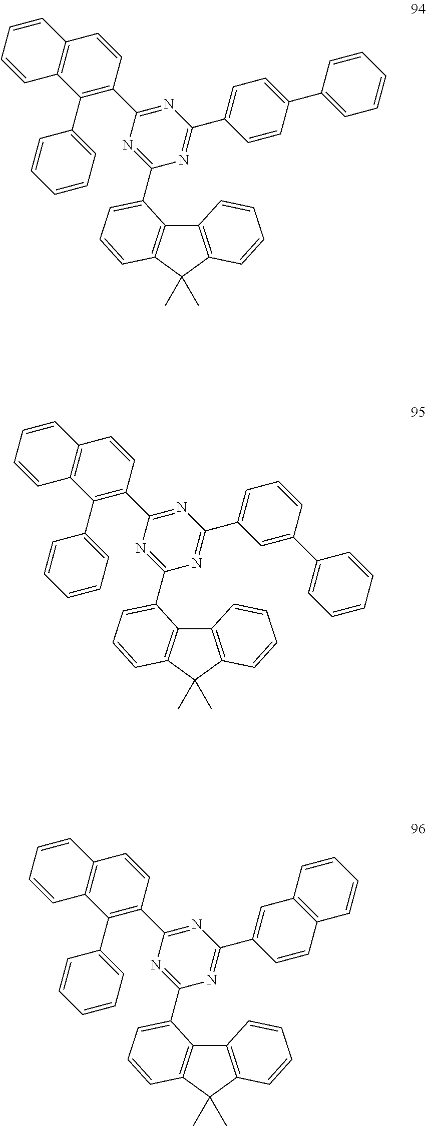 [SMILES: CC1(C)c2ccccc2-c2c(-c3nc(-c4ccc(-c5ccccc5)cc4)nc(-c4ccc5ccccc5c4-c4ccccc4)n3)cccc21 ; CC1(C)c2ccccc2-c2c(-c3nc(-c4cccc(-c5ccccc5)c4)nc(-c4ccc5ccccc5c4-c4ccccc4)n3)cccc21 ; CC1(C)c2ccccc2-c2c(-c3nc(-c4ccc5ccccc5c4)nc(-c4ccc5ccccc5c4-c4ccccc4)n3)cccc21]

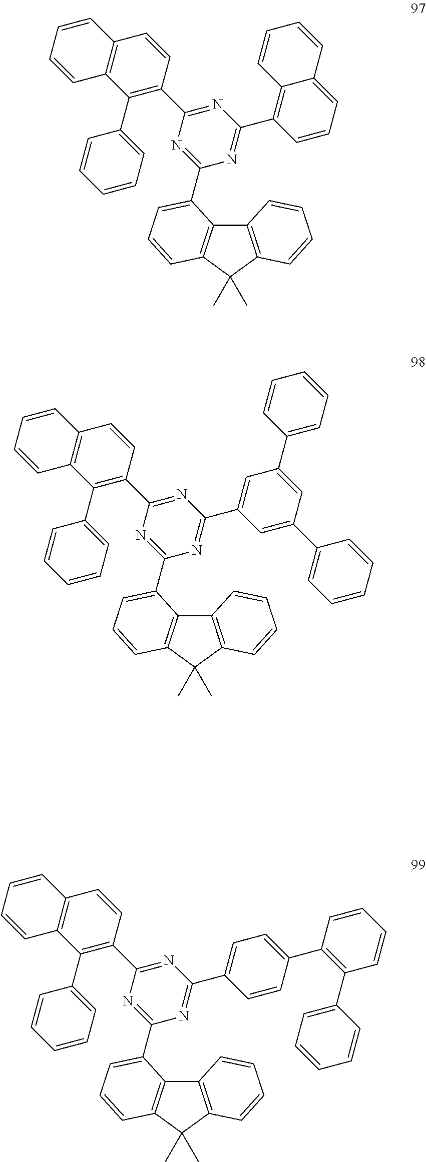 [SMILES: CC1(C)c2ccccc2-c2c(-c3nc(-c4ccc5ccccc5c4-c4ccccc4)nc(-c4cccc5ccccc45)n3)cccc21 ; CC1(C)c2ccccc2-c2c(-c3nc(-c4cc(-c5ccccc5)cc(-c5ccccc5)c4)nc(-c4ccc5ccccc5c4-c4ccccc4)n3)cccc21 ; CC1(C)c2ccccc2-c2c(-c3nc(-c4ccc(-c5ccccc5-c5ccccc5)cc4)nc(-c4ccc5ccccc5c4-c4ccccc4)n3)cccc21]

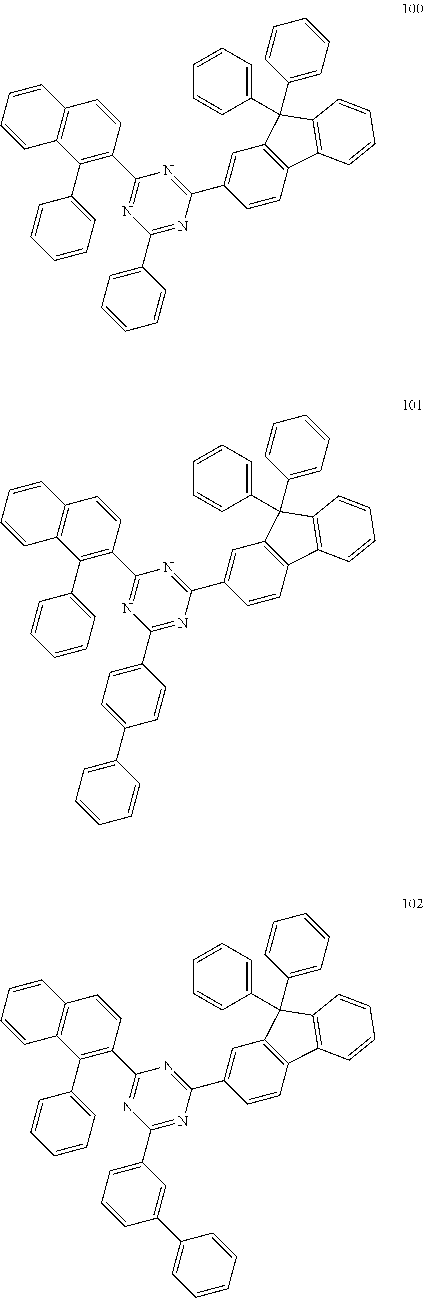 [SMILES: c1ccc(-c2nc(-c3ccc4c(c3)C(c3ccccc3)(c3ccccc3)c3ccccc3-4)nc(-c3ccc4ccccc4c3-c3ccccc3)n2)cc1 ; c1ccc(-c2ccc(-c3nc(-c4ccc5c(c4)C(c4ccccc4)(c4ccccc4)c4ccccc4-5)nc(-c4ccc5ccccc5c4-c4ccccc4)n3)cc2)cc1 ; c1ccc(-c2cccc(-c3nc(-c4ccc5c(c4)C(c4ccccc4)(c4ccccc4)c4ccccc4-5)nc(-c4ccc5ccccc5c4-c4ccccc4)n3)c2)cc1]

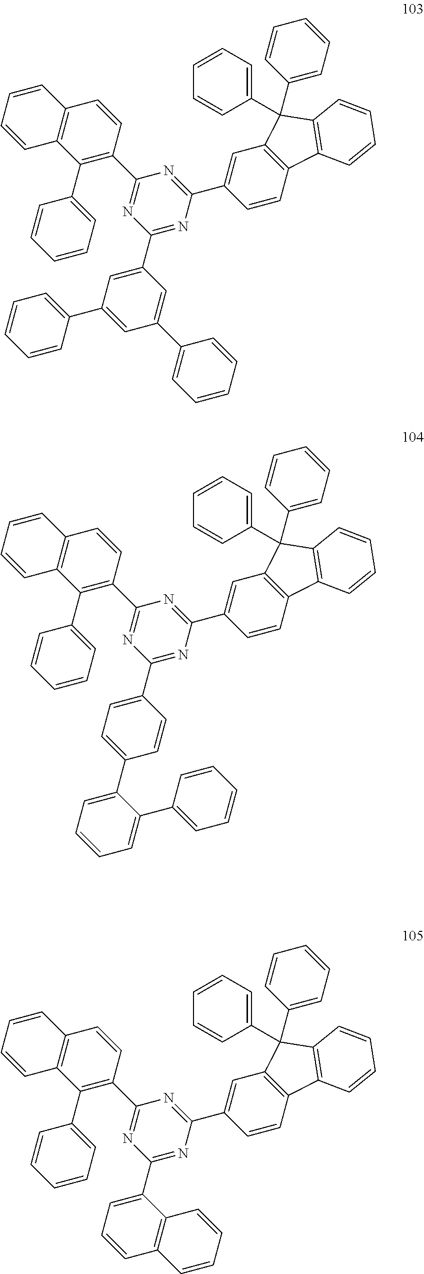 [SMILES: c1ccc(-c2cc(-c3ccccc3)cc(-c3nc(-c4ccc5c(c4)C(c4ccccc4)(c4ccccc4)c4ccccc4-5)nc(-c4ccc5ccccc5c4-c4ccccc4)n3)c2)cc1 ; c1ccc(-c2ccccc2-c2ccc(-c3nc(-c4ccc5c(c4)C(c4ccccc4)(c4ccccc4)c4ccccc4-5)nc(-c4ccc5ccccc5c4-c4ccccc4)n3)cc2)cc1 ; c1ccc(-c2c(-c3nc(-c4ccc5c(c4)C(c4ccccc4)(c4ccccc4)c4ccccc4-5)nc(-c4cccc5ccccc45)n3)ccc3ccccc23)cc1]

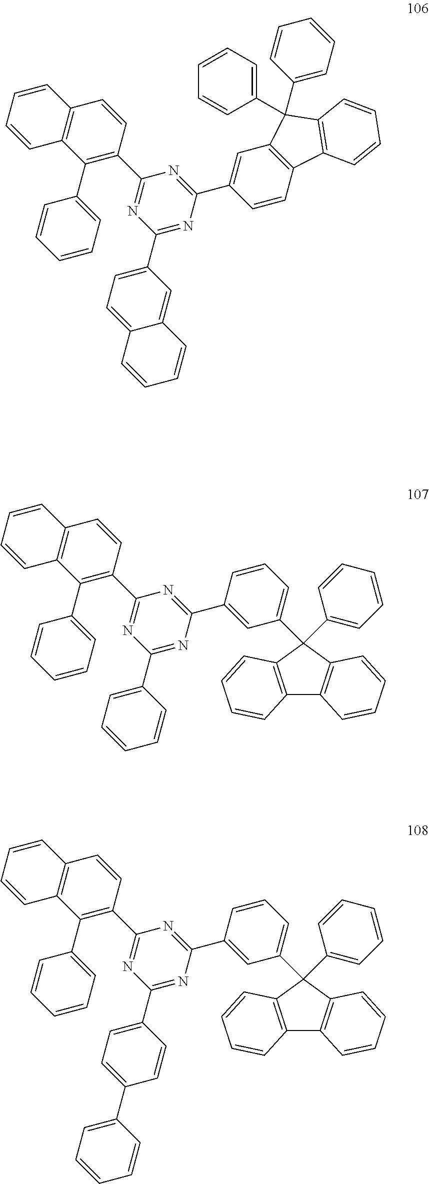 [SMILES: c1ccc(-c2c(-c3nc(-c4ccc5c(c4)C(c4ccccc4)(c4ccccc4)c4ccccc4-5)nc(-c4ccc5ccccc5c4)n3)ccc3ccccc23)cc1 ; c1ccc(-c2nc(-c3cccc(C4(c5ccccc5)c5ccccc5-c5ccccc54)c3)nc(-c3ccc4ccccc4c3-c3ccccc3)n2)cc1 ; c1ccc(-c2ccc(-c3nc(-c4cccc(C5(c6ccccc6)c6ccccc6-c6ccccc65)c4)nc(-c4ccc5ccccc5c4-c4ccccc4)n3)cc2)cc1]

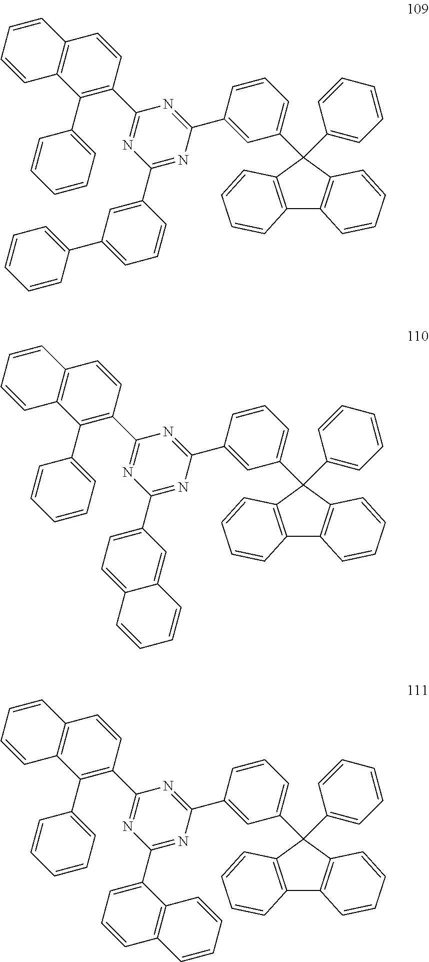 [SMILES: c1ccc(-c2cccc(-c3nc(-c4cccc(C5(c6ccccc6)c6ccccc6-c6ccccc65)c4)nc(-c4ccc5ccccc5c4-c4ccccc4)n3)c2)cc1 ; c1ccc(-c2c(-c3nc(-c4cccc(C5(c6ccccc6)c6ccccc6-c6ccccc65)c4)nc(-c4ccc5ccccc5c4)n3)ccc3ccccc23)cc1 ; c1ccc(-c2c(-c3nc(-c4cccc(C5(c6ccccc6)c6ccccc6-c6ccccc65)c4)nc(-c4cccc5ccccc45)n3)ccc3ccccc23)cc1]

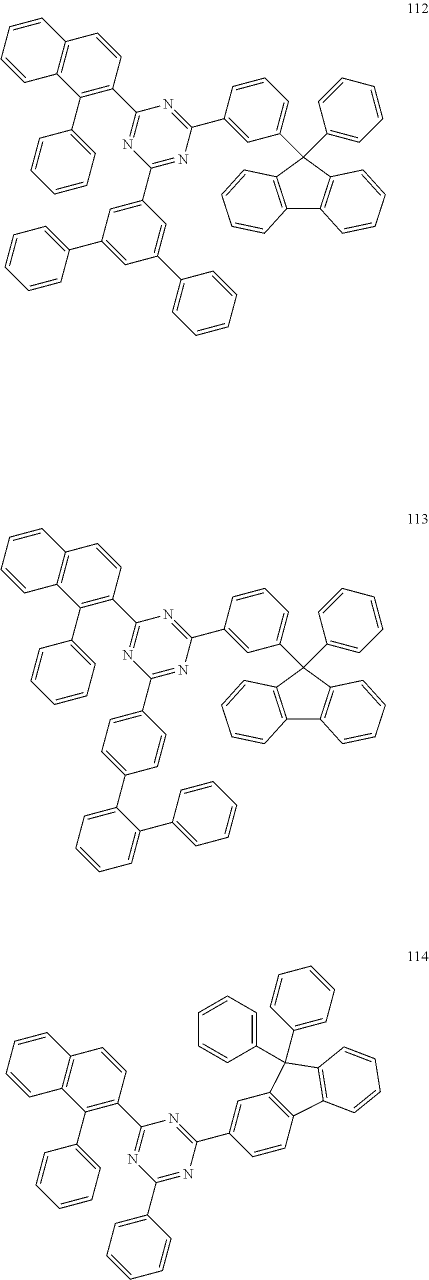 [SMILES: c1ccc(-c2cc(-c3ccccc3)cc(-c3nc(-c4cccc(C5(c6ccccc6)c6ccccc6-c6ccccc65)c4)nc(-c4ccc5ccccc5c4-c4ccccc4)n3)c2)cc1 ; c1ccc(-c2ccccc2-c2ccc(-c3nc(-c4cccc(C5(c6ccccc6)c6ccccc6-c6ccccc65)c4)nc(-c4ccc5ccccc5c4-c4ccccc4)n3)cc2)cc1 ; c1ccc(-c2nc(-c3ccc4c(c3)C(c3ccccc3)(c3ccccc3)c3ccccc3-4)nc(-c3ccc4ccccc4c3-c3ccccc3)n2)cc1]

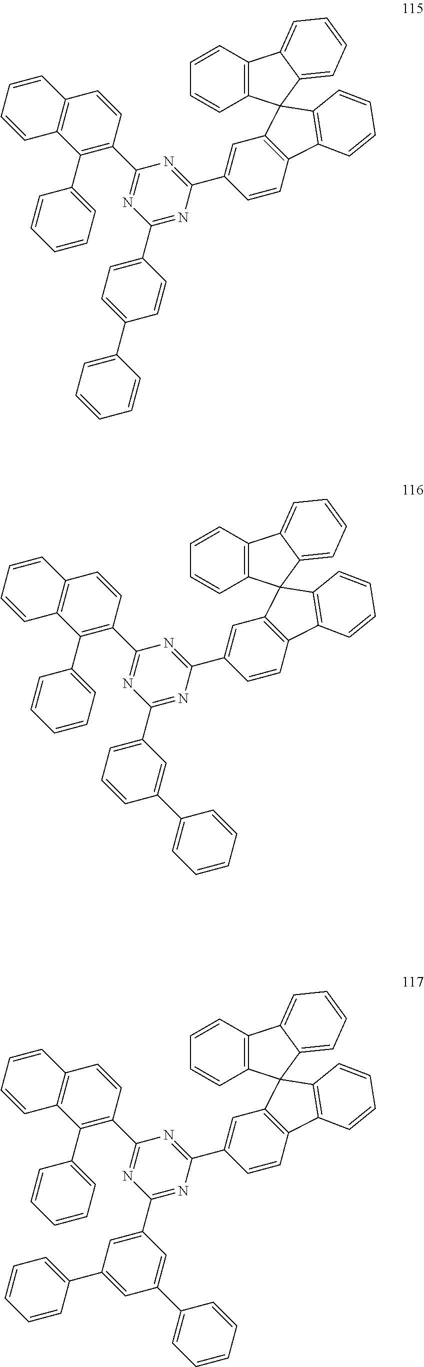 [SMILES: c1ccc(-c2ccc(-c3nc(-c4ccc5c(c4)C4(c6ccccc6-c6ccccc64)c4ccccc4-5)nc(-c4ccc5ccccc5c4-c4ccccc4)n3)cc2)cc1 ; c1ccc(-c2cccc(-c3nc(-c4ccc5c(c4)C4(c6ccccc6-c6ccccc64)c4ccccc4-5)nc(-c4ccc5ccccc5c4-c4ccccc4)n3)c2)cc1 ; c1ccc(-c2cc(-c3ccccc3)cc(-c3nc(-c4ccc5c(c4)C4(c6ccccc6-c6ccccc64)c4ccccc4-5)nc(-c4ccc5ccccc5c4-c4ccccc4)n3)c2)cc1]

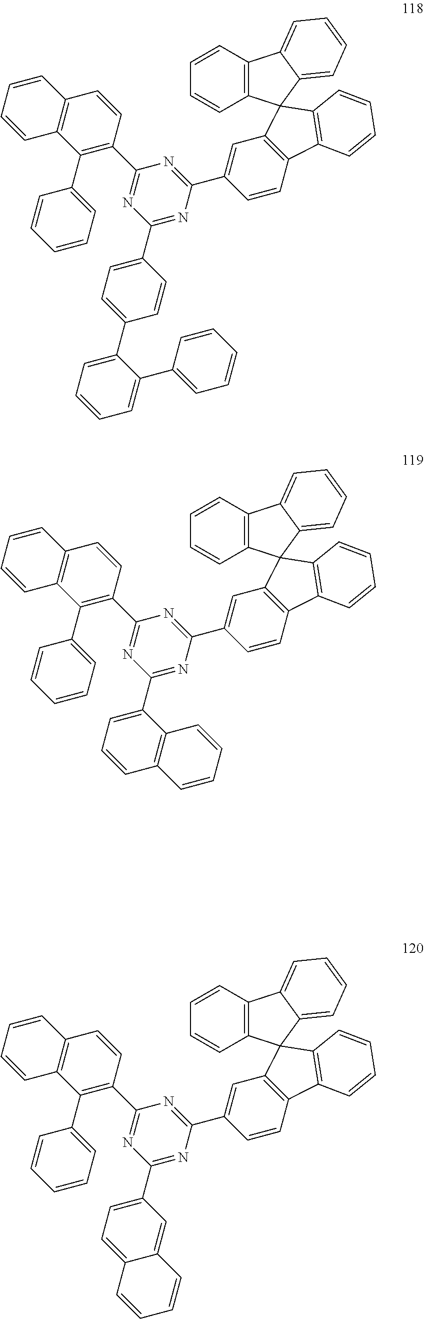 [SMILES: c1ccc(-c2ccccc2-c2ccc(-c3nc(-c4ccc5c(c4)C4(c6ccccc6-c6ccccc64)c4ccccc4-5)nc(-c4ccc5ccccc5c4-c4ccccc4)n3)cc2)cc1 ; c1ccc(-c2c(-c3nc(-c4ccc5c(c4)C4(c6ccccc6-c6ccccc64)c4ccccc4-5)nc(-c4cccc5ccccc45)n3)ccc3ccccc23)cc1 ; c1ccc(-c2c(-c3nc(-c4ccc5c(c4)C4(c6ccccc6-c6ccccc64)c4ccccc4-5)nc(-c4ccc5ccccc5c4)n3)ccc3ccccc23)cc1]

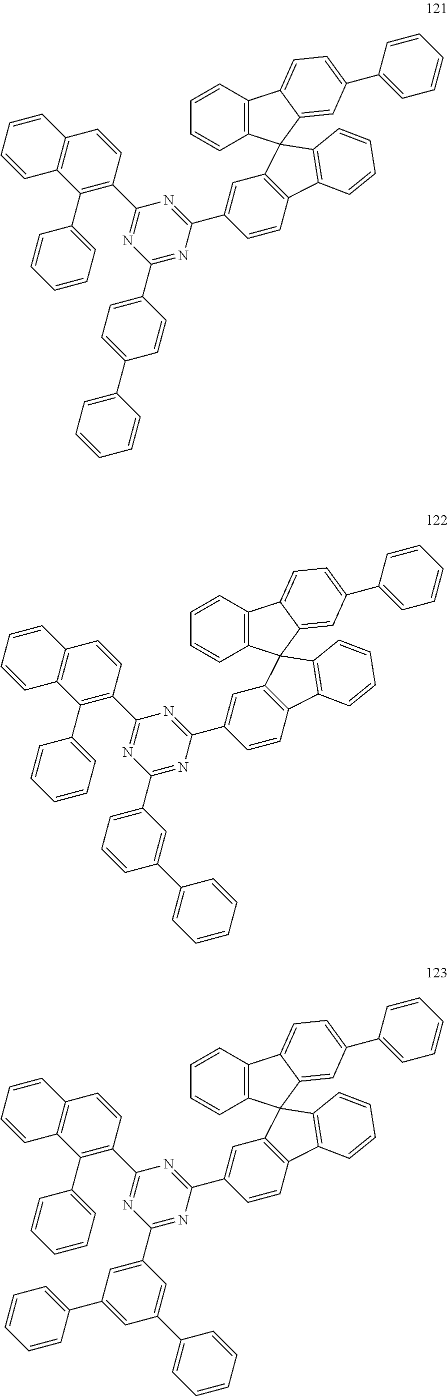 [SMILES: c1ccc(-c2ccc(-c3nc(-c4ccc5c(c4)C4(c6ccccc6-c6ccc(-c7ccccc7)cc64)c4ccccc4-5)nc(-c4ccc5ccccc5c4-c4ccccc4)n3)cc2)cc1 ; c1ccc(-c2cccc(-c3nc(-c4ccc5c(c4)C4(c6ccccc6-c6ccc(-c7ccccc7)cc64)c4ccccc4-5)nc(-c4ccc5ccccc5c4-c4ccccc4)n3)c2)cc1 ; c1ccc(-c2cc(-c3ccccc3)cc(-c3nc(-c4ccc5c(c4)C4(c6ccccc6-c6ccc(-c7ccccc7)cc64)c4ccccc4-5)nc(-c4ccc5ccccc5c4-c4ccccc4)n3)c2)cc1]

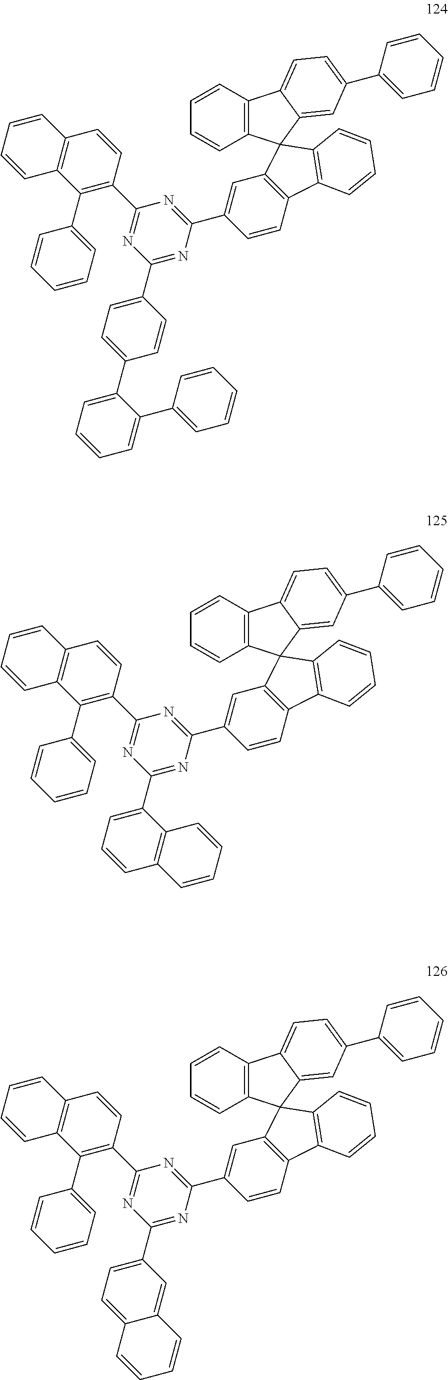 [SMILES: c1ccc(-c2ccc3c(c2)C2(c4ccccc4-3)c3ccccc3-c3ccc(-c4nc(-c5ccc(-c6ccccc6-c6ccccc6)cc5)nc(-c5ccc6ccccc6c5-c5ccccc5)n4)cc32)cc1 ; c1ccc(-c2ccc3c(c2)C2(c4ccccc4-3)c3ccccc3-c3ccc(-c4nc(-c5ccc6ccccc6c5-c5ccccc5)nc(-c5cccc6ccccc56)n4)cc32)cc1 ; c1ccc(-c2ccc3c(c2)C2(c4ccccc4-3)c3ccccc3-c3ccc(-c4nc(-c5ccc6ccccc6c5)nc(-c5ccc6ccccc6c5-c5ccccc5)n4)cc32)cc1]

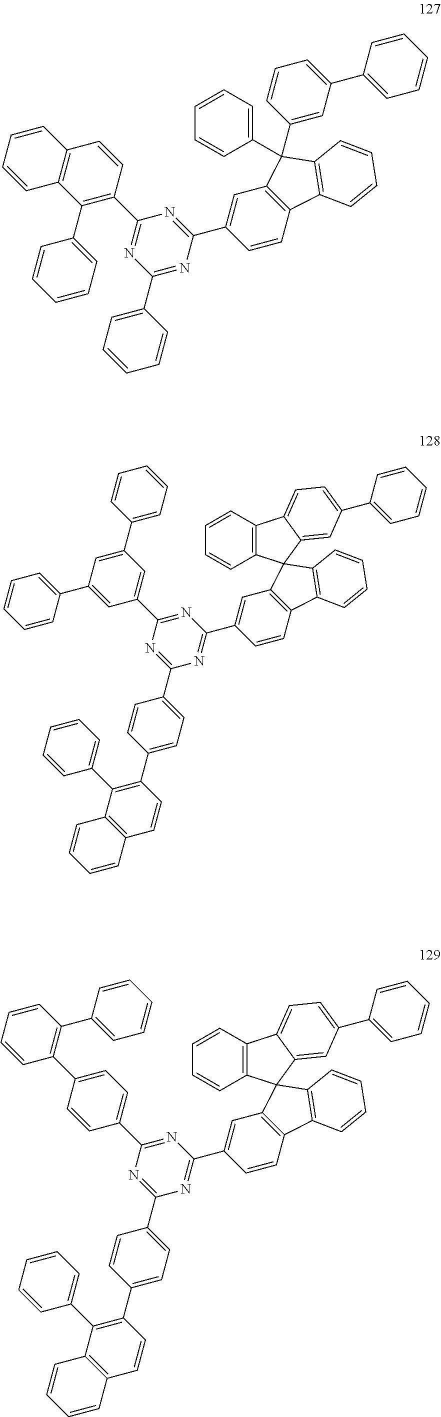 [SMILES: c1ccc(-c2cccc(C3(c4ccccc4)c4ccccc4-c4ccc(-c5nc(-c6ccccc6)nc(-c6ccc7ccccc7c6-c6ccccc6)n5)cc43)c2)cc1 ; c1ccc(-c2cc(-c3ccccc3)cc(-c3nc(-c4ccc(-c5ccc6ccccc6c5-c5ccccc5)cc4)nc(-c4ccc5c(c4)C4(c6ccccc6-c6ccc(-c7ccccc7)cc64)c4ccccc4-5)n3)c2)cc1 ; c1ccc(-c2ccc3c(c2)C2(c4ccccc4-3)c3ccccc3-c3ccc(-c4nc(-c5ccc(-c6ccccc6-c6ccccc6)cc5)nc(-c5ccc(-c6ccc7ccccc7c6-c6ccccc6)cc5)n4)cc32)cc1]

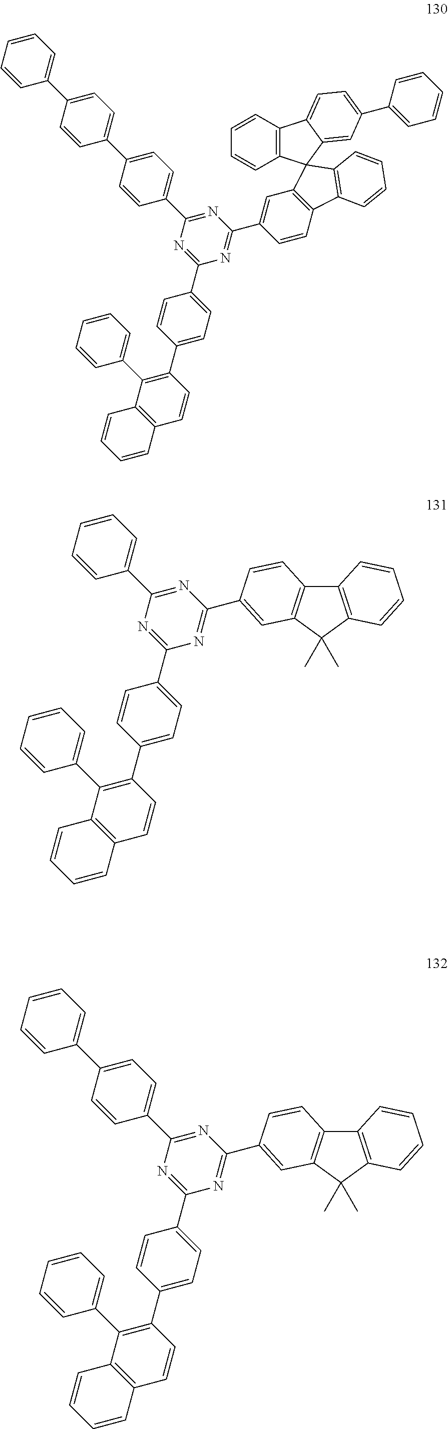 [SMILES: c1ccc(-c2ccc(-c3ccc(-c4nc(-c5ccc(-c6ccc7ccccc7c6-c6ccccc6)cc5)nc(-c5ccc6c(c5)C5(c7ccccc7-c7ccc(-c8ccccc8)cc75)c5ccccc5-6)n4)cc3)cc2)cc1 ; CC1(C)c2ccccc2-c2ccc(-c3nc(-c4ccccc4)nc(-c4ccc(-c5ccc6ccccc6c5-c5ccccc5)cc4)n3)cc21 ; CC1(C)c2ccccc2-c2ccc(-c3nc(-c4ccc(-c5ccccc5)cc4)nc(-c4ccc(-c5ccc6ccccc6c5-c5ccccc5)cc4)n3)cc21]

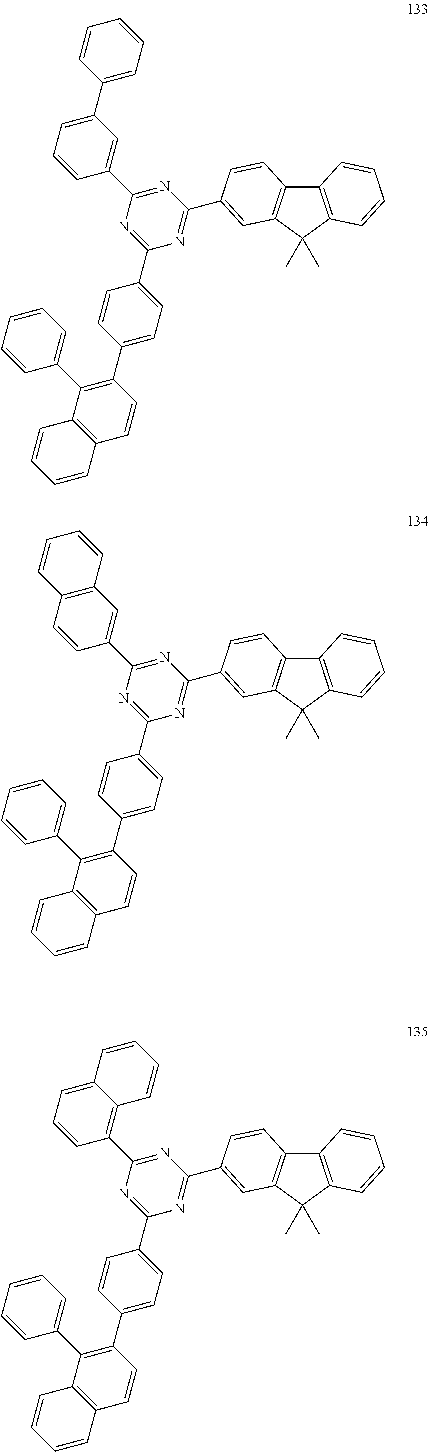 [SMILES: CC1(C)c2ccccc2-c2ccc(-c3nc(-c4ccc(-c5ccc6ccccc6c5-c5ccccc5)cc4)nc(-c4cccc(-c5ccccc5)c4)n3)cc21 ; CC1(C)c2ccccc2-c2ccc(-c3nc(-c4ccc(-c5ccc6ccccc6c5-c5ccccc5)cc4)nc(-c4ccc5ccccc5c4)n3)cc21 ; CC1(C)c2ccccc2-c2ccc(-c3nc(-c4ccc(-c5ccc6ccccc6c5-c5ccccc5)cc4)nc(-c4cccc5ccccc45)n3)cc21]

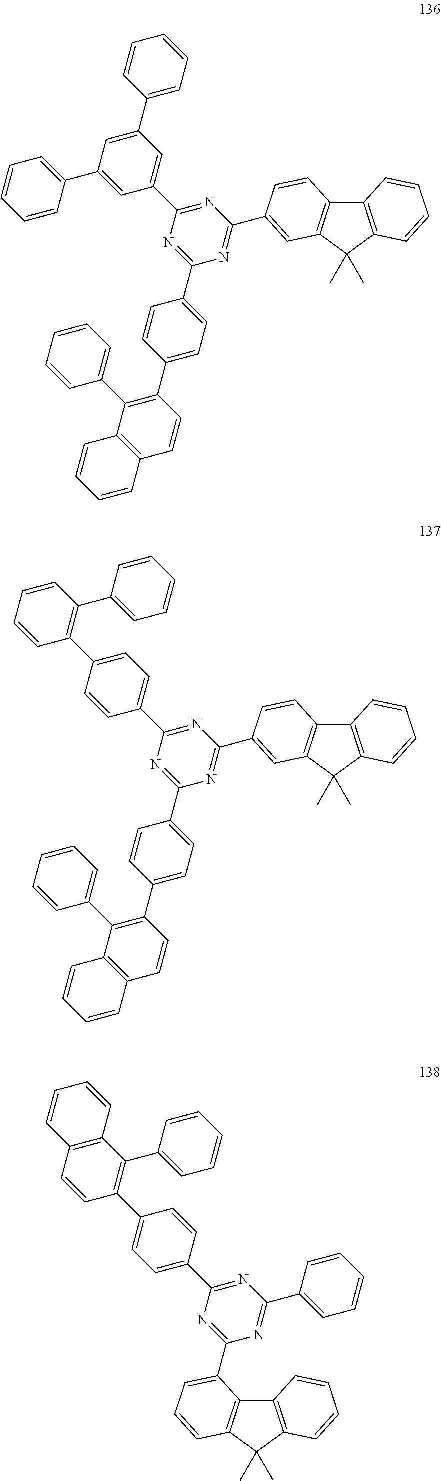 [SMILES: CC1(C)c2ccccc2-c2ccc(-c3nc(-c4ccc(-c5ccc6ccccc6c5-c5ccccc5)cc4)nc(-c4cc(-c5ccccc5)cc(-c5ccccc5)c4)n3)cc21 ; CC1(C)c2ccccc2-c2ccc(-c3nc(-c4ccc(-c5ccccc5-c5ccccc5)cc4)nc(-c4ccc(-c5ccc6ccccc6c5-c5ccccc5)cc4)n3)cc21 ; CC1(C)c2ccccc2-c2c(-c3nc(-c4ccccc4)nc(-c4ccc(-c5ccc6ccccc6c5-c5ccccc5)cc4)n3)cccc21]

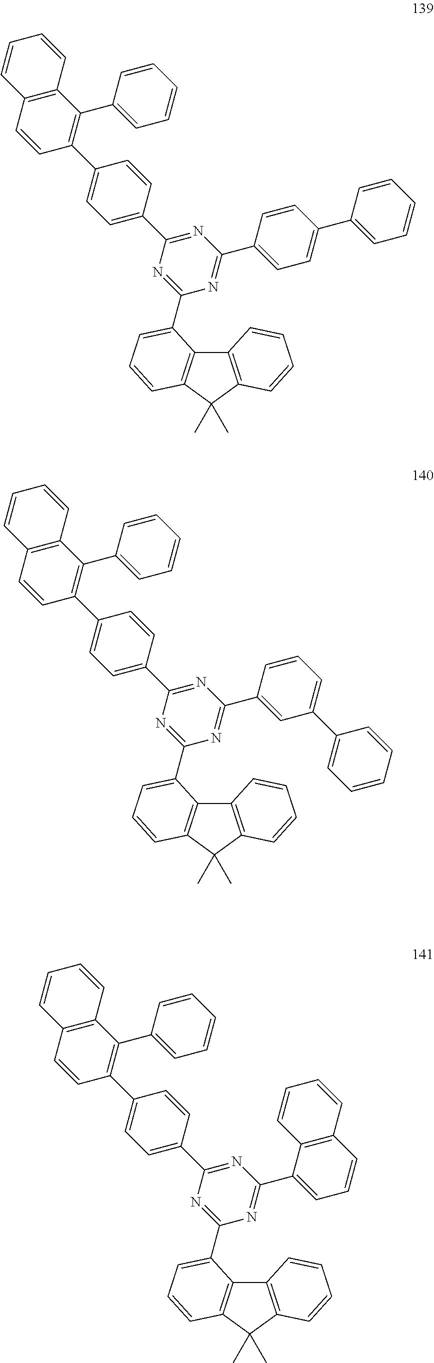 [SMILES: CC1(C)c2ccccc2-c2c(-c3nc(-c4ccc(-c5ccccc5)cc4)nc(-c4ccc(-c5ccc6ccccc6c5-c5ccccc5)cc4)n3)cccc21 ; CC1(C)c2ccccc2-c2c(-c3nc(-c4ccc(-c5ccc6ccccc6c5-c5ccccc5)cc4)nc(-c4cccc(-c5ccccc5)c4)n3)cccc21 ; CC1(C)c2ccccc2-c2c(-c3nc(-c4ccc(-c5ccc6ccccc6c5-c5ccccc5)cc4)nc(-c4cccc5ccccc45)n3)cccc21]

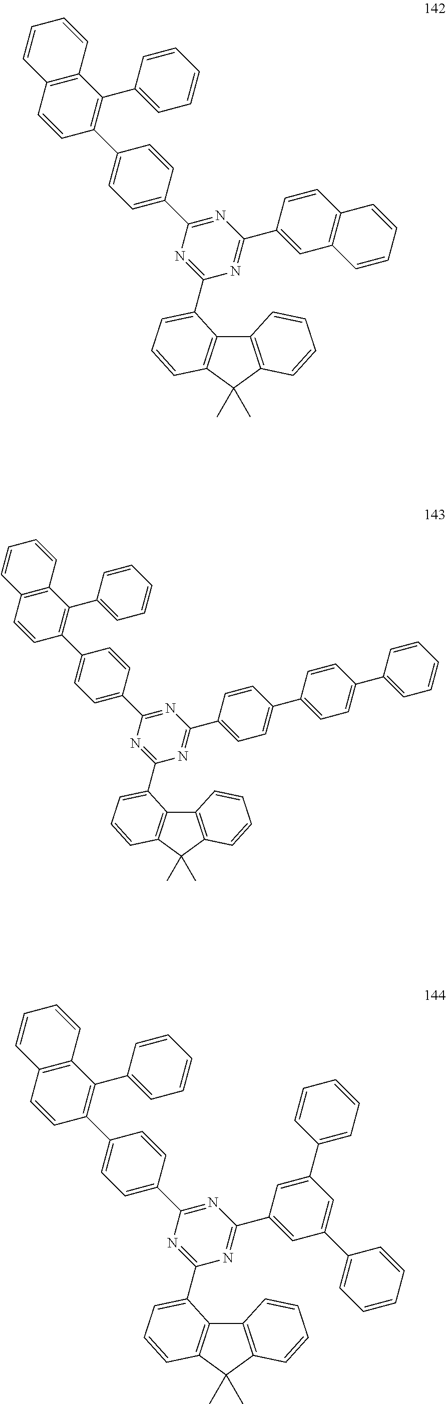 [SMILES: CC1(C)c2ccccc2-c2c(-c3nc(-c4ccc(-c5ccc6ccccc6c5-c5ccccc5)cc4)nc(-c4ccc5ccccc5c4)n3)cccc21 ; CC1(C)c2ccccc2-c2c(-c3nc(-c4ccc(-c5ccc(-c6ccccc6)cc5)cc4)nc(-c4ccc(-c5ccc6ccccc6c5-c5ccccc5)cc4)n3)cccc21 ; CC1(C)c2ccccc2-c2c(-c3nc(-c4ccc(-c5ccc6ccccc6c5-c5ccccc5)cc4)nc(-c4cc(-c5ccccc5)cc(-c5ccccc5)c4)n3)cccc21]

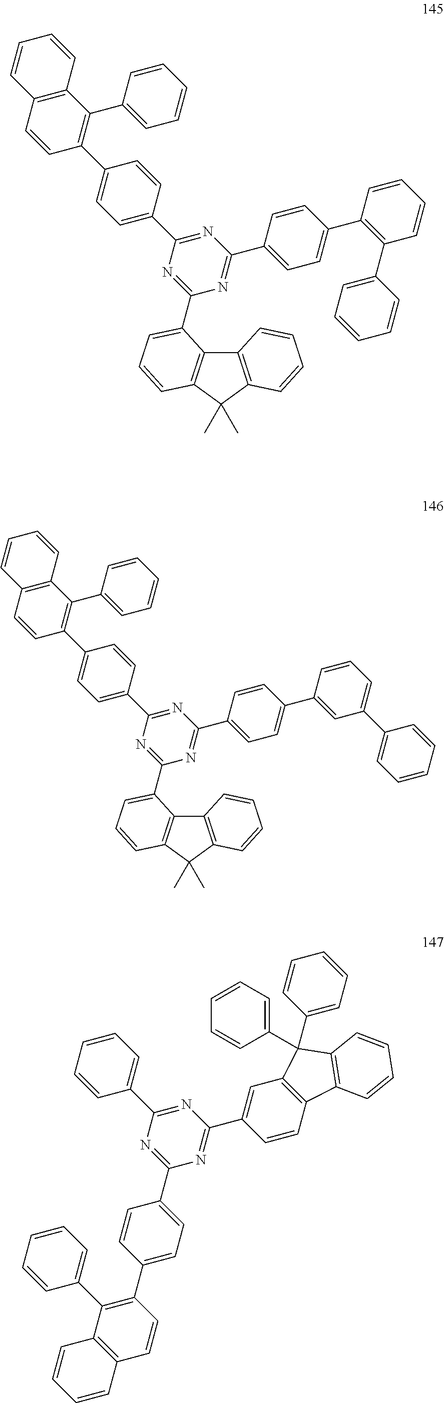 [SMILES: CC1(C)c2ccccc2-c2c(-c3nc(-c4ccc(-c5ccccc5-c5ccccc5)cc4)nc(-c4ccc(-c5ccc6ccccc6c5-c5ccccc5)cc4)n3)cccc21 ; CC1(C)c2ccccc2-c2c(-c3nc(-c4ccc(-c5cccc(-c6ccccc6)c5)cc4)nc(-c4ccc(-c5ccc6ccccc6c5-c5ccccc5)cc4)n3)cccc21 ; c1ccc(-c2nc(-c3ccc(-c4ccc5ccccc5c4-c4ccccc4)cc3)nc(-c3ccc4c(c3)C(c3ccccc3)(c3ccccc3)c3ccccc3-4)n2)cc1]

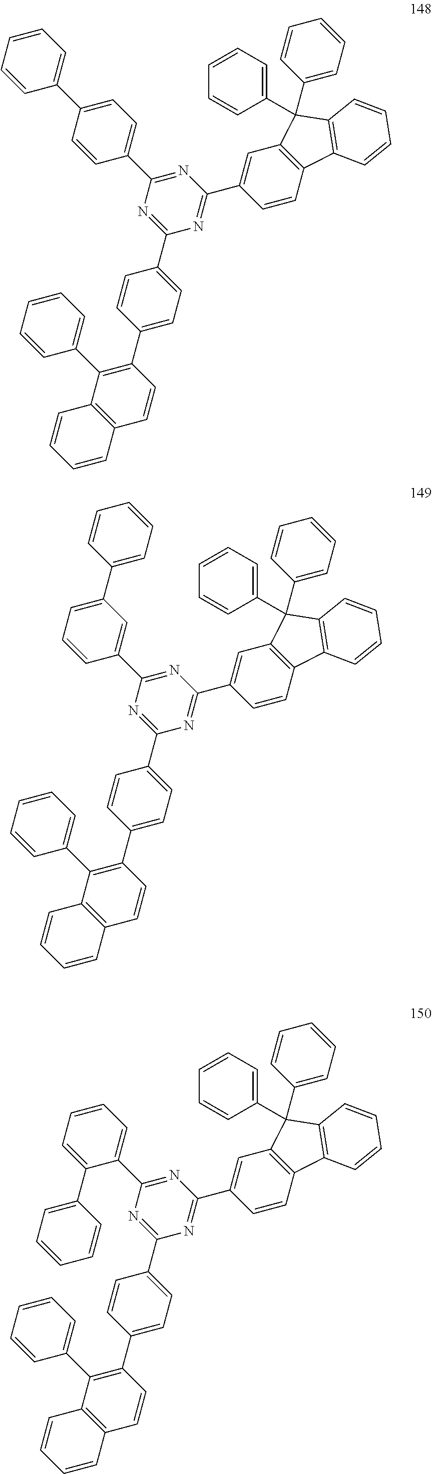 [SMILES: c1ccc(-c2ccc(-c3nc(-c4ccc(-c5ccc6ccccc6c5-c5ccccc5)cc4)nc(-c4ccc5c(c4)C(c4ccccc4)(c4ccccc4)c4ccccc4-5)n3)cc2)cc1 ; c1ccc(-c2cccc(-c3nc(-c4ccc(-c5ccc6ccccc6c5-c5ccccc5)cc4)nc(-c4ccc5c(c4)C(c4ccccc4)(c4ccccc4)c4ccccc4-5)n3)c2)cc1 ; c1ccc(-c2ccccc2-c2nc(-c3ccc(-c4ccc5ccccc5c4-c4ccccc4)cc3)nc(-c3ccc4c(c3)C(c3ccccc3)(c3ccccc3)c3ccccc3-4)n2)cc1]

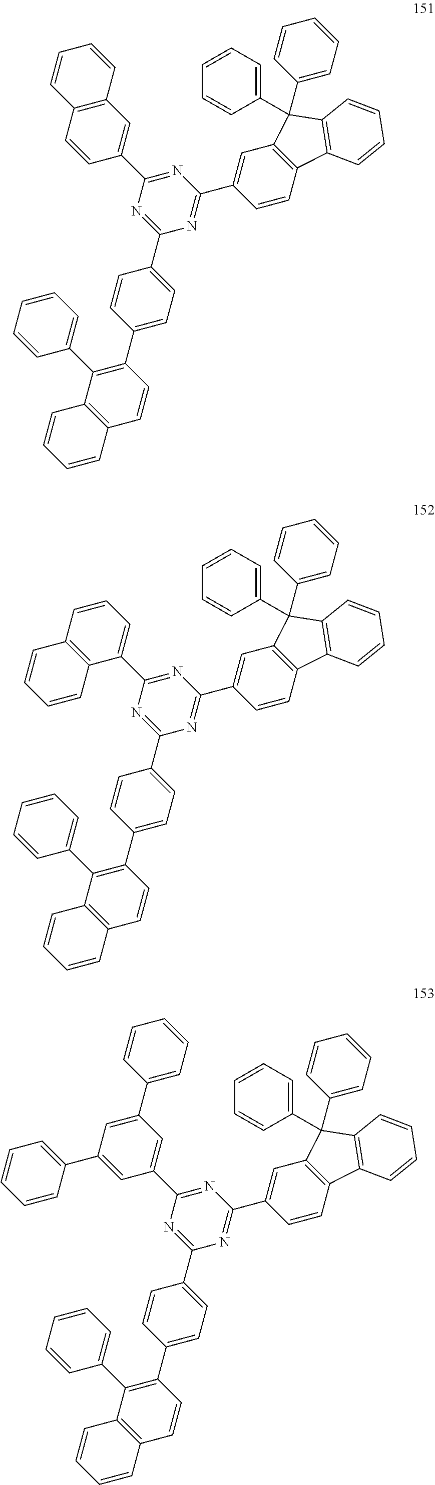 [SMILES: c1ccc(-c2c(-c3ccc(-c4nc(-c5ccc6c(c5)C(c5ccccc5)(c5ccccc5)c5ccccc5-6)nc(-c5ccc6ccccc6c5)n4)cc3)ccc3ccccc23)cc1 ; c1ccc(-c2c(-c3ccc(-c4nc(-c5ccc6c(c5)C(c5ccccc5)(c5ccccc5)c5ccccc5-6)nc(-c5cccc6ccccc56)n4)cc3)ccc3ccccc23)cc1 ; c1ccc(-c2cc(-c3ccccc3)cc(-c3nc(-c4ccc(-c5ccc6ccccc6c5-c5ccccc5)cc4)nc(-c4ccc5c(c4)C(c4ccccc4)(c4ccccc4)c4ccccc4-5)n3)c2)cc1]

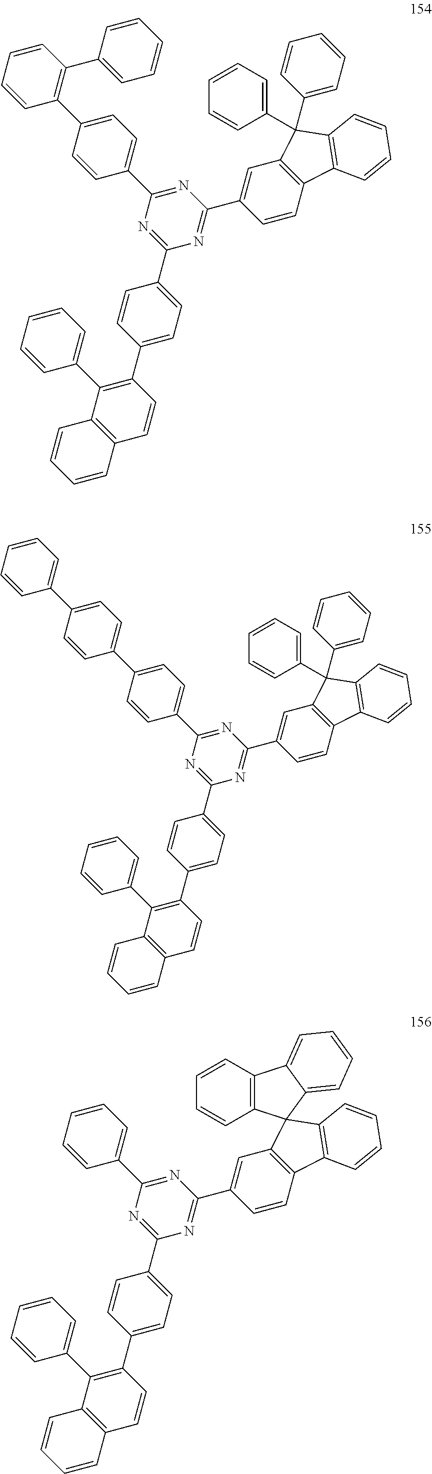 [SMILES: c1ccc(-c2ccccc2-c2ccc(-c3nc(-c4ccc(-c5ccc6ccccc6c5-c5ccccc5)cc4)nc(-c4ccc5c(c4)C(c4ccccc4)(c4ccccc4)c4ccccc4-5)n3)cc2)cc1 ; c1ccc(-c2ccc(-c3ccc(-c4nc(-c5ccc(-c6ccc7ccccc7c6-c6ccccc6)cc5)nc(-c5ccc6c(c5)C(c5ccccc5)(c5ccccc5)c5ccccc5-6)n4)cc3)cc2)cc1 ; c1ccc(-c2nc(-c3ccc(-c4ccc5ccccc5c4-c4ccccc4)cc3)nc(-c3ccc4c(c3)C3(c5ccccc5-c5ccccc53)c3ccccc3-4)n2)cc1]

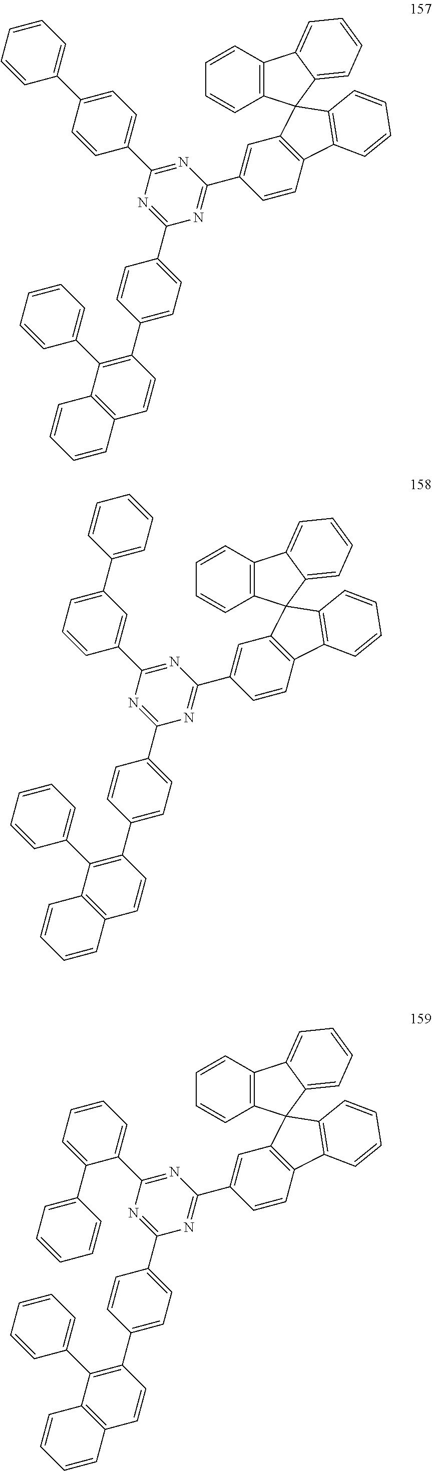 [SMILES: c1ccc(-c2ccc(-c3nc(-c4ccc(-c5ccc6ccccc6c5-c5ccccc5)cc4)nc(-c4ccc5c(c4)C4(c6ccccc6-c6ccccc64)c4ccccc4-5)n3)cc2)cc1 ; c1ccc(-c2cccc(-c3nc(-c4ccc(-c5ccc6ccccc6c5-c5ccccc5)cc4)nc(-c4ccc5c(c4)C4(c6ccccc6-c6ccccc64)c4ccccc4-5)n3)c2)cc1 ; c1ccc(-c2ccccc2-c2nc(-c3ccc(-c4ccc5ccccc5c4-c4ccccc4)cc3)nc(-c3ccc4c(c3)C3(c5ccccc5-c5ccccc53)c3ccccc3-4)n2)cc1]

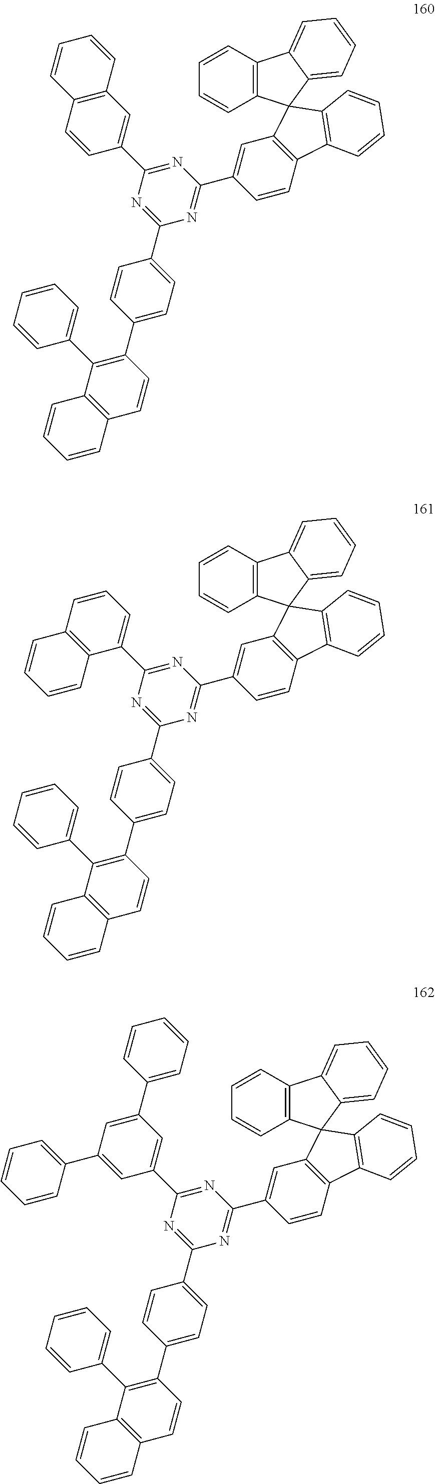 [SMILES: c1ccc(-c2c(-c3ccc(-c4nc(-c5ccc6c(c5)C5(c7ccccc7-c7ccccc75)c5ccccc5-6)nc(-c5ccc6ccccc6c5)n4)cc3)ccc3ccccc23)cc1 ; c1ccc(-c2c(-c3ccc(-c4nc(-c5ccc6c(c5)C5(c7ccccc7-c7ccccc75)c5ccccc5-6)nc(-c5cccc6ccccc56)n4)cc3)ccc3ccccc23)cc1 ; c1ccc(-c2cc(-c3ccccc3)cc(-c3nc(-c4ccc(-c5ccc6ccccc6c5-c5ccccc5)cc4)nc(-c4ccc5c(c4)C4(c6ccccc6-c6ccccc64)c4ccccc4-5)n3)c2)cc1]

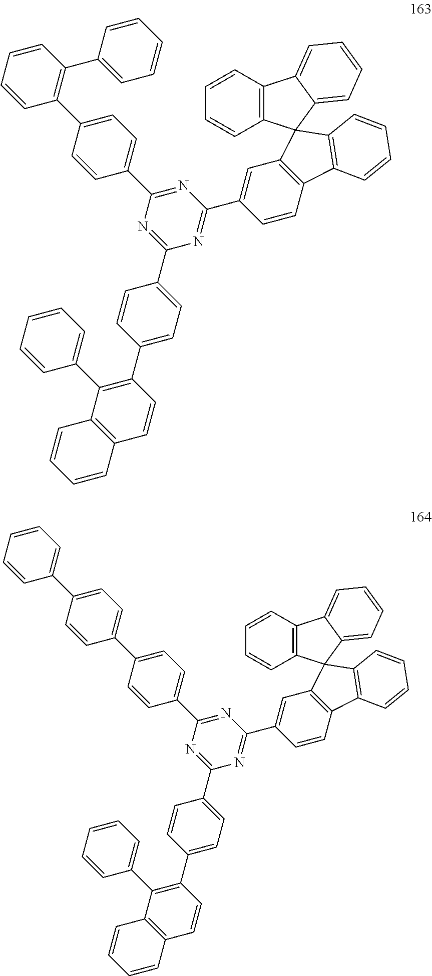 [SMILES: c1ccc(-c2ccccc2-c2ccc(-c3nc(-c4ccc(-c5ccc6ccccc6c5-c5ccccc5)cc4)nc(-c4ccc5c(c4)C4(c6ccccc6-c6ccccc64)c4ccccc4-5)n3)cc2)cc1 ; c1ccc(-c2ccc(-c3ccc(-c4nc(-c5ccc(-c6ccc7ccccc7c6-c6ccccc6)cc5)nc(-c5ccc6c(c5)C5(c7ccccc7-c7ccccc75)c5ccccc5-6)n4)cc3)cc2)cc1]

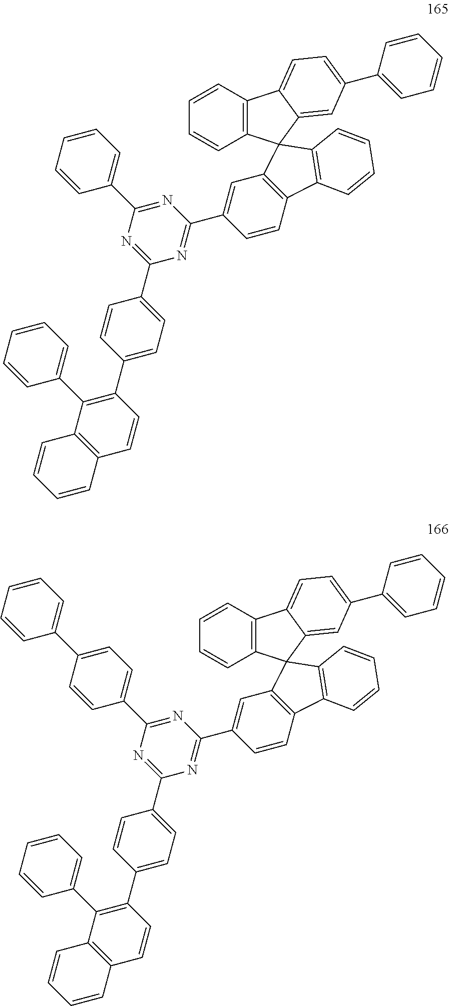 [SMILES: c1ccc(-c2ccc3c(c2)C2(c4ccccc4-3)c3ccccc3-c3ccc(-c4nc(-c5ccccc5)nc(-c5ccc(-c6ccc7ccccc7c6-c6ccccc6)cc5)n4)cc32)cc1 ; c1ccc(-c2ccc(-c3nc(-c4ccc(-c5ccc6ccccc6c5-c5ccccc5)cc4)nc(-c4ccc5c(c4)C4(c6ccccc6-c6ccc(-c7ccccc7)cc64)c4ccccc4-5)n3)cc2)cc1]

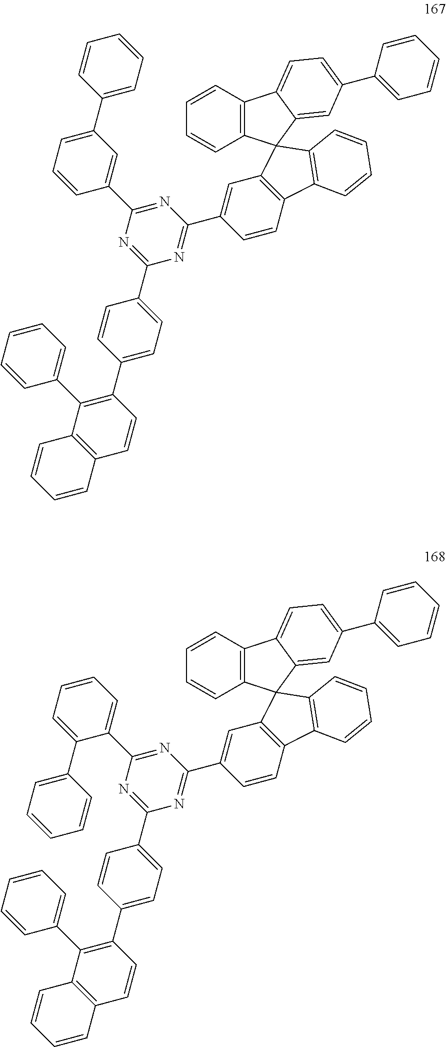 [SMILES: c1ccc(-c2cccc(-c3nc(-c4ccc(-c5ccc6ccccc6c5-c5ccccc5)cc4)nc(-c4ccc5c(c4)C4(c6ccccc6-c6ccc(-c7ccccc7)cc64)c4ccccc4-5)n3)c2)cc1 ; c1ccc(-c2ccc3c(c2)C2(c4ccccc4-3)c3ccccc3-c3ccc(-c4nc(-c5ccc(-c6ccc7ccccc7c6-c6ccccc6)cc5)nc(-c5ccccc5-c5ccccc5)n4)cc32)cc1]

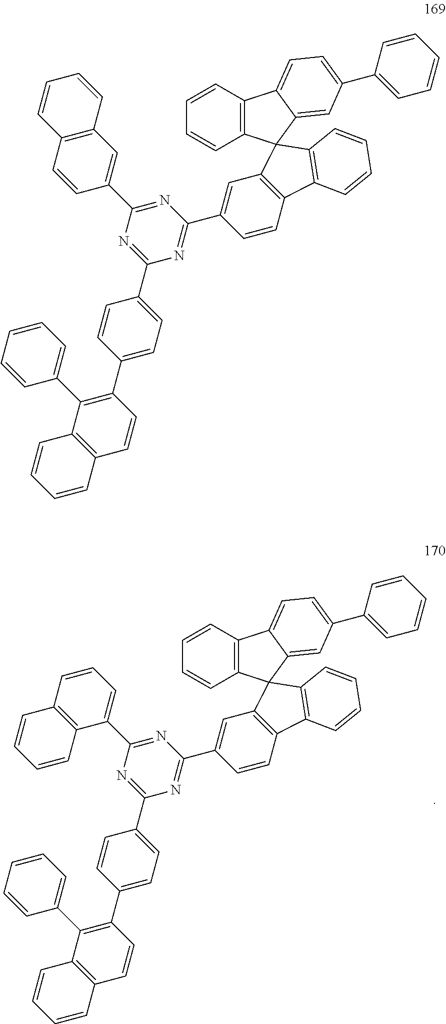 [SMILES: c1ccc(-c2ccc3c(c2)C2(c4ccccc4-3)c3ccccc3-c3ccc(-c4nc(-c5ccc(-c6ccc7ccccc7c6-c6ccccc6)cc5)nc(-c5ccc6ccccc6c5)n4)cc32)cc1 ; c1ccc(-c2ccc3c(c2)C2(c4ccccc4-3)c3ccccc3-c3ccc(-c4nc(-c5ccc(-c6ccc7ccccc7c6-c6ccccc6)cc5)nc(-c5cccc6ccccc56)n4)cc32)cc1]

11. The light emitting element of claim 1, wherein the emission layer further comprises a compound represented by Formula F-c: 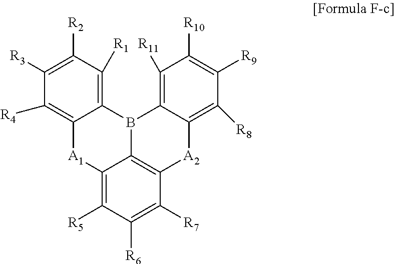
wherein in Formula F-c,
A₁ and A₂ are each independently, O, S, Se, or N(Rₘ),
R₁ to R₁₁ are each independently a hydrogen atom, a deuterium atom, a halogen atom, a cyano group, a substituted or unsubstituted oxy group, a substituted or unsubstituted thio group, a substituted or unsubstituted boron group, a substituted or unsubstituted amine group, a substituted or unsubstituted alkyl group having 1 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring, and
Rₘ is a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group of 2 to 30 ring-forming carbon atoms.

12. A nitrogen-containing compound represented by Formula 1: 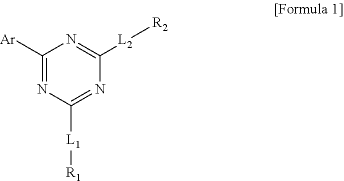
wherein in Formula 1,
Ar is a substituted or unsubstituted aryl group having 6 to 30 ring-forming carbon atoms or a substituted or unsubstituted heteroaryl group having 2 to 30 ring-forming carbon atoms,
L₁ and L₂ are each independently a direct linkage or a substituted or unsubstituted aryl group having 6 to 30 ring-forming carbon atoms,
R₁ is a group represented by Formula 2, and
R₂ is a group represented by Formula 3-1, Formula 3-2, or Formula 3-3: 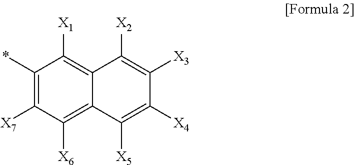
wherein in Formula 2,
X₁ to X₇ are each independently a hydrogen atom, a deuterium atom, a halogen atom, a cyano group, a hydroxy group, a substituted or unsubstituted oxy group, a substituted or unsubstituted thio group, a substituted or unsubstituted boron group, a substituted or unsubstituted silyl group, a substituted or unsubstituted amine group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms,
except that X₁ or X₇ is a substituted or unsubstituted phenyl group, and 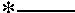 [SMILES: *C]
is a position linked to L₁ in Formula 1: 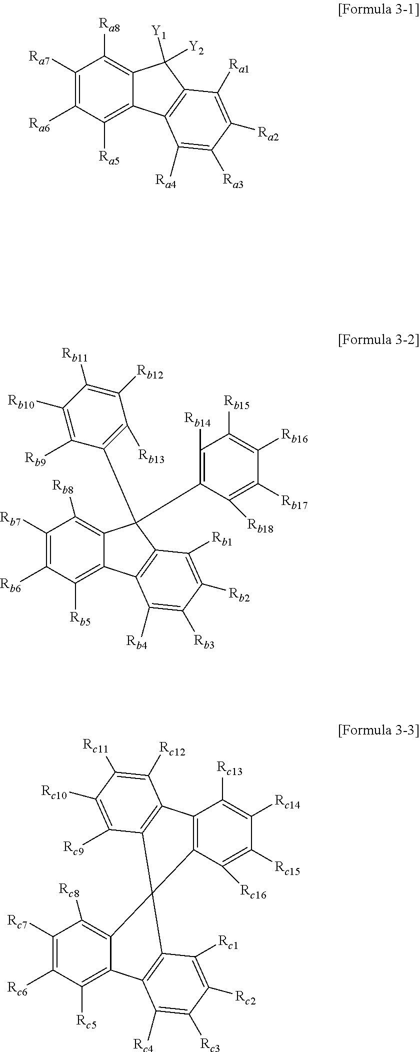
wherein in Formula 3-1,
Y₁ and Y₂ are each independently a hydrogen atom, a deuterium atom, a halogen atom, a cyano group, a hydroxy group, or a substituted or unsubstituted alkyl group having 1 to 20 carbon atoms,
wherein in Formula 3-1 to Formula 3-3,
Rₐ₁ to Rₐ₈, R_{b1} to R_{b18}, and R_{c1} to R_{c16} are each independently a hydrogen atom, a deuterium atom, a halogen atom, a cyano group, a hydroxy group, a substituted or unsubstituted oxy group, a substituted or unsubstituted thio group, a substituted or unsubstituted boron group, a substituted or unsubstituted silyl group, a substituted or unsubstituted amine group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms,
except that
when R₂ in Formula 1 is a group represented by Formula 3-1, one of Rₐ₁ to Rₐ₈ in Formula 3-1 is a position linked to L₂ in Formula 1,
when R₂ in Formula 1 is a group represented by Formula 3-2, one of R_{b1} to R_{b18} in Formula 3-2 is a position linked to L₂ in Formula 1, and
when R₂ in Formula 1 is a group represented by Formula 3-3, one of R_{c1} to R_{c16} in Formula 3-3 is a position linked to L₂ in Formula 1.

13. The nitrogen-containing compound of claim 12, wherein
L₁ is a direct linkage or a substituted or unsubstituted phenyl group, and
L₂ is a direct linkage.

14. The nitrogen-containing compound of claim 12, wherein Ar is a substituted or unsubstituted phenyl group, a substituted or unsubstituted biphenyl group, a substituted or unsubstituted terphenyl group, or a substituted or unsubstituted naphthyl group.

15. The nitrogen-containing compound of claim 12, wherein the nitrogen-containing compound represented by Formula 1 is represented by one of Formula 1-1 to Formula 1-4: 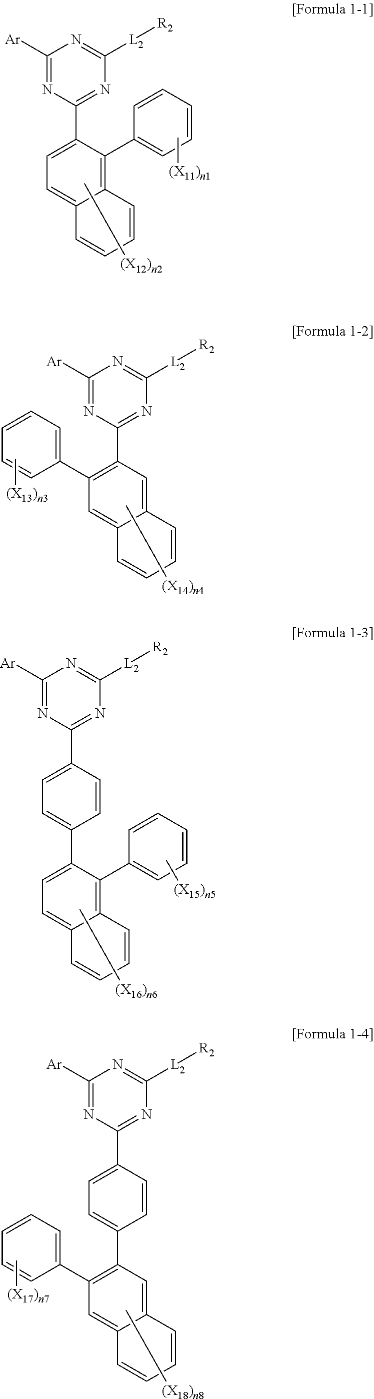
wherein in Formula 1-1 to Formula 1-4,
X₁₁ to X₁₈ are each independently a hydrogen atom or a deuterium atom,
n1, n3, n5, and n7 are each independently an integer from 0 to 5,
n2, n4, n6, and n8 are each independently an integer from 0 to 6, and
Ar, L₂, and R₂ are the same as defined in Formula 1.

16. The nitrogen-containing compound of claim 12, wherein the nitrogen-containing compound represented by Formula 1 is represented by one of Formula 1-5 to Formula 1-9: 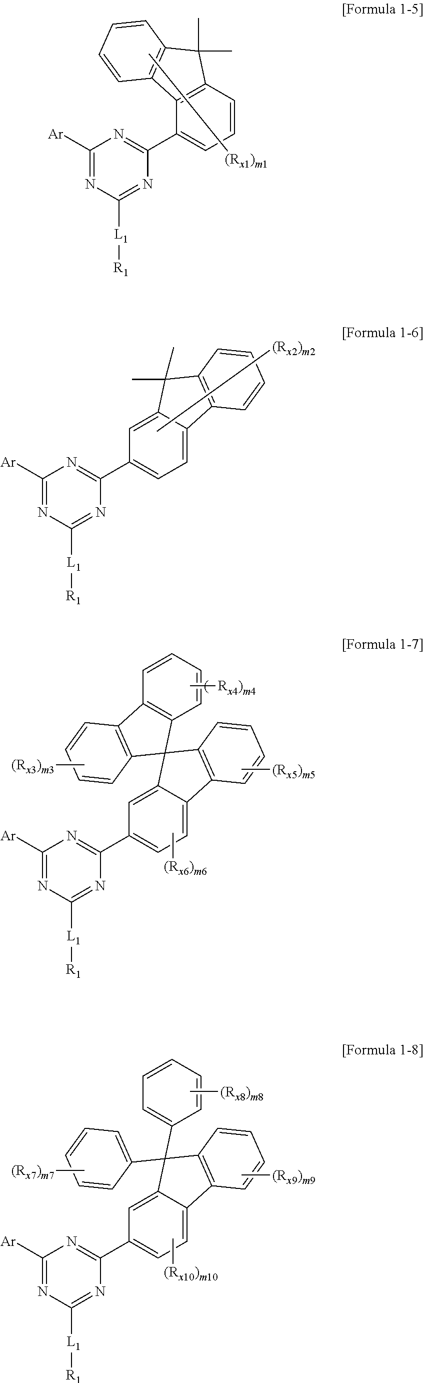 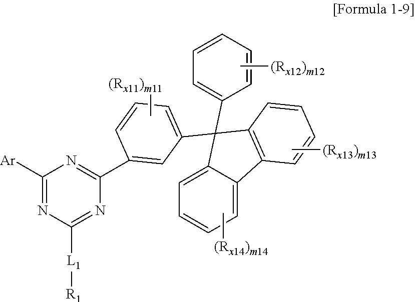
wherein in Formula 1-5 to Formula 1-9,
Rₓ₁ to Rₓ₁₄ are each independently a hydrogen atom, a deuterium atom, a halogen atom, a cyano group, or a substituted or unsubstituted aryl group having 6 to 12 ring-forming carbon atoms,
m1 and m2 are each independently an integer from 0 to 7,
m3 to m5, m9, m11, m13, and m14 are each independently an integer from 0 to 4,
m6 and m10 are each independently an integer from 0 to 3,
m7, m8, and m12 are each independently an integer from 0 to 5, and
Ar, L₁, and R₁ are the same as defined in Formula 1.

17. The nitrogen-containing compound of claim 16, wherein
Rₓ₁ to Rₓ₃ and Rₓ₅ to Rₓ₁₄ are each a hydrogen atom, and
Rₓ₄ is a hydrogen atom or a substituted or unsubstituted phenyl group.

18. The nitrogen-containing compound of claim 12, wherein the nitrogen-containing compound represented by Formula 1 is represented by one of Formula 1-10 to Formula 1-14: 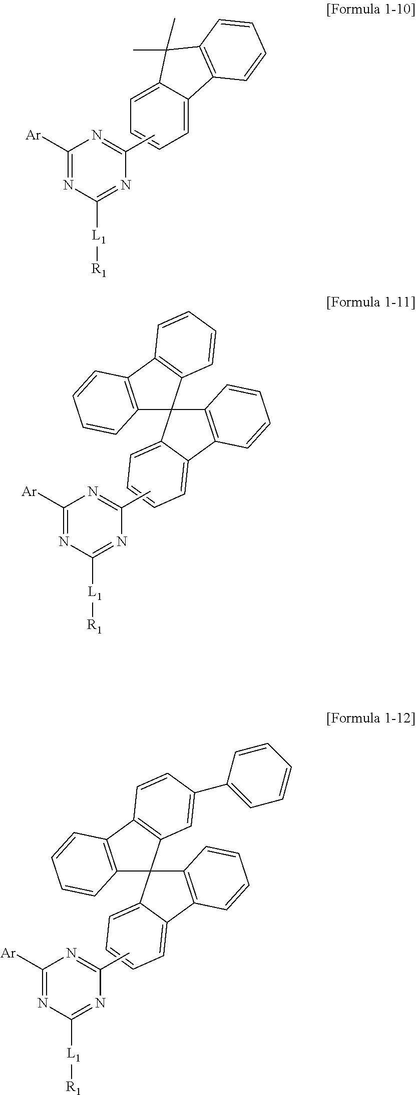 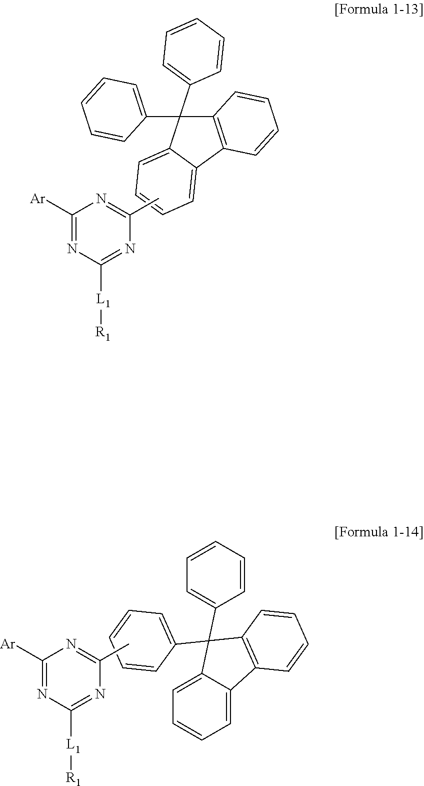
wherein in Formula 1-10 to Formula 1-14,
Ar, L₁, and R₁ are the same as defined in Formula 1.

19. The nitrogen-containing compound of claim 12, wherein the nitrogen-containing compound represented by Formula 1 is selected from Compound Group 1:

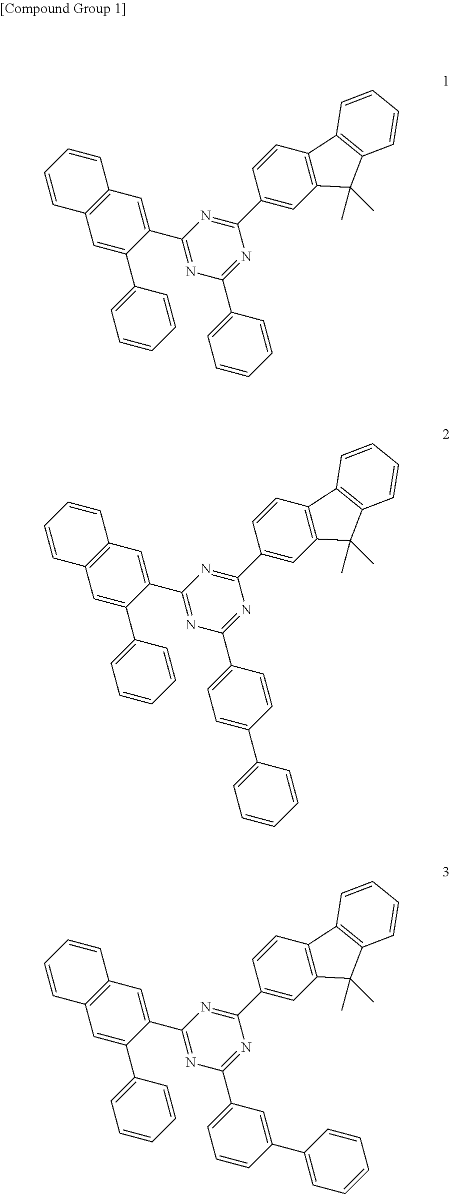 [SMILES: CC1(C)c2ccccc2-c2ccc(-c3nc(-c4ccccc4)nc(-c4cc5ccccc5cc4-c4ccccc4)n3)cc21 ; CC1(C)c2ccccc2-c2ccc(-c3nc(-c4ccc(-c5ccccc5)cc4)nc(-c4cc5ccccc5cc4-c4ccccc4)n3)cc21 ; CC1(C)c2ccccc2-c2ccc(-c3nc(-c4cccc(-c5ccccc5)c4)nc(-c4cc5ccccc5cc4-c4ccccc4)n3)cc21]

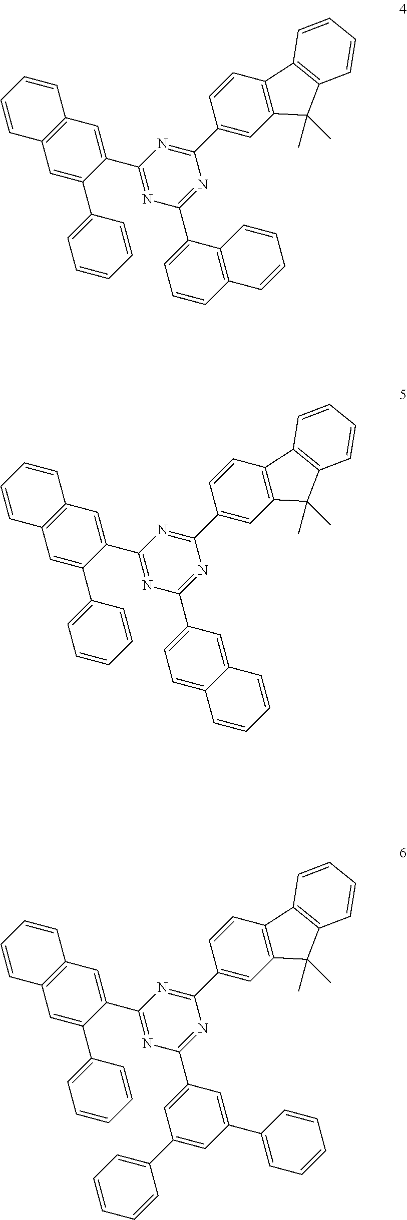 [SMILES: CC1(C)c2ccccc2-c2ccc(-c3nc(-c4cc5ccccc5cc4-c4ccccc4)nc(-c4cccc5ccccc45)n3)cc21 ; CC1(C)c2ccccc2-c2ccc(-c3nc(-c4ccc5ccccc5c4)nc(-c4cc5ccccc5cc4-c4ccccc4)n3)cc21 ; CC1(C)c2ccccc2-c2ccc(-c3nc(-c4cc(-c5ccccc5)cc(-c5ccccc5)c4)nc(-c4cc5ccccc5cc4-c4ccccc4)n3)cc21]

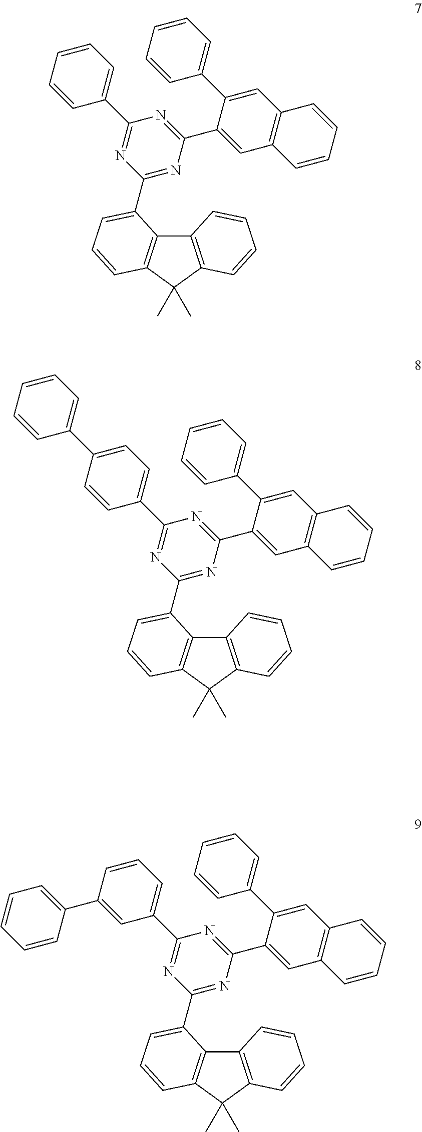 [SMILES: CC1(C)c2ccccc2-c2c(-c3nc(-c4ccccc4)nc(-c4cc5ccccc5cc4-c4ccccc4)n3)cccc21 ; CC1(C)c2ccccc2-c2c(-c3nc(-c4ccc(-c5ccccc5)cc4)nc(-c4cc5ccccc5cc4-c4ccccc4)n3)cccc21 ; CC1(C)c2ccccc2-c2c(-c3nc(-c4cccc(-c5ccccc5)c4)nc(-c4cc5ccccc5cc4-c4ccccc4)n3)cccc21]

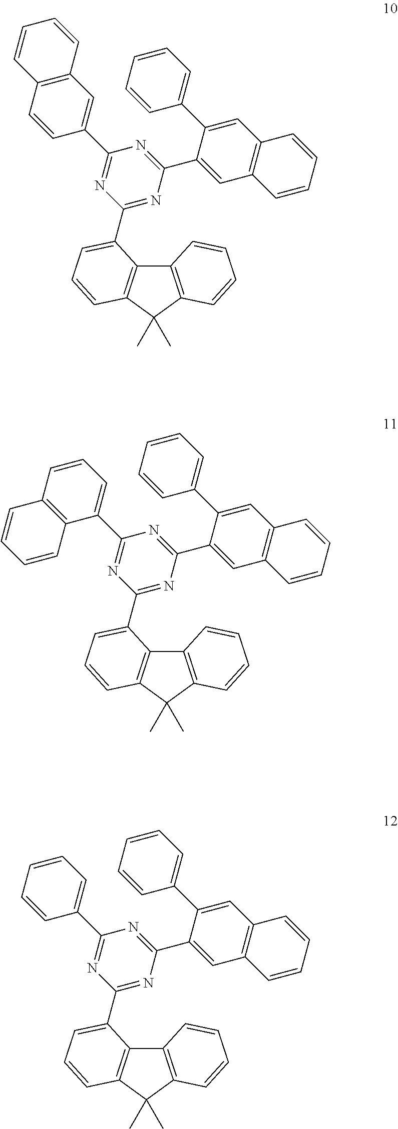 [SMILES: CC1(C)c2ccccc2-c2c(-c3nc(-c4ccc5ccccc5c4)nc(-c4cc5ccccc5cc4-c4ccccc4)n3)cccc21 ; CC1(C)c2ccccc2-c2c(-c3nc(-c4cc5ccccc5cc4-c4ccccc4)nc(-c4cccc5ccccc45)n3)cccc21 ; CC1(C)c2ccccc2-c2c(-c3nc(-c4ccccc4)nc(-c4cc5ccccc5cc4-c4ccccc4)n3)cccc21]

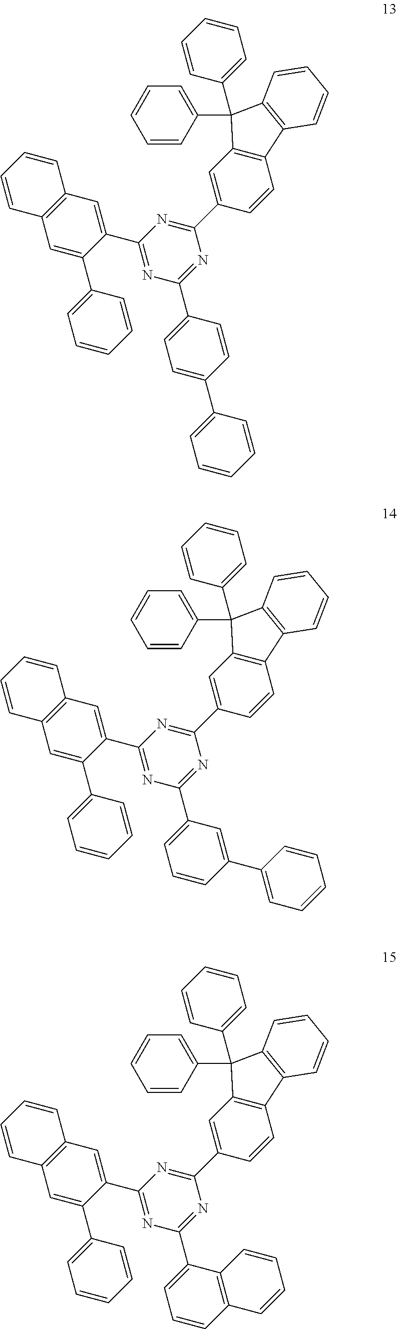 [SMILES: c1ccc(-c2ccc(-c3nc(-c4ccc5c(c4)C(c4ccccc4)(c4ccccc4)c4ccccc4-5)nc(-c4cc5ccccc5cc4-c4ccccc4)n3)cc2)cc1 ; c1ccc(-c2cccc(-c3nc(-c4ccc5c(c4)C(c4ccccc4)(c4ccccc4)c4ccccc4-5)nc(-c4cc5ccccc5cc4-c4ccccc4)n3)c2)cc1 ; c1ccc(-c2cc3ccccc3cc2-c2nc(-c3ccc4c(c3)C(c3ccccc3)(c3ccccc3)c3ccccc3-4)nc(-c3cccc4ccccc34)n2)cc1]

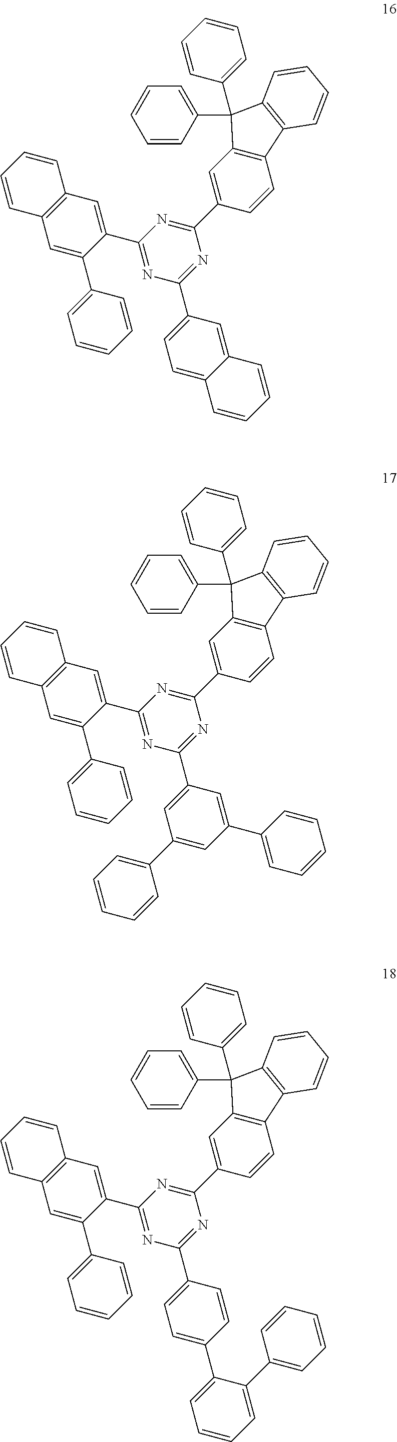 [SMILES: c1ccc(-c2cc3ccccc3cc2-c2nc(-c3ccc4c(c3)C(c3ccccc3)(c3ccccc3)c3ccccc3-4)nc(-c3ccc4ccccc4c3)n2)cc1 ; c1ccc(-c2cc(-c3ccccc3)cc(-c3nc(-c4ccc5c(c4)C(c4ccccc4)(c4ccccc4)c4ccccc4-5)nc(-c4cc5ccccc5cc4-c4ccccc4)n3)c2)cc1 ; c1ccc(-c2ccccc2-c2ccc(-c3nc(-c4ccc5c(c4)C(c4ccccc4)(c4ccccc4)c4ccccc4-5)nc(-c4cc5ccccc5cc4-c4ccccc4)n3)cc2)cc1]

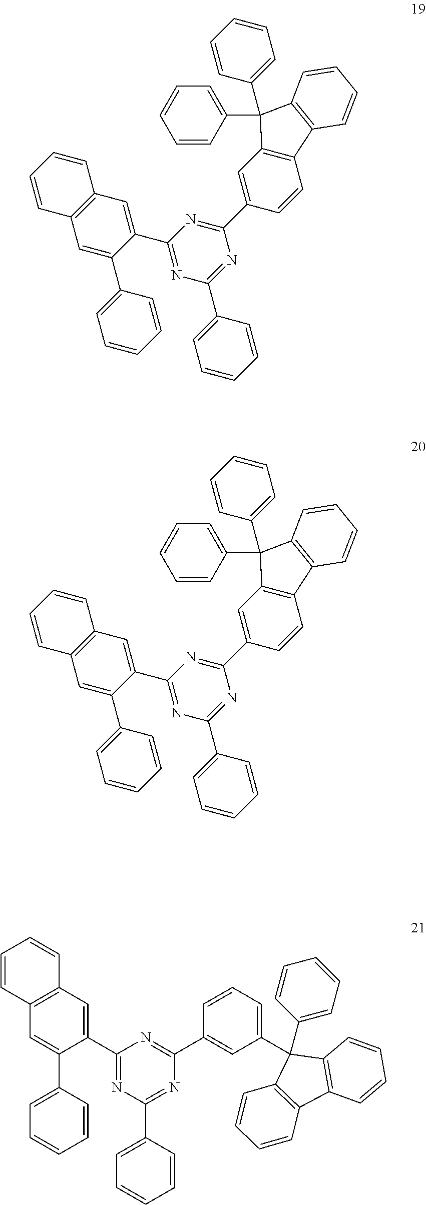 [SMILES: c1ccc(-c2nc(-c3ccc4c(c3)C(c3ccccc3)(c3ccccc3)c3ccccc3-4)nc(-c3cc4ccccc4cc3-c3ccccc3)n2)cc1 ; c1ccc(-c2nc(-c3ccc4c(c3)C(c3ccccc3)(c3ccccc3)c3ccccc3-4)nc(-c3cc4ccccc4cc3-c3ccccc3)n2)cc1 ; c1ccc(-c2nc(-c3cccc(C4(c5ccccc5)c5ccccc5-c5ccccc54)c3)nc(-c3cc4ccccc4cc3-c3ccccc3)n2)cc1]

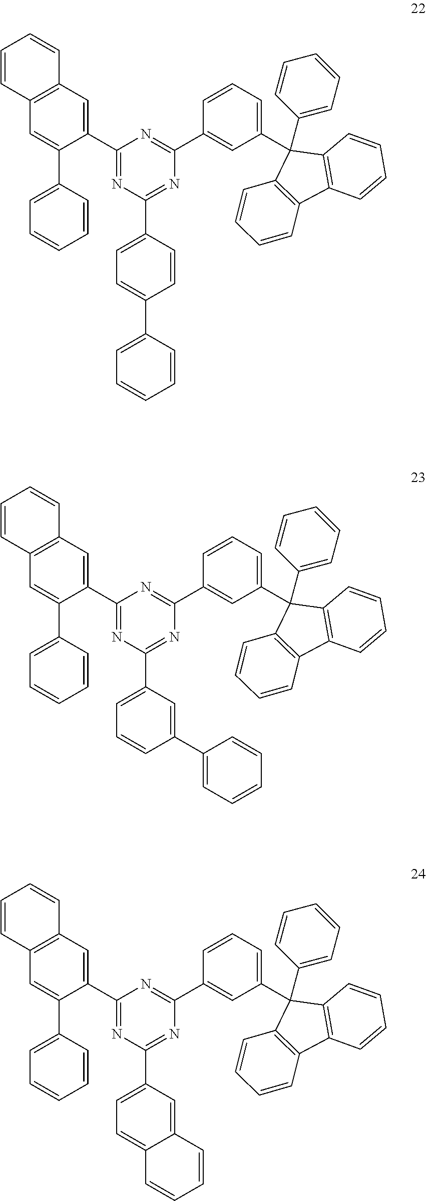 [SMILES: c1ccc(-c2ccc(-c3nc(-c4cccc(C5(c6ccccc6)c6ccccc6-c6ccccc65)c4)nc(-c4cc5ccccc5cc4-c4ccccc4)n3)cc2)cc1 ; c1ccc(-c2cccc(-c3nc(-c4cccc(C5(c6ccccc6)c6ccccc6-c6ccccc65)c4)nc(-c4cc5ccccc5cc4-c4ccccc4)n3)c2)cc1 ; c1ccc(-c2cc3ccccc3cc2-c2nc(-c3cccc(C4(c5ccccc5)c5ccccc5-c5ccccc54)c3)nc(-c3ccc4ccccc4c3)n2)cc1]

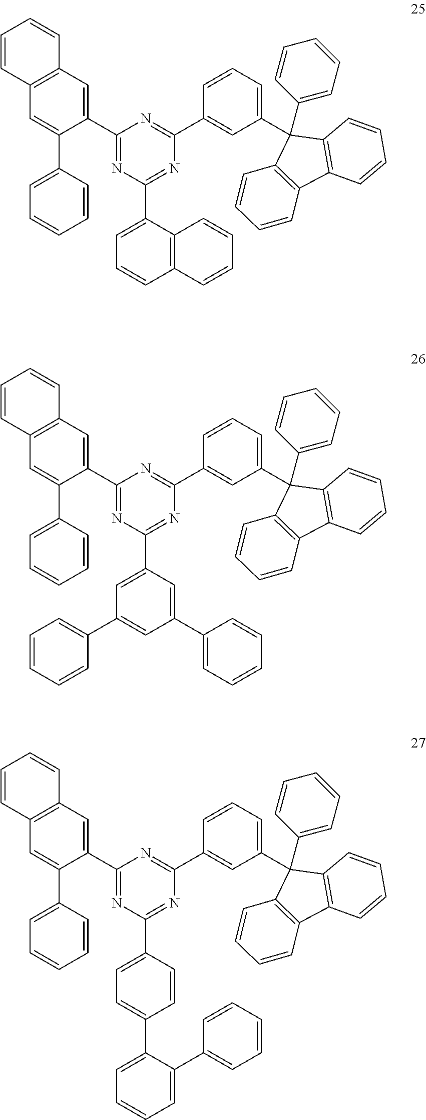 [SMILES: c1ccc(-c2cc3ccccc3cc2-c2nc(-c3cccc(C4(c5ccccc5)c5ccccc5-c5ccccc54)c3)nc(-c3cccc4ccccc34)n2)cc1 ; c1ccc(-c2cc(-c3ccccc3)cc(-c3nc(-c4cccc(C5(c6ccccc6)c6ccccc6-c6ccccc65)c4)nc(-c4cc5ccccc5cc4-c4ccccc4)n3)c2)cc1 ; c1ccc(-c2ccccc2-c2ccc(-c3nc(-c4cccc(C5(c6ccccc6)c6ccccc6-c6ccccc65)c4)nc(-c4cc5ccccc5cc4-c4ccccc4)n3)cc2)cc1]

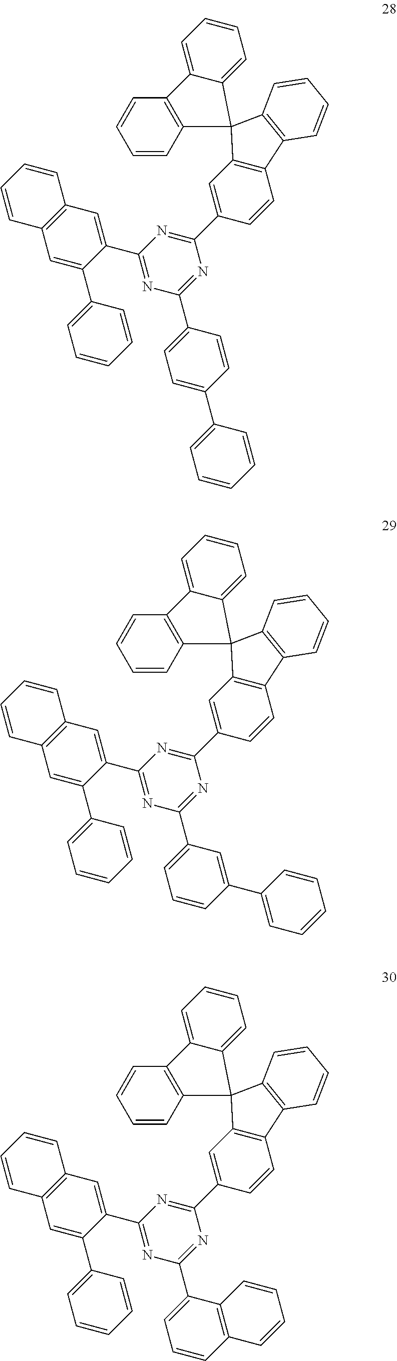 [SMILES: c1ccc(-c2ccc(-c3nc(-c4ccc5c(c4)C4(c6ccccc6-c6ccccc64)c4ccccc4-5)nc(-c4cc5ccccc5cc4-c4ccccc4)n3)cc2)cc1 ; c1ccc(-c2cccc(-c3nc(-c4ccc5c(c4)C4(c6ccccc6-c6ccccc64)c4ccccc4-5)nc(-c4cc5ccccc5cc4-c4ccccc4)n3)c2)cc1 ; c1ccc(-c2cc3ccccc3cc2-c2nc(-c3ccc4c(c3)C3(c5ccccc5-c5ccccc53)c3ccccc3-4)nc(-c3cccc4ccccc34)n2)cc1]

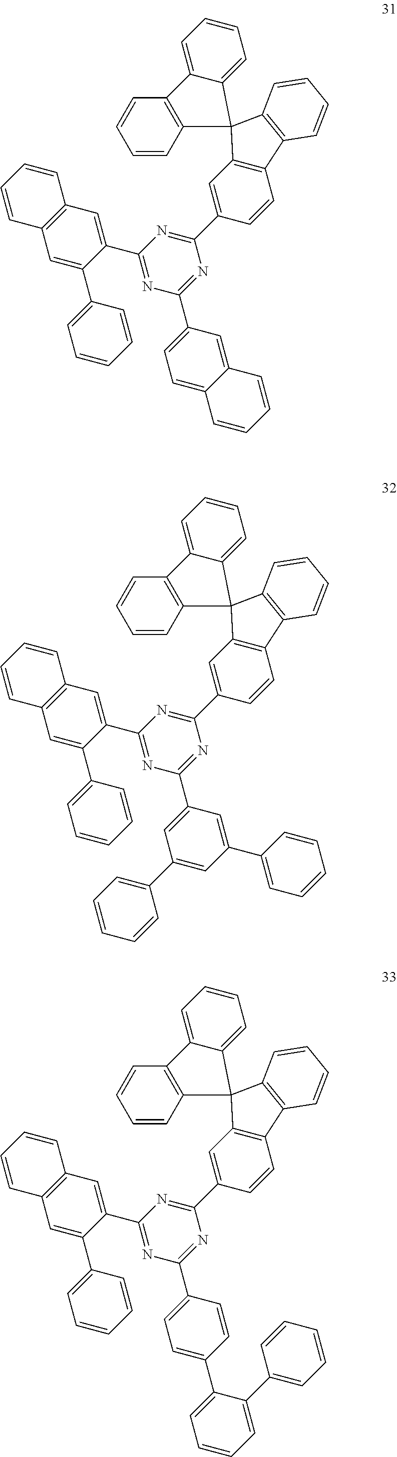 [SMILES: c1ccc(-c2cc3ccccc3cc2-c2nc(-c3ccc4c(c3)C3(c5ccccc5-c5ccccc53)c3ccccc3-4)nc(-c3ccc4ccccc4c3)n2)cc1 ; c1ccc(-c2cc(-c3ccccc3)cc(-c3nc(-c4ccc5c(c4)C4(c6ccccc6-c6ccccc64)c4ccccc4-5)nc(-c4cc5ccccc5cc4-c4ccccc4)n3)c2)cc1 ; c1ccc(-c2ccccc2-c2ccc(-c3nc(-c4ccc5c(c4)C4(c6ccccc6-c6ccccc64)c4ccccc4-5)nc(-c4cc5ccccc5cc4-c4ccccc4)n3)cc2)cc1]

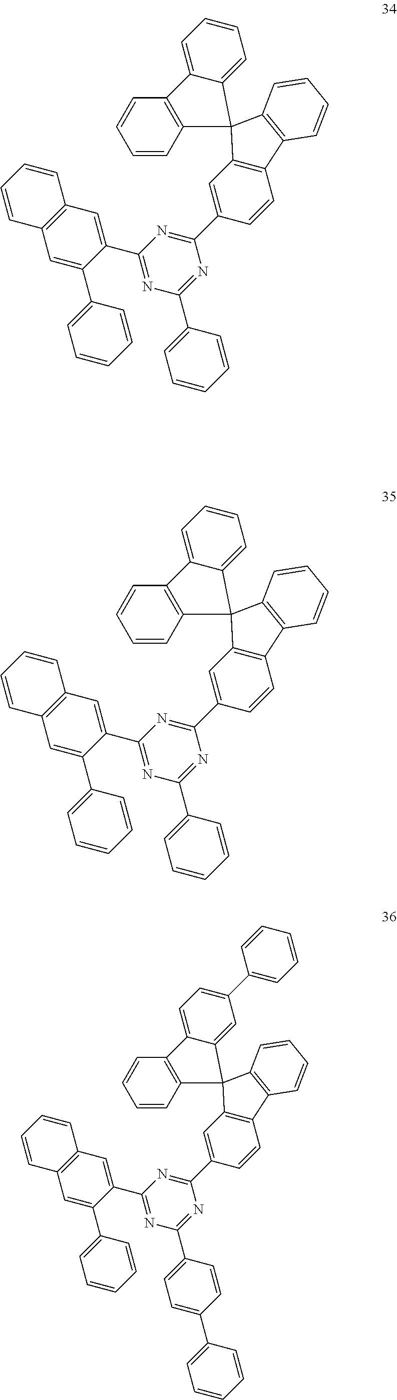 [SMILES: c1ccc(-c2nc(-c3ccc4c(c3)C3(c5ccccc5-c5ccccc53)c3ccccc3-4)nc(-c3cc4ccccc4cc3-c3ccccc3)n2)cc1 ; c1ccc(-c2nc(-c3ccc4c(c3)C3(c5ccccc5-c5ccccc53)c3ccccc3-4)nc(-c3cc4ccccc4cc3-c3ccccc3)n2)cc1 ; c1ccc(-c2ccc(-c3nc(-c4ccc5c(c4)C4(c6ccccc6-c6ccc(-c7ccccc7)cc64)c4ccccc4-5)nc(-c4cc5ccccc5cc4-c4ccccc4)n3)cc2)cc1]

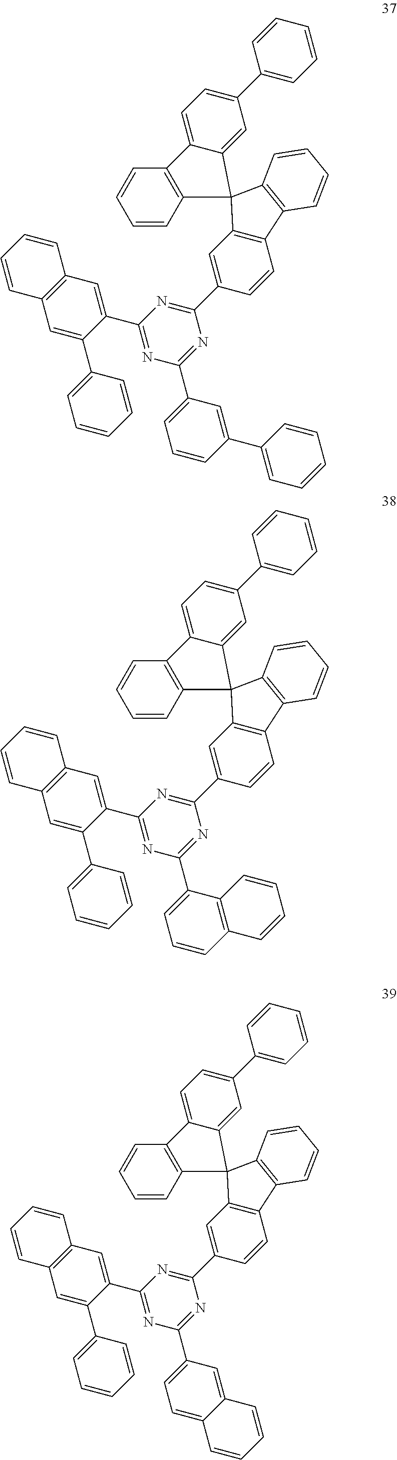

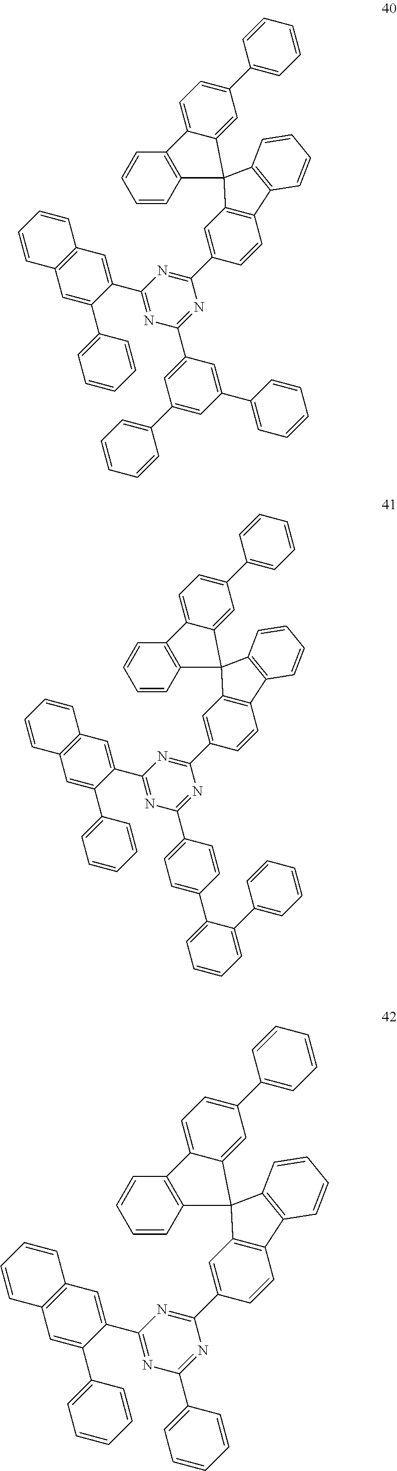 [SMILES: c1ccc(-c2cc(-c3ccccc3)cc(-c3nc(-c4ccc5c(c4)C4(c6ccccc6-c6ccc(-c7ccccc7)cc64)c4ccccc4-5)nc(-c4cc5ccccc5cc4-c4ccccc4)n3)c2)cc1 ; c1ccc(-c2ccc3c(c2)C2(c4ccccc4-3)c3ccccc3-c3ccc(-c4nc(-c5ccc(-c6ccccc6-c6ccccc6)cc5)nc(-c5cc6ccccc6cc5-c5ccccc5)n4)cc32)cc1 ; c1ccc(-c2ccc3c(c2)C2(c4ccccc4-3)c3ccccc3-c3ccc(-c4nc(-c5ccccc5)nc(-c5cc6ccccc6cc5-c5ccccc5)n4)cc32)cc1]

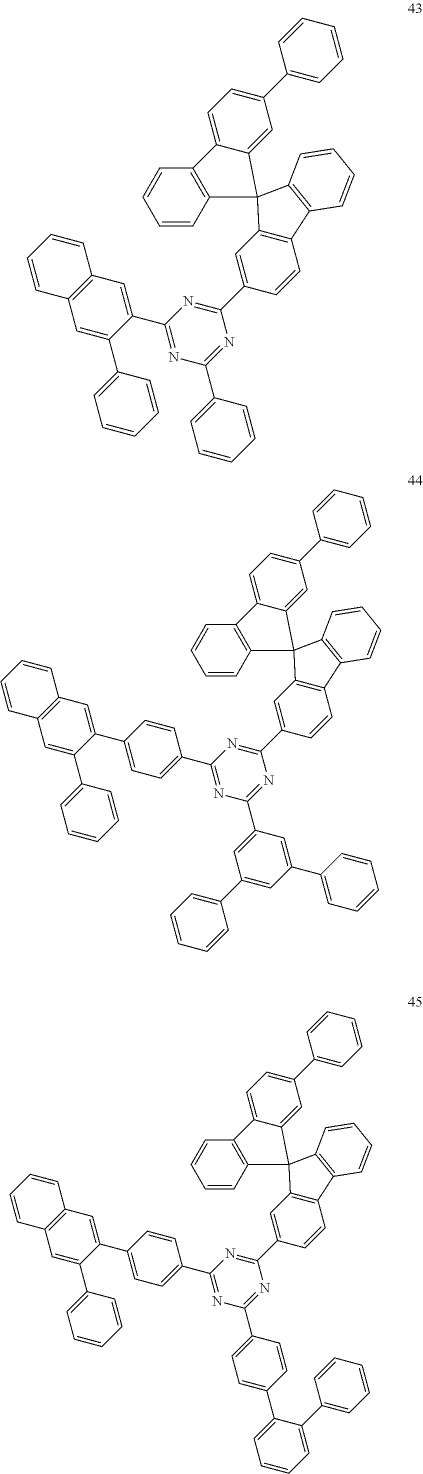 [SMILES: c1ccc(-c2ccc3c(c2)C2(c4ccccc4-3)c3ccccc3-c3ccc(-c4nc(-c5ccccc5)nc(-c5cc6ccccc6cc5-c5ccccc5)n4)cc32)cc1 ; c1ccc(-c2cc(-c3ccccc3)cc(-c3nc(-c4ccc(-c5cc6ccccc6cc5-c5ccccc5)cc4)nc(-c4ccc5c(c4)C4(c6ccccc6-c6ccc(-c7ccccc7)cc64)c4ccccc4-5)n3)c2)cc1 ; c1ccc(-c2ccc3c(c2)C2(c4ccccc4-3)c3ccccc3-c3ccc(-c4nc(-c5ccc(-c6ccccc6-c6ccccc6)cc5)nc(-c5ccc(-c6cc7ccccc7cc6-c6ccccc6)cc5)n4)cc32)cc1]

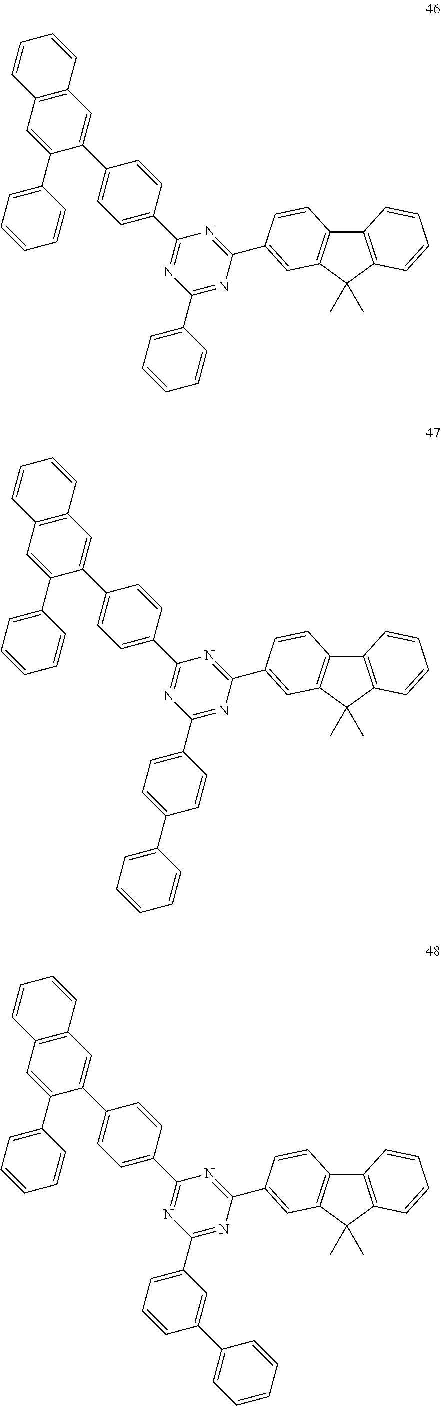 [SMILES: CC1(C)c2ccccc2-c2ccc(-c3nc(-c4ccccc4)nc(-c4ccc(-c5cc6ccccc6cc5-c5ccccc5)cc4)n3)cc21 ; CC1(C)c2ccccc2-c2ccc(-c3nc(-c4ccc(-c5ccccc5)cc4)nc(-c4ccc(-c5cc6ccccc6cc5-c5ccccc5)cc4)n3)cc21 ; CC1(C)c2ccccc2-c2ccc(-c3nc(-c4ccc(-c5cc6ccccc6cc5-c5ccccc5)cc4)nc(-c4cccc(-c5ccccc5)c4)n3)cc21]

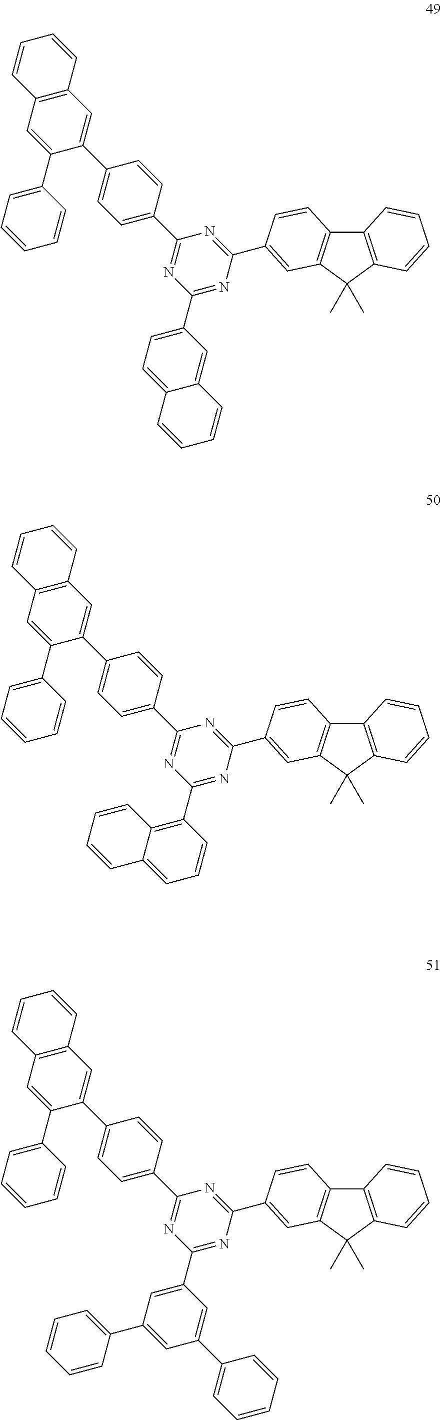 [SMILES: CC1(C)c2ccccc2-c2ccc(-c3nc(-c4ccc(-c5cc6ccccc6cc5-c5ccccc5)cc4)nc(-c4ccc5ccccc5c4)n3)cc21 ; CC1(C)c2ccccc2-c2ccc(-c3nc(-c4ccc(-c5cc6ccccc6cc5-c5ccccc5)cc4)nc(-c4cccc5ccccc45)n3)cc21 ; CC1(C)c2ccccc2-c2ccc(-c3nc(-c4ccc(-c5cc6ccccc6cc5-c5ccccc5)cc4)nc(-c4cc(-c5ccccc5)cc(-c5ccccc5)c4)n3)cc21]

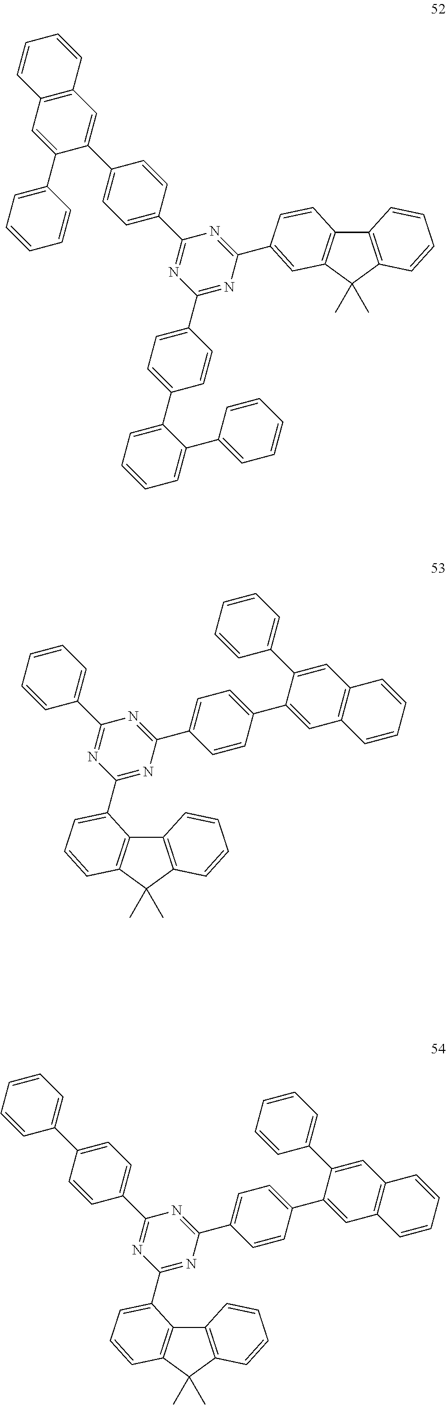 [SMILES: CC1(C)c2ccccc2-c2ccc(-c3nc(-c4ccc(-c5ccccc5-c5ccccc5)cc4)nc(-c4ccc(-c5cc6ccccc6cc5-c5ccccc5)cc4)n3)cc21 ; CC1(C)c2ccccc2-c2c(-c3nc(-c4ccccc4)nc(-c4ccc(-c5cc6ccccc6cc5-c5ccccc5)cc4)n3)cccc21 ; CC1(C)c2ccccc2-c2c(-c3nc(-c4ccc(-c5ccccc5)cc4)nc(-c4ccc(-c5cc6ccccc6cc5-c5ccccc5)cc4)n3)cccc21]

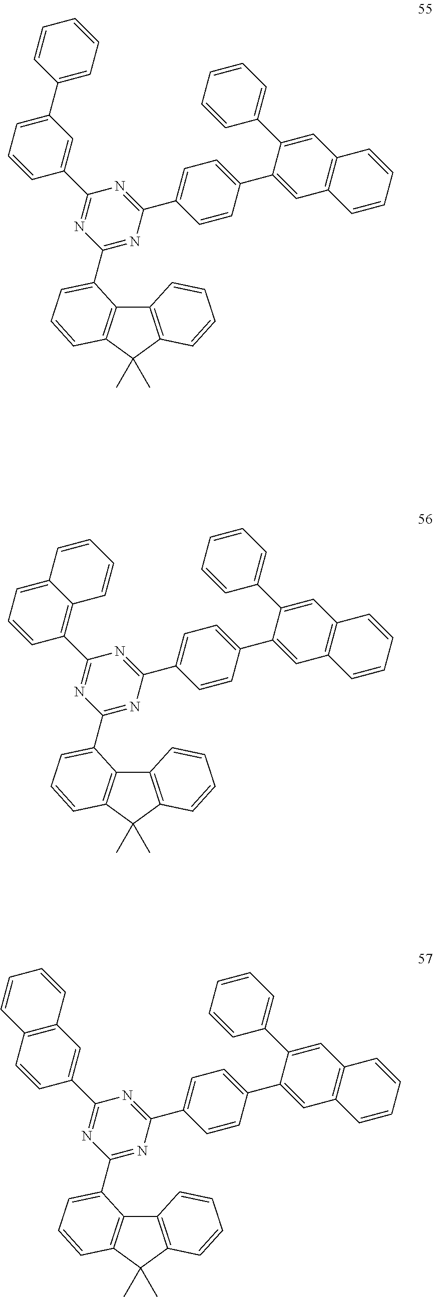 [SMILES: CC1(C)c2ccccc2-c2c(-c3nc(-c4ccc(-c5cc6ccccc6cc5-c5ccccc5)cc4)nc(-c4cccc(-c5ccccc5)c4)n3)cccc21 ; CC1(C)c2ccccc2-c2c(-c3nc(-c4ccc(-c5cc6ccccc6cc5-c5ccccc5)cc4)nc(-c4cccc5ccccc45)n3)cccc21 ; CC1(C)c2ccccc2-c2c(-c3nc(-c4ccc(-c5cc6ccccc6cc5-c5ccccc5)cc4)nc(-c4ccc5ccccc5c4)n3)cccc21]

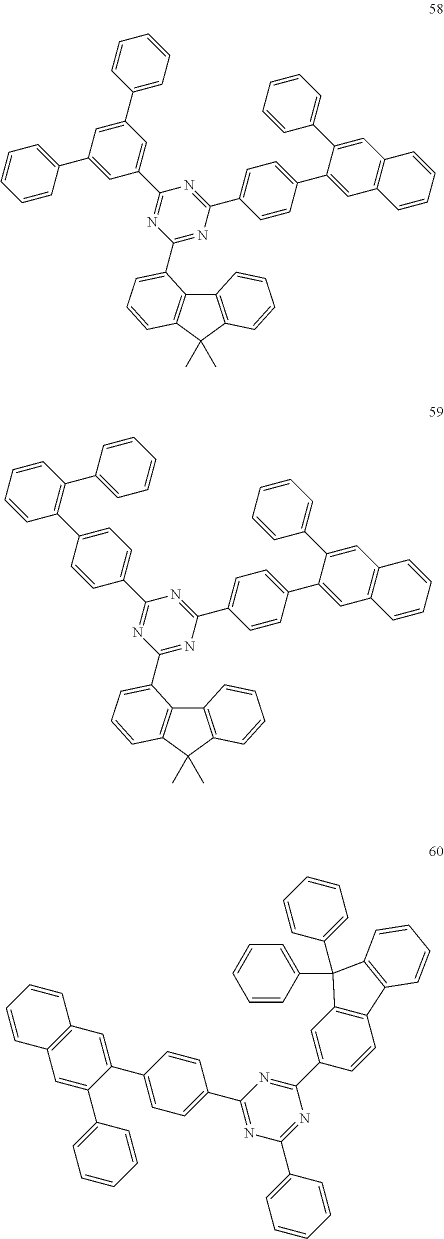 [SMILES: CC1(C)c2ccccc2-c2c(-c3nc(-c4ccc(-c5cc6ccccc6cc5-c5ccccc5)cc4)nc(-c4cc(-c5ccccc5)cc(-c5ccccc5)c4)n3)cccc21 ; CC1(C)c2ccccc2-c2c(-c3nc(-c4ccc(-c5ccccc5-c5ccccc5)cc4)nc(-c4ccc(-c5cc6ccccc6cc5-c5ccccc5)cc4)n3)cccc21 ; c1ccc(-c2nc(-c3ccc(-c4cc5ccccc5cc4-c4ccccc4)cc3)nc(-c3ccc4c(c3)C(c3ccccc3)(c3ccccc3)c3ccccc3-4)n2)cc1]

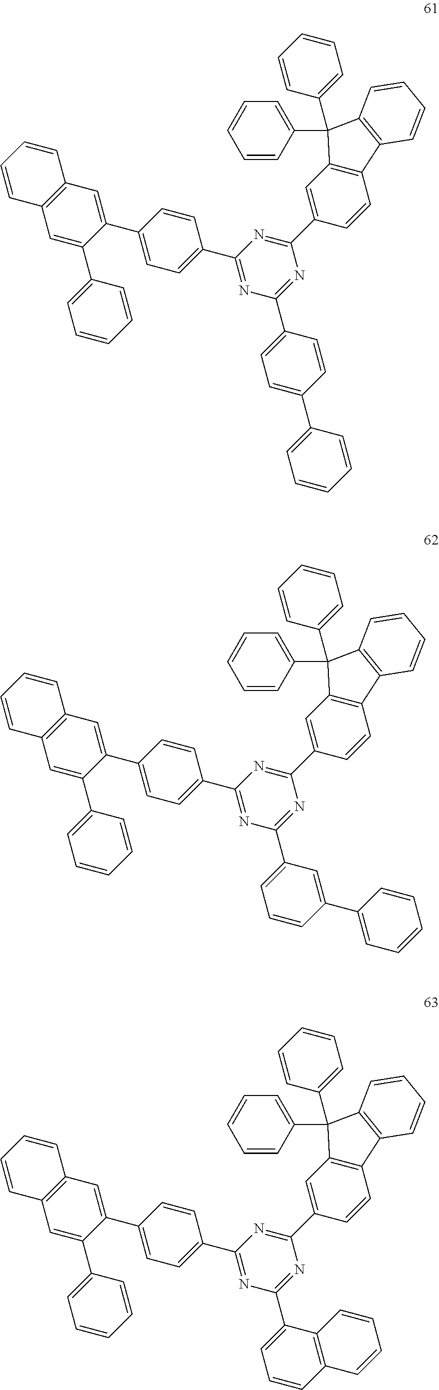 [SMILES: c1ccc(-c2ccc(-c3nc(-c4ccc(-c5cc6ccccc6cc5-c5ccccc5)cc4)nc(-c4ccc5c(c4)C(c4ccccc4)(c4ccccc4)c4ccccc4-5)n3)cc2)cc1 ; c1ccc(-c2cccc(-c3nc(-c4ccc(-c5cc6ccccc6cc5-c5ccccc5)cc4)nc(-c4ccc5c(c4)C(c4ccccc4)(c4ccccc4)c4ccccc4-5)n3)c2)cc1 ; c1ccc(-c2cc3ccccc3cc2-c2ccc(-c3nc(-c4ccc5c(c4)C(c4ccccc4)(c4ccccc4)c4ccccc4-5)nc(-c4cccc5ccccc45)n3)cc2)cc1]

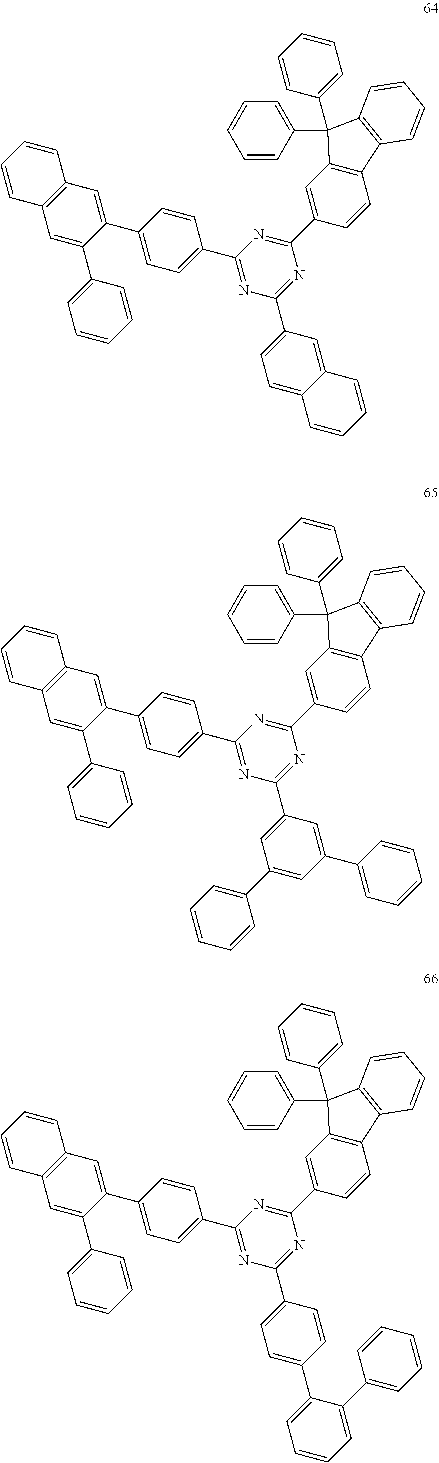 [SMILES: c1ccc(-c2cc3ccccc3cc2-c2ccc(-c3nc(-c4ccc5c(c4)C(c4ccccc4)(c4ccccc4)c4ccccc4-5)nc(-c4ccc5ccccc5c4)n3)cc2)cc1 ; c1ccc(-c2cc(-c3ccccc3)cc(-c3nc(-c4ccc(-c5cc6ccccc6cc5-c5ccccc5)cc4)nc(-c4ccc5c(c4)C(c4ccccc4)(c4ccccc4)c4ccccc4-5)n3)c2)cc1 ; c1ccc(-c2ccccc2-c2ccc(-c3nc(-c4ccc(-c5cc6ccccc6cc5-c5ccccc5)cc4)nc(-c4ccc5c(c4)C(c4ccccc4)(c4ccccc4)c4ccccc4-5)n3)cc2)cc1]

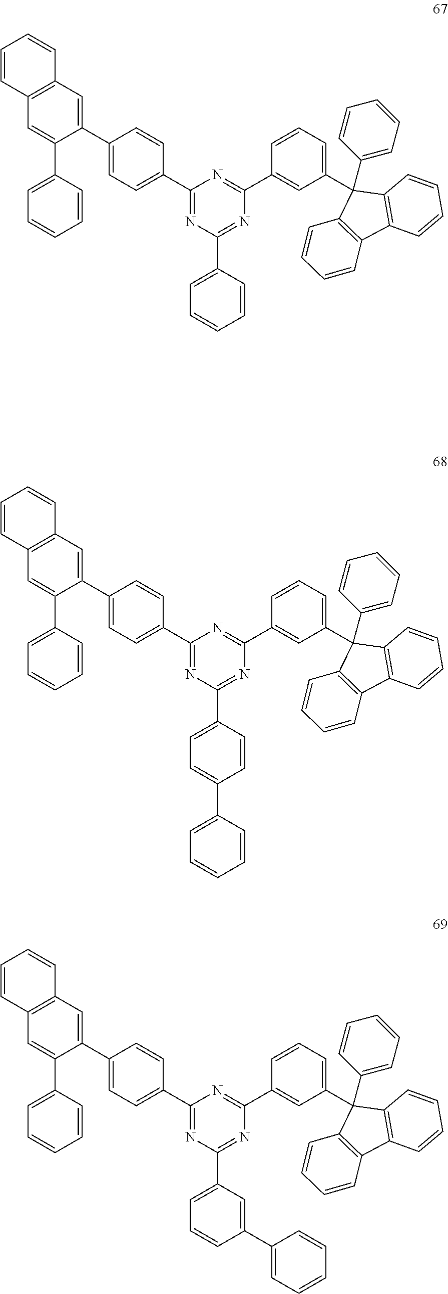 [SMILES: c1ccc(-c2nc(-c3ccc(-c4cc5ccccc5cc4-c4ccccc4)cc3)nc(-c3cccc(C4(c5ccccc5)c5ccccc5-c5ccccc54)c3)n2)cc1 ; c1ccc(-c2ccc(-c3nc(-c4ccc(-c5cc6ccccc6cc5-c5ccccc5)cc4)nc(-c4cccc(C5(c6ccccc6)c6ccccc6-c6ccccc65)c4)n3)cc2)cc1 ; c1ccc(-c2cccc(-c3nc(-c4ccc(-c5cc6ccccc6cc5-c5ccccc5)cc4)nc(-c4cccc(C5(c6ccccc6)c6ccccc6-c6ccccc65)c4)n3)c2)cc1]

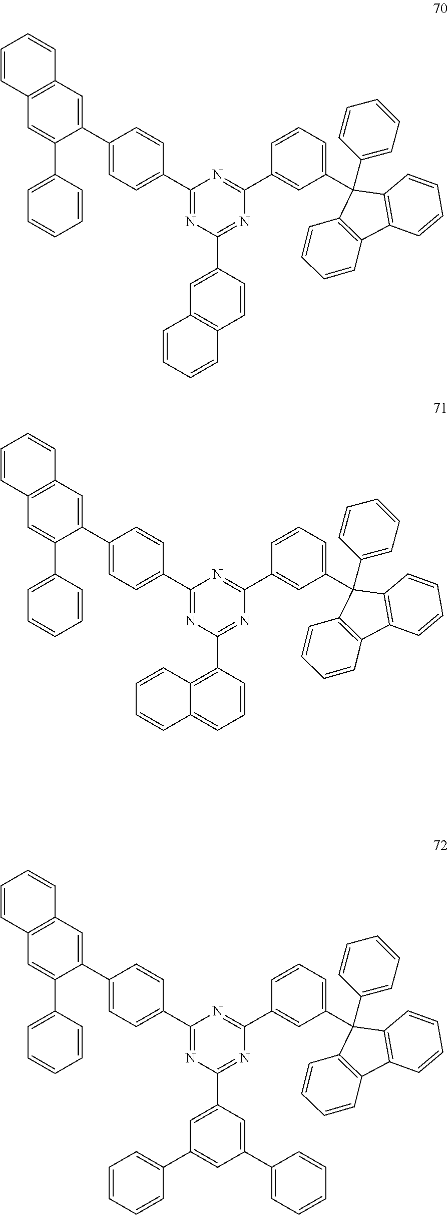 [SMILES: c1ccc(-c2cc3ccccc3cc2-c2ccc(-c3nc(-c4cccc(C5(c6ccccc6)c6ccccc6-c6ccccc65)c4)nc(-c4ccc5ccccc5c4)n3)cc2)cc1 ; c1ccc(-c2cc3ccccc3cc2-c2ccc(-c3nc(-c4cccc(C5(c6ccccc6)c6ccccc6-c6ccccc65)c4)nc(-c4cccc5ccccc45)n3)cc2)cc1 ; c1ccc(-c2cc(-c3ccccc3)cc(-c3nc(-c4ccc(-c5cc6ccccc6cc5-c5ccccc5)cc4)nc(-c4cccc(C5(c6ccccc6)c6ccccc6-c6ccccc65)c4)n3)c2)cc1]

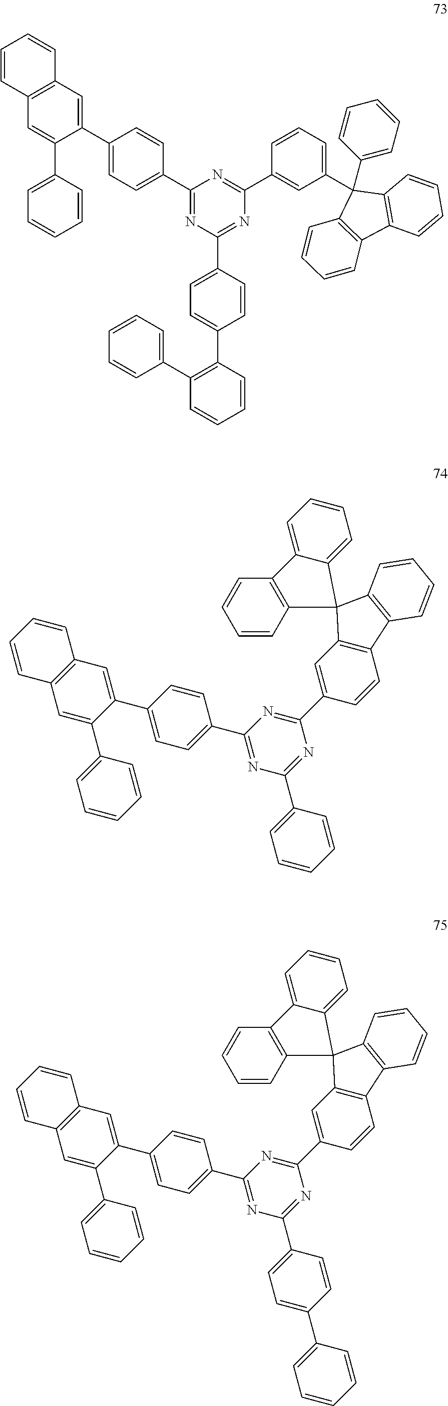 [SMILES: c1ccc(-c2ccccc2-c2ccc(-c3nc(-c4ccc(-c5cc6ccccc6cc5-c5ccccc5)cc4)nc(-c4cccc(C5(c6ccccc6)c6ccccc6-c6ccccc65)c4)n3)cc2)cc1 ; c1ccc(-c2nc(-c3ccc(-c4cc5ccccc5cc4-c4ccccc4)cc3)nc(-c3ccc4c(c3)C3(c5ccccc5-c5ccccc53)c3ccccc3-4)n2)cc1 ; c1ccc(-c2ccc(-c3nc(-c4ccc(-c5cc6ccccc6cc5-c5ccccc5)cc4)nc(-c4ccc5c(c4)C4(c6ccccc6-c6ccccc64)c4ccccc4-5)n3)cc2)cc1]

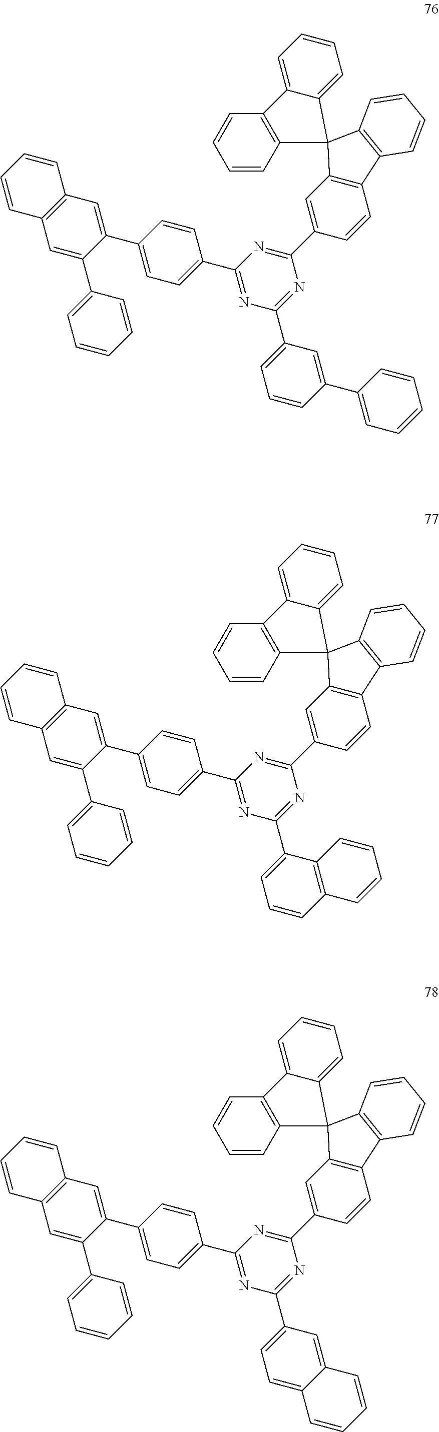 [SMILES: c1ccc(-c2cccc(-c3nc(-c4ccc(-c5cc6ccccc6cc5-c5ccccc5)cc4)nc(-c4ccc5c(c4)C4(c6ccccc6-c6ccccc64)c4ccccc4-5)n3)c2)cc1 ; c1ccc(-c2cc3ccccc3cc2-c2ccc(-c3nc(-c4ccc5c(c4)C4(c6ccccc6-c6ccccc64)c4ccccc4-5)nc(-c4cccc5ccccc45)n3)cc2)cc1 ; c1ccc(-c2cc3ccccc3cc2-c2ccc(-c3nc(-c4ccc5c(c4)C4(c6ccccc6-c6ccccc64)c4ccccc4-5)nc(-c4ccc5ccccc5c4)n3)cc2)cc1]

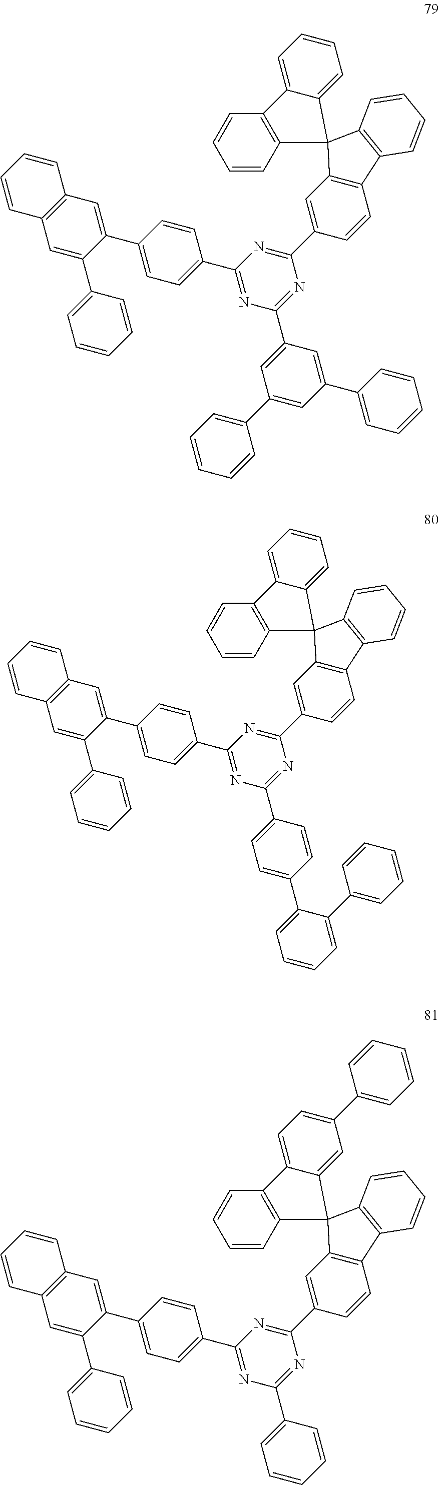 [SMILES: c1ccc(-c2cc(-c3ccccc3)cc(-c3nc(-c4ccc(-c5cc6ccccc6cc5-c5ccccc5)cc4)nc(-c4ccc5c(c4)C4(c6ccccc6-c6ccccc64)c4ccccc4-5)n3)c2)cc1 ; c1ccc(-c2ccccc2-c2ccc(-c3nc(-c4ccc(-c5cc6ccccc6cc5-c5ccccc5)cc4)nc(-c4ccc5c(c4)C4(c6ccccc6-c6ccccc64)c4ccccc4-5)n3)cc2)cc1 ; c1ccc(-c2ccc3c(c2)C2(c4ccccc4-3)c3ccccc3-c3ccc(-c4nc(-c5ccccc5)nc(-c5ccc(-c6cc7ccccc7cc6-c6ccccc6)cc5)n4)cc32)cc1]

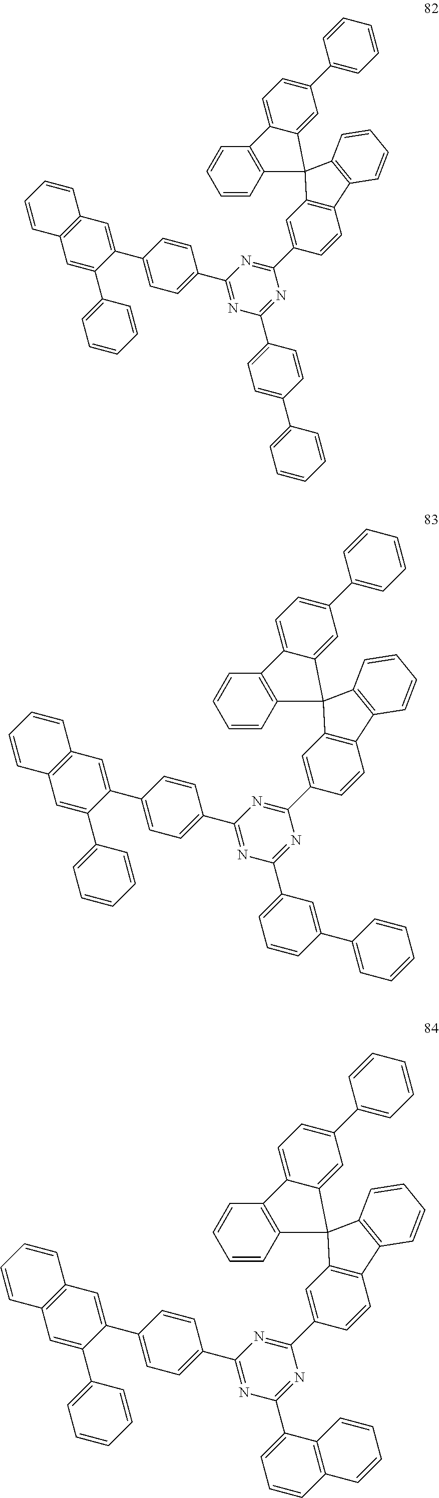 [SMILES: c1ccc(-c2ccc(-c3nc(-c4ccc(-c5cc6ccccc6cc5-c5ccccc5)cc4)nc(-c4ccc5c(c4)C4(c6ccccc6-c6ccc(-c7ccccc7)cc64)c4ccccc4-5)n3)cc2)cc1 ; c1ccc(-c2cccc(-c3nc(-c4ccc(-c5cc6ccccc6cc5-c5ccccc5)cc4)nc(-c4ccc5c(c4)C4(c6ccccc6-c6ccc(-c7ccccc7)cc64)c4ccccc4-5)n3)c2)cc1 ; c1ccc(-c2ccc3c(c2)C2(c4ccccc4-3)c3ccccc3-c3ccc(-c4nc(-c5ccc(-c6cc7ccccc7cc6-c6ccccc6)cc5)nc(-c5cccc6ccccc56)n4)cc32)cc1]

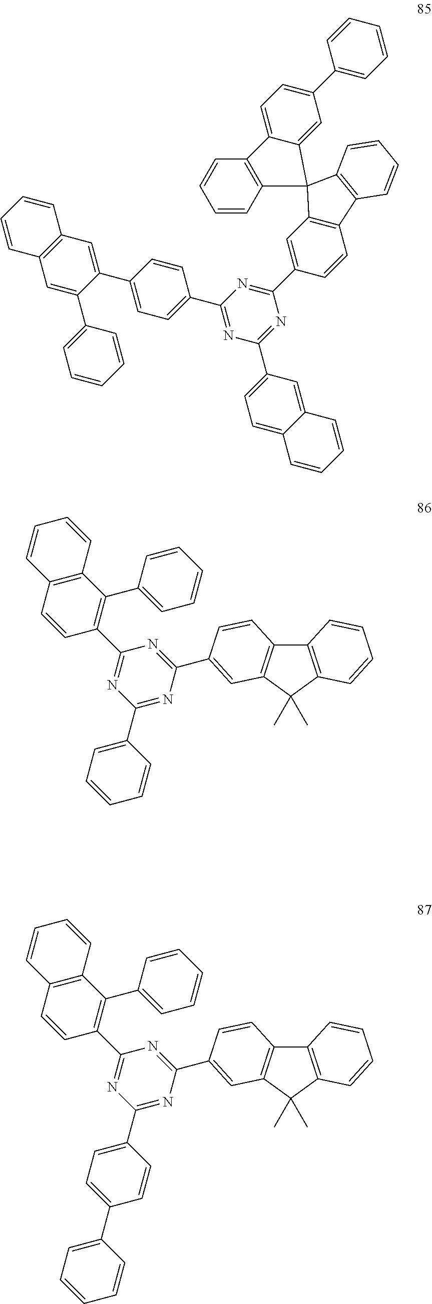 [SMILES: c1ccc(-c2ccc3c(c2)C2(c4ccccc4-3)c3ccccc3-c3ccc(-c4nc(-c5ccc(-c6cc7ccccc7cc6-c6ccccc6)cc5)nc(-c5ccc6ccccc6c5)n4)cc32)cc1 ; CC1(C)c2ccccc2-c2ccc(-c3nc(-c4ccccc4)nc(-c4ccc5ccccc5c4-c4ccccc4)n3)cc21 ; CC1(C)c2ccccc2-c2ccc(-c3nc(-c4ccc(-c5ccccc5)cc4)nc(-c4ccc5ccccc5c4-c4ccccc4)n3)cc21]

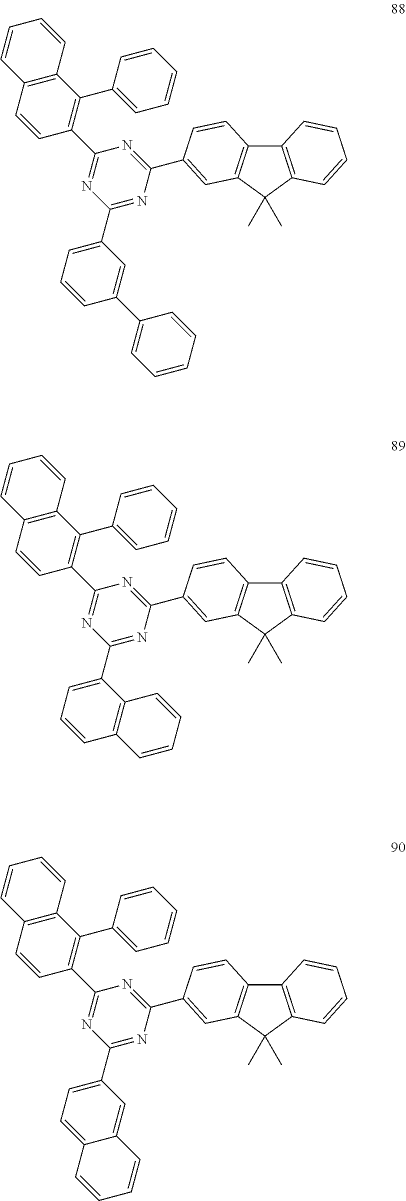 [SMILES: CC1(C)c2ccccc2-c2ccc(-c3nc(-c4cccc(-c5ccccc5)c4)nc(-c4ccc5ccccc5c4-c4ccccc4)n3)cc21 ; CC1(C)c2ccccc2-c2ccc(-c3nc(-c4ccc5ccccc5c4-c4ccccc4)nc(-c4cccc5ccccc45)n3)cc21 ; CC1(C)c2ccccc2-c2ccc(-c3nc(-c4ccc5ccccc5c4)nc(-c4ccc5ccccc5c4-c4ccccc4)n3)cc21]

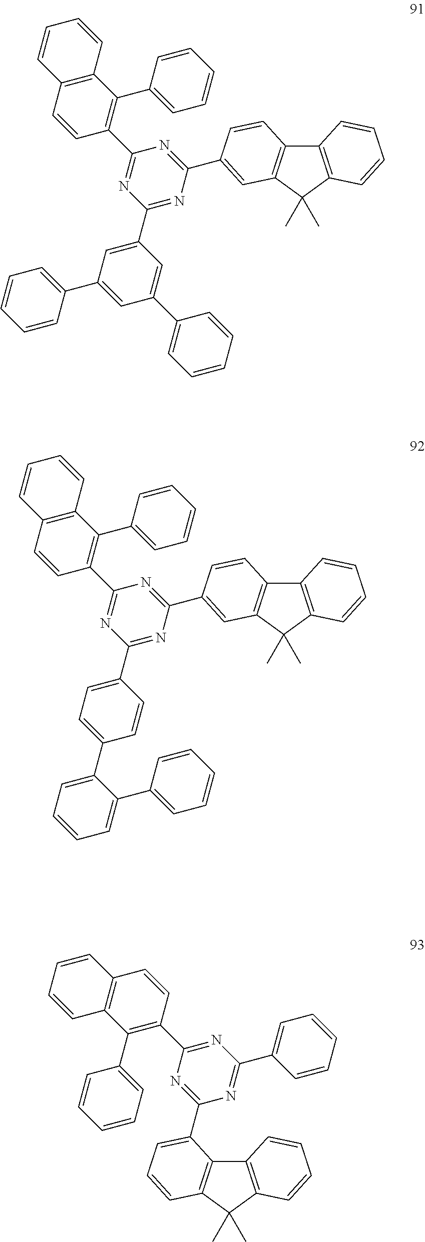 [SMILES: CC1(C)c2ccccc2-c2ccc(-c3nc(-c4cc(-c5ccccc5)cc(-c5ccccc5)c4)nc(-c4ccc5ccccc5c4-c4ccccc4)n3)cc21 ; CC1(C)c2ccccc2-c2ccc(-c3nc(-c4ccc(-c5ccccc5-c5ccccc5)cc4)nc(-c4ccc5ccccc5c4-c4ccccc4)n3)cc21 ; CC1(C)c2ccccc2-c2c(-c3nc(-c4ccccc4)nc(-c4ccc5ccccc5c4-c4ccccc4)n3)cccc21]

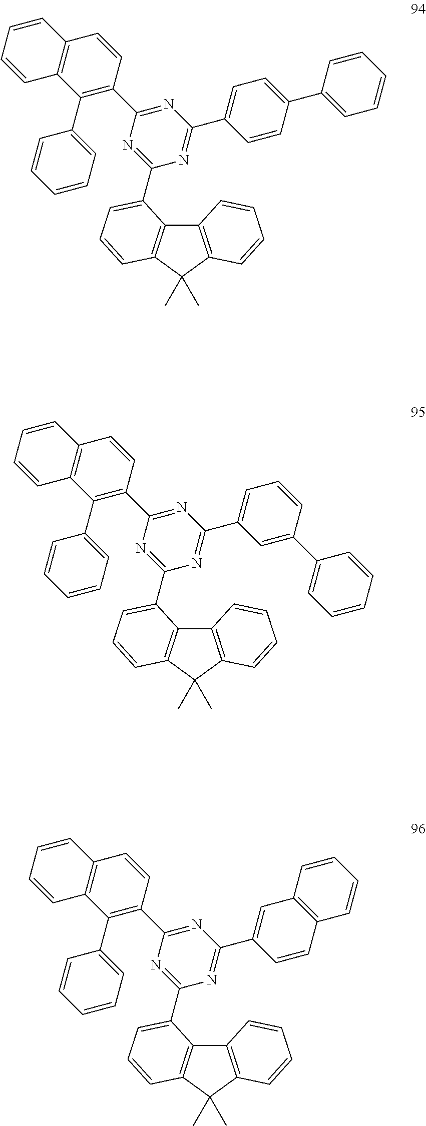 [SMILES: CC1(C)c2ccccc2-c2c(-c3nc(-c4ccc(-c5ccccc5)cc4)nc(-c4ccc5ccccc5c4-c4ccccc4)n3)cccc21 ; CC1(C)c2ccccc2-c2c(-c3nc(-c4cccc(-c5ccccc5)c4)nc(-c4ccc5ccccc5c4-c4ccccc4)n3)cccc21 ; CC1(C)c2ccccc2-c2c(-c3nc(-c4ccc5ccccc5c4)nc(-c4ccc5ccccc5c4-c4ccccc4)n3)cccc21]

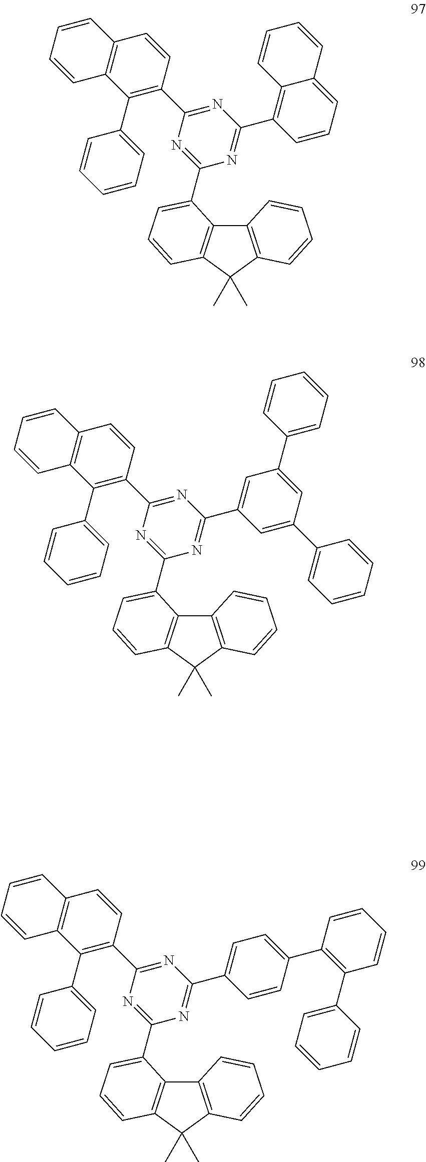 [SMILES: CC1(C)c2ccccc2-c2c(-c3nc(-c4ccc5ccccc5c4-c4ccccc4)nc(-c4cccc5ccccc45)n3)cccc21 ; CC1(C)c2ccccc2-c2c(-c3nc(-c4cc(-c5ccccc5)cc(-c5ccccc5)c4)nc(-c4ccc5ccccc5c4-c4ccccc4)n3)cccc21 ; CC1(C)c2ccccc2-c2c(-c3nc(-c4ccc(-c5ccccc5-c5ccccc5)cc4)nc(-c4ccc5ccccc5c4-c4ccccc4)n3)cccc21]

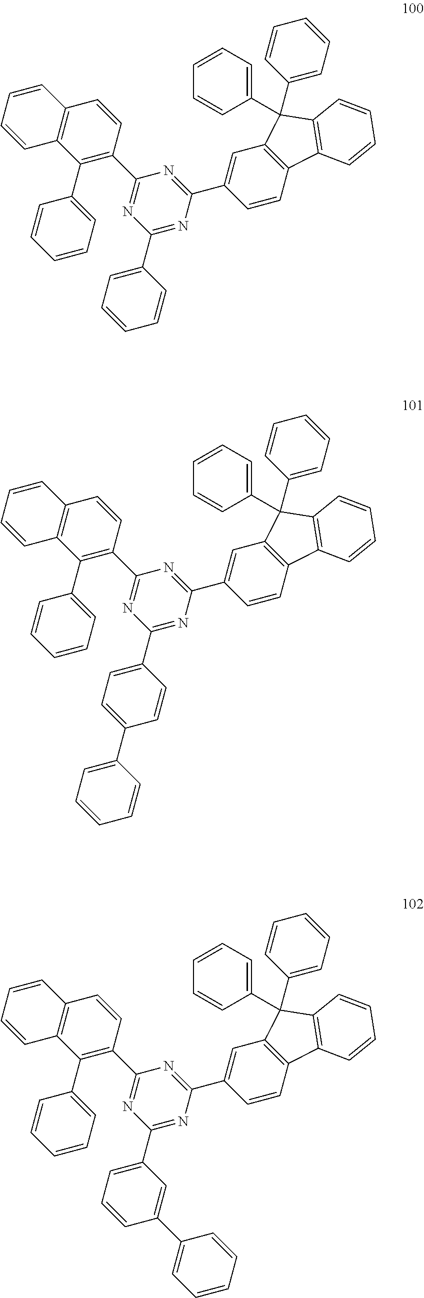 [SMILES: c1ccc(-c2nc(-c3ccc4c(c3)C(c3ccccc3)(c3ccccc3)c3ccccc3-4)nc(-c3ccc4ccccc4c3-c3ccccc3)n2)cc1 ; c1ccc(-c2ccc(-c3nc(-c4ccc5c(c4)C(c4ccccc4)(c4ccccc4)c4ccccc4-5)nc(-c4ccc5ccccc5c4-c4ccccc4)n3)cc2)cc1 ; c1ccc(-c2cccc(-c3nc(-c4ccc5c(c4)C(c4ccccc4)(c4ccccc4)c4ccccc4-5)nc(-c4ccc5ccccc5c4-c4ccccc4)n3)c2)cc1]

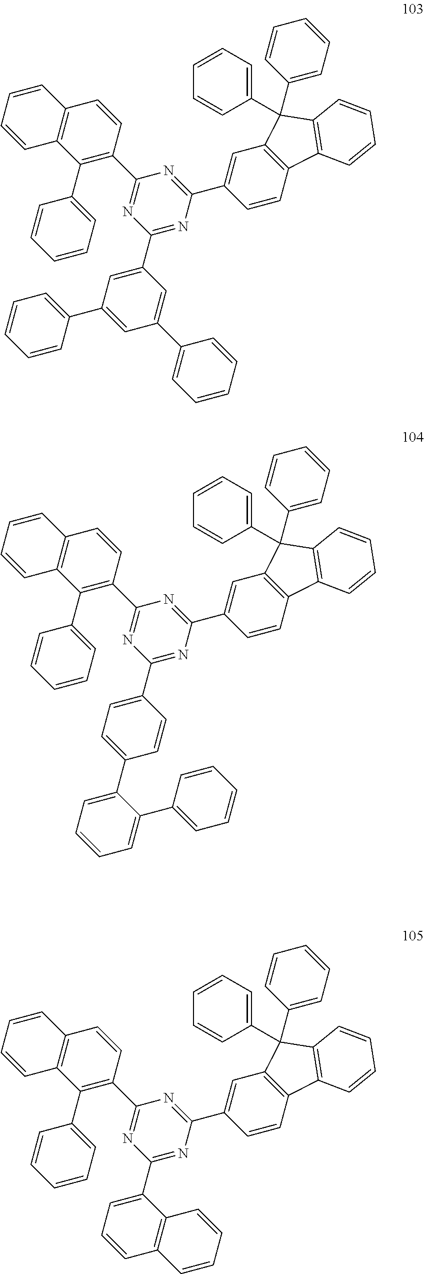 [SMILES: c1ccc(-c2cc(-c3ccccc3)cc(-c3nc(-c4ccc5c(c4)C(c4ccccc4)(c4ccccc4)c4ccccc4-5)nc(-c4ccc5ccccc5c4-c4ccccc4)n3)c2)cc1 ; c1ccc(-c2ccccc2-c2ccc(-c3nc(-c4ccc5c(c4)C(c4ccccc4)(c4ccccc4)c4ccccc4-5)nc(-c4ccc5ccccc5c4-c4ccccc4)n3)cc2)cc1 ; c1ccc(-c2c(-c3nc(-c4ccc5c(c4)C(c4ccccc4)(c4ccccc4)c4ccccc4-5)nc(-c4cccc5ccccc45)n3)ccc3ccccc23)cc1]

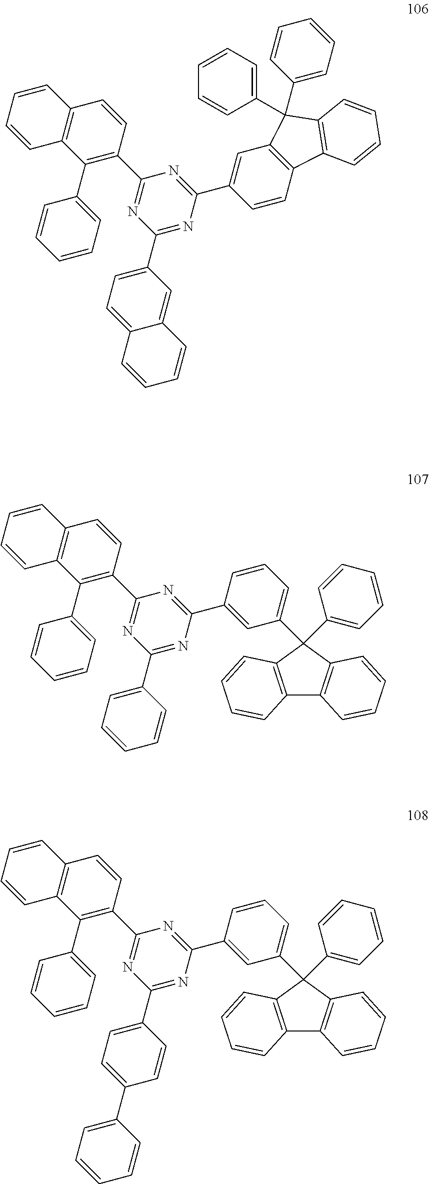 [SMILES: c1ccc(-c2c(-c3nc(-c4ccc5c(c4)C(c4ccccc4)(c4ccccc4)c4ccccc4-5)nc(-c4ccc5ccccc5c4)n3)ccc3ccccc23)cc1 ; c1ccc(-c2nc(-c3cccc(C4(c5ccccc5)c5ccccc5-c5ccccc54)c3)nc(-c3ccc4ccccc4c3-c3ccccc3)n2)cc1 ; c1ccc(-c2ccc(-c3nc(-c4cccc(C5(c6ccccc6)c6ccccc6-c6ccccc65)c4)nc(-c4ccc5ccccc5c4-c4ccccc4)n3)cc2)cc1]

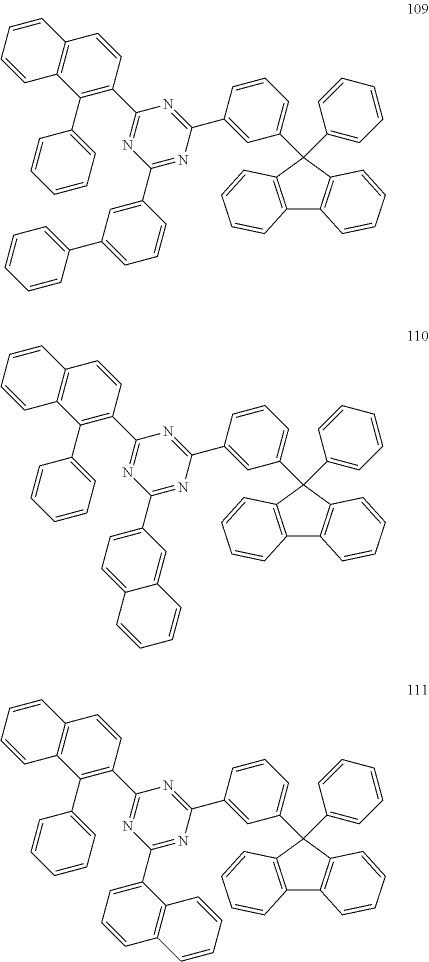 [SMILES: c1ccc(-c2cccc(-c3nc(-c4cccc(C5(c6ccccc6)c6ccccc6-c6ccccc65)c4)nc(-c4ccc5ccccc5c4-c4ccccc4)n3)c2)cc1 ; c1ccc(-c2c(-c3nc(-c4cccc(C5(c6ccccc6)c6ccccc6-c6ccccc65)c4)nc(-c4ccc5ccccc5c4)n3)ccc3ccccc23)cc1 ; c1ccc(-c2c(-c3nc(-c4cccc(C5(c6ccccc6)c6ccccc6-c6ccccc65)c4)nc(-c4cccc5ccccc45)n3)ccc3ccccc23)cc1]

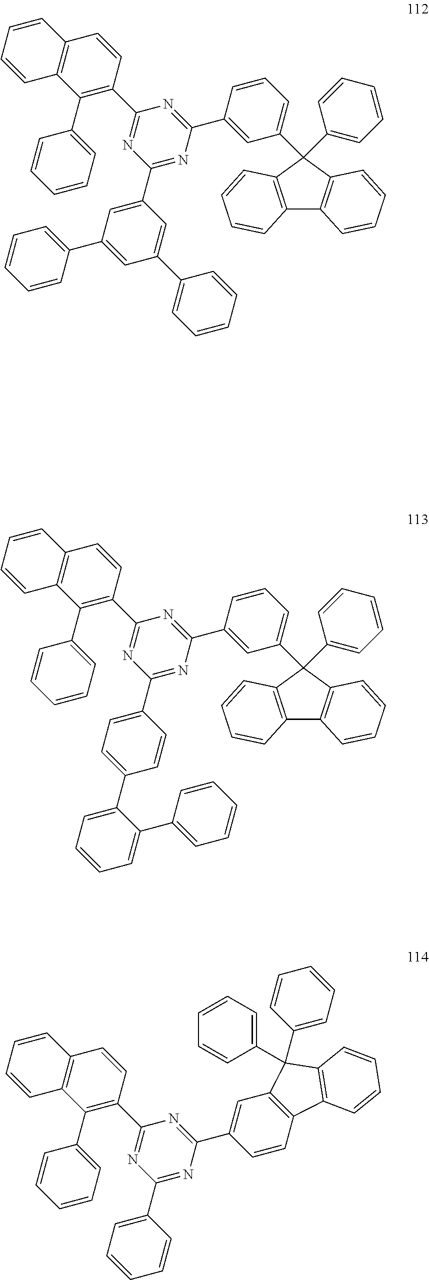 [SMILES: c1ccc(-c2cc(-c3ccccc3)cc(-c3nc(-c4cccc(C5(c6ccccc6)c6ccccc6-c6ccccc65)c4)nc(-c4ccc5ccccc5c4-c4ccccc4)n3)c2)cc1 ; c1ccc(-c2ccccc2-c2ccc(-c3nc(-c4cccc(C5(c6ccccc6)c6ccccc6-c6ccccc65)c4)nc(-c4ccc5ccccc5c4-c4ccccc4)n3)cc2)cc1 ; c1ccc(-c2nc(-c3ccc4c(c3)C(c3ccccc3)(c3ccccc3)c3ccccc3-4)nc(-c3ccc4ccccc4c3-c3ccccc3)n2)cc1]

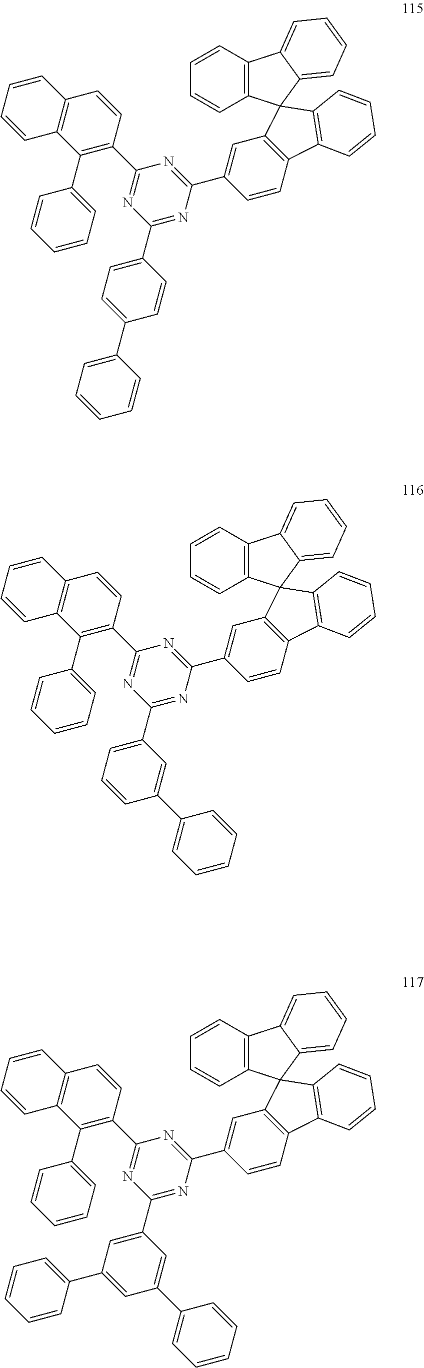 [SMILES: c1ccc(-c2ccc(-c3nc(-c4ccc5c(c4)C4(c6ccccc6-c6ccccc64)c4ccccc4-5)nc(-c4ccc5ccccc5c4-c4ccccc4)n3)cc2)cc1 ; c1ccc(-c2cccc(-c3nc(-c4ccc5c(c4)C4(c6ccccc6-c6ccccc64)c4ccccc4-5)nc(-c4ccc5ccccc5c4-c4ccccc4)n3)c2)cc1 ; c1ccc(-c2cc(-c3ccccc3)cc(-c3nc(-c4ccc5c(c4)C4(c6ccccc6-c6ccccc64)c4ccccc4-5)nc(-c4ccc5ccccc5c4-c4ccccc4)n3)c2)cc1]

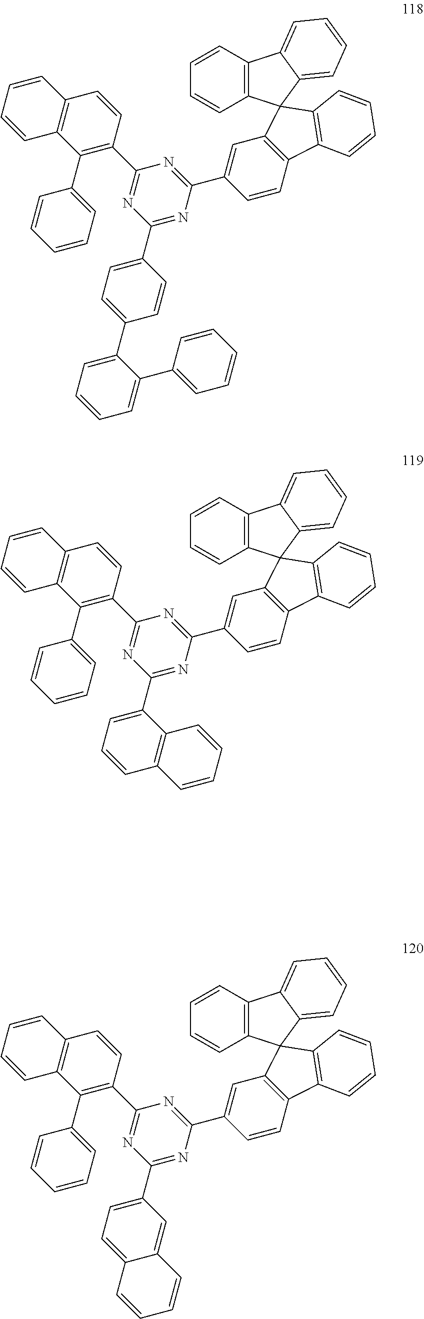 [SMILES: c1ccc(-c2ccccc2-c2ccc(-c3nc(-c4ccc5c(c4)C4(c6ccccc6-c6ccccc64)c4ccccc4-5)nc(-c4ccc5ccccc5c4-c4ccccc4)n3)cc2)cc1 ; c1ccc(-c2c(-c3nc(-c4ccc5c(c4)C4(c6ccccc6-c6ccccc64)c4ccccc4-5)nc(-c4cccc5ccccc45)n3)ccc3ccccc23)cc1 ; c1ccc(-c2c(-c3nc(-c4ccc5c(c4)C4(c6ccccc6-c6ccccc64)c4ccccc4-5)nc(-c4ccc5ccccc5c4)n3)ccc3ccccc23)cc1]

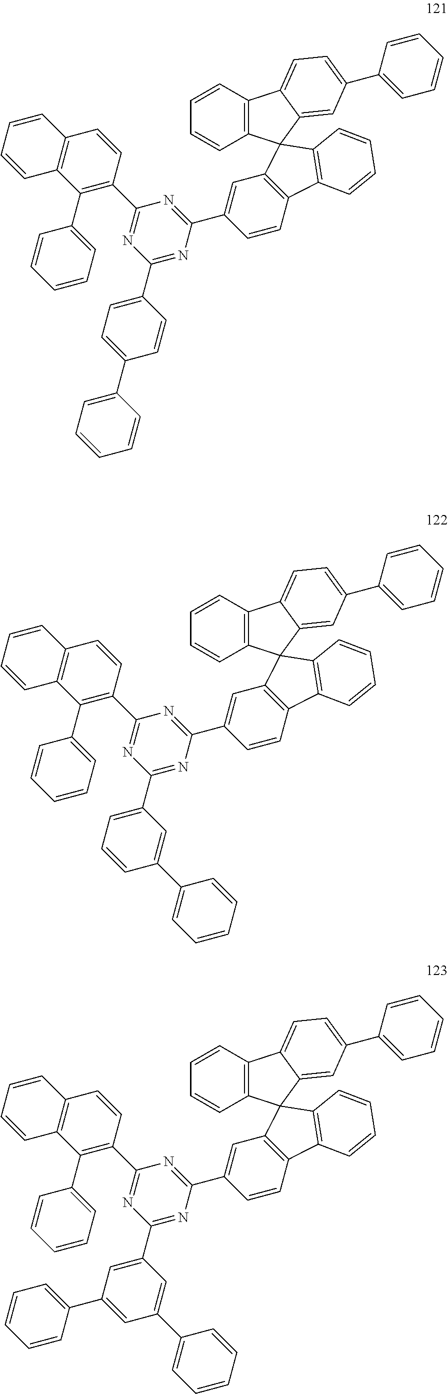 [SMILES: c1ccc(-c2ccc(-c3nc(-c4ccc5c(c4)C4(c6ccccc6-c6ccc(-c7ccccc7)cc64)c4ccccc4-5)nc(-c4ccc5ccccc5c4-c4ccccc4)n3)cc2)cc1 ; c1ccc(-c2cccc(-c3nc(-c4ccc5c(c4)C4(c6ccccc6-c6ccc(-c7ccccc7)cc64)c4ccccc4-5)nc(-c4ccc5ccccc5c4-c4ccccc4)n3)c2)cc1 ; c1ccc(-c2cc(-c3ccccc3)cc(-c3nc(-c4ccc5c(c4)C4(c6ccccc6-c6ccc(-c7ccccc7)cc64)c4ccccc4-5)nc(-c4ccc5ccccc5c4-c4ccccc4)n3)c2)cc1]

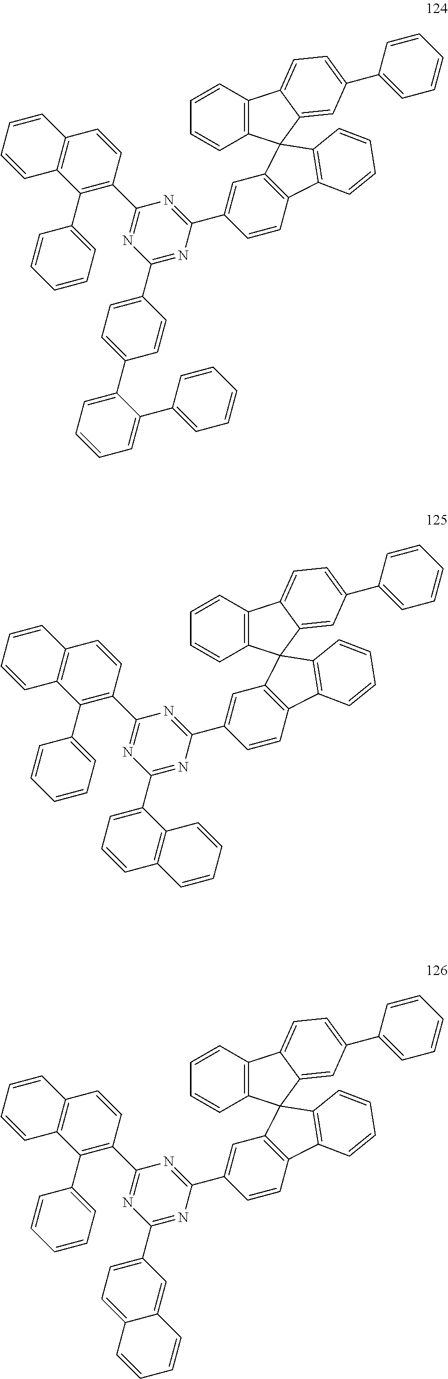 [SMILES: c1ccc(-c2ccc3c(c2)C2(c4ccccc4-3)c3ccccc3-c3ccc(-c4nc(-c5ccc(-c6ccccc6-c6ccccc6)cc5)nc(-c5ccc6ccccc6c5-c5ccccc5)n4)cc32)cc1 ; c1ccc(-c2ccc3c(c2)C2(c4ccccc4-3)c3ccccc3-c3ccc(-c4nc(-c5ccc6ccccc6c5-c5ccccc5)nc(-c5cccc6ccccc56)n4)cc32)cc1 ; c1ccc(-c2ccc3c(c2)C2(c4ccccc4-3)c3ccccc3-c3ccc(-c4nc(-c5ccc6ccccc6c5)nc(-c5ccc6ccccc6c5-c5ccccc5)n4)cc32)cc1]

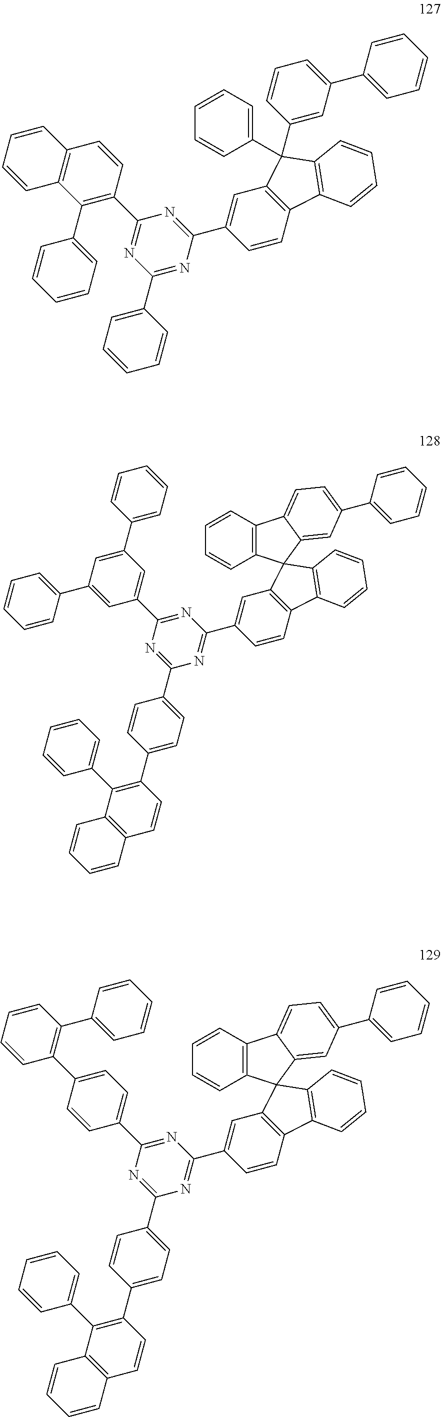 [SMILES: c1ccc(-c2cccc(C3(c4ccccc4)c4ccccc4-c4ccc(-c5nc(-c6ccccc6)nc(-c6ccc7ccccc7c6-c6ccccc6)n5)cc43)c2)cc1 ; c1ccc(-c2cc(-c3ccccc3)cc(-c3nc(-c4ccc(-c5ccc6ccccc6c5-c5ccccc5)cc4)nc(-c4ccc5c(c4)C4(c6ccccc6-c6ccc(-c7ccccc7)cc64)c4ccccc4-5)n3)c2)cc1 ; c1ccc(-c2ccc3c(c2)C2(c4ccccc4-3)c3ccccc3-c3ccc(-c4nc(-c5ccc(-c6ccccc6-c6ccccc6)cc5)nc(-c5ccc(-c6ccc7ccccc7c6-c6ccccc6)cc5)n4)cc32)cc1]

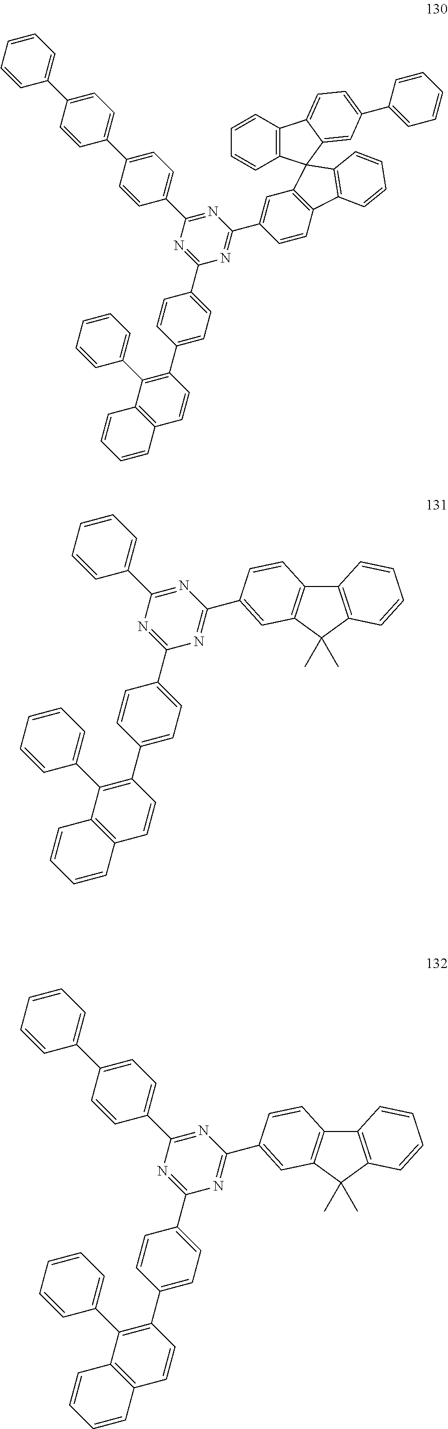 [SMILES: c1ccc(-c2ccc(-c3ccc(-c4nc(-c5ccc(-c6ccc7ccccc7c6-c6ccccc6)cc5)nc(-c5ccc6c(c5)C5(c7ccccc7-c7ccc(-c8ccccc8)cc75)c5ccccc5-6)n4)cc3)cc2)cc1 ; CC1(C)c2ccccc2-c2ccc(-c3nc(-c4ccccc4)nc(-c4ccc(-c5ccc6ccccc6c5-c5ccccc5)cc4)n3)cc21 ; CC1(C)c2ccccc2-c2ccc(-c3nc(-c4ccc(-c5ccccc5)cc4)nc(-c4ccc(-c5ccc6ccccc6c5-c5ccccc5)cc4)n3)cc21]

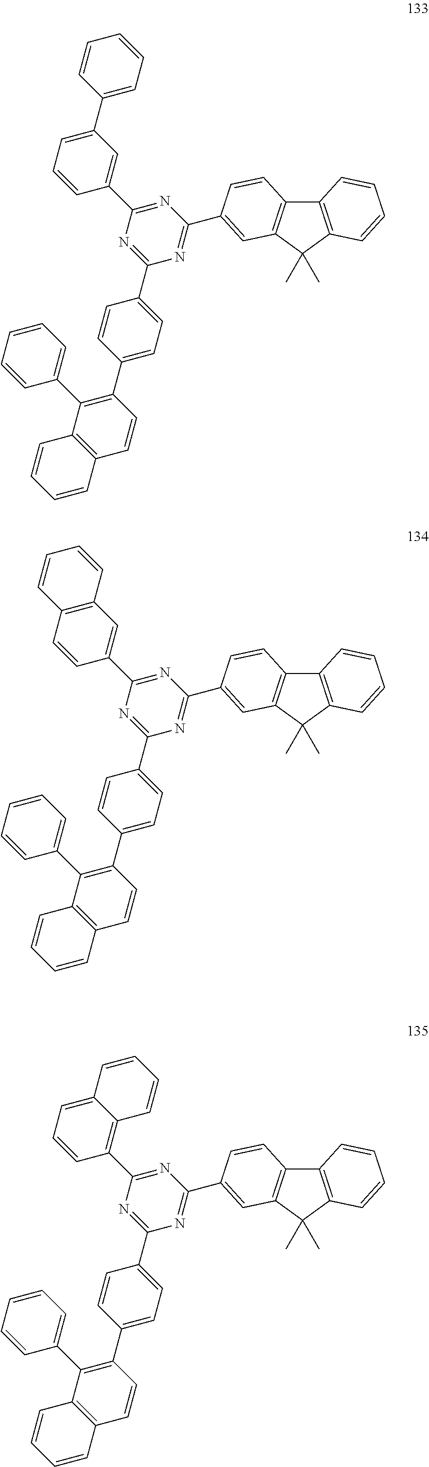 [SMILES: CC1(C)c2ccccc2-c2ccc(-c3nc(-c4ccc(-c5ccc6ccccc6c5-c5ccccc5)cc4)nc(-c4cccc(-c5ccccc5)c4)n3)cc21 ; CC1(C)c2ccccc2-c2ccc(-c3nc(-c4ccc(-c5ccc6ccccc6c5-c5ccccc5)cc4)nc(-c4ccc5ccccc5c4)n3)cc21 ; CC1(C)c2ccccc2-c2ccc(-c3nc(-c4ccc(-c5ccc6ccccc6c5-c5ccccc5)cc4)nc(-c4cccc5ccccc45)n3)cc21]

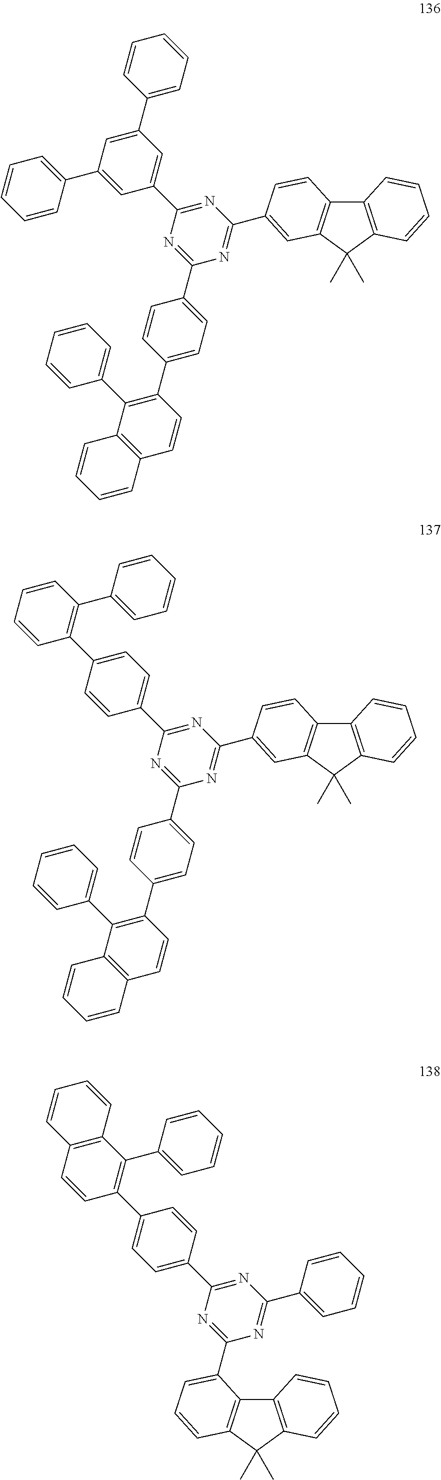 [SMILES: CC1(C)c2ccccc2-c2ccc(-c3nc(-c4ccc(-c5ccc6ccccc6c5-c5ccccc5)cc4)nc(-c4cc(-c5ccccc5)cc(-c5ccccc5)c4)n3)cc21 ; CC1(C)c2ccccc2-c2ccc(-c3nc(-c4ccc(-c5ccccc5-c5ccccc5)cc4)nc(-c4ccc(-c5ccc6ccccc6c5-c5ccccc5)cc4)n3)cc21 ; CC1(C)c2ccccc2-c2c(-c3nc(-c4ccccc4)nc(-c4ccc(-c5ccc6ccccc6c5-c5ccccc5)cc4)n3)cccc21]

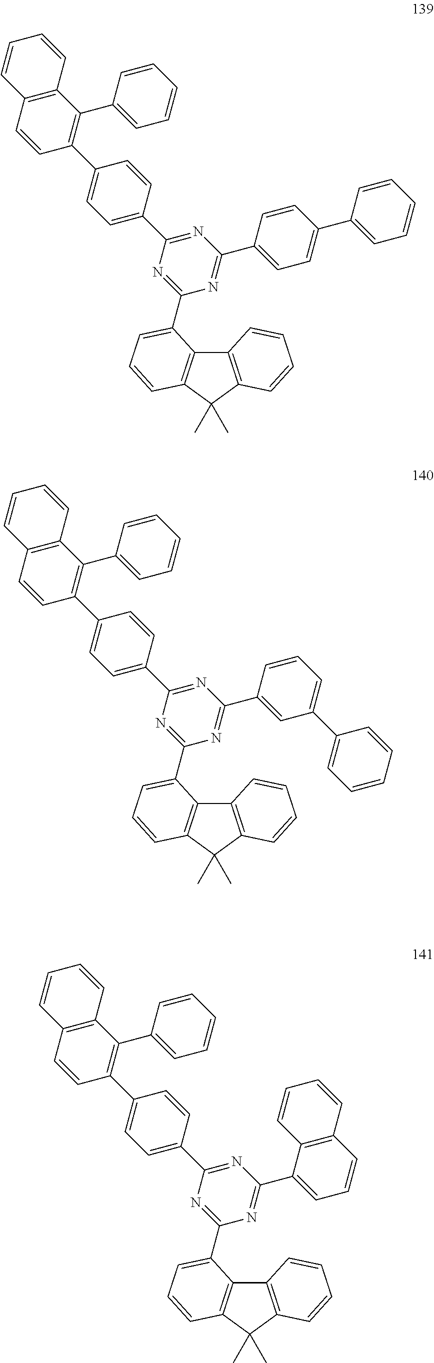 [SMILES: CC1(C)c2ccccc2-c2c(-c3nc(-c4ccc(-c5ccccc5)cc4)nc(-c4ccc(-c5ccc6ccccc6c5-c5ccccc5)cc4)n3)cccc21 ; CC1(C)c2ccccc2-c2c(-c3nc(-c4ccc(-c5ccc6ccccc6c5-c5ccccc5)cc4)nc(-c4cccc(-c5ccccc5)c4)n3)cccc21 ; CC1(C)c2ccccc2-c2c(-c3nc(-c4ccc(-c5ccc6ccccc6c5-c5ccccc5)cc4)nc(-c4cccc5ccccc45)n3)cccc21]

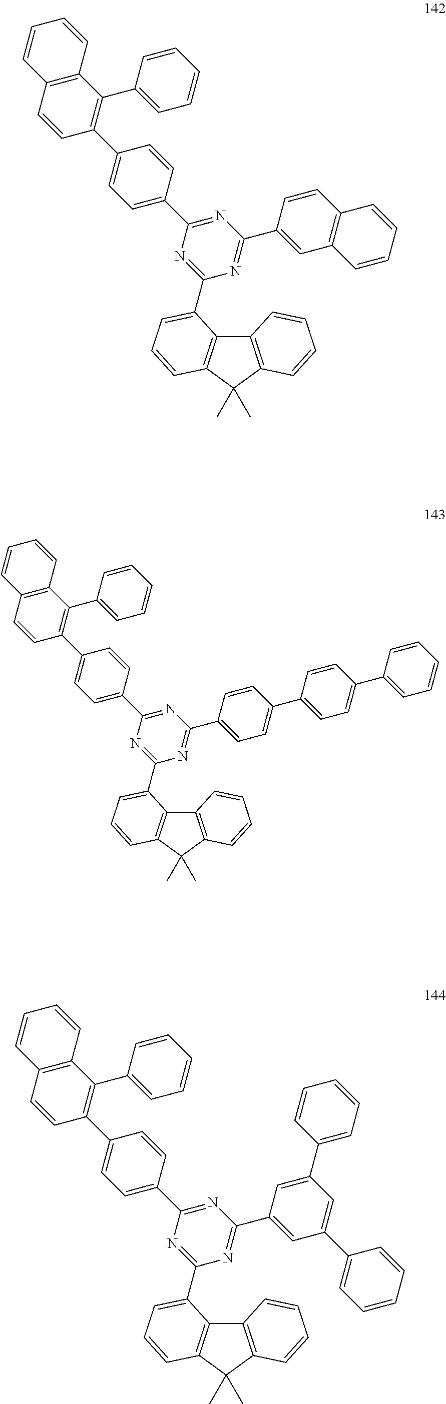 [SMILES: CC1(C)c2ccccc2-c2c(-c3nc(-c4ccc(-c5ccc6ccccc6c5-c5ccccc5)cc4)nc(-c4ccc5ccccc5c4)n3)cccc21 ; CC1(C)c2ccccc2-c2c(-c3nc(-c4ccc(-c5ccc(-c6ccccc6)cc5)cc4)nc(-c4ccc(-c5ccc6ccccc6c5-c5ccccc5)cc4)n3)cccc21 ; CC1(C)c2ccccc2-c2c(-c3nc(-c4ccc(-c5ccc6ccccc6c5-c5ccccc5)cc4)nc(-c4cc(-c5ccccc5)cc(-c5ccccc5)c4)n3)cccc21]

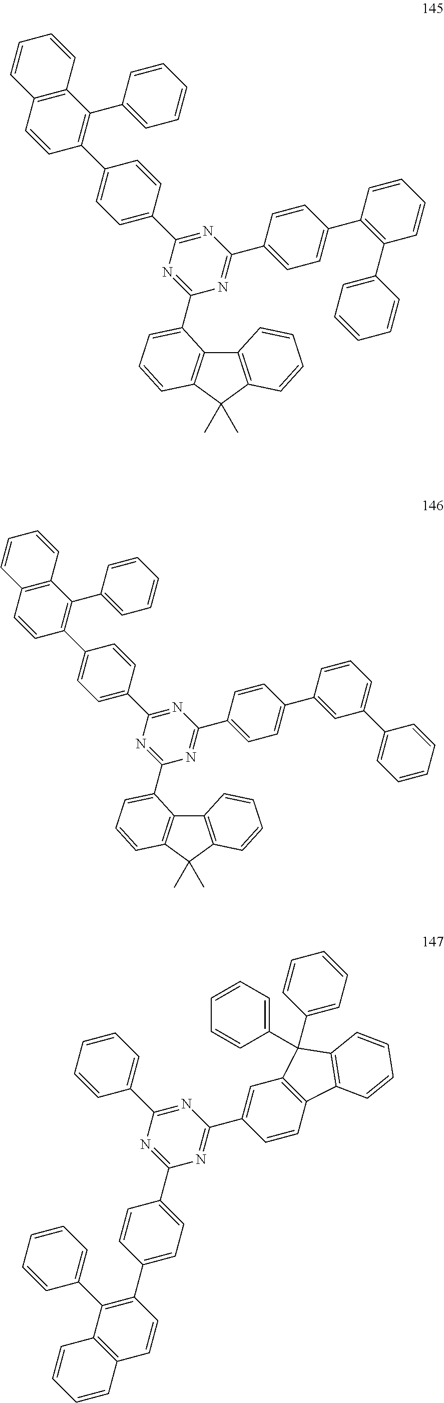 [SMILES: CC1(C)c2ccccc2-c2c(-c3nc(-c4ccc(-c5ccccc5-c5ccccc5)cc4)nc(-c4ccc(-c5ccc6ccccc6c5-c5ccccc5)cc4)n3)cccc21 ; CC1(C)c2ccccc2-c2c(-c3nc(-c4ccc(-c5cccc(-c6ccccc6)c5)cc4)nc(-c4ccc(-c5ccc6ccccc6c5-c5ccccc5)cc4)n3)cccc21 ; c1ccc(-c2nc(-c3ccc(-c4ccc5ccccc5c4-c4ccccc4)cc3)nc(-c3ccc4c(c3)C(c3ccccc3)(c3ccccc3)c3ccccc3-4)n2)cc1]

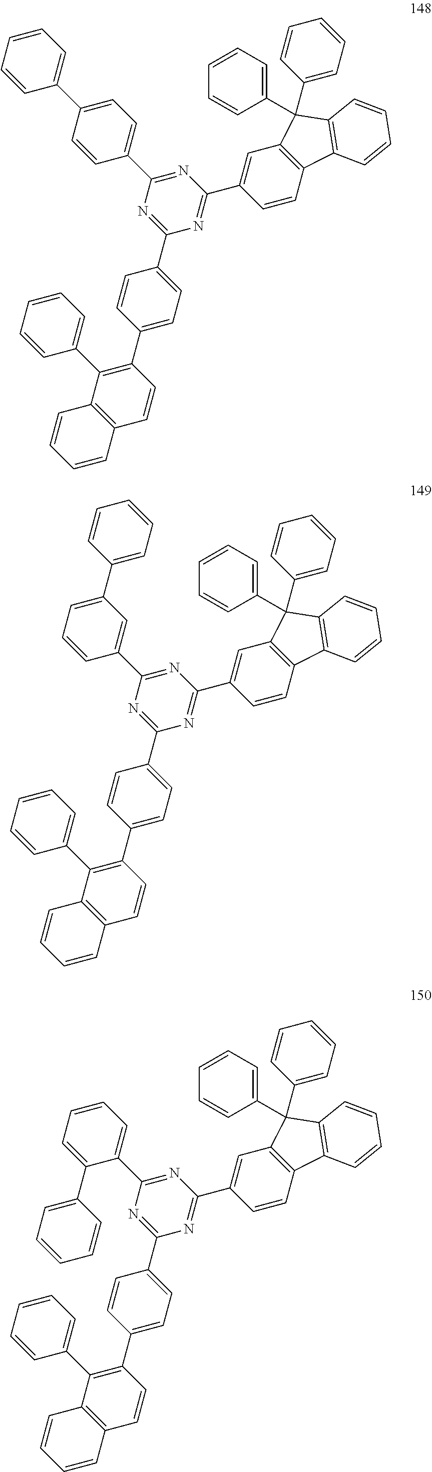

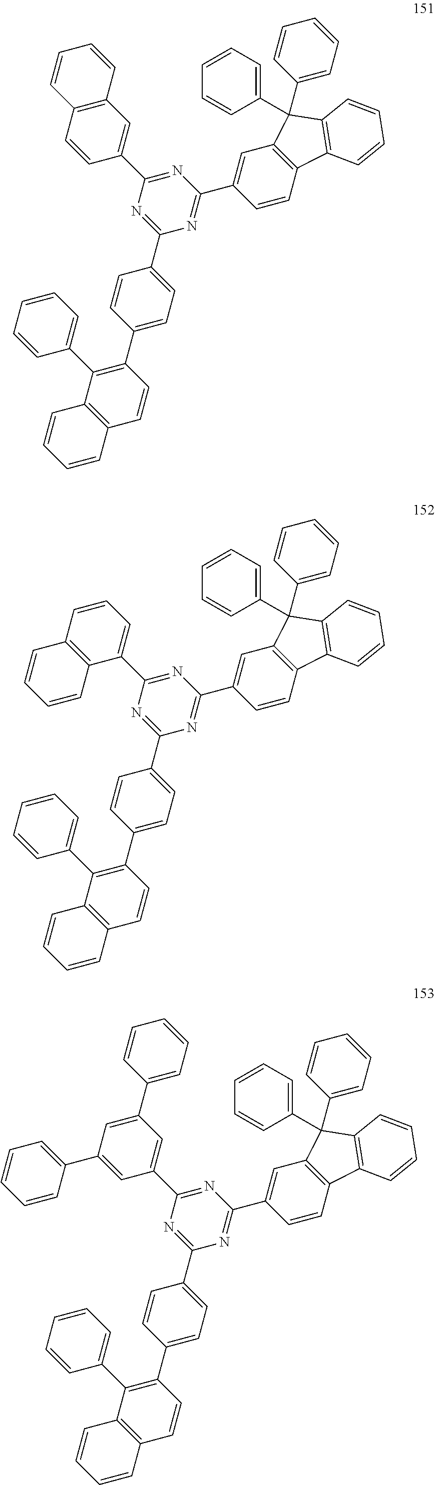 [SMILES: c1ccc(-c2c(-c3ccc(-c4nc(-c5ccc6c(c5)C(c5ccccc5)(c5ccccc5)c5ccccc5-6)nc(-c5ccc6ccccc6c5)n4)cc3)ccc3ccccc23)cc1 ; c1ccc(-c2c(-c3ccc(-c4nc(-c5ccc6c(c5)C(c5ccccc5)(c5ccccc5)c5ccccc5-6)nc(-c5cccc6ccccc56)n4)cc3)ccc3ccccc23)cc1 ; c1ccc(-c2cc(-c3ccccc3)cc(-c3nc(-c4ccc(-c5ccc6ccccc6c5-c5ccccc5)cc4)nc(-c4ccc5c(c4)C(c4ccccc4)(c4ccccc4)c4ccccc4-5)n3)c2)cc1]

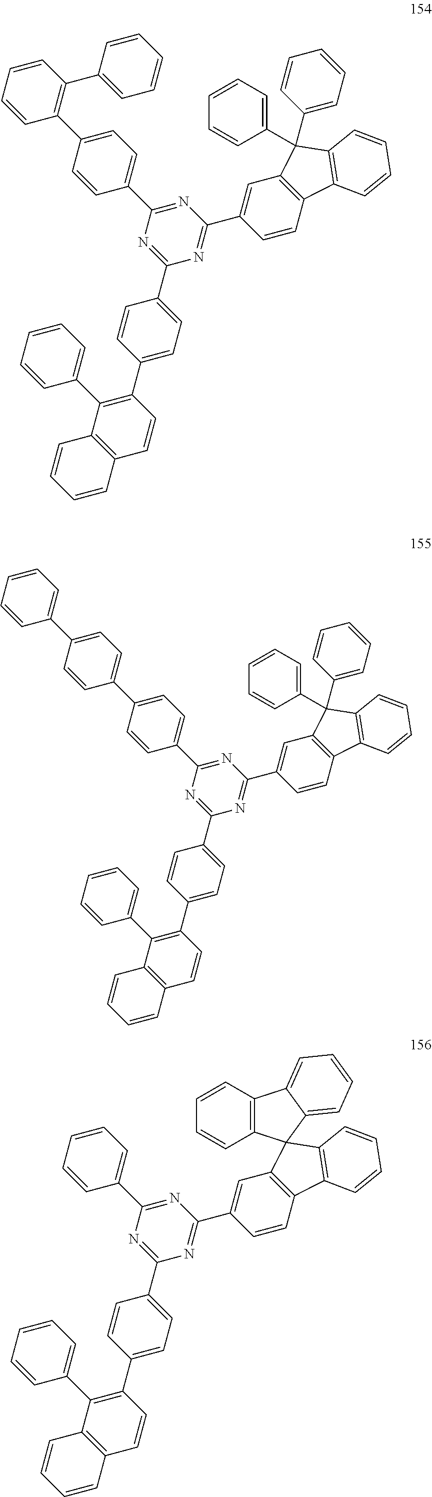 [SMILES: c1ccc(-c2ccccc2-c2ccc(-c3nc(-c4ccc(-c5ccc6ccccc6c5-c5ccccc5)cc4)nc(-c4ccc5c(c4)C(c4ccccc4)(c4ccccc4)c4ccccc4-5)n3)cc2)cc1 ; c1ccc(-c2ccc(-c3ccc(-c4nc(-c5ccc(-c6ccc7ccccc7c6-c6ccccc6)cc5)nc(-c5ccc6c(c5)C(c5ccccc5)(c5ccccc5)c5ccccc5-6)n4)cc3)cc2)cc1 ; c1ccc(-c2nc(-c3ccc(-c4ccc5ccccc5c4-c4ccccc4)cc3)nc(-c3ccc4c(c3)C3(c5ccccc5-c5ccccc53)c3ccccc3-4)n2)cc1]

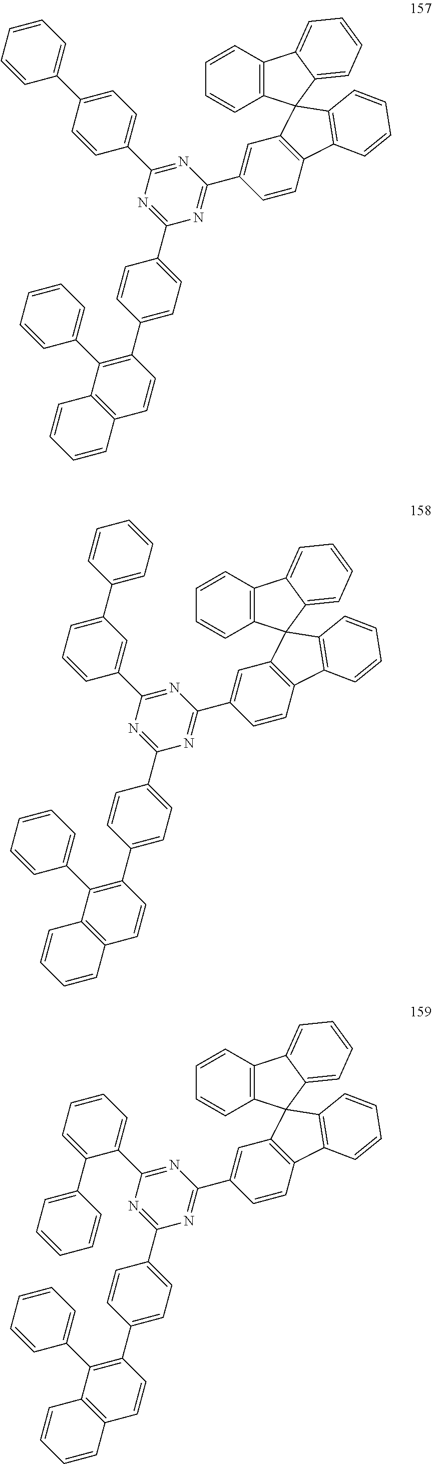 [SMILES: c1ccc(-c2ccc(-c3nc(-c4ccc(-c5ccc6ccccc6c5-c5ccccc5)cc4)nc(-c4ccc5c(c4)C4(c6ccccc6-c6ccccc64)c4ccccc4-5)n3)cc2)cc1 ; c1ccc(-c2cccc(-c3nc(-c4ccc(-c5ccc6ccccc6c5-c5ccccc5)cc4)nc(-c4ccc5c(c4)C4(c6ccccc6-c6ccccc64)c4ccccc4-5)n3)c2)cc1 ; c1ccc(-c2ccccc2-c2nc(-c3ccc(-c4ccc5ccccc5c4-c4ccccc4)cc3)nc(-c3ccc4c(c3)C3(c5ccccc5-c5ccccc53)c3ccccc3-4)n2)cc1]

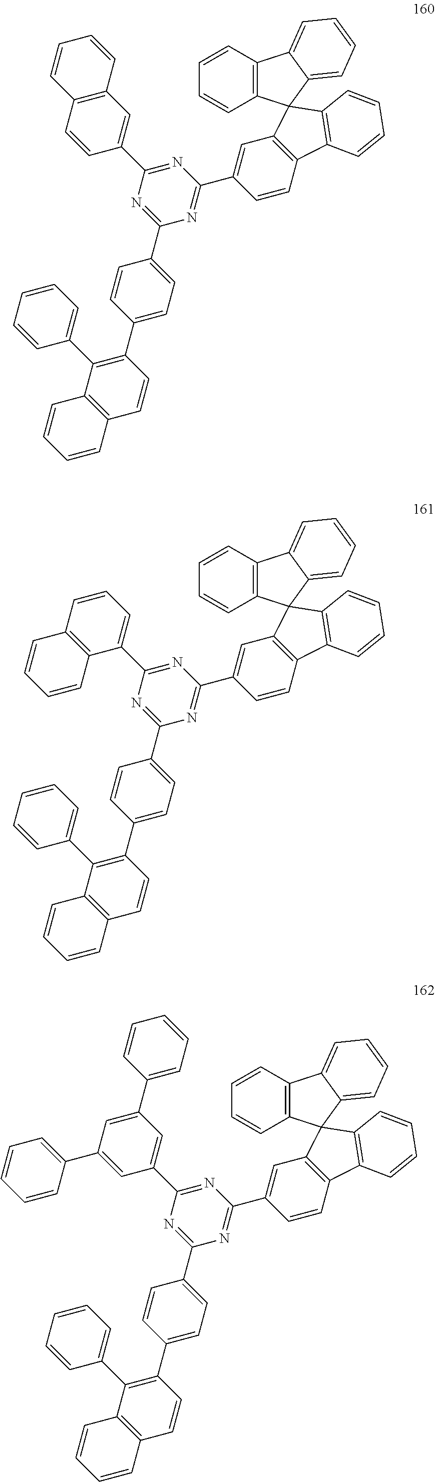 [SMILES: c1ccc(-c2c(-c3ccc(-c4nc(-c5ccc6c(c5)C5(c7ccccc7-c7ccccc75)c5ccccc5-6)nc(-c5ccc6ccccc6c5)n4)cc3)ccc3ccccc23)cc1 ; c1ccc(-c2c(-c3ccc(-c4nc(-c5ccc6c(c5)C5(c7ccccc7-c7ccccc75)c5ccccc5-6)nc(-c5cccc6ccccc56)n4)cc3)ccc3ccccc23)cc1 ; c1ccc(-c2cc(-c3ccccc3)cc(-c3nc(-c4ccc(-c5ccc6ccccc6c5-c5ccccc5)cc4)nc(-c4ccc5c(c4)C4(c6ccccc6-c6ccccc64)c4ccccc4-5)n3)c2)cc1]

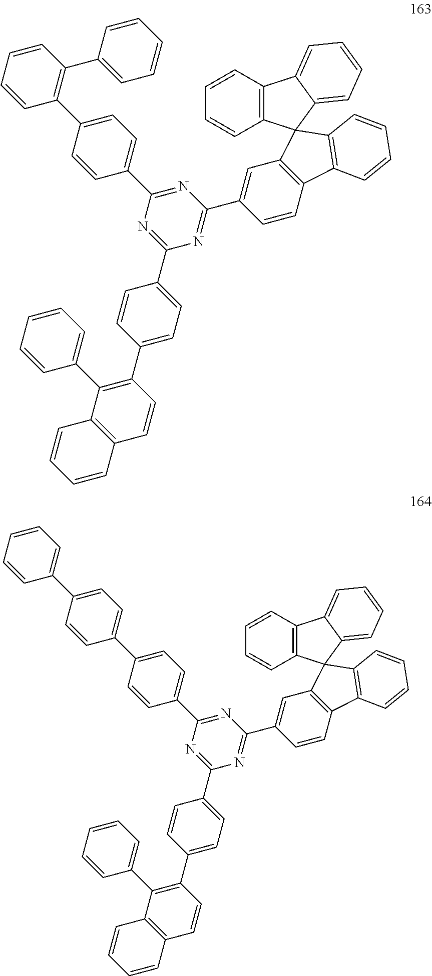 [SMILES: c1ccc(-c2ccccc2-c2ccc(-c3nc(-c4ccc(-c5ccc6ccccc6c5-c5ccccc5)cc4)nc(-c4ccc5c(c4)C4(c6ccccc6-c6ccccc64)c4ccccc4-5)n3)cc2)cc1 ; c1ccc(-c2ccc(-c3ccc(-c4nc(-c5ccc(-c6ccc7ccccc7c6-c6ccccc6)cc5)nc(-c5ccc6c(c5)C5(c7ccccc7-c7ccccc75)c5ccccc5-6)n4)cc3)cc2)cc1]

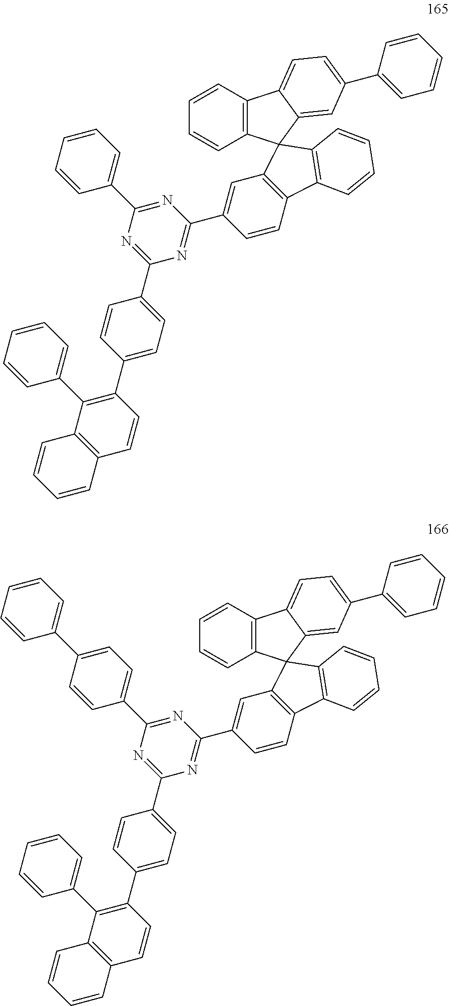 [SMILES: c1ccc(-c2ccc3c(c2)C2(c4ccccc4-3)c3ccccc3-c3ccc(-c4nc(-c5ccccc5)nc(-c5ccc(-c6ccc7ccccc7c6-c6ccccc6)cc5)n4)cc32)cc1 ; c1ccc(-c2ccc(-c3nc(-c4ccc(-c5ccc6ccccc6c5-c5ccccc5)cc4)nc(-c4ccc5c(c4)C4(c6ccccc6-c6ccc(-c7ccccc7)cc64)c4ccccc4-5)n3)cc2)cc1]

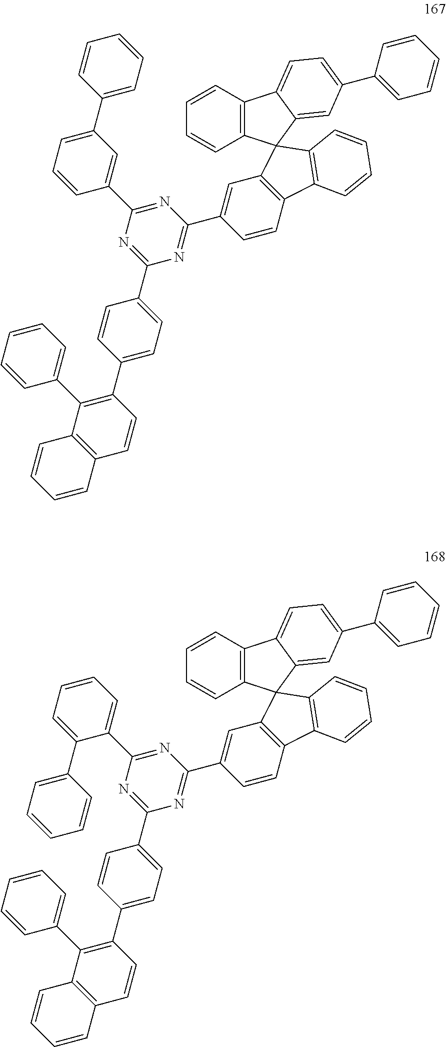 [SMILES: c1ccc(-c2cccc(-c3nc(-c4ccc(-c5ccc6ccccc6c5-c5ccccc5)cc4)nc(-c4ccc5c(c4)C4(c6ccccc6-c6ccc(-c7ccccc7)cc64)c4ccccc4-5)n3)c2)cc1 ; c1ccc(-c2ccc3c(c2)C2(c4ccccc4-3)c3ccccc3-c3ccc(-c4nc(-c5ccc(-c6ccc7ccccc7c6-c6ccccc6)cc5)nc(-c5ccccc5-c5ccccc5)n4)cc32)cc1]

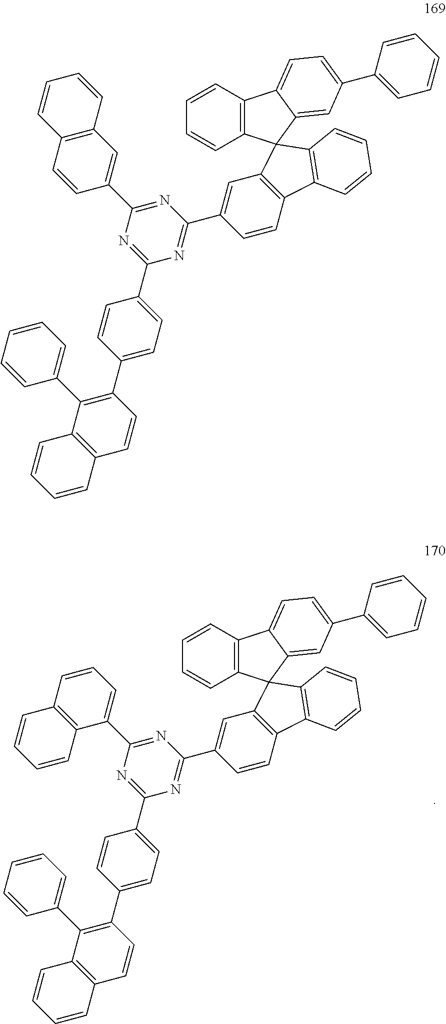 [SMILES: c1ccc(-c2ccc3c(c2)C2(c4ccccc4-3)c3ccccc3-c3ccc(-c4nc(-c5ccc(-c6ccc7ccccc7c6-c6ccccc6)cc5)nc(-c5ccc6ccccc6c5)n4)cc32)cc1 ; c1ccc(-c2ccc3c(c2)C2(c4ccccc4-3)c3ccccc3-c3ccc(-c4nc(-c5ccc(-c6ccc7ccccc7c6-c6ccccc6)cc5)nc(-c5cccc6ccccc56)n4)cc32)cc1]

20. A display device comprising:
a circuit layer disposed on a base layer; and
a display element layer disposed on the circuit layer and comprising a light emitting element, wherein
the light emitting element comprises:
a first electrode;
a second electrode disposed on the first electrode; and
an electron transport region disposed between the first electrode and the second electrode, and
the electron transport region comprises a nitrogen-containing compound represented by Formula 1: 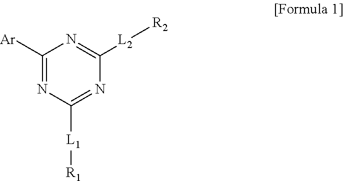
wherein in Formula 1,
Ar is a substituted or unsubstituted aryl group having 6 to 30 ring-forming carbon atoms or a substituted or unsubstituted heteroaryl group having 2 to 30 ring-forming carbon atoms,
L₁ and L₂ are each independently a direct linkage or a substituted or unsubstituted aryl group having 6 to 30 ring-forming carbon atoms,
R₁ is a group represented by Formula 2, and
R₂ is a group represented by Formula 3-1, Formula 3-2, or Formula 3-3: 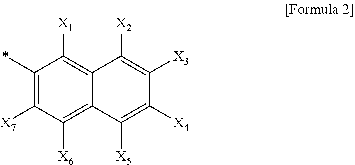
wherein in Formula 2,
X₁ to X₇ are each independently a hydrogen atom, a deuterium atom, a halogen atom, a cyano group, a hydroxy group, a substituted or unsubstituted oxy group, a substituted or unsubstituted thio group, a substituted or unsubstituted boron group, a substituted or unsubstituted silyl group, a substituted or unsubstituted amine group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms,
except that X₁ or X₇ is a substituted or unsubstituted phenyl group, and 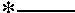 [SMILES: *C]
is a position linked to L₁ in Formula 1: 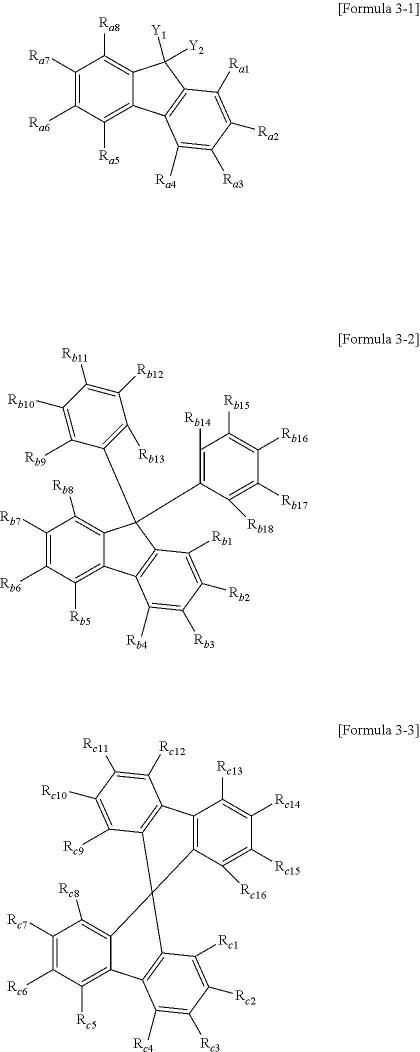
wherein in Formula 3-1,
Y₁ and Y₂ are each independently a hydrogen atom, a deuterium atom, a halogen atom, a cyano group, a hydroxy group, or a substituted or unsubstituted alkyl group having 1 to 20 carbon atoms, and
wherein in Formula 3-1 to Formula 3-3,
Rₐ₁ to Rₐ₈, R_{b1} to R_{c18}, and R_{b1} to R_{c16} are each independently a hydrogen atom, a deuterium atom, a halogen atom, a cyano group, a hydroxy group, a substituted or unsubstituted oxy group, a substituted or unsubstituted thio group, a substituted or unsubstituted boron group, a substituted or unsubstituted silyl group, a substituted or unsubstituted amine group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms,
except that
when R₂ in Formula 1 is a group represented by Formula 3-1, one of Rₐ₁ to Rₐ₈ in Formula 3-1 is a position linked to L₂ in Formula 1,
when R₂ in Formula 1 is a group represented by Formula 3-2, one of R_{b1} to R_{b18} in Formula 3-2 is a position linked to L₂ in Formula 1, and
when R₂ in Formula 1 is a group represented by Formula 3-3, one of R_{c1} to R_{c16} in Formula 3-3 is a position linked to L₂ in Formula 1.
